SEQUENCE LISTING (63 sequences)

SEQ ID NO: 1 - protein, 845 aa, Homo sapiens
MDYYFGPPPPQKPDAERQTDEEKEELKKPKKEDSSPKEETDKWAVEKGKDHKEPRKGEELEEEWTPTEKVKCPPIGMESHRIEDNQIRASSMLRHGLGAQRGRLNMQTGATEDDYYDGAWCAEDDARTQWIEVDTRRTTRFTGVITQGRDSSIHDDFVTTFFVGFSNDSQTWVMYTNGYEEMTFHGNVDKDTPVLSELPEPVVARFIRIYPLTWNGSLCMRLEVLGCSVAPVYSYYAQNEVVATDDLDFRHHSYKDMRQLMKVVNEECPTITRTYSLGKSSRGLKIYAMEISDNPGEHELGEPEFRYTAGIHGNEVLGRELLLLLMQYLCREYRDGNPRVRSLVQDTRIHLVPSLNPDGYEVAAQMGSEFGNWALGLWTEEGFDIFEDFPDLNSVLWGAEERKWVPYRVPNNNLPIPERYLSPDATVSTEVRAIIAWMEKNPFVLGANLNGGERLVSYPYDMARTPTQEQLLAAAMAAARGEDEDEVSEAQETPDHAIFRWLAISFASAHLTLTEPYRGGCQAQDYTGGMGIVNGAKWNPRTGTINDFSYLHTNCLELSFYLGCDKFPHESELPREWENNKEALLTFMEQVHRGIKGVVTDEQGIPIANATISVSGINHGVKTASGGDYWRILNPGEYRVTAHAEGYTPSAKTCNVDYDIGATQCNFILARSNWKRIREIMAMNGNRPIPHIDPSRPMTPQQRRLQQRRLQHRLRLRAQMRLRRLNATTTLGPHTVPPTLPPAPATTLSTTIEPWGLIPPTTAGWEESETETYTEVVTEFGTEVEPEFGTKVEPEFETQLEPEFETQLEPEFEEEEEEEKEEEIATGQAFPFTTVETYTVNFGDF

SEQ ID NO: 2 - protein, 518 aa, Homo sapiens
MGALARALLLPLLAQWLLRAAPELAPAPFTLPLRVAAATNRVVAPTPGPGTPAERHADGLALALEPALASPAGAANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILWIVSYALMSVCGAILLVLIVLLLLPFRCQRRPRDPEVVNDESSLVRHRWK

SEQ ID NO: 3 - protein, 180 aa, Homo sapiens
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 4 - protein, 329 aa, Homo sapiens
MTHFNKGPSYGLSAEVKNKIASKYDHQAEEDLRNWIEEVTGMSIGPNFQLGLKDGIILCELINKLQPGSVKKVNESSLNWPQLENIGNFIKAIQAYGMKPHDIFEANDLFENGNMTQVQTTLVALAGLAKTKGFHTTIDIGVKYAEKQTRRFDEGKLKAGQSVIGLQMGTNKCASQAGMTAYGTRRHLYDPKMQTDKPFDQTTISLQMGTNKGASQAGMLAPGTRRDIYDQKLTLQPVDNSTISLQMGTNKVASQKGMSVYGLGRQVYDPKYCAAPTEPVIHNGSQGTGTNGSEISDSDYQAEYPDEYHGEYQDDYPRDYQYSDQGIDY

SEQ ID NO: 5 - protein, 1669 aa, Homo sapiens
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMPGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 6 - protein, 142 aa, Homo sapiens
MPRCRWLSLILLTIPLALVARKDPKKNETGVLRKLKPVNASNANVKQCLWFAMQEYNKESEDKYVFLVVKTLQAQLQVTNLLEYLIDVEIARSDCRKPLSTNEICAIQENSKLKRKLSCSFLVGALPWNGEFTVMEKKCEDA

SEQ ID NO: 7 - protein, 333 aa, Homo sapiens [sequence omitted]

SEQ ID NO: 8 - protein, 327 aa, Homo sapiens
MRALPGLLEARARTPRLLLLQCLLAAARPSSADGSAPDSPFTSPPLREEIMANNFSLESHNISLTEHSSMPVEKNITLERPSNVNLTCQFTTSGDLNAVNVTWKKDGEQLENNYLVSATGSTLYTQYRFTIINSKQMGSYSCFFREEKEQRGTFNFKVPELHGKNKPLISYVGDSTVLTCKCQNCFPLNWTWYSSNGSVKVPVGVQMNKYVINGTYANETKLKITQLLEEDGESYWCRALFQLGESEEHIELVVLSYLVPLKPFLVIVAEVILLVATILLCEKYTQKKKKHSDEGKEFEQIEQLKSDDSNGIENNVPRHRKNESLGQ

SEQ ID NO: 9 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 10 - protein, 353 aa, Homo sapiens [sequence omitted]

SEQ ID NO: 11 - protein, 1204 aa, Homo sapiens
MYVTMMMTDQIPLELPPLLNGEVAMMPHLVNGDAAQQVILVQVNPGETFTIRAEDGTLQCIQGPAEVPMMSPNGSIPPIHVPPGYISQVIEDSTGVRRVVVTPQSPECYPPSYPSAMSPTHHLPPYLTHHPHFIHNSHTAYYPPVTGPGDMPPQFFPQHHLPHTIYGEQEIIPFYGMSTYITREDQYSKPPHKKLKDRQIDRQNRLNSPPSSIYKSSCTTVYNGYGKGHSGGSGGGGSGSGPGIKKTERRARSSPKSNDSDLQEYELEVKRVQDILSGIEKPQVSNIQARAVVLSWAPPVGLSCGPHSGLSFPYSYEVALSDKGRDGKYKIIYSGEELECNLKDLRPATDYHVRVYAMYNSVKGSCSEPVSFTTHSCAPECPFPPKLAHRSKSSLTLQWKAPIDNGSKITNYLLEWDEGKRNSGFRQCFFGSQKHCKLTKLCPAMGYTFRLAARNDIGTSGYSQEVVCYTLGNIPQMPSAPRLVRAGITWVTLQWSKPEGCSPEEVITYTLEIQEDENDNLFHPKYTGEDLTCTVKNLKRSTQYKFRLTASNTEGKSCPSEVLVCTTSPDRPGPPTRPLVKGPVTSHGFSVKWDPPKDNGGSEILKYLLEITDGNSEANQWEVAYSGSATEYTFTHLKPGTLYKLRACCISTGGHSQCSESLPVRTLSIAPGQCRPPRVLGRPKHKEVHLEWDVPASESGCEVSEYSVEMTEPEDVASEVYHGPELECTVGNLLPGTVYRFRVRALNDGGYGPYSDVSEITTAAGPPGQCKAPCISCTPDGCVLVGWESPDSSGADISEYRLEWGEDEESLELIYHGTDTRFEIRDLLPAAQYCCRLQAFNQAGAGPYSELVLCQTPASAPDPVSTLCVLEEEPLDAYPDSPSACLVLNWEEPCNNGSEILAYTIDLGDTSITVGNTTMHVMKDLLPETTYRIRIQAINEIGAGPFSQFIKAKTRPLPPLPPRLECAAAGPQSLKLKWGDSNSKTHAAEDIVYTLQLEDRNKRFISIYRGPSHTYKVQRLTEFTCYSFRIQAASEAGEGPFSETYTFSTTKSVPPTIKAPRVTQLEGNSCEILWETVPSMKGDPVNYILQVLVGRESEYKQVYKGEEATFQISGLQTNTDYRFRVCACRRCLDTSQELSGAFSPSAAFVLQRSEVMLTGDMGSLDDPKMKSMMPTDEQFAAIIVLGFATLSILFAFILQYFLMK

SEQ ID NO: 12 - protein, 678 aa, Homo sapiens
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 13 - protein, 222 aa, Homo sapiens
MAARPKLHYPNGRGRMESVRWVLAAAGVEFDEEFLETKEQLYKLQDGNHLLFQQVPMVEIDGMKLVQTRSILHYIADKHNLFGKNLKERTLIDMYVEGTLDLLELLIMHPFLKPDDQQKEVVNMAQKAIIRYFPVFEKILRGHGQSFLVGNQLSLADVILLQTILALEEKIPNILSAFPFLQEYTVKLSNIPTIKRFLEPGSKKKPPPDEIYVRTVYNIFRP

SEQ ID NO: 14 - protein, 520 aa, Homo sapiens
MPGSLPLNAEACWPKDVGIVALEIYFPSQYVDQAELEKYDGVDAGKYTIGLGQAKMGFCTDREDINSLCMTVVQNLMERNNLSYDCIGRLEVGTETIIDKSKSVKTNLMQLFEESGNTDIEGIDTTNACYGGTAAVFNAVNWIESSSWDGRYALVVAGDIAVYATGNARPTGGVGAVALLIGPNAPLIFERGLRGTHMQHAYDFYKPDMLSEYPIVDGKLSIQCYLSALDRCYSVYCKKIHAQWQKEANDNDFTLNDFGFMIFHSPYCKLVQKSLARMLLNDFLNDQNRDKNSIYSGLKAFGDVKLEDTYFDRDVEKAFMKASSELFSQKTKASLLVSNQNGNMYTSSVYGSLASVLAQYSPQHLAGKRIGVFSYGSGLAATLYSLKVTQDATPGSALDKITASLCDLKSRLDSRTGVAQDVFAENMKLREDTHHLVNYIPQGSIDSLFEGTWYLVRVDEKHRRTYARRPTPNDDTLDEGVGLVHSNIATEHIPSPAKKVPRLPATAAEPEAAVISNGVW

SEQ ID NO: 15 - protein, 630 aa, Homo sapiens
MAAGVAAWLPFARAAAIGWMPVASGPMPAPPRQERKRTQDALIVLNVSGTRFQTWQDTLERYPDTLLGSSERDFFYHPETQQYFFDRDPDIFRHILNFYRTGKLHYPRHECISAYDEELAFFGLIPEIIGDCCYEEYKDRRRENAERLQDDADTDTAGESALPTMTARQRVWRAFENPHTSTMALVFYYVTGFFIAVSVIANVVETVPCGSSPGHIKELPCGERYAVAFFCLDTACVMIFTVEYLLRLAAAPSRYRFVRSVMSIIDVVAILPYYIGLVMTDNEDVSGAFVTLRVFRVFRIFKFSRHSQGLRILGYTLKSCASELGFLLFSLTMAIIIFATVMFYAEKGSSASKFTSIPAAFWYTIVTMTTLGYGDMVPKTIAGKIFGSICSLSGVLVIALPVPVIVSNFSRIYHQNQRADKRRAQKKARLARIRAAKSGSANAYMQSKRSGLLSNQLQSSEDEPAFVSKSGSSFETQHHHLLHCLEKTTNHEFVDEQVFEESCMEVATVNRPSSHSPSLSSQQGVTSTCCSRRHKKTFRIPNANVSGSHRGSVQELSTIQIRCVERTPLSNSRSSLNAKMEECVKLNCEQPYVTTAIISIPTPPVTTPEGDDRPESPEYSGGNIVRVSAL

SEQ ID NO: 16 - protein, 976 aa, Homo sapiens [sequence omitted]

SEQ ID NO: 17 - protein, 275 aa, Homo sapiens
MVMLQGVVPLDAHRFQVDFQCGCSLCPRPDIAFHFNPRFHTTKPHVICNTLHGGRWQREARWPHLALRRGSSFLILFLFGNEEVKVSVNGQHFLHFRYRLPLSHVDTLGIFGDILVEAVGFLNINPFVEGSREYPAGHPFLLMSPRLEVPCSHALPQGLSPGQVIIVRGLVLQEPKHFTVSLRDQAAHAPVTLRASFADRTLAWISRWGQKKLISAPFLFYPQRFFEVLLLFQEGGLKLALNGQGLGATSMNQQALEQLRELRISGSVQLYCVHS

SEQ ID NO: 18 - protein, 250 aa, Homo sapiens
MGEKVSEAPEPVPRGCSGHGSRTPASALVAASSPGASSAESSSGSETLSEEGEPGGFSREHQPPPPPPLGGTLGARAPAAWAPASVLLERGVLALPPPLPGGAVPPAPRGSSASQEEQDEELDHILSPPPMPFRKCSNPDVASGPGKSLKYKRQLSEDGRQLRRGSLGGALTGRYLLPNPVAGQAWPASAETSNLVRMRSQALGQSAPSLTASLKELSLPRRGSLIDSQKWNCLVKRPVCPNAGRTSPLG

SEQ ID NO: 19 - protein, 362 aa, Homo sapiens
MSENGSFANCCEAGGWAVRPGWSGAGSARPSRTPRPPWVAPALSAVLIVTTAVDVVGNLLVILSVLRNRKLRNAGNLFLVSLALADLVVAFYPYPLILVAIFYDGWALGEEHCKASAFVMGLSVIGSVFNITAIAINRYCYICHSMAYHRIYRRWHTPLHICLIWLLTVVALLPNFFVGSLEYDPRIYSCTFIQTASTQYTAAVVVIHFLLPIAVVSFCYLRIWVLVLQARRKAKPESRLCLKPSDLRSFLTMFVVFVIFAICWAPLNCIGLAVAINPQEMAPQIPEGLFVTSYLLAYFNSCLNAIVYGLLNQNFRREYKRILLALWNPRHCIQDASKGSHAEGLQSPAPPIIGVQHQADAL

SEQ ID NO: 20 - protein, 544 aa, Homo sapiens
MSDGLDNEEKPPAPPLRMNSNNRDSSALNHSSKPLPMAPEEKNKKARLRSIFPGGGDKTNKKKEKERPEISLPSDFEHTIHVGFDAVTGEFTGIPEQWARLLQTSNITKLEQKKNPQAVLDVLKFYDSKETVNNQKYMSFTSGDKSAHGYIAAHPSSTKTASEPPLAPPVSEEEDEEEEEEEDENEPPPVIAPRPEHTKSIYTRSVVESIASPAVPNKEVTPPSAENANSSTLYRNTDRQRKKSKMTDEEILEKLRSIVSVGDPKKKYTRFEKIGQGASGTVYTALDIATGQEVAIKQMNLQQQPKKELIINEILVMRENKNPNIVNYLDSYLVGDELWVVMEYLAGGSLTDVVTETCMDEGQIAAVCRECLQALDFLHSNQVIHRDIKSDNILLGMDGSVKLTDFGFCAQITPEQSKRSTMVGTPYWMAPEVVTRKAYGPKVDIWSLGIMAIEMVEGEPPYLNENPLRALYLIATNGTPELQNPERLSAVFRDFLNRCLEMDVDRRGSAKELLQHPFLKLAKPLSSLTPLIIAAKEAIKNSSR

SEQ ID NO: 21 - protein, 1448 aa, Homo sapiens
MAYSWQTDPNPNESHEKQYEHQEFLFVNQPHSSSQVSLGFDQIVDEISGKIPHYESEIDENTFFVPTAPKWDSTGHSLNEAHQISLNEFTSKSRELSWHQVSKAPAIGFSPSVLPKPQNTNKECSWGSPIGKHHGADDSRFSILAPSFTSLDKINLEKELENENHNYHIGFESSIPPTNSSFSSDFMPKEENKRSGHVNIVEPSLMLLKGSLQPGMWESTWQKNIESIGCSIQLVEVPQSSNTSLASFCNKVKKIRERYHAADVNFNSGKIWSTTTAFPYQLFSKTKFNIHIFIDNSTQPLHFMPCANYLVKDLIAEILHFCTNDQLLPKDHILSVCGSEEFLQNDHCLGSHKMFQKDKSVIQLHLQKSREAPGKLSRKHEEDHSQFYLNQLLEFMHIWKVSRQCLLTLIRKYDFHLKYLLKTQENVYNIIEEVKKICSVLGCVETKQITDAVNELSLILQRKGENFYQSSETSAKGLIEKVTTELSTSIYQLINVYCNSFYADFQPVNVPRCTSYLNPGLPSHLSFTVYAAHNIPETWVHRINFPLEIKSLPRESMLTVKLFGIACATNNANLLAWTCLPLFPKEKSILGSMLFSMTLQSEPPVEMITPGVWDVSQPSPVTLQIDFPATGWEYMKPDSEENRSNLEEPLKECIKHIARLSQKQTPLLLSEEKKRYLWFYRFYCNNENCSLPLVLGSAPGWDERTVSEMHTILRRWTFSQPLEALGLLTSSFPDQEIRKVAVQQLDNLLNDELLEYLPQLVQAVKFEWNLESPLVQLLLHRSLQSIQVAHRLYWLLKNAENEAYFKSWYQKLLAALQFCAGKALNDEFSKEQKLIKILGDIGERVKSASDHQRQEVLKKEIGRLEEFFQDVNTCHLPLNPALCIKGIDHDACSYFTSNALPLKITFINANLMGKNISIIFKAGDDLRQDMLVLQLIQVMDNIWLQEGLDMQMIIYRCLSTGKDQRLVQMVPDAVTLAKIHRHSGLIGPLKENTIKKWFSQHNHLKADYEKALRNFFYSCAGWCVVTFILGVCDRHNDNIMLTKSGHMFHIDFGKFLGHAQTFGGIKRDRAPFIFTSEMEYFITEGGKNPQHFQDFVELCCRAYNIIRKHSQLLLNLLEMMLYAGLPELSGIQDLKYVYNNLRPQDTDLEATSHFTKKIKESLECFPVKLNNLIHTLAQMSAISPAKSTSQTFPQESCLLSTTRSIERAILGFSKKSSNLYLIQVTHSNNETSLTEKSFEQFSKLHSQLQKQFASLTLPEFPHWWHLPFTNSDHRRFRDLNHYMEQILNVSHEVTNSDCVLSFFLSEAVQQTVESSPVYLGEKKFPDKKPKVQLVISYEDVKLTILVKHMKNIHLPDGSAPSAHVEFYLLPYPSEVLRRKTKSVPKCTDPTYNEIVVYDEVTELQGHVLMLIVKSKTVFVGAINIRLCSVPLDKEKWYPLGNSIISPLL

SEQ ID NO: 22 - protein, 148 aa, Homo sapiens
MARALCRLPRRGLWLLLAHHLFMTTACQEANYGALLRELCLTQFQVDMEAVGETLWCDWGRTIRSYRELADCTWHMAEKLGCFWPNAEVDRFFLAVHGRYFRSCPISGRAVRDPPGSILYPFIVVPITVTLLVTALVVWQSKRTEGIV

SEQ ID NO: 23 - protein, 446 aa, Homo sapiens [sequence omitted]

SEQ ID NO: 24 - protein, 129 aa, Homo sapiens
MKILVALAVFFLVSTQLFAEEIGANDDLNYWSDWYDSDQIKEELPEPFEHLLQRIARRPKPQQFFGLMGKRDADSSIEKQVALLKALYGHGQISHKRHKTDSFVGLMGKRALNSVAYERSAMQNYERRR

SEQ ID NO: 25 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 26 - protein, 44 aa, Homo sapiens
MSDKPDMAEIEKFDKSKLKKTETQEKNPLPSKETIEQEKQAGES

SEQ ID NO: 27 - protein, 248 aa, Homo sapiens
MAGLNSLEAVKRKIQALQQQADEAEDRAQGLQRELDGERERREKAEGDVAALNRRIQLFEEELDRAQERLATALQKLEEAEKAADESERGMKVIENRAMKDEEKMEIQEMQLKEAKHIAEEADRKYEEVARKLVILEGELERAEERAEVSELKCGDLEEELKNVTNNLKSLEAASEKYSEKEDKYEEEIKLLSDKLKEAETRAEFAERTVAKLEKTIDDLEEKLAQAKEENVGLHQTLDQTLNELNCI

SEQ ID NO: 28 - protein, 343 aa, Homo sapiens
MNIHRSTPITIARYGRSRNKTQDFEELSSIRSAEPSQSFSPNLGSPSPPETPNLSHCVSCIGKYLLLEPLEGDHVFRAVHLHSGEELVCKVFDISCYQESLAPCFCLSAHSNINQITEIILGETKAYVFFERSYGDMHSFVRTCKKLREEEAARLFYQIASAVAHCHDGGLVLRDLKLRKFIFKDEERTRVKLESLEDAYILRGDDDSLSDKHGCPAYVSPEILNTSGSYSGKAADVWSLGVMLYTMLVGRYPFHDIEPSSLFSKIRRGQFNIPETLSPKAKCLIRSILRREPSERLTSQEILDHPWFSTDFSVSNSAYGAKEVSDQLVPDVNMEENLDPFFN

SEQ ID NO: 29 - protein, 466 aa, Homo sapiens [sequence omitted]

SEQ ID NO: 30 - protein, 317 aa, Homo sapiens
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGEEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSRSRG

SEQ ID NO: 31 - protein, 1209 aa, Homo sapiens
MECYYIVISSTHLSNGHFRNIKGVFRGPLSKNGNKTLDYAEKENTIAKALEDLKANFYCELCDKQYYKHQEFDNHINSYDHAHKQRLKELKQREFARNVASKSRKDERKQEKALQRLHKLAELRKETVCAPGSGPMFKSTTVTVRENCNEISQRVVVDSVNNQQDFKYTLIHSEENTKDATTVAEDPESANNYTAKNNQVGDQAQGIHRHKIGFSFAFPKKASVKLESSAAAFSEYSDDASVGKGFSRKSRFVPSACHLQQSSPTDVLLSSEEKTNSFHPPEAMCRDKETVQTQEIKEVSSEKDALLLPSFCKFQLQLSSDADNCQNSVPLADQIPLESVVINEDIPVSGNSFELLGNKSTVLDMSNDCISVQATTEENVKHNEASTTEVENKNGPETLAPSNTEEVNITIHKKTNFCKRQCEPFVPVLNKHRSTVLQWPSEMLVYTTTKPSISYSCNPLCFDFKSTKVNNNLDKNKPDLKDLCSQQKQEDICMGPLSDYKDVSTEGLTDYEIGSSKNKCSQVTPLLADDILSSSCDSGKNENTGQRYKNISCKIRETEKYNFTKSQIKQDTLDEKYNKIRLKETHEYWFHKSRRKKKRKKLCQHHHMEKTKESETRCKMEAENSYTENAGKYLLEPISEKQYLAAEQLLDSHQLLDKRPKSESISLSDNEEMCKTWNTEYNTYDTISSKNHCKKNTILLNGQSNATMIHSGKHNLTYSRTYCCWKTKMSSCSQDHRSLVLQNDMKHMSQNQAVKRGYNSVMNESERFYRKRRQHSHSYSSDESLNRQNHLPEEFLRPPSTSVAPCKPKKKRRRKRGRFHPGFETLELKENTDYPVKDNSSLNPLDRLISEDKKEKMKPQEVAKIERNSEQTNQLRNKLSFHPNNLLPSETNGETEHLEMETTSGELSDVSNDPTTSVCVASAPTKEAIDNTLLEHKERSENINLNEKQIPFQVPNIERNFRQSQPKSYLCHYELAEALPQGKMNETPTEWLRYNSGILNTQPPLPFKEAHVSGHTFVTAEQILAPLALPEQALLIPLENHDKFKNVPCEVYQHILQPNMLANKVKFTFPPAALPPPSTPLQPLPLQQSLCSTSVTTIHHTVLQQHAAAAAAAAAAAAAGTFKVLQPHQQFLSQIPALTRTSLPQLSVGPVGPRLCPGNQPTFVAPPQMPIIPASVLHPSHLAFPSLPHALFPSLLSPHPTVIPLQPLF

SEQ ID NO: 32 - DNA, 2538 bp, Homo sapiens
  source 1..2538: mol_type = genomic DNA
atggactattactttgggcctcctccgccccagaagcccgatgctgagcgccagacggacgaagagaaggaggagctgaagaaacccaaaaaggaggacagcagccccaaggaggagaccgacaagtgggcagtggagaagggcaaggaccacaaagagccccgaaagggcgaggagttggaggaggagtggacgcctacggagaaagtcaagtgtccccccattgggatggagtcacaccgtattgaggacaaccagatccgagcctcctccatgctgcgccacggcctgggggcacagcgcggccggctcaacatgcagaccggtgccactgaggacgactactatgatggtgcgtggtgtgccgaggacgatgccaggacccagtggatagaggtggacaccaggaggactacccggttcacaggcgtcatcacccagggcagagactccagcatccatgacgattttgtgaccaccttcttcgtgggcttcagcaatgacagccagacatgggtgatgtacaccaacggctatgaggaaatgacctttcatgggaacgtggacaaggacacacccgtgctgagtgagctcccagagccggtggtggctcgtttcatccgcatctacccactcacctggaatggcagcctgtgcatgcgcctggaggtgctggggtgctctgtggcccctgtctacagctactacgcacagaatgaggtggtggccaccgatgacctggatttccggcaccacagctacaaggacatgcgccagctcatgaaggtggtgaacgaggagtgccccaccatcacccgcacttacagcctgggcaagagctcacgaggcctcaagatctatgccatggagatctcagacaaccctggggagcatgaactgggggagcccgagttccgctacactgctgggatccatggcaacgaggtgctgggccgagagctgttgctgctgctcatgcagtacctgtgccgagagtaccgcgatgggaacccacgtgtgcgcagcctggtgcaggacacacgcatccacctggtgccctcactgaaccctgatggctacgaggtggcagcgcagatgggctcagagtttgggaactgggcgctgggactgtggactgaggagggctttgacatctttgaagatttcccggatctcaactctgtgctctggggagctgaggagaggaaatgggtcccctaccgggtccccaacaataacttgcccatccctgaacgctacctttcgccagatgccacggtatccacggaggtccgggccatcattgcctggatggagaagaaccccttcgtgctgggagcaaatctgaacggcggcgagcggctagtatcctacccctacgatatggcccgcacgcctacccaggagcagctgctggccgcagccatggcagcagcccggggggaggatgaggacgaggtctccgaggcccaggagactccagaccacgccatcttccggtggcttgccatctccttcgcctccgcacacctcaccttgaccgagccctaccgcggaggctgccaagcccaggactacaccggcggcatgggcatcgtcaacggggccaagtggaacccccggaccgggactatcaatgacttcagttacctgcataccaactgcctggagctctccttctacctgggctgtgacaagttccctcatgagagtgagctgccccgcgagtgggagaacaacaaggaggcgctgctcaccttcatggagcaggtgcaccgcggcattaagggggtggtgacggacgagcaaggcatccccattgccaacgccaccatctctgtgagtggcattaatcacggcgtgaagacagccagtggtggtgattactggcgaatcttgaacccgggtgagtaccgcgtgacagcccacgcggagggctacaccccgagcgccaagacctgcaatgttgactatgacatcggggccactcagtgcaacttcatcctggctcgctccaactggaagcgcatccgggagatcatggccatgaacgggaaccggcctatcccacacatagacccatcgcgccctatgaccccccaacagcgacgcctgcagcagcgacgcctacaacaccgcctgcggcttcgggcacagatgcggctgcggcgcctcaacgccaccaccaccctaggcccccacactgtgcctcccacgctgccccctgcccctgccaccaccctgagcactaccatagagccctggggcctcataccgccaaccaccgctggctgggaggagtcggagactgagacctacacagaggtggtgacagagtttgggaccgaggtggagcccgagtttgggaccaaggtggagcccgagtttgagacccagttggagcctgagtttgagacccagctggaacccgagtttgaggaagaggaggaggaggagaaagaggaggagatagccactggccaggcattccccttcacaacagtagagacctacacagtgaactttggggacttctga

SEQ ID NO: 33 - DNA, 1557 bp, Homo sapiens
  source 1..1557: mol_type = genomic DNA
atgggcgcactggcccgggcgctgctgctgcctctgctggcccagtggctcctgcgcgccgccccggagctggcccccgcgcccttcacgctgcccctccgggtggccgcggccacgaaccgcgtagttgcgcccaccccgggacccgggacccctgccgagcgccacgccgacggcttggcgctcgccctggagcctgccctggcgtcccccgcgggcgccgccaacttcttggccatggtagacaacctgcagggggactctggccgcggctactacctggagatgctgatcgggacccccccgcagaagctacagattctcgttgacactggaagcagtaactttgccgtggcaggaaccccgcactcctacatagacacgtactttgacacagagaggtctagcacataccgctccaagggctttgacgtcacagtgaagtacacacaaggaagctggacgggcttcgttggggaagacctcgtcaccatccccaaaggcttcaatacttcttttcttgtcaacattgccactatttttgaatcagagaatttctttttgcctgggattaaatggaatggaatacttggcctagcttatgccacacttgccaagccatcaagttctctggagaccttcttcgactccctggtgacacaagcaaacatccccaacgttttctccatgcagatgtgtggagccggcttgcccgttgctggatctgggaccaacggaggtagtcttgtcttgggtggaattgaaccaagtttgtataaaggagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctggacgaattcggaaacaccttggtcttacttccctaaaatctccatctacctgagagatgagaactccagcaggtcattccgtatcacaatcctgcctcagctttacattcagcccatgatgggggccggcctgaattatgaatgttaccgattcggcatttccccatccacaaatgcgctggtgatcggtgccacggtgatggagggcttctacgtcatcttcgacagagcccagaagagggtgggcttcgcagcgagcccctgtgcagaaattgcaggtgctgcagtgtctgaaatttccgggcctttctcaacagaggatgtagccagcaactgtgtccccgctcagtctttgagcgagcccattttgtggattgtgtcctatgcgctcatgagcgtctgtggagccatcctccttgtcttaatcgtcctgctgctgctgccgttccggtgtcagcgtcgcccccgtgaccctgaggtcgtcaatgatgagtcctctctggtcagacatcgctggaaatga

SEQ ID NO: 34 - DNA, 543 bp, Homo sapiens
  source 1..543: mol_type = genomic DNA
atggcatctacttcgtatgactattgcagagtgcccatggaagacggggataagcgctgtaagcttctgctggggataggaattctggtgctcctgatcatcgtgattctgggggtgcccttgattatcttcaccatcaaggccaacagcgaggcctgccgggacggccttcgggcagtgatggagtgtcgcaatgtcacccatctcctgcaacaagagctgaccgaggcccagaagggctttcaggatgtggaggcccaggccgccacctgcaaccacactgtgatggccctaatggcttccctggatgcagagaaggcccaaggacaaaagaaagtggaggagcttgagggagagatcactacattaaaccataagcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtga

SEQ ID NO: 35 - DNA, 990 bp, Homo sapiens
  source 1..990: mol_type = genomic DNA
atgacccacttcaacaagggcccttcctatgggctctcggccgaagtcaagaacaagattgcttccaagtatgatcatcaggcagaagaagatcttcgcaattggatagaagaggtgacaggcatgagcattggccccaacttccagctgggcttaaaggatggcatcatcctctgcgaacttataaacaagctacagccaggctcagtgaagaaggtcaacgagtcctcactgaactggcctcagttggagaatattggcaactttattaaagctattcaggcttatggtatgaagccacatgacatattcgaagcaaatgatctttttgagaatggaaacatgacccaggttcagactactctggtggctctagcaggtctggctaaaacaaaaggattccatacaaccattgacattggagttaagtatgcagaaaaacaaacaagacgttttgatgaaggaaaattaaaagctggccaaagtgtaattggtctgcagatgggaaccaacaaatgtgccagccaggcaggtatgacagcttacgggactaggaggcatctttatgatcccaaaatgcaaactgacaaaccttttgaccagaccacaattagtctgcagatgggcactaataaaggagccagccaggcagggatgttagcaccaggtaccagaagagacatctatgatcagaagctaacattacagccggtggacaactcgacaatttccctacagatgggtaccaacaaagttgcttcccagaaaggaatgagtgtgtatgggcttgggcggcaagtatatgatcccaaatactgtgctgctcctacagaacctgtcattcacaacggaagccaaggaacaggaacaaatggttcggaaatcagtgatagtgattatcaggcagaataccctgatgagtatcatggcgagtaccaggatgactaccccagagattaccaatatagcgaccaaggcattgattattag

SEQ ID NO: 36 - DNA, 5010 bp, Homo sapiens
  source 1..5010: mol_type = genomic DNA
atggggccccggctcagcgtctggctgctgctgctgcccgccgcccttctgctccacgaggagcacagccgggccgctgcgaagggtggctgtgctggctctggctgtggcaaatgtgactgccatggagtgaagggacaaaagggtgaaagaggcctcccggggttacaaggtgtcattgggtttcctggaatgcaaggacctgaggggccacagggaccaccaggacaaaagggtgatactggagaaccaggactacctggaacaaaagggacaagaggacctccgggagcatctggctaccctggaaacccaggacttcccggaattcctggccaagacggcccgccaggccccccaggtattccaggatgcaatggcacaaagggggagagagggccgctcgggcctcctggcttgcctggtttcgcaggaaatcccggaccaccaggcttaccagggatgaagggtgatccaggtgagatacttggccatgtgcccgggatgctgttgaaaggtgaaagaggatttcccggaatcccagggactccaggcccaccaggactgccagggcttcaaggtcctgttgggcctccaggatttaccggaccaccaggtcccccaggccctcccggccctccaggtgaaaagggacaaatgggcttaagttttcaaggaccaaaaggtgacaagggtgaccaaggggtcagtgggcctccaggagtaccaggacaagctcaagttcaagaaaaaggagacttcgccaccaagggagaaaagggccaaaaaggtgaacctggatttcaggggatgccaggggtcggagagaaaggtgaacccggaaaaccaggacccagaggcaaacccggaaaagatggtgacaaaggggaaaaagggagtcccggttttcctggtgaacccgggtacccaggactcataggccgccagggcccgcagggagaaaagggtgaagcaggtcctcctggcccacctggaattgttataggcacaggacctttgggagaaaaaggagagaggggctaccctggaactccggggccaagaggagagccaggcccaaaaggtttcccaggactaccaggccaacccggacctccaggcctccctgtacctgggcaggctggtgcccctggcttccctggtgaaagaggagaaaaaggtgaccgaggatttcctggtacatctctgccaggaccaagtggaagagatgggctcccgggtcctcctggttcccccgggccccctgggcagcctggctacacaaatggaattgtggaatgtcagcccggacctccaggtgaccagggtcctcctggaattccagggcagccaggatttataggcgaaattggagagaaaggtcaaaaaggagagagttgcctcatctgtgatatagacggatatcgggggcctcccgggccacagggacccccgggagaaataggtttcccagggcagccaggggccaagggcgacagaggtttgcctggcagagatggtgttgcaggagtgccaggccctcaaggtacaccagggctgataggccagccaggagccaagggggagcctggtgagttttatttcgacttgcggctcaaaggtgacaaaggagacccaggctttccaggacagcccggcatgccagggagagcgggttctcctggaagagatggccatccgggtcttcctggccccaagggctcgccgggttctgtaggattgaaaggagagcgtggcccccctggaggagttggattcccaggcagtcgtggtgacaccggcccccctgggcctccaggatatggtcctgctggtcccattggtgacaaaggacaagcaggctttcctggaggccctggatccccaggcctgccaggtccaaagggtgaaccaggaaaaattgttcctttaccaggcccccctggagcagaaggactgccggggtccccaggcttcccaggtccccaaggagaccgaggctttcccggaaccccaggaaggccaggcctgccaggagagaagggcgctgtgggccagccaggcattggatttccagggccccccggccccaaaggtgttgacggcttacctggagacatggggccaccggggactccaggtcgcccgggatttaatggcttacctgggaacccaggtgtgcagggccagaagggagagcctggagttggtctaccgggactcaaaggtttgccaggtcttcccggcattcctggcacacccggggagaaggggagcattggggtaccaggcgttcctggagaacatggagcgatcggaccccctgggcttcaggggatcagaggtgaaccgggacctcctggattgccaggctccgtggggtctccaggagttccaggaataggcccccctggagctaggggtccccctggaggacagggaccaccggggttgtcaggccctcctggaataaaaggagagaagggtttccccggattccctggactggacatgccgggccctaaaggagataaaggggctcaaggactccctggcataacgggacagtcggggctccctggccttcctggacagcagggggctcctgggattcctgggtttccaggttccaagggagaaatgggcgtcatggggacccccgggcagccgggctcaccaggaccagtgggtgctcctggattaccgggtgaaaaaggggaccatggctttccgggctcctcaggacccaggggagaccctggcttgaaaggtgataagggggatgtcggtctccctggcaagcctggctccatggataaggtggacatgggcagcatgaagggccagaaaggagaccaaggagagaaaggacaaattggaccaattggtgagaagggatcccgaggagaccctgggaccccaggagtgcctggaaaggacgggcaggcaggacagcctgggcagccaggacctaaaggtgatccaggtataagtggaaccccaggtgctccaggacttccgggaccaaaaggatctgttggtggaatgggcttgccaggaacacctggagagaaaggtgtgcctggcatccctggcccacaaggttcacctggcttacctggagacaaaggtgcaaaaggagagaaagggcaggcaggcccacctggcataggcatcccaggactgcgtggtgaaaagggagatcaagggatagcgggtttcccaggaagccctggagagaagggagaaaaaggaagcattgggatcccaggaatgccagggtccccaggccttaaagggtctcccgggagtgttggctatccaggaagtcctgggctacctggagaaaaaggtgacaaaggcctcccaggattggatggcatccctggtgtcaaaggagaagcaggtcttcctgggactcctggccccacaggcccagctggccagaaaggggagccaggcagtgatggaatcccggggtcagcaggagagaagggtgaaccaggtctaccaggaagaggattcccagggtttccaggggccaaaggagacaaaggttcaaagggtgaggtgggtttcccaggattagccgggagcccaggaattcctggatccaaaggagagcaaggattcatgggtcctccggggccccagggacagccggggttaccgggatccccaggccatgccacggaggggcccaaaggagaccgcggacctcagggccagcctggcctgccaggacttccgggacccatggggcctccagggcttcctgggattgatggagttaaaggtgacaaaggaaatccaggctggccaggagcacccggtgtcccagggcccaagggagaccctggattccagggcatgcctggtattggtggctctccaggaatcacaggctctaagggtgatatggggcctccaggagttccaggatttcaaggtccaaaaggtcttcctggcctccagggaattaaaggtgatcaaggcgatcaaggcgtcccgggagctaaaggtctcccgggtcctcctggccccccaggtccttacgacatcatcaaaggggagcccgggctccctggtcctgagggccccccagggctgaaagggcttcagggactgccaggcccgaaaggccagcaaggtgttacaggattggtgggtatacctggacctccaggtattcctgggtttgacggtgcccctggccagaaaggagagatgggacctgccgggcctactggtccaagaggatttccaggtccaccaggccccgatgggttgccaggatccatggggcccccaggcaccccatctgttgatcacggcttccttgtgaccaggcatagtcaaacaatagatgacccacagtgtccttctgggaccaaaattctttaccacgggtactctttgctctacgtgcaaggcaatgaacgggcccatggacaggacttgggcacggccggcagctgcctgcgcaagttcagcacaatgcccttcctgttctgcaatattaacaacgtgtgcaactttgcatcacgaaatgactactcgtactggctgtccacccctgagcccatgcccatgtcaatggcacccatcacgggggaaaacataagaccatttattagtaggtgtgctgtgtgtgaggcgcctgccatggtgatggccgtgcacagccagaccattcagatcccaccgtgccccagcgggtggtcctcgctgtggatcggctactcttttgtgatgcacaccagcgctggtgcagaaggctctggccaagccctggcgtcccccggctcctgcctggaggagtttagaagtgcgccattcatcgagtgtcacggccgtgggacctgcaattactacgcaaacgcttacagcttttggctcgccaccatagagaggagcgagatgttcaagaagcctacgccgtccaccttgaaggcaggggagctgcgcacgcacgtcagccgctgccaagtctgtatgagaagaacataa

SEQ ID NO: 37 - DNA, 429 bp, Homo sapiens
  source 1..429: mol_type = genomic DNA
atgcccaggtgccggtggctctccctgatcctcctcaccattcccctggccctggtggccaggaaagacccaaaaaagaatgagacgggggtgctgaggaaattaaaacccgtcaatgcctcaaatgccaacgtgaagcagtgtctgtggtttgccatgcaagaatacaacaaagagagcgaggacaagtatgtcttcctggtggtcaagacactgcaagcccagcttcaggtcacaaatcttctggaataccttattgatgtagaaattgcccgcagcgattgcagaaagcctttaagcactaatgaaatctgcgccattcaagaaaactccaagctgaaaaggaaattaagctgcagctttttggtaggagcacttccctggaatggtgaattcactgtgatggagaaaaagtgtgaagatgcttaa

SEQ ID NO: 38 - DNA, 1002 bp, Homo sapiens
  source 1..1002: mol_type = genomic DNA
atgaatcctacactcatccttgctgccttttgcctgggaattgcctcagctactctaacatttgatcacagtttagaggcacagtggaccaagtggaaggcgatgcacaacagattatacggcatgaatgaagaaggatggaggagagcagtgtgggagaagaacatgaagatgattgaactgcacaatcaggaatacagggaagggaaacacagcttcacaatggccatgaacgcctttggagacatgaccagtgaagaattcaggcaggtgatgaatggctttcaaaaccgtaagcccaggaaggggaaagtgttccaggaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtga

SEQ ID NO: 39 - DNA, 984 bp, Homo sapiens
  source 1..984: mol_type = genomic DNA
atgcgcgccctccccggcctgctggaggccagggcgcgtacgccccggctgctcctcctccagtgccttctcgctgccgcgcgcccaagctcggcggacggcagtgccccagattcgccttttacaagtccacctctcagagaagaaataatggcaaataacttttccttggagagtcataacatatcactgactgaacattctagtatgccagtagaaaaaaatatcactttagaaaggccttctaatgtaaatctcacatgccagttcacaacatctggggatttgaatgcagtaaatgtgacttggaaaaaagatggtgaacaacttgagaataattatcttgtcagtgcaacaggaagcaccttgtatacccaatacaggttcaccatcattaatagcaaacaaatgggaagttattcttgtttctttcgagaggaaaaggaacaaaggggaacatttaatttcaaagtccctgaacttcatgggaaaaacaagccattgatctcttacgtaggggattctactgtcttgacatgtaaatgtcaaaattgttttcctttaaattggacctggtacagtagtaatgggagtgtaaaggttcctgttggtgttcaaatgaataaatatgtgatcaatggaacatatgctaacgaaacaaagctgaagataacacaacttttggaggaagatggggaatcttactggtgccgtgcactattccaattaggcgagagtgaagaacacattgagcttgtggtgctgagctatttggtgcccctcaaaccatttcttgtaatagtggctgaggtgattcttttagtggccaccattctgctttgtgaaaagtacacacaaaagaaaaagaagcactcagatgaggggaaagaatttgagcagattgaacagctgaaatcagatgatagcaatggtatagaaaataatgtccccaggcatagaaaaaatgagtctctgggccagtga

SEQ ID NO: 40 - DNA, 888 bp, Homo sapiens
  source 1..888: mol_type = genomic DNA
atggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataa

SEQ ID NO: 41 - DNA, 1062 bp, Homo sapiens
  source 1..1062: mol_type = genomic DNA
atgaacggagaccagaattcagatgtttatgcccaagaaaagcaggatttcgttcagcacttctcccagatcgttagggtgctgactgaggatgagatggggcacccagagataggagatgctattgcccggctcaaggaggtcctggagtacaatgccattggaggcaagtataaccggggtttgacggtggtagtagcattccgggagctggtggagccaaggaaacaggatgctgatagtctccagcgggcctggactgtgggctggtgtgtggaactgctgcaagctttcttcctggtggcagatgacatcatggattcatcccttacccgccggggacagacctgctggtatcagaagccgggcgtgggtttggatgccatcaatgatgctaacctcctggaagcatgtatctaccgcctgctgaagctctattgccgggagcagccctattacctgaacctgatcgagctcttcctgcagagttcctatcagactgagattgggcagaccctggacctcctcacagccccccagggcaatgtggatcttgtcagattcactgaaaagaggtacaaatctattgtcaagtacaagacagctttctactccttctaccttcctatagctgcagccatgtacatggcaggaattgatggcgagaaggagcacgccaatgccaagaagatcctgctggagatgggggagttctttcagattcaggatgattaccttgacctctttggggaccccagtgtgaccggcaaaattggcactgacatccaggacaacaaatgcagctggctggtggttcagtgtctgcaacgggccactccagaacagtaccagatcctgaaggaaaattacgggcagaaggaggctgagaaagtggcccgggtgaaggcgctatatgaggagctggatctgccagcagtgttcttgcaatatgaggaagacagttacagccacattatggctctcattgaacagtacgcagcacccctgcccccagccgtctttctggggcttgcgcgcaaaatctacaagcggagaaagtga

SEQ ID NO: 42 - DNA, 3615 bp, Homo sapiens
  source 1..3615: mol_type = genomic DNA
atgtacgtcacaatgatgatgaccgaccaaatccctctggaactgccaccattgctgaacggagaggtagccatgatgccccacttggtgaatggagatgcagctcagcaggttattctcgttcaagttaatccaggtgagactttcacaataagagcagaggatggaacacttcagtgcattcaaggacctgctgaagttcccatgatgtcacccaatggatccattcctcccattcatgtgcctccaggttatatctcacaggtgattgaagatagtactggagtccgccgggtggtggtcacaccccagtctcctgagtgttatcccccaagctacccctcagccatgtctccaacccatcatctccctccctatctgactcaccatccacattttattcataactcacacacggcttactacccacctgttaccggacctggagatatgccgcctcagttttttccccagcatcatcttccccacacaatatatggtgagcaagaaattataccattttatggaatgtcaacctacatcacccgagaagaccagtacagcaagcctccgcacaaaaaactgaaagaccgccagatcgatcgccagaaccgcctcaacagccctccttcttctatctacaaaagcagctgcacaacagtatacaatggctatgggaagggccatagtggtggaagtggcggaggcggcagcggtagtggtcccggaattaagaaaacagagcgacgagcaagaagcagcccaaagtcgaatgattcagacttgcaagaatatgagttggaagtaaagagggtgcaagacattctttcgggaatagagaaaccacaggtttctaatattcaggcaagagcagttgtgttgtcctgggctccccctgttggactttcctgtggaccccacagtggtctttccttcccctacagttacgaggtggccttatcagacaaaggacgagatggaaaatacaagataatttacagtggagaagaattagaatgtaacctgaaagatcttagaccagcaacagattatcatgtgagggtgtatgccatgtacaattccgtaaagggatcctgctccgagcctgttagcttcaccacccacagctgtgcacccgagtgtcctttcccccctaagctggcacataggagcaaaagttcactaaccctgcagtggaaggcaccaattgacaacggttcaaaaatcaccaactaccttttagagtgggatgagggaaaaagaaatagtggtttcagacagtgcttcttcgggagccagaagcactgcaagttgacaaagctttgtccggcaatggggtacacattcaggctggccgctcgaaacgacattggtaccagtggttatagccaagaggtggtgtgctacacattaggaaatatccctcagatgccttctgcaccaaggctggttcgagctggcatcacatgggtcacgttgcagtggagtaagccagaaggctgttcacccgaggaagtgatcacctacaccttggaaattcaggaggatgaaaatgataaccttttccacccaaaatacactggagaggatttaacctgtactgtgaaaaatctcaaaagaagcacacagtataaattcaggctgactgcttctaatacggaaggaaaaagctgtccaagcgaagttcttgtttgtacgacgagtcctgacaggcctggacctcctaccagaccgcttgtcaaaggcccagttacatctcatggctttagtgtcaaatgggatccccctaaggacaatggtggttcagaaatcctcaagtacttgctagagattactgatggaaattctgaagcgaatcagtgggaagtggcctacagtgggtcggctaccgaatacaccttcacccacttgaaaccaggcactttgtacaaactccgagcatgctgcatcagtaccggcggacacagccagtgttctgaaagtctccctgttcgcacactaagcattgcaccaggtcaatgtcgaccaccgagggttttgggtagaccaaagcacaaagaagtccacttagagtgggatgttcctgcatcggaaagtggctgtgaggtctcagagtacagcgtggagatgacggagcccgaagacgtagcctcggaagtgtaccatggcccagagctggagtgcaccgtcggcaacctgcttcctggaaccgtgtatcgcttccgggtgagggctctgaatgatggagggtatggtccctattctgatgtctcagaaattaccactgctgcagggcctcctggacaatgcaaagcaccttgtatttcttgtacacctgatggatgtgtcttagtgggttgggagagtcctgatagttctggtgctgacatctcagagtacaggttggaatggggagaagatgaagaatccttagaactcatttatcatgggacagacacccgttttgaaataagagacctgttgcctgctgcacagtattgctgtagactacaggccttcaatcaagcaggggcagggccgtacagtgaacttgtcctttgccagacgccagcgtctgcccctgaccccgtctccactctctgtgtcctggaggaggagccccttgatgcctaccctgattcaccttctgcgtgccttgtactgaactgggaagagccgtgcaataacggatctgaaatccttgcttacaccattgatctaggagacactagcattaccgtgggcaacaccaccatgcatgttatgaaagatctccttccagaaaccacctaccggatcagaattcaggctataaatgaaattggagctggaccatttagtcagttcattaaagcaaaaactcggccattaccacccttgcctcctaggctagaatgtgctgctgctggtcctcagagcctgaagctaaaatggggagacagtaactccaagacacatgctgctgaggacattgtgtacacactacagctggaggacagaaacaagaggtttatttcaatctacagaggacccagccacacctacaaggtccagagactgacggaattcacatgctactccttcagaatccaggcagcaagcgaggctggagaagggcccttctcagaaacctataccttcagcacaaccaaaagtgtcccccccaccatcaaagcacctcgagtaacacagttagaaggaaattcatgtgaaattttatgggagacggtaccatcaatgaaaggtgaccctgttaactacattctgcaggtattggttggaagagaatctgagtacaaacaggtgtacaagggagaagaagccacattccaaatctcaggcctccagaccaacacagactacaggttccgcgtatgtgcgtgtcgtcgctgtttagacacctctcaggagctaagcggagccttcagcccctctgcggcttttgtattacaacgaagtgaggtcatgcttacaggggacatggggagcttagatgatcccaaaatgaagagcatgatgcctactgatgaacagtttgcagccatcattgtgcttggctttgcaactttgtccattttatttgcctttatattacagtacttcttaatgaagtaa

SEQ ID NO: 43 - DNA, 2037 bp, Homo sapiens
  source 1..2037: mol_type = genomic DNA
atggccccttcgctctcgcccgggcccgccgccctgcgccgcgcgccgcagctgctgctgctgctgctggccgcggagtgcgcgcttgccgcgctgttgccggcgcgcgaggccacgcagttcctgcggcccaggcagcgccgcgcctttcaggtcttcgaggaggccaagcagggccacctggagagggagtgcgtggaggagctgtgcagccgcgaggaggcgcgggaggtgttcgagaacgaccccgagacggattatttttacccaagatacttagactgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctag

SEQ ID NO: 44 - DNA, 669 bp, Homo sapiens
  source 1..669: mol_type = genomic DNA
atggcagcaaggcccaagctccactatcccaacggaagaggccggatggagtccgtgagatgggttttagctgccgccggagtcgagtttgatgaagaatttctggaaacaaaagaacagttgtacaagttgcaggatggtaaccacctgctgttccaacaagtgcccatggttgaaattgacgggatgaagttggtacagacccgaagcattctccactacatagcagacaagcacaatctctttggcaagaacctcaaggagagaaccctgattgacatgtacgtggaggggacactggatctgctggaactgcttatcatgcatcctttcttaaaaccagatgatcagcaaaaggaagtggttaacatggcccagaaggctataattagatactttcctgtgtttgaaaagattttaaggggtcacggacaaagctttcttgttggtaatcagctgagccttgcagatgtgattttactccaaaccattttagctctagaagagaaaattcctaatatcctgtctgcatttcctttcctccaggaatacacagtgaaactaagtaatatccctacaattaagagattccttgaacctggcagcaagaagaagcctccccctgatgaaatttatgtgagaaccgtctacaacatctttaggccataa

SEQ ID NO: 45 - DNA, 1563 bp, Homo sapiens
  source 1..1563: mol_type = genomic DNA
atgcctggatcacttcctttgaatgcagaagcttgctggccaaaagatgtgggaattgttgcccttgagatctattttccttctcaatatgttgatcaagcagagttggaaaaatatgatggtgtagatgctggaaagtataccattggcttgggccaggccaagatgggcttctgcacagatagagaagatattaactctctttgcatgactgtggttcagaatcttatggagagaaataacctttcctatgattgcattgggcggctggaagttggaacagagacaatcatcgacaaatcaaagtctgtgaagactaatttgatgcagctgtttgaagagtctgggaatacagatatagaaggaatcgacacaactaatgcatgctatggaggcacagctgctgtcttcaatgctgttaactggattgagtccagctcttgggatggacggtatgccctggtagttgcaggagatattgctgtatatgccacaggaaatgctagacctacaggtggagttggagcagtagctctgctaattgggccaaatgctcctttaatttttgaacgagggcttcgtgggacacatatgcaacatgcctatgatttttacaagcctgatatgctatctgaatatcctatagtagatggaaaactctccatacagtgctacctcagtgcattagaccgctgctattctgtctactgcaaaaagatccatgcccagtggcagaaagaggcaaatgataacgattttaccttgaatgattttggcttcatgatctttcactcaccatattgtaaactggttcagaaatctctagctcggatgttgctgaatgacttccttaatgaccagaatagagataaaaatagtatctatagtggcctgaaggcctttggggatgttaagttagaagacacctactttgatagagatgtggagaaggcatttatgaaggctagctctgaactcttcagtcagaaaacaaaggcatctttacttgtatcaaatcaaaatggaaatatgtacacatcttcagtatatggttcccttgcatctgttctagcacagtactcacctcagcatttagcagggaagagaattggagtgttttcttatggttctggtttggctgccactctgtactctcttaaagtcacacaagatgctacaccggggtctgctcttgataaaataacagcaagtttatgtgatcttaaatcaaggcttgattcaagaactggtgtggcacaagatgtcttcgctgaaaacatgaagctcagagaggacacccatcatttggtcaactatattccccagggttcaatagattcactctttgaaggaacgtggtacttagttagggtggatgaaaagcacagaagaacttacgctcggcgtcccactccaaatgatgacactttggatgaaggagtaggacttgtgcattcaaacatagcaactgagcatattccaagccctgccaagaaagtaccaagactccctgctacagcagcagaacctgaagcagcagttattagtaatggggtatggtaa

SEQ ID NO: 46 - DNA, 1893 bp, Homo sapiens
  source 1..1893: mol_type = genomic DNA
atggcggcgggggtggcagcgtggctgccttttgcaagggcagcggctatcgggtggatgcctgtggcctcggggcctatgccggctcccccgaggcaggagaggaaaaggacccaagatgctctcattgtgctgaatgtgagtggcacccgcttccagacgtggcaggacaccctggaacgttacccagacactctactgggcagttctgagagggactttttctaccacccagaaactcagcagtatttctttgaccgtgacccagacatcttccgccacatcctgaatttctaccgcactgggaagctccactatcctcgccacgagtgcatctctgcttacgatgaagaactggccttctttggcctcatcccggaaatcatcggcgactgctgttatgaggagtacaaggatcgcaggcgagagaacgccgagcgcctgcaggacgacgcggataccgacaccgctggggagagcgccttgcccaccatgactgcaaggcagagggtctggagggccttcgagaacccccacaccagcacgatggccctggtgttctactatgtcacggggtttttcattgccgtctctgtcatcgcgaatgtggtggaaacagtgccgtgcggatcaagcccaggtcacattaaagaactgccctgtggagagcggtatgctgtggccttcttctgcttggacacggcctgcgtcatgatcttcacagttgagtatttgcttcgcctggctgcagcgcctagtcgttaccgttttgtgcgtagtgtcatgagtatcatcgacgtggtggccatcctgccttattacattgggctggtgatgacagacaatgaggacgtcagcggagcctttgtcacactccgagtcttccgggtcttcaggatctttaagttttcccgccactctcaaggcctgcgcatcctggggtacacactgaagagttgtgcctcagaattgggcttcttgcttttctcgctcaccatggctatcatcattttcgctacggttatgttctacgcagagaagggctcttcagcaagcaagttcaccagcatccctgcagccttctggtacaccatcgtcaccatgacaacactggggtatggcgacatggtaccaaaaaccatagcagggaagattttcgggtctatctgctcactgagcggagtcttggtcatcgcgctacccgtgcctgtgatcgtgtctaacttcagtcggatctaccaccaaaaccaacgagcggacaaacgaagggcacagaagaaagcgaggctggccaggatccgggcagccaaaagtggaagtgcaaatgcctacatgcagagcaagcggagtgggttactgagcaaccaactgcagtcctcggaggatgaaccggccttcgttagcaaatctggatccagcttcgagacgcaacaccaccacctgcttcactgcctggagaaaaccacgaaccatgagtttgtggatgaacaagtctttgaagaaagctgcatggaagtggccactgttaatcgcccttcaagtcacagcccctccctctcttcccaacaaggagtcaccagcacttgctgctcacggagacacaaaaaaactttccgaatcccaaatgccaatgtgtcaggaagtcatagaggcagcgtgcaagaactcagtacaattcagatcagatgtgtggagagaactccactatccaacagccgatccagcttaaatgccaaaatggaagagtgtgtcaaactaaactgtgaacaaccttacgtgaccacagcaataattagcattccaacacctccagtaaccaccccagaaggcgacgacaggcccgagtctcctgagtattcgggaggaaacatcgtcagggtgtctgctttgtaa

SEQ ID NO: 47 - DNA, 743 bp, Homo sapiens
  source 1..743: mol_type = genomic DNA
gggctcaatttcctaacgctcccctccccatccccatgccacctccacgagcagcggcgtccagcctcctcccgcccgaacgtgctcgaggggcgggcagtcgacctttattgtctggggagcacctggcaggtggcgggcccgtgccctaacgtgtgcgtggtgcccagcttcacaaagcgagcgggcagcacctccttggtccgggaacgcctcagcctggccgtccacatcccaggggtggaaaggtggagagagaaaggggctccggagtcaagagcggggagagagggcgcgcgcgccctcctcctcccggcgggcacagccccccggcattaacacgtcgaaagagcaggggccagacgccgccgggaagaagcgagacccgggcgggcgcgagggaggggaggcgaggaggggcgtggccggcgcgcagagggagggcgctgggaggaggggctgctgctcgccgctcgcggctctgggggctcggctttgccgcgctcgctgcacttgggcgagagctggaacgtggaccagagctcggatcccatcgcagctaccgcgatgagaggcgctcgcggcgcctgggattttctctgcgttctgctcctactgcttcgcgtccagacaggtgggacaccgcggctggcaccccgaccgtgcgactactcggcgaagcctgtgcccgggaggtggtacccgccagggtgcatccggagagaggactgcgggccctcagt

SEQ ID NO: 48 - DNA, 828 bp, Homo sapiens
  source 1..828: mol_type = genomic DNA
atggtcatgctgcaaggagtggtccctctagatgcacacaggtttcaggtggacttccagtgtggctgcagcctgtgtccccggccagatatcgccttccacttcaaccctcgcttccataccaccaagccccatgtcatctgcaacaccctgcatggtggacgctggcaaagggaggcccggtggccccacctggccctgcgaagaggctccagcttcctcatcctctttctcttcgggaatgaggaagtgaaggtgagtgtgaatggacagcactttctccacttccgctaccggctcccactgtctcatgtggacacgctgggtatatttggtgacatcctggtagaggctgttggattcctgaacatcaatccatttgtggagggcagcagagagtacccagctggacatcctttcctgctgatgagccccaggctggaggtgccctgctcacatgctcttccccagggtctctcgcctgggcaggtcatcatagtacggggactggtcttgcaagagccgaagcattttactgtgagcctgagggaccaggctgcccatgctcctgtgacactcagggcctccttcgcagacagaactctggcctggatctcccgctgggggcagaagaaactgatctcagcccccttcctcttttacccccagagattctttgaggtgctgctcctgttccaggagggagggctgaagctggcgctcaatgggcaggggctgggggccaccagcatgaaccagcaggccctggagcagctgcgggagctccggatcagtggaagtgtccagctctactgtgtccactcctga

SEQ ID NO: 49 - DNA, 753 bp, Homo sapiens
  source 1..753: mol_type = genomic DNA
atgggggagaaagtttcggaggcgccagagccggtgccccgcggctgcagtggccacggcagccggactccagcctctgcgctggtcgccgcgtcctctccgggtgcttcctcggccgagtcctcctcgggctcagaaactctgtcggaggaaggggagcccggcggcttctccagagagcatcagccgccgccgccgccgccgttgggaggcaccctgggcgcccgggcgcccgccgcgtgggctccggcaagcgtgctgctggagcgcggagtccttgcgctgccgccgccgcttcccggaggagctgtgccgcccgcgccccggggcagcagcgcgtcccaggaggagcaggacgaggagcttgaccacatattatcccctccacccatgccgtttcggaaatgcagcaacccagatgtggcttctggccctggaaaatcactgaagtataaaagacagctgagtgaggatggaagacagctaaggcgagggagcctgggaggagccctgactgggaggtaccttcttccaaacccggtggcgggacaggcctggccggcctctgcagagacgtccaacctcgtgcgcatgcgcagccaggccctgggccagtcggcgccctcgctcaccgccagcctgaaggagctgagtctccccagaagaggaagtttgatagattcccagaagtggaattgcttggtcaaacgccctgtgtgtccaaatgctgggagaacatcaccccttggatga

SEQ ID NO: 50 - DNA, 1105 bp, Homo sapiens
  source 1..1105: mol_type = genomic DNA
ggagagtctgcgatgtcagagaacggctccttcgccaactgctgcgaggcgggcgggtgggcagtgcgcccgggctggtcgggggctggcagcgcgcggccctccaggacccctcgacctccctgggtggctccagcgctgtccgcggtgctcatcgtcaccaccgccgtggacgtcgtgggcaacctcctggtgatcctctccgtgctcaggaaccgcaagctccggaacgcaggtaatttgttcttggtgagtctggcattggctgacctggtggtggccttctacccctacccgctaatcctcgtggccatcttctatgacggctgggccctgggggaggagcactgcaaggccagcgcctttgtgatgggcctgagcgtcatcggctctgtcttcaatatcactgccatcgccattaaccgctactgctacatctgccacagcatggcctaccaccgaatctaccggcgctggcacacccctctgcacatctgcctcatctggctcctcaccgtggtggccttgctgcccaacttctttgtggggtccctggagtacgacccacgcatctattcctgcaccttcatccagaccgccagcacccagtacacggcggcagtggtggtcatccacttcctcctccctatcgctgtcgtgtccttctgctacctgcgcatctgggtgctggtgcttcaggcccgcaggaaagccaagccagagagcaggctgtgcctgaagcccagcgacttgcggagctttctaaccatgtttgtggtgtttgtgatctttgccatctgctgggctccacttaactgcatcggcctcgctgtggccatcaacccccaagaaatggctccccagatccctgaggggctatttgtcactagctacttactggcttatttcaacagctgcctgaatgccattgtctatgggctcttgaaccaaaacttccgcagggaatacaagaggatcctcttggccctttggaacccacggcactgcattcaagatgcttccaagggcagccacgcggaggggctgcagagcccagctccacccatcattggtgtgcagcaccaggcagatgctctctagcctg

SEQ ID NO: 51 - DNA, 1635 bp, Homo sapiens
  source 1..1635: mol_type = genomic DNA
atgtctgacggtctggataatgaagagaaacccccggctcctccactgaggatgaatagtaacaaccgggattcttcagcactcaaccacagctccaaaccacttcccatggcccctgaagagaagaataagaaagccaggcttcgctctatcttcccaggaggaggggataaaaccaataagaagaaggagaaagagcgcccagagatctctcttccttcagactttgagcatacgattcatgtggggtttgatgcagtcaccggggaattcactggaattccagagcaatgggcacgattactccaaacttccaacataacaaaattggaacagaagaagaacccacaagctgttctagatgttctcaaattctatgattccaaagaaacagtcaacaaccagaaatacatgagctttacatcaggagataaaagtgcacatggatacatagcagcccatccttcgagtacaaaaacagcatctgagcctccattggcccctcctgtgtctgaagaagaagatgaagaggaagaagaagaagaagatgaaaatgagccaccaccagttatcgcaccaagaccagagcatacaaaatcaatctatactcgttctgtggttgaatccattgcttcaccagcagtaccaaataaagaggtcacaccaccctctgctgaaaatgccaattccagtactttgtacaggaacacagatcggcaaagaaaaaaatccaagatgacagatgaggagatcttagagaagctaagaagcattgtgagtgttggggacccaaagaaaaaatacacaagatttgaaaaaattggtcaaggggcatcaggtactgtttatacagcactagacattgcaacaggacaagaggtggccataaagcagatgaaccttcaacagcaacccaagaaggaattaattattaatgaaattctggtcatgagggaaaataagaaccctaatattgttaattatttagatagctacttggtgggtgatgaactatgggtagtcatggaatacttggctggtggctctctgactgatgtggtcacagagacctgtatggatgaaggacagatagcagctgtctgcagagagtgcctgcaagctttggatttcctgcactcaaaccaggtgatccatagagatataaagagtgacaatattcttctcgggatggatggctctgttaaattgactgactttgggttctgtgcccagatcactcctgagcaaagtaaacgaagcactatggtgggaaccccatattggatggcacctgaggtggtgactcgaaaagcttatggtccgaaagttgatatctggtctcttggaattatggcaattgaaatggtggaaggtgaacccccttaccttaatgaaaatccactcagggcattgtatctgatagccactaatggaactccagagctccagaatcctgagagactgtcagctgtattccgtgactttttaaatcgctgtcttgagatggatgtggataggcgaggatctgccaaggagcttttgcagcatccatttttaaaattagccaagcctctctccagcctgactcctctgattatcgctgcaaaggaagcaattaagaacagcagccgctaa

SEQ ID NO: 52 - DNA, 4347 bp, Homo sapiens
  source 1..4347: mol_type = genomic DNA
atggcatattcttggcaaacggatccaaatcctaatgaatcacacgaaaagcagtatgaacaccaagaatttctctttgtaaatcaaccccattcttctagccaagtcagtctgggttttgatcagatagtagatgagatcagtggcaaaattccacactacgagagtgaaattgatgaaaacaccttttttgtgcccactgcaccaaaatgggactcaacagggcattcattaaatgaagcacaccaaatatccttgaatgaattcacttctaaaagccgtgaactctcctggcatcaagttagcaaagcaccagcaattggttttagtccttctgtgttaccaaaacctcaaaatacgaataaagaatgctcctggggaagccccataggaaaacatcatggtgctgatgattccagattcagtattttagctccatcattcacaagtttggataaaattaatctagagaaagaattagaaaatgaaaatcataactaccatataggatttgaaagtagcattcctccaacaaattcatccttctcaagtgacttcatgccgaaagaagagaataaaaggagtggacatgtgaacattgtggaaccatctttgatgcttttgaaaggctctcttcaacccggaatgtgggaaagtacatggcagaagaatatagagtcaataggttgttccattcagctagtggaagtacctcaaagcagcaatacgagtctggcctctttttgcaacaaagtaaaaaaaatcagagaaagatatcatgcagctgatgttaatttcaattctgggaagatctggagcactactacagcatttccgtatcagctcttttctaagaccaagtttaatatacatatttttattgataactcaacacaacctcttcattttatgccatgtgctaattatcttgtcaaagatctaattgcagaaattctgcatttttgcacaaatgaccagctactccccaaagatcatattctaagtgtatgtggctctgaagaatttttacaaaacgaccactgtttggggagccacaaaatgtttcaaaaagataaatctgttattcagctccacctgcagaaaagtagggaagctccaggaaagctatctcgaaagcatgaagaggaccacagtcagttttatctgaatcaacttctagaatttatgcatatttggaaagtatccagacaatgtctcttaacactcatcagaaaatatgacttccacctgaaatacctattgaaaacccaggaaaacgtgtataatattattgaagaagttaaaaaaatatgcagtgttctagggtgtgtggaaaccaaacaaattacagatgcagtaaatgaactaagtctaattcttcagagaaaaggagagaatttttatcaaagttcagagacttcagcaaaaggcttgatagagaaggtaacaactgaactatccacatccatctaccagctaatcaatgtctactgtaacagcttttatgcagattttcagcctgtaaatgtacctagatgcacttcctatctaaatcccgggcttccttcccacctcagcttcacagtgtatgcagcacacaacattccagaaacctgggtgcacaggatcaattttccccttgaaataaagtcacttccaagggaatccatgctcactgtaaaactgtttgggattgcctgtgcaaccaacaatgcaaatttactggcgtggacttgtcttccactgtttccaaaagaaaaatccattctcgggtctatgctgttcagcatgacattacagagtgagcctcccgtagaaatgataactccaggagtgtgggatgtaagtcagccatccccggtgaccctgcagattgattttccagctactgggtgggagtatatgaaacctgattctgaagagaatagaagtaatcttgaagagccactaaaggagtgtataaaacatattgccagactttcacagaaacagactcccctactactctctgaagaaaagaaaagatatttatggttttatcgcttctactgcaataatgaaaactgctcccttcctttagtcctgggtagtgcccctggatgggatgaaaggactgtttcagaaatgcataccattttgagaagatggacattttctcaacctttagaggctcttgggcttttgacttccagttttccagatcaagaaattcgtaaagtggcagttcaacaattagacaacctcttgaatgatgaactactggaatatctcccacagctagttcaggctgtcaagtttgaatggaaccttgagagtcctttagtgcaacttctactccaccgctccttgcagagcatccaggttgcccatcgtctttactggctgctaaaaaatgcagaaaatgaagcttattttaaaagctggtatcagaagctactagctgctctccaattctgtgcaggtaaagccttgaatgatgagttttccaaggagcagaaacttatcaaaattctgggagatattggggaaagagtcaagtctgccagtgaccatcaaagacaggaggtactgaagaaagaaattggcagactagaagagttctttcaagatgtaaatacttgtcatcttcctctgaaccctgccctatgtataaaagggattgatcacgatgcatgttcatattttacatctaatgctttgccattgaagattactttcatcaatgctaatctgatgggcaaaaacatcagcattatttttaaggctggagatgatcttcgtcaggatatgcttgttctgcagcttattcaagtgatggacaatatttggctgcaggaaggcttggatatgcaaatgatcatttatagatgtctatccacaggaaaagaccaacgattggtgcagatggtacctgatgctgtgaccctagcaaagattcatcgccattctggactgataggaccattgaaagaaaataccattaaaaagtggttcagtcagcacaaccacttaaaggcagattatgaaaaggccttgaggaactttttctactcctgtgctggctggtgtgtggtaacattcatcctgggagtatgtgaccgtcacaatgataatatcatgctgacaaagtcgggccacatgtttcatattgactttggaaaattcttaggtcatgcacaaacatttggagggataaaaagggaccgagctcctttcatttttacttcagagatggaatactttattacagagggtgggaaaaacccacagcattttcaagattttgtggaactttgctgtcgtgcttataatattatcagaaagcacagccaactgctcttgaacctgctggaaatgatgctgtatgcaggactgcctgagctaagtggaattcaagacctgaaatatgtgtataataatcttcgtccacaagacacagacctggaagcaacaagtcattttaccaagaaaataaaggaaagtctggagtgtttccctgttaaattgaataacttgatccacacacttgcacaaatgtcagccataagccctgccaaatctacttcacagacttttcctcaggaatcctgtttgctgagtacaactaggtcgattgaaagagcaattttagggttcagcaagaaatccagtaatctgtatctgatccaggtgacacacagcaacaacgaaacaagcctgacagaaaaatcatttgagcagttttcaaaacttcacagccaacttcagaagcagtttgcatcactgactctcccagagtttcctcattggtggcacctaccttttacaaattcagatcacagaagattcagagatctaaatcattacatggaacagatattaaatgtatcacatgaagttacaaacagtgattgtgtacttagctttttcctctctgaggctgtgcaacaaacagttgaatcatcacctgtgtacctaggtgagaagaagtttccagacaagaagcctaaggtgcagttagtcatatcctacgaggatgtgaagctgaccatactagtgaaacacatgaaaaacattcatctcccagatggctctgcgcccagtgcacatgttgaattttatcttttaccatatcccagtgaagttctgaggaggaaaacaaaatctgttccaaaatgtacggaccccacttacaatgaaattgtagtatatgatgaagtcacagagctccaaggacatgtcttaatgcttattgtgaagagtaaaactgtatttgtgggagcaattaacatccgactctgtagtgtcccactcgataaagaaaaatggtatccattaggaaacagtataatttcaccattgctatga

SEQ ID NO: 53 - DNA, 447 bp, Homo sapiens
  source 1..447: mol_type = genomic DNA
atggcccgggccctgtgccgcctcccgcggcgcggcctctggctgctcctggcccatcacctcttcatgaccactgcctgccaggaggctaactacggtgccctcctccgggagctctgcctcacccagttccaggtagacatggaggccgtcggggagacgctgtggtgtgactggggcaggaccatcaggagctacagggagctggccgactgcacctggcacatggcggagaagctgggctgcttctggcccaatgcagaggtggacaggttcttcctggcagtgcatggccgctacttcaggagctgccccatctcaggcagggccgtgcgggacccgcccggcagcatcctctaccccttcatcgtggtccccatcacggtgaccctgctggtgacggcactggtggtctggcagagcaagcgcactgagggcattgtgtag

SEQ ID NO: 54 - DNA, 1341 bp, Homo sapiens
  source 1..1341: mol_type = genomic DNA
atgctgctcccccagctctgctggctgccgctgctcgctgggctgctcccgccggtgcccgcgcagaagttctcggcgctcacgtttttgagagtggatcaagataaagacaaggattgtagcttggactgtgcgggttcgccccagaaacctctctgcgcatctgacggaaggaccttcctttcccgttgtgaatttcaacgtgccaagtgcaaagatccccagctagagattgcatatcgaggaaactgcaaagacgtgtccaggtgtgtggccgaaaggaagtatacccaggagcaagcccggaaggagtttcagcaagtgttcattcctgagtgcaatgacgacggcacctacagtcaggtccagtgtcacagctacacgggatactgctggtgcgtcacgcccaacgggaggcccatcagcggcactgccgtggcccacaagacgccccggtgcccgggttccgtaaatgaaaagttaccccaacgcgaaggcacaggaaaaacagatgatgccgcagctccagcgttggagactcagcctcaaggagatgaagaagatattgcatcacgttaccctaccctttggactgaacaggttaaaagtcggcagaacaaaaccaataagaattcagtgtcatcctgtgaccaagagcaccagtctgccctggaggaagccaagcagcccaagaacgacaatgtggtgatccctgagtgtgcgcacggcggcctctacaagccagtgcagtgccacccctccacggggtactgctggtgcgtcctggtggacacggggcgccccattcccggcacatccacaaggtacgagcagccgaaatgtgacaacacggccagggcccacccagccaaagcccgggacctgtacaagggccgccagctacaaggttgtccgggtgccaaaaagcatgagtttctgaccagcgttctggacgcgctgtccacggacatggtccacgccgcctccgacccctcctcctcgtcaggcaggctctcagaacccgaccccagccataccctagaggagcgggtggtgcactggtacttcaaactactggataaaaactccagtggagacatcggcaaaaaggaaatcaaacccttcaagaggttccttcgcaaaaaatcaaagcccaaaaaatgtgtgaagaagtttgttgaatactgtgacgtgaataatgacaaatccatctccgtacaagaactgatgggctgcctgggcgtggcgaaagaggacggcaaagcggacaccaagaaacgccacacccccagaggtcatgttgaaagtacgtctaatagacagccaaggaaacaaggataa

SEQ ID NO: 55 - DNA, 390 bp, Homo sapiens
  source 1..390: mol_type = genomic DNA
atgaaaatcctcgtggccttggcagtcttttttcttgtctccactcagctgtttgcagaagaaataggagccaatgatgatctgaattactggtccgactggtacgacagcgaccagatcaaggaggaactgccggagccctttgagcatcttctgcagagaatcgcccggagacccaagcctcagcagttctttggattaatgggcaaacgggatgctgattcctcaattgaaaaacaagtggccctgttaaaggctctttatggacatggccagatctctcacaaaagacataaaacagattcctttgttggactaatgggcaaaagagctttaaattctgtggcttatgaaaggagtgcaatgcagaattatgaaagaagacgttaa

SEQ ID NO: 56 - DNA, 624 bp, Homo sapiens
  source 1..624: mol_type = genomic DNA
atggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagtttcgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctga

SEQ ID NO: 57 - DNA, 135 bp, Homo sapiens
  source 1..135: mol_type = genomic DNA
atgtctgacaaacccgatatggctgagatcgagaaattcgataagtcgaaactgaagaagacagagacgcaagagaaaaatccactgccttccaaagaaacgattgaacaggagaagcaagcaggcgaatcgtaa

SEQ ID NO: 58 - DNA, 747 bp, Homo sapiens
  source 1..747: mol_type = genomic DNA
atggccggcctcaactccctggaggcggtgaaacgcaagatccaggccctgcagcagcaggcggacgaggcggaagaccgcgcgcagggcctgcagcgggagctggacggcgagcgcgagcggcgcgagaaagctgaaggtgatgtggccgccctcaaccgacgcatccagctctttgaggaggagttggacagggctcaggaacgactggccacggccctgcagaagctggaggaggcagaaaaagctgcagatgagagtgagagaggaatgaaggtgatagaaaaccgggccatgaaggatgaggagaagatggagattcaggagatgcagctcaaagaggccaagcacattgcggaagaggctgaccgcaaatacgaggaggtagctcgtaagctggtcatcctggagggtgagctggagagggcagaggagcgtgcggaggtgtctgaactaaaatgtggtgacctggaagaagaactcaagaatgttactaacaatctgaaatctctggaggctgcatctgaaaagtattctgaaaaggaggacaaatatgaagaagaaattaaacttctgtctgacaaactgaaagaggctgagacccgtgctgaatttgcagagagaacggttgcaaaactggaaaagacaattgatgacctggaagagaaacttgcccaggccaaagaagagaacgtgggcttacatcagacactggatcagacactaaacgaacttaactgtatataa

SEQ ID NO: 59 - DNA, 1032 bp, Homo sapiens
  source 1..1032: mol_type = genomic DNA
atgaacatacacaggtctacccccatcacaatagcgagatatgggagatcgcggaacaaaacccaggatttcgaagagttgtcgtctataaggtccgcggagcccagccagagtttcagcccgaacctcggctccccgagcccgcccgagactccgaacttgtcgcattgcgtttcttgtatcgggaaatacttattgttggaacctctggagggagaccacgtttttcgtgccgtgcatctgcacagcggagaggagctggtgtgcaaggtgtttgatatcagctgctaccaggaatccctggcaccgtgcttttgcctgtctgctcatagtaacatcaaccaaatcactgaaattatcctgggtgagaccaaagcctatgtgttctttgagcgaagctatggggacatgcattccttcgtccgcacctgcaagaagctgagagaggaggaggcagccagactgttctaccagattgcctcggcagtggcccactgccatgacggggggctggtgctgcgggacctcaagctgcggaaattcatctttaaggacgaagagaggactcgggtcaagctggaaagcctggaagacgcctacattctgcggggagatgatgattccctctccgacaagcatggctgcccggcttacgtaagcccagagatcttgaacaccagtggcagctactcgggcaaagcagccgacgtgtggagcctgggggtgatgctgtacaccatgttggtggggcggtaccctttccatgacattgaacccagctccctcttcagcaagatccggcgtggccagttcaacattccagagactctgtcgcccaaggccaagtgcctcatccgaagcattctgcgtcgggagccctcagagcggctgacctcgcaggaaattctggaccatccttggttttctacagattttagcgtctcgaattcagcatatggtgctaaggaagtgtctgaccagctggtgccggacgtcaacatggaagagaacttggaccctttctttaactga

SEQ ID NO: 60 - DNA, 1401 bp, Homo sapiens
  source 1..1401: mol_type = genomic DNA
atgtccaccaggtccgtgtcctcgtcctcctaccgcaggatgttcggcggcccgggcaccgcgagccggccgagctccagccggagctacgtgactacgtccacccgcacctacagcctgggcgacgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctaggggacctctacgaggaggagatgcgggagctgcgccggcaggtggaccagctaaccaacgacaaagcccgcgtcgaggtggagcgcgacaacctggccgaggacatcatgcgcctccgggaaaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacattcctgattaagacggttgaaactagagatggacaggttatcaacgaaacttctcagcatcacgatgaccttgaataa

SEQ ID NO: 61 - DNA, 954 bp, Homo sapiens
  source 1..954: mol_type = genomic DNA
atgagcagcgaggccgagacccagcagccgcccgccgccccccccgccgcccccgccctcagcgccgccgacaccaagcccggcactacgggcagcggcgcagggagcggtggcccgggcggcctcacatcggcggcgcctgccggcggggacaagaaggtcatcgcaacgaaggttttgggaacagtaaaatggttcaatgtaaggaacggatatggtttcatcaacaggaatgacaccaaggaagatgtatttgtacaccagactgccataaagaagaataaccccaggaagtaccttcgcagtgtaggagatggagagactgtggagtttgatgttgttgaaggagaaaagggtgaggaggcagcaaatgttacaggtcctggtggtgttccagttcaaggcagtaaatatgcagcagaccgtaaccattatagacgctatccacgtcgtaggggtcctccacgcaattaccagcaaaattaccagaatagtgagagtggggaaaagaacgagggatcggagagtgctcccgaaggccaggcccaacaacgccggccctaccgcaggcgaaggttcccaccttactacatgcggagaccctatgggcgtcgaccacagtattccaaccctcctgtgcagggagaagtgatggagggtgctgacaaccagggtgcaggagaacaaggtagaccagtgaggcagaatatgtatcggggatatagaccacgattccgcaggggccctcctcgccaaagacagcctagagaggacggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccagctgagaattcccgctcccgaggctga

SEQ ID NO: 62 - DNA, 3630 bp, Homo sapiens
  source 1..3630: mol_type = genomic DNA
atggagtgttactacattgtcatcagctccacgcatctcagcaacggacactttcgcaacatcaagggagttttccggggccctctcagcaagaacgggaacaaaactctggactatgctgagaaggaaaataccatagcaaaagctctggaagatctgaaggcaaatttttactgtgaactctgtgacaagcagtactataagcaccaggagtttgacaatcacattaattcatatgaccatgctcacaagcagaggctcaaggaactgaaacaaagggaatttgctcgaaatgtagcatctaaatccaggaaagatgaaagaaaacaggaaaaggcactccaacgcctgcacaagctggctgagctaagaaaggaaactgtatgtgctcctggaagtggccccatgttcaaatcaacaactgttactgtgagagaaaactgtaatgaaatttcccaacgagttgttgtggattcagttaataaccagcaagatttcaaatatactttgattcatagtgaagagaatactaaagatgctaccactgttgctgaagatccagaaagtgcaaataattatacagcaaaaaataaccaagttggggatcaagcccaggggattcacagacacaaaatcggcttttcttttgcatttccaaagaaagcgtccgtgaagctagagtcctcagctgcagccttctctgaatacagtgatgatgcctcagtgggaaaaggatttagcagaaaaagtagatttgtccccagtgcttgtcatcttcaacaatcttcaccaacagatgtgcttttgagttctgaggagaaaactaactcttttcatccaccagaggcaatgtgcagagacaaagaaactgttcaaactcaagagataaaagaagtctctagtgaaaaagatgcattattattaccttcattttgcaagtttcaacttcagttatcttctgatgcagataattgtcaaaattcagtcccattagcagatcaaataccactagagagtgttgttattaatgaagacatacctgttagtggtaacagttttgagttgttaggaaataaatccacagttcttgacatgtctaatgattgcatatctgtgcaagctaccacagaggaaaatgttaagcataacgaggcatccacaactgaggttgaaaataaaaatggtcccgagacattggccccttcaaatactgaagaggttaacataactatacataagaaaacaaatttctgcaaaagacaatgtgagccatttgtacctgtccttaacaaacacagatctacagttcttcagtggccatcagaaatgctggtttatacaactacgaaaccatcaatttcctatagctgtaatcctctatgttttgacttcaagtctactaaagtaaataataatctagataaaaataagccagacttaaaagatctttgttctcagcagaagcaggaagacatttgcatgggaccactttcagattacaaggatgtatctacagaaggactcactgattatgaaattggaagtagcaaaaataaatgcagccaagtcactcctcttttggctgatgatattctctccagtagttgtgattctggaaaaaatgagaacacaggtcagaggtataaaaacatttcctgtaagatcagagaaacagaaaagtataattttactaaaagtcaaataaaacaggacactctagatgaaaaatacaacaaaataaggttgaaagagacccatgaatactggttccataaaagtagaagaaagaaaaaaagaaaaaagttatgtcagcatcatcatatggagaaaaccaaagaatcagaaactcgctgcaaaatggaagcagagaatagttacactgaaaatgctgggaaatatctattggaaccaatttcagaaaagcagtatttagctgcagagcaattattagactcacatcagttacttgataaaaggcccaaatcagaatccatatccttaagtgacaatgaagaaatgtgtaaaacatggaatactgaatacaacacttatgatactatcagttctaaaaaccactgtaaaaagaacacaatacttttaaatggacaatcaaatgcaacaatgatacattctgggaaacataatttaacatattctagaacttactgttgttggaaaaccaaaatgtcaagctgtagtcaggatcacagaagcttagttcttcaaaatgatatgaaacacatgagtcagaatcaggctgttaaaagaggttacaattctgtcatgaatgaatcagaaagattctatcgaaaacgtagacaacattcacattcttattcttcagatgaaagtttaaatcgacagaatcatttaccagaagaatttttgaggccaccaagtacttcagttgctccctgcaagcctaaaaagaaacggaggcgaaaaagaggcagattccaccccggatttgaaactttagaactcaaagaaaatacagattatcccgtgaaagacaattcttccttaaatcctctggataggttaataagtgaagacaaaaaagagaaaatgaaaccacaagaagttgcaaaaatcgaaaggaactcagaacaaacaaaccaattaagaaacaaactgtctttccaccctaacaatctccttccttctgaaaccaatggtgaaactgagcatttagaaatggagaccacttctggtgaattgtcagatgtttccaatgatcccaccacatctgtctgtgtagctagtgccccaacaaaagaagcaattgacaataccctgcttgaacacaaagaaagaagtgagaatataaatcttaatgaaaagcaaattccttttcaggtgcctaatattgaaaggaactttagacagtcacagcctaaatcctatctttgccattatgaactggctgaggcccttccacaaggaaagatgaatgagacaccaactgagtggctgcgttataattcaggaatccttaacacacaaccaccattaccattcaaagaagcacatgtcagtggtcatacttttgtaacagctgagcaaatcctggctccattagctttaccagagcaagcattattgatcccactagaaaaccatgacaaattcaaaaatgtaccatgtgaggtctaccagcacattctgcagccaaacatgctggccaacaaggttaaatttacctttcctccagctgccctcccaccccctagcacacctctgcagcctttgcctttgcagcagtccttatgttctacctctgtaaccactatccatcacactgttttgcagcagcacgctgcagctgctgcagctgcagctgcagccgcagctgcaggaacctttaaagtgcttcagccacaccaacagtttctttcccaaatcccagctctcaccagaacctcattacctcagctctcagtaggaccagtaggaccgaggctttgtcctgggaaccagccaacttttgttgctcctcctcagatgccaatcattccagcttccgttcttcatcctagccatctggctttcccatctttaccccatgcactctttccttcactgctttccccacaccctactgtcatccctttgcaacctctcttctag

SEQ ID NO: 63 - protein, 6 aa, synthetic construct
  SITE 3: note = Hydroxyproline
GFXGER

(19) United States
(12) Patent Application Publication
(10) Pub. No.: US 2026/0034249 A1
(43) Pub. Date: Feb. 5, 2026

(54) COMPOSITIONS AND METHODS FOR MODIFYING FERTILITY

(71) Applicants: The Broad Institute, Inc., Cambridge, MA (US); Trustees of Dartmouth College, Hanover, NH (US); Northwestern University, Evanston, IL (US); The General Hospital Corporation, Boston, MA (US); Massachusetts Institute of Technology, Cambridge, MA (US)

(72) Inventors: Brittany Thomas, Hanover, NH (US); Ruixu Huang, Hanover, NH (US); Caroline E. Kratka, Evanston, IL (US); Yiru Zhu, Evanston, IL (US); Samouil L. Farhi, Cambridge, MA (US); Daniela D. Russo, Boston, MA (US); Cai L. S. McCann, Cambridge, MA (US); Alexander K. Shalek, Cambridge, MA (US); Francesca Elizabeth Duncan, Evanston, IL (US); Hoi Chang Lee, Evanston, IL (US); Luhan Tracy Zhou, Evanston, IL (US); Emily Zaniker, Evanston, IL (US); Jack Nelson, Cambridge, MA (US)

(73) Assignees: The Broad Institute, Inc., Cambridge, MA (US); Trustees of Dartmouth College, Hanover, NH (US); Northwestern University, Evanston, IL (US); The General Hospital Corporation, Boston, MA (US); Massachusetts Institute of Technology, Cambridge, MA (US)

(21) Appl. No.: 19/363,303

(22) Filed: Oct. 20, 2025

Related U.S. Application Data
(63) Continuation of application No. PCT/US2024/025824, filed on Apr. 23, 2024.
(60) Provisional application No. 63/497,951, filed on Apr. 24, 2023.

(51) Int. Cl. A61K 48/00

(52) U.S. Cl. CPC A61K 48/0058

(57) ABSTRACT

The present disclosure features methods for modifying fertility. In some embodiments, the disclosure provides contraceptive compositions and methods of using the same.

### CROSS-REFERENCE TO RELATED APPLICATIONS

This application is a continuation under 35 U.S.C. § 111(a) of PCT International Patent Application No. PCT/US2024/025824, filed Apr. 23, 2024, designating the United States and published in English, which claims priority to and the benefit of U.S. Provisional Application No. 63/497,951, filed Apr. 24, 2023, the entire contents of each of which are incorporated by reference herein.

### STATEMENT OF RIGHTS TO INVENTIONS MADE UNDER FEDERALLY SPONSORED RESEARCH

This invention was made with government support under Grant No. 1S100D025120 awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on Apr. 23, 2024, is named 167741-051501PCT_SL.xml and is 115,776 bytes in size.

### BACKGROUND

About 40% of women in low- and middle-income countries who use a contraceptive method stop within the first year of use due to unpleasant side-effects or issues of convenience. For example, breakthrough uterine bleeding and spotting cause unnecessary anxiety, taking a daily medication may be inconvenient, and the need to travel to a clinic every three months for a new prescription can be an overwhelming burden for many women. It's clear that current contraception options are not working for many women. Accordingly, improved compositions and methods of controlling ovulation are urgently required.

### SUMMARY

As described below, the present disclosure features compositions and methods for altering ovulatory processes (e.g., follicle activation and/or development) in a subject using non-hormonal agents. In particular embodiments, the methods involve administering to a female subject an agent that selectively reduces or eliminates the expression and/or activity of a target polypeptide and/or selectively kills and/or reduces the development, proliferation, or metabolism of a cell in the ovary of the female subject. In some embodiments, the methods involve administering to a female subject an agent that selectively increases the expression and/or activity of a target polypeptide and/or selectively increases the development, proliferation, or metabolism of a cell in an ovary of the female subject. In various embodiments, the method is a contraceptive method. In one aspect, the disclosure provides a method of reducing or eliminating expression or activity of a polypeptide encoded by a gene related to ovulation. In various embodiments of any of the aspects delineated herein, an agent is administered whereby ovulation-related cells are selectively killed or rendered non-functional.

In one aspect, the present disclosure provides a method for altering ovulation in a female subject, the method involves administering to the subject an agent that selectively modulates the expression and/or activity in an ovary of the subject of a polypeptide encoded by a gene selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17b1, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9, thereby altering ovulation in the female subject.

In another aspect, the present disclosure provides a method for reducing or eliminating ovulation in a female subject. The method involves administering to the subject an agent that selectively disrupts the development or function of a cell in an ovary of the subject. The cell is selected from one or more of cumulus cells, endothelial cells, epithelial cells, granulosa cells, luteal cells, myeloid cells, oocyte cells, stroma cells, and theca cells. The agent selectively modulates the expression and/or activity in an ovary of the subject of a polypeptide encoded by a gene selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Den, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmges1, Hsd17b1, Hsd3b1, Ildr2, Kend2, Kdr, Kit, Krt18, Krt19, Krt7, Laptm5, Lgals1, Lgals7, Lhegr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptpre, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9.

In another aspect, the present disclosure provides a method for altering fertility in a female subject. The method involves administering to the subject an agent that selectively increases the expression and/or activity in an ovary of the subject of a polypeptide encoded by a gene selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Den, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmges1, Hsd17b1, Hsd3b1, Ildr2, Kend2, Kdr, Kit, Krt18, Krt19, Krt7, Laptm5, Lgals1, Lgals7, Lhegr, Lox, Lum, Lyz2, Mast4, Mgp, Mt2, Nap115, Nppc, Nts, Nuprl, Ogn, Onecut2, Pak3, Parm1, Pdgfra, Pecam1, Pgr, Pik3c2g, Plxna4, Ptgs2, Ptpre, Ptx3, Rampl, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9, thereby increasing fertility in the female subject.

In another aspect, the present disclosure provides a method for reducing or eliminating ovulation in a female subject. The method involves administering to the subject a non-hormonal agent that selectively modulates the expression and/or activity in an ovary of the subject of a polypeptide encoded by a gene selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Den, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmges1, Hsd17b1, Hsd3b1, Ildr2, Kend2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9. The agent selectively kills and/or reduces the development, proliferation, or metabolism of a cell in an ovary of the subject. The cell is selected from one or more of cumulus cells, endothelial cells, epithelial cells, granulosa cells, luteal cells, myeloid cells, oocyte cells, stroma cells, and theca cells.

In any aspect of the disclosure delineated herein, or embodiments thereof, the agent includes a polynucleotide. In various embodiments, the polynucleotide encodes or includes an inhibitory nucleic acid molecule. In various embodiments, the method includes administering to the subject a vector containing the polynucleotide. In various embodiments, the polynucleotide encodes or includes an inhibitory nucleic acid molecule. In any aspect of the disclosure delineated herein, or embodiments thereof, the polynucleotide encodes the polypeptide.

In any aspect of the disclosure delineated herein, or embodiments thereof, the gene is selected from one or more of Sdc1, Pgr, Spp1, Frmd5, Chchd10, Spsb1, Tspo, Gm10076, and Rnfl80. In any aspect of the disclosure delineated herein, or embodiments thereof, the gene is selected from one or more of Tac1, Gsta4, Mast4, F3, Kcnd2, Timp1, Pik3c2g, Fdps, Lgals1, Ramp1, and Emb. In any aspect of the disclosure delineated herein, or embodiments thereof, the gene is selected from one or more of Sdc1, Hsd3b1, Onecut2, Cnn3, Rplp1, Sox5, Frmd5, Cst8, Aebp1, and Rpl13a. In any aspect of the disclosure delineated herein, or embodiments thereof, the gene is selected from one or more of Tmsb4x, Timp1, Ybx1, Vim, Col4a1, Gas6, Pak3, Trib2, Smoc2, Kit, Tpm4, Fndc3b, Cnn3, and Zfp804a. In any aspect of the disclosure delineated herein, or embodiments thereof, the gene is selected from one or more of Sdc1, Akrc1, Star, Fdx1, Scarb1, Acsbg1, Ybx1, Gas6, Cobll1, and Acly. In any aspect of the disclosure delineated herein, or embodiments thereof, the gene is selected from one or more of Tmsb4x, Timp1, Ybx1, Mt2, Ctsl, Cst8, Bst2, Aebp1, Bace2, and Hmgcs1. In any aspect of the disclosure delineated herein, or embodiments thereof, the agent is selected from those agents listed in Tables 2-7.

In any aspect of the disclosure delineated herein, or embodiments thereof, the subject is a mammal. In embodiments, the mammal is a human.

In any aspect of the disclosure delineated herein, or embodiments thereof, the method reduces follicle activation or maturation in the ovary. In any aspect of the disclosure delineated herein, or embodiments thereof, the method reduces or increases conception in the female subject. In any aspect of the disclosure delineated herein, or embodiments thereof, the method increases pregnancy in the female subject. In any aspect of the disclosure delineated herein, or embodiments thereof, the method increases follicle activation or maturation in the ovary. In any aspect of the disclosure delineated herein, or embodiments thereof, the method reduces the occurrence of pregnancy in the female subject.

In any aspect of the disclosure delineated herein, or embodiments thereof, the agent includes a small molecule compound. In various embodiments, the small molecule compound reduces an activity of the polypeptide in a cell. In various embodiments, the small molecule compound specifically reduces an activity of the polypeptide in a cell. In various embodiments, the small molecule compound selectively increases proliferation and/or mediates development of the cells. In various embodiments, the small molecule compound selectively prevents proliferation of the cells. In various embodiments, the small molecule selectively kills the cells. In various embodiments, the small molecule selectively prevents development of the cells.

In any aspect of the disclosure delineated herein, or embodiments thereof, the agent includes a polypeptide. In various embodiments, the polypeptide contains an antibody that specifically binds the polypeptide. In various embodiments, the polypeptide contains an antibody that specifically binds the gene.

In any aspect of the disclosure delineated herein, or embodiments thereof, the cells include cumulus cells. In any aspect of the disclosure delineated herein, or embodiments thereof, the cells include luteal cells, stroma cells, and/or thecal cells.

Compositions and articles defined by the disclosure were isolated or otherwise manufactured in connection with the examples provided below. Other features and advantages of the disclosure will be apparent from the detailed description, and from the claims.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Tables A and B below provide exemplary polynucleotide and polypeptide sequences for target genes of the disclosure and the polypeptides encoded thereby, respectively. In various embodiments, a polypeptide encoded by a target gene of the disclosure comprises a sequence having at least 85% identity to a sequence listed in Table A, or a fragment thereof having a function listed in any one of Tables 2 to 7 for the encoded polypeptide. In some embodiments, a target gene of the disclosure encodes one or more of the polypeptides listed in Table A, or a fragment thereof having a function listed in any one of Tables 2 to 7 for the encoded polypeptide. In various embodiments, a target gene of the disclosure comprises a sequence having at least 85% identity to a sequence listed in Table B.

TABLE A
Exemplary polypeptide sequences encoded by representative
target genes of the disclosure.

| Gene | |
|---|---|
| name | Exemplary polypeptide sequence |
| Aebp1 | >BAA13094.1 AEBP1 [Homo sapiens] |
| | MDYYFGPPPPQKPDAERQTDEEKEELKKPKKEDSSPKEETDKWAVEKGKDHKEPRKGEE |
| | LEEEWTPTEKVKCPPIGMESHRIEDNQIRASSMLRHGLGAQRGRLNMQTGATEDDYYDG |
| | AWCAEDDARTQWIEVDTRRTTRFTGVITQGRDSSIHDDFVTTFFVGFSNDSQTWVMYTN |
| | GYEEMTFHGNVDKDTPVLSELPEPVVARFIRIYPLTWNGSLCMRLEVLGCSVAPVYSYY |
| | AQNEVVATDDLDFRHHSYKDMRQLMKVVNEECPTITRTYSLGKSSRGLKIYAMEISDNP |
| | GEHELGEPEFRYTAGIHGNEVLGRELLLLLMQYLCREYRDGNPRVRSLVQDTRIHLVPS |
| | LNPDGYEVAAQMGSEFGNWALGLWTEEGFDIFEDFPDLNSVLWGAEERKWVPYRVPNNN |
| | LPIPERYLSPDATVSTEVRAIIAWMEKNPFVLGANLNGGERLVSYPYDMARTPTQEQLL |
| | AAAMAAARGEDEDEVSEAQETPDHAIFRWLAISFASAHLTLTEPYRGGCQAQDYTGGMG |
| | IVNGAKWNPRTGTINDFSYLHTNCLELSFYLGCDKFPHESELPREWENNKEALLTFMEQ |
| | VHRGIKGVVTDEQGIPIANATISVSGINHGVKTASGGDYWRILNPGEYRVTAHAEGYTP |
| | SAKTCNVDYDIGATQCNFILARSNWKRIREIMAMNGNRPIPHIDPSRPMTPQQRRLQQR |
| | RLQHRLRLRAQMRLRRLNATTTLGPHTVPPTLPPAPATTLSTTIEPWGLIPPTTAGWEE |
| | SETETYTEVVTEFGTEVEPEFGTKVEPEFETQLEPEFETQLEPEFEEEEEEEKEEEIAT |
| | GQAFPFTTVETYTVNFGDF |
| Bace2 | >AAD45240.1 aspartic-like protease [Homo sapiens] |
| | MGALARALLLPLLAQWLLRAAPELAPAPFTLPLRVAAATNRVVAPTPGPGTPAERHADG |
| | LALALEPALASPAGAANFLAMVDNLQGDSGRGYYLEMLIGTPPQKLQILVDTGSSNFAV |
| | AGTPHSYIDTYFDTERSSTYRSKGFDVTVKYTQGSWTGFVGEDLVTIPKGFNTSFLVNI |
| | ATIFESENFFLPGIKWNGILGLAYATLAKPSSSLETFFDSLVTQANIPNVFSMQMCGAG |
| | LPVAGSGTNGGSLVLGGIEPSLYKGDIWYTPIKEEWYYQIEILKLEIGGQSLNLDCREY |
| | NADKAIVDSGTTLLRLPQKVFDAVVEAVARASLIPEFSDGFWTGSQLACWTNSETPWSY |
| | FPKISIYLRDENSSRSFRITILPQLYIQPMMGAGLNYECYRFGISPSTNALVIGATVME |
| | GFYVIFDRAQKRVGFAASPCAEIAGAAVSEISGPFSTEDVASNCVPAQSLSEPILWIVS |
| | YALMSVCGAILLVLIVLLLLPFRCORRPRDPEVVNDESSLVRHRWK |
| Bst2 | >BAA05679.1 BST-2 [Homo sapiens] |
| | MASTSYDYCRVPMEDGDKRCKLLLGIGILVLLIIVILGVPLIIFTIKANSEACRDGLRA |
| | VMECRNVTHLLQQELTEAQKGFQDVEAQAATCNHTVMALMASLDAEKAQGQKKVEELEG |
| | EITTLNHKLQDASAEVERLRRENQVLSVRIADKKYYPSSQDSSSAAAPQLLIVLLGLSA |
| | LLQ |
| Cnn3 | >NP_001830.1 calponin-3 isoform 1 [Homo sapiens] |
| | MTHFNKGPSYGLSAEVKNKIASKYDHQAEEDLRNWIEEVTGMSIGPNFQLGLKDGIILC |
| | ELINKLQPGSVKKVNESSLNWPQLENIGNFIKAIQAYGMKPHDIFEANDLFENGNMTQV |
| | QTTLVALAGLAKTKGFHTTIDIGVKYAEKQTRRFDEGKLKAGQSVIGLQMGTNKCASQA |
| | GMTAYGTRRHLYDPKMQTDKPFDQTTISLQMGTNKGASQAGMLAPGTRRDIYDQKLTLQ |
| | PVDNSTISLQMGTNKVASQKGMSVYGLGRQVYDPKYCAAPTEPVIHNGSQGTGTNGSEI |
| | SDSDYQAEYPDEYHGEYQDDYPRDYQYSDQGIDY |
| Col4a1 | >AAA53098.1 alpha-1 type IV collagen [Homo sapiens] |
| | MGPRLSVWLLLLPAALLLHEEHSRAAAKGGCAGSGCGKCDCHGVKGQKGERGLPGLQGV |
| | IGFPGMQGPEGPQGPPGQKGDTGEPGLPGTKGTRGPPGASGYPGNPGLPGIPGQDGPPG |
| | PPGIPGCNGTKGERGPLGPPGLPGFAGNPGPPGLPGMKGDPGEILGHVPGMLLKGERGF |
| | PGIPGTPGPPGLPGLQGPVGPPGFTGPPGPPGPPGPPGEKGQMGLSFQGPKGDKGDQGV |
| | SGPPGVPGQAQVQEKGDFATKGEKGQKGEPGFQGMPGVGEKGEPGKPGPRGKPGKDGDK |
| | GEKGSPGFPGEPGYPGLIGRQGPQGEKGEAGPPGPPGIVIGTGPLGEKGERGYPGTPGP |
| | RGEPGPKGFPGLPGQPGPPGLPVPGQAGAPGFPGERGEKGDRGFPGTSLPGPSGRDGLP |
| | GPPGSPGPPGQPGYTNGIVECQPGPPGDQGPPGIPGQPGFIGEIGEKGQKGESCLICDI |
| | DGYRGPPGPQGPPGEIGFPGQPGAKGDRGLPGRDGVAGVPGPQGTPGLIGQPGAKGEPG |
| | EFYFDLRLKGDKGDPGFPGQPGMPGRAGSPGRDGHPGLPGPKGSPGSVGLKGERGPPGG |
| | VGFPGSRGDTGPPGPPGYGPAGPIGDKGQAGFPGGPGSPGLPGPKGEPGKIVPLPGPPG |
| | AEGLPGSPGFPGPQGDRGFPGTPGRPGLPGEKGAVGQPGIGFPGPPGPKGVDGLPGDMG |
| | PPGTPGRPGFNGLPGNPGVQGQKGEPGVGLPGLKGLPGLPGIPGTPGEKGSIGVPGVPG |
| | EHGAIGPPGLQGIRGEPGPPGLPGSVGSPGVPGIGPPGARGPPGGQGPPGLSGPPGIKG |
| | EKGFPGFPGLDMPGPKGDKGAQGLPGITGQSGLPGLPGQQGAPGIPGFPGSKGEMGVMG |
| | TPGQPGSPGPVGAPGLPGEKGDHGFPGSSGPRGDPGLKGDKGDVGLPGKPGSMDKVDMG |
| | SMKGQKGDQGEKGQIGPIGEKGSRGDPGTPGVPGKDGQAGQPGQPGPKGDPGISGTPGA |
| | PGLPGPKGSVGGMGLPGTPGEKGVPGIPGPQGSPGLPGDKGAKGEKGQAGPPGIGIPGL |
| | RGEKGDQGIAGFPGSPGEKGEKGSIGIPGMPGSPGLKGSPGSVGYPGSPGLPGEKGDKG |
| | LPGLDGIPGVKGEAGLPGTPGPTGPAGQKGEPGSDGIPGSAGEKGEPGLPGRGFPGFPG |
| | AKGDKGSKGEVGFPGLAGSPGIPGSKGEQGFMGPPGPQGQPGLPGSPGHATEGPKGDRG |
| | PQGQPGLPGLPGPMGPPGLPGIDGVKGDKGNPGWPGAPGVPGPKGDPGFQGMPGIGGSP |
| | GITGSKGDMGPPGVPGFQGPKGLPGLQGIKGDQGDQGVPGAKGLPGPPGPPGPYDIIKG |
| | EPGLPGPEGPPGLKGLQGLPGPKGQQGVTGLVGIPGPPGIPGFDGAPGQKGEMGPAGPT |
| | GPRGFPGPPGPDGLPGSMGPPGTPSVDHGFLVTRHSQTIDDPQCPSGTKILYHGYSLLY |
| | VQGNERAHGQDLGTAGSCLRKFSTMPFLFCNINNVCNFASRNDYSYWLSTPEPMPMSMA |
| | PITGENIRPFISRCAVCEAPAMVMAVHSQTIQIPPCPSGWSSLWIGYSFVMHTSAGAEG |
| | SGQALASPGSCLEEFRSAPFIECHGRGTCNYYANAYSFWLATIERSEMFKKPTPSTLKA |
| | GELRTHVSRCQVCMRRT |
| Cst8 | >AAH69496.1 Cystatin 8 (cystatin-related epididymal specific) |
| | [Homo sapiens] |
| | MPRCRWLSLILLTIPLALVARKDPKKNETGVLRKLKPVNASNANVKQCLWFAMQEYNKE |
| | SEDKYVFLVVKTLQAQLQVINLLEYLIDVEIARSDCRKPLSTNEICAIQENSKLKRKLS |
| | CSFLVGALPWNGEFTVMEKKCEDA |
| Cts1 | >NP_001903.1 procathepsin L isoform 1 preproprotein |
| | [Homo sapiens] |
| | MNPTLILAAFCLGIASATLTFDHSLEAQWTKWKAMHNRLYGMNEEGWRRAVWEKNMKMI |
| | ELHNQEYREGKHSFTMAMNAFGDMTSEEFRQVMNGFQNRKPRKGKVFQEPLFYEAPRSV |
| | DWREKGYVTPVKNQGQCGSCWAFSATGALEGQMFRKTGRLISLSEQNLVDCSGPQGNEG |
| | CNGGLMDYAFQYVQDNGGLDSEESYPYEATEESCKYNPKYSVANDTGFVDIPKQEKALM |
| | KAVATVGPISVAIDAGHESFLFYKEGIYFEPDCSSEDMDHGVLVVGYGFESTESDNNKY |
| | WLVKNSWGEEWGMGGYVKMAKDRRNHCGIASAASYPTV |
| Emb | >NP_940851.1 embigin precursor [Homo sapiens] |
| | MRALPGLLEARARTPRLLLLQCLLAAARPSSADGSAPDSPFTSPPLREEIMANNFSLES |
| | HNISLTEHSSMPVEKNITLERPSNVNLTCQFTTSGDLNAVNVTWKKDGEQLENNYLVSA |
| | TGSTLYTQYRFTIINSKQMGSYSCFFREEKEQRGTFNFKVPELHGKNKPLISYVGDSTV |
| | LTCKCQNCFPLNWTWYSSNGSVKVPVGVQMNKYVINGTYANETKLKITQLLEEDGESYW |
| | CRALFQLGESEEHIELVVLSYLVPLKPFLVIVAEVILLVATILLCEKYTQKKKKHSDEG |
| | KEFEQIEQLKSDDSNGIENNVPRHRKNESLGQ |
| F3 | >AAA61152.1 tissue factor [Homo sapiens] |
| | METPAWPRVPRPETAVARTLLLGWVFAQVAGASGTTNTVAAYNLTWKSTNFKTILEWEP |
| | KPVNQVYTVQISTKSGDWKSKCFYTTDTECDLTDEIVKDVKQTYLARVFSYPAGNVEST |
| | GSAGEPLYENSPEFTPYLETNLGQPTIQSFEQVGTKVNVTVEDERTLVRRNNTFLSLRD |
| | VFGKDLIYTLYYWKSSSSGKKTAKTNTNEFLIDVDKGENYCFSVQAVIPSRTVNRKSTD |
| | SPVECMGQEKGEFREIFYIIGAVVFVVIILVIILAISLHKCRKAGVGQSWKENSPLNVS |
| Fdps | >AAA52423.1 farnesyl pyrophosphate synthetase (EC 2.5.1.1) |
| | [Homo sapiens] |
| | MNGDQNSDVYAQEKQDFVQHFSQIVRVLTEDEMGHPEIGDAIARLKEVLEYNAIGGKYN |
| | RGLTVVVAFRELVEPRKQDADSLQRAWTVGWCVELLQAFFLVADDIMDSSLTRRGQTCW |
| | YQKPGVGLDAINDANLLEACIYRLLKLYCREQPYYLNLIELFLQSSYQTEIGQTLDLLT |
| | APQGNVDLVRFTEKRYKSIVKYKTAFYSFYLPIAAAMYMAGIDGEKEHANAKKILLEMG |
| | EFFQIQDDYLDLFGDPSVTGKIGTDIQDNKCSWLVVQCLQRATPEQYQILKENYGQKEA |
| | EKVARVKALYEELDLPAVFLQYEEDSYSHIMALIEQYAAPLPPAVFLGLARKIYKRRK |
| Fndc3b | >XP 024309484.1 fibronectin type III domain-containing |
| | protein 3B isoform X1 [Homo sapiens] |
| | MYVTMMMTDQIPLELPPLLNGEVAMMPHLVNGDAAQQVILVQVNPGETFTIRAEDGTLQ |
| | CIQGPAEVPMMSPNGSIPPIHVPPGYISQVIEDSTGVRRVVVTPQSPECYPPSYPSAMS |
| | PTHHLPPYLTHHPHFIHNSHTAYYPPVTGPGDMPPQFFPQHHLPHTIYGEQEIIPFYGM |
| | STYITREDQYSKPPHKKLKDRQIDRQNRLNSPPSSIYKSSCTTVYNGYGKGHSGGSGGG |
| | GSGSGPGIKKTERRARSSPKSNDSDLQEYELEVKRVQDILSGIEKPQVSNIQARAVVLS |
| | WAPPVGLSCGPHSGLSFPYSYEVALSDKGRDGKYKIIYSGEELECNLKDLRPATDYHVR |
| | VYAMYNSVKGSCSEPVSFTTHSCAPECPFPPKLAHRSKSSLTLQWKAPIDNGSKITNYL |
| | LEWDEGKRNSGFRQCFFGSQKHCKLTKLCPAMGYTFRLAARNDIGTSGYSQEVVCYTLG |
| | NIPQMPSAPRLVRAGITWVTLQWSKPEGCSPEEVITYTLEIQEDENDNLFHPKYTGEDL |
| | TCTVKNLKRSTQYKFRLTASNTEGKSCPSEVLVCTTSPDRPGPPTRPLVKGPVTSHGFS |
| | VKWDPPKDNGGSEILKYLLEITDGNSEANQWEVAYSGSATEYTFTHLKPGTLYKLRACC |
| | ISTGGHSQCSESLPVRTLSIAPGQCRPPRVLGRPKHKEVHLEWDVPASESGCEVSEYSV |
| | EMTEPEDVASEVYHGPELECTVGNLLPGTVYRFRVRALNDGGYGPYSDVSEITTAAGPP |
| | GQCKAPCISCTPDGCVLVGWESPDSSGADISEYRLEWGEDEESLELIYHGTDTRFEIRD |
| | LLPAAQYCCRLQAFNQAGAGPYSELVLCQTPASAPDPVSTLCVLEEEPLDAYPDSPSAC |
| | LVLNWEEPCNNGSEILAYTIDLGDTSITVGNTTMHVMKDLLPETTYRIRIQAINEIGAG |
| | PFSQFIKAKTRPLPPLPPRLECAAAGPQSLKLKWGDSNSKTHAAEDIVYTLQLEDRNKR |
| | FISIYRGPSHTYKVQRLTEFTCYSFRIQAASEAGEGPFSETYTFSTTKSVPPTIKAPRV |
| | TQLEGNSCEILWETVPSMKGDPVNYILQVLVGRESEYKQVYKGEEATFQISGLQTNTDY |
| | RFRVCACRRCLDTSQELSGAFSPSAAFVLQRSEVMLTGDMGSLDDPKMKSMMPTDEQFA |
| | AIIVLGFATLSILFAFILQYFLMK |
| Gas6 | >AAA58494.1 growth-arrest-specific protein [Homo sapiens] |
| | MAPSLSPGPAALRRAPQLLLLLLAAECALAALLPAREATQFLRPRQRRAFQVFEEAKQG |
| | HLERECVEELCSREEAREVFENDPETDYFYPRYLDCINKYGSPYTKNSGFATCVQNLPD |
| | QCTPNPCDRKGTQACQDLMGNFFCLCKAGWGGRLCDKDVNECSQENGGCLQICHNKPGS |
| | FHCSCHSGFELSSDGRTCQDIDECADSEACGEARCKNLPGSYSCLCDEGFAYSSQEKAC |
| | RDVDECLQGRCEQVCVNSPGSYTCHCDGRGGLKLSQDMDTCEDILPCVPFSVAKSVKSL |
| | YLGRMFSGTPVIRLRFKRLQPTRLVAEFDFRTFDPEGILLFAGGHQDSTWIVLALRAGR |
| | LELQLRYNGVGRVTSSGPVINHGMWQTISVEELARNLVIKVNRDAVMKIAVAGDLFQPE |
| | RGLYHLNLTVGGIPFHEKDLVQPINPRLDGCMRSWNWLNGEDTTIQETVKVNTRMQCFS |
| | VTERGSFYPGSGFAFYSLDYMRTPLDVGTESTWEVEVVAHIRPAADTGVLFALWAPDLR |
| | AVPLSVALVDYHSTKKLKKQLVVLAVEHTALALMEIKVCDGQEHVVTVSLRDGEATLEV |
| | DGTRGQSEVSAAQLQERLAVLERHLRSPVLTFAGGLPDVPVTSAPVTAFYRGCMTLEVN |
| | RRLLDLDEAAYKHSDITAHSCPPVEPAAA |
| Gsta4 | >CAA73482.1 glutathione transferase A4-4 [Homo sapiens] |
| | MAARPKLHYPNGRGRMESVRWVLAAAGVEFDEEFLETKEQLYKLQDGNHLLFQQVPMVE |
| | IDGMKLVQTRSILHYIADKHNLFGKNLKERTLIDMYVEGTLDLLELLIMHPFLKPDDQQ |
| | KEVVNMAQKAIIRYFPVFEKILRGHGQSFLVGNQLSLADVILLQTILALEEKIPNILSA |
| | FPFLQEYTVKLSNIPTIKRFLEPGSKKKPPPDEIYVRTVYNIFRP |
| Hmgcs1 | >CAA47061.1 Hydroxymethylglutaryl CoA Synthase [Homo sapiens] |
| | MPGSLPLNAEACWPKDVGIVALEIYFPSQYVDQAELEKYDGVDAGKYTIGLGQAKMGFC |
| | TDREDINSLCMTVVQNLMERNNLSYDCIGRLEVGTETIIDKSKSVKTNLMQLFEESGNT |
| | DIEGIDTTNACYGGTAAVFNAVNWIESSSWDGRYALVVAGDIAVYATGNARPTGGVGAV |
| | ALLIGPNAPLIFERGLRGTHMQHAYDFYKPDMLSEYPIVDGKLSIQCYLSALDRCYSVY |
| | CKKIHAQWQKEANDNDFTLNDFGFMIFHSPYCKLVQKSLARMLLNDFLNDQNRDKNSIY |
| | SGLKAFGDVKLEDTYFDRDVEKAFMKASSELFSQKTKASLLVSNQNGNMYTSSVYGSLA |
| | SVLAQYSPQHLAGKRIGVFSYGSGLAATLYSLKVTQDATPGSALDKITASLCDLKSRLD |
| | SRTGVAQDVFAENMKLREDTHHLVNYIPQGSIDSLFEGTWYLVRVDEKHRRTYARRPTP |
| | NDDTLDEGVGLVHSNIATEHIPSPAKKVPRLPATAAEPEAAVISNGVW |
| Kcnd2 | >AAD22053.1 potassium channel KV4.2 [Homo sapiens] |
| | MAAGVAAWLPFARAAAIGWMPVASGPMPAPPRQERKRTQDALIVLNVSGTRFQTWQDTL |
| | ERYPDTLLGSSERDFFYHPETQQYFFDRDPDIFRHILNFYRTGKLHYPRHECISAYDEE |
| | LAFFGLIPEIIGDCCYEEYKDRRRENAERLQDDADTDTAGESALPTMTARQRVWRAFEN |
| | PHTSTMALVFYYVTGFFIAVSVIANVVETVPCGSSPGHIKELPCGERYAVAFFCLDTAC |
| | VMIFTVEYLLRLAAAPSRYRFVRSVMSIIDVVAILPYYIGLVMTDNEDVSGAFVTLRVF |
| | RVFRIFKFSRHSQGLRILGYTLKSCASELGFLLFSLTMAIIIFATVMFYAEKGSSASKF |
| | TSIPAAFWYTIVTMTTLGYGDMVPKTIAGKIFGSICSLSGVLVIALPVPVIVSNFSRIY |
| | HQNQRADKRRAQKKARLARIRAAKSGSANAYMQSKRSGLLSNQLQSSEDEPAFVSKSGS |
| | SFETQHHHLLHCLEKTTNHEFVDEQVFEESCMEVATVNRPSSHSPSLSSQQGVTSTCCS |
| | RRHKKTFRIPNANVSGSHRGSVQELSTIQIRCVERTPLSNSRSSLNAKMEECVKLNCEQ |
| | PYVTTAIISIPTPPVTTPEGDDRPESPEYSGGNIVRVSAL |
| Kit | >CAA49159.1 mast/stem cell growth factor receptor |
| | [Homo sapiens] |
| | MRGARGAWDFLCVLLLLLRVQTGSSQPSVSPGEPSPPSIHPGKSDLIVRVGDEIRLLCT |
| | DPGFVKWTFEILDETNENKQNEWITEKAEATNTGKYTCTNKHGLSNSIYVFVRDPAKLF |
| | LVDRSLYGKEDNDTLVRCPLTDPEVTNYSLKGCQGKPLPKDLRFIPDPKAGIMIKSVKR |
| | AYHRLCLHCSVDQEGKSVLSEKFILKVRPAFKAVPVVSVSKASYLLREGEEFTVTCTIK |
| | DVSSSVYSTWKRENSQTKLQEKYNSWHHGDFNYERQATLTISSARVNDSGVFMCYANNT |
| | FGSANVTTTLEVVDKGFINIFPMINTTVFVNDGENVDLIVEYEAFPKPEHQQWIYMNRT |
| | FTDKWEDYPKSENESNIRYVSELHLTRLKGTEGGTYTFLVSNSDVNAAIAFNVYVNTKP |
| | EILTYDRLVNGMLQCVAAGFPEPTIDWYFCPGTEQRCSASVLPVDVQTLNSSGPPFGKL |
| | VVQSSIDSSAFKHNGTVECKAYNDVGKTSAYFNFAFKGNNKEQIHPHTLFTPLLIGFVI |
| | VAGMMCIIVMILTYKYLQKPMYEVQWKVVEEINGNNYVYIDPTQLPYDHKWEFPRNRLS |
| | FGKTLGAGAFGKVVEATAYGLIKSDAAMTVAVKMLKPSAHLTEREALMSELKVLSYLGN |
| | HMNIVNLLGACTIGGPTLVITEYCCYGDLLNFLRRKRDSFICSKQEDHAEAALYKNLLH |
| | SKESSCSDSTNEYMDMKPGVSYVVPTKADKRRSVRIGSYIERDVTPAIMEDDELALDLE |
| | DLLSFSYQVAKGMAFLASKNCIHRDLAARNILLTHGRITKICDFGLARDIKNDSNYVVK |
| | GNARLPVKWMAPESIFNCVYTFESDVWSYGIFLWELFSLGSSPYPGMPVDSKFYKMIKE |
| | GFRMLSPEHAPAEMYDIMKTCWDADPLKRPTFKQIVQLIEKQISESTNHIYSNLANCSP |
| | NRQKPVVDHSVRINSVGSTASSSQPLLVHDDV |
| Lgals1 | >AAF34677.1 galectin-related inhibitor of proliferation |
| | isoform a [Homo sapiens] |
| | MVMLQGVVPLDAHRFQVDFQCGCSLCPRPDIAFHFNPRFHTTKPHVICNTLHGGRWQRE |
| | ARWPHLALRRGSSFLILFLFGNEEVKVSVNGQHFLHFRYRLPLSHVDTLGIFGDILVEA |
| | VGFLNINPFVEGSREYPAGHPFLLMSPRLEVPCSHALPQGLSPGQVIIVRGLVLQEPKH |
| | FTVSLRDQAAHAPVTLRASFADRTLAWISRWGQKKLISAPFLFYPQRFFEVLLLFQEGG |
| | LKLALNGQGLGATSMNQQALEQLRELRISGSVQLYCVHS |
| Mast4 | >AAH33215.1 Microtubule associated serine/threonine kinase |
| | family member 4 [Homo sapiens] |
| | MGEKVSEAPEPVPRGCSGHGSRTPASALVAASSPGASSAESSSGSETLSEEGEPGGFSR |
| | EHQPPPPPPLGGTLGARAPAAWAPASVLLERGVLALPPPLPGGAVPPAPRGSSASQEEQ |
| | DEELDHILSPPPMPFRKCSNPDVASGPGKSLKYKRQLSEDGRQLRRGSLGGALTGRYLL |
| | PNPVAGQAWPASAETSNLVRMRSQALGQSAPSLTASLKELSLPRRGSLIDSQKWNCLVK |
| | RPVCPNAGRTSPLG |
| Mt2 | >AAC50612.1 Mellb-melatonin receptor [Homo sapiens] |
| | MSENGSFANCCEAGGWAVRPGWSGAGSARPSRTPRPPWVAPALSAVLIVTTAVDVVGNL |
| | LVILSVLRNRKLRNAGNLFLVSLALADLVVAFYPYPLILVAIFYDGWALGEEHCKASAF |
| | VMGLSVIGSVFNITALAINRYCYICHSMAYHRIYRRWHTPLHICLIWLLTVVALLPNFF |
| | VGSLEYDPRIYSCTFIQTASTQYTAAVVVIHFLLPIAVVSFCYLRIWVLVLQARRKAKP |
| | ESRLCLKPSDLRSFLTMFVVFVIFAICWAPLNCIGLAVAINPQEMAPQIPEGLFVTSYL |
| | LAYFNSCLNAIVYGLLNQNFRREYKRILLALWNPRHCIQDASKGSHAEGLQSPAPPIIG |
| | VQHQADAL |
| Pak3 | >AAC36097.1 p21-activated kinase 3 [Homo sapiens] |
| | MSDGLDNEEKPPAPPLRMNSNNRDSSALNHSSKPLPMAPEEKNKKARLRSIFPGGGDKT |
| | NKKKEKERPEISLPSDFEHTIHVGFDAVTGEFTGIPEQWARLLQTSNITKLEQKKNPQA |
| | VLDVLKFYDSKETVNNQKYMSFTSGDKSAHGYIAAHPSSTKTASEPPLAPPVSEEEDEE |
| | EEEEEDENEPPPVIAPRPEHTKSIYTRSVVESIASPAVPNKEVTPPSAENANSSTLYRN |
| | TDRQRKKSKMTDEEILEKLRSIVSVGDPKKKYTRFEKIGQGASGTVYTALDIATGQEVA |
| | IKQMNLQQQPKKELIINEILVMRENKNPNIVNYLDSYLVGDELWVVMEYLAGGSLTDVV |
| | TETCMDEGQIAAVCRECLQALDFLHSNQVIHRDIKSDNILLGMDGSVKLTDFGFCAQIT |
| | PEQSKRSTMVGTPYWMAPEVVTRKAYGPKVDIWSLGIMAIEMVEGEPPYLNENPLRALY |
| | LIATNGTPELQNPERLSAVFRDFLNRCLEMDVDRRGSAKELLQHPFLKLAKPLSSLTPL |
| | IIAAKEAIKNSSR |
| Pik3c2g | >CAA03853.1 PI3-kinase [Homo sapiens] |
| | MAYSWQTDPNPNESHEKQYEHQEFLFVNQPHSSSQVSLGFDQIVDEISGKIPHYESEID |
| | ENTFFVPTAPKWDSTGHSLNEAHQISLNEFTSKSRELSWHQVSKAPAIGFSPSVLPKPQ |
| | NTNKECSWGSPIGKHHGADDSRFSILAPSFTSLDKINLEKELENENHNYHIGFESSIPP |
| | TNSSFSSDFMPKEENKRSGHVNIVEPSLMLLKGSLQPGMWESTWQKNIESIGCSIQLVE |
| | VPQSSNTSLASFCNKVKKIRERYHAADVNFNSGKIWSTTTAFPYQLFSKTKFNIHIFID |
| | NSTQPLHFMPCANYLVKDLIAEILHFCTNDQLLPKDHILSVCGSEEFLQNDHCLGSHKM |
| | FQKDKSVIQLHLQKSREAPGKLSRKHEEDHSQFYLNQLLEFMHIWKVSRQCLLTLIRKY |
| | DFHLKYLLKTQENVYNIIEEVKKICSVLGCVETKQITDAVNELSLILQRKGENFYQSSE |
| | TSAKGLIEKVTTELSTSIYQLINVYCNSFYADFQPVNVPRCTSYLNPGLPSHLSFTVYA |
| | AHNIPETWVHRINFPLEIKSLPRESMLTVKLFGIACATNNANLLAWTCLPLFPKEKSIL |
| | GSMLFSMTLQSEPPVEMITPGVWDVSQPSPVTLQIDFPATGWEYMKPDSEENRSNLEEP |
| | LKECIKHIARLSQKQTPLLLSEEKKRYLWFYRFYCNNENCSLPLVLGSAPGWDERTVSE |
| | MHTILRRWTFSQPLEALGLLTSSFPDQEIRKVAVQQLDNLLNDELLEYLPQLVQAVKFE |
| | WNLESPLVQLLLHRSLQSIQVAHRLYWLLKNAENEAYFKSWYQKLLAALQFCAGKALND |
| | EFSKEQKLIKILGDIGERVKSASDHQRQEVLKKEIGRLEEFFQDVNTCHLPLNPALCIK |
| | GIDHDACSYFTSNALPLKITFINANLMGKNISIIFKAGDDLRQDMLVLQLIQVMDNIWL |
| | QEGLDMQMIIYRCLSTGKDQRLVQMVPDAVTLAKIHRHSGLIGPLKENTIKKWFSQHNH |
| | LKADYEKALRNFFYSCAGWCVVTFILGVCDRHNDNIMLTKSGHMFHIDFGKFLGHAQTF |
| | GGIKRDRAPFIFTSEMEYFITEGGKNPQHFQDFVELCCRAYNIIRKHSQLLLNLLEMML |
| | YAGLPELSGIQDLKYVYNNLRPQDTDLEATSHFTKKIKESLECFPVKLNNLIHTLAQMS |
| | AISPAKSTSQTFPQESCLLSTTRSIERAILGFSKKSSNLYLIQVTHSNNETSLTEKSFE |
| | QFSKLHSQLQKQFASLTLPEFPHWWHLPFTNSDHRRFRDLNHYMEQILNVSHEVTNSDC |
| | VLSFFLSEAVQQTVESSPVYLGEKKFPDKKPKVQLVISYEDVKLTILVKHMKNIHLPDG |
| | SAPSAHVEFYLLPYPSEVLRRKTKSVPKCTDPTYNEIVVYDEVTELQGHVLMLIVKSKT |
| | VFVGAINIRLCSVPLDKEKWYPLGNSIISPLL |
| Rampl | >CAA04472.1 RAMP1 [Homo sapiens] |
| | MARALCRLPRRGLWLLLAHHLFMTTACQEANYGALLRELCLTQFQVDMEAVGETLWCDW |
| | GRTIRSYRELADCTWHMAEKLGCFWPNAEVDRFFLAVHGRYFRSCPISGRAVRDPPGSI |
| | LYPFIVVPITVTLLVTALVVWQSKRTEGIV |
| Smoc2 | >AAH47583.1 SMOC2 protein [Homo sapiens] |
| | MLLPQLCWLPLLAGLLPPVPAQKFSALTFLRVDQDKDKDCSLDCAGSPQKPLCASDGRT |
| | FLSRCEFQRAKCKDPQLEIAYRGNCKDVSRCVAERKYTQEQARKEFQQVFIPECNDDGT |
| | YSQVQCHSYTGYCWCVTPNGRPISGTAVAHKTPRCPGSVNEKLPQREGTGKTDDAAAPA |
| | LETQPQGDEEDIASRYPTLWTEQVKSRQNKTNKNSVSSCDQEHQSALEEAKQPKNDNVV |
| | IPECAHGGLYKPVQCHPSTGYCWCVLVDTGRPIPGTSTRYEQPKCDNTARAHPAKARDL |
| | YKGRQLQGCPGAKKHEFLTSVLDALSTDMVHAASDPSSSSGRLSEPDPSHTLEERVVHW |
| | YFKLLDKNSSGDIGKKEIKPFKRFLRKKSKPKKCVKKFVEYCDVNNDKSISVQELMGCL |
| | GVAKEDGKADTKKRHTPRGHVESTSNRQPRKQG |
| Tac1 | >CAA38351.1 beta-preprotachykinin [Homo sapiens] |
| | MKILVALAVFFLVSTQLFAEEIGANDDLNYWSDWYDSDQIKEELPEPFEHLLQRIARRP |
| | KPQQFFGLMGKRDADSSIEKQVALLKALYGHGQISHKRHKTDSFVGLMGKRALNSVAYE |
| | RSAMQNYERRR |
| Timp1 | >AAA52436.1 prefibroblast collagenase inhibitor |
| | [Homo sapiens] |
| | MAPFEPLASGILLLLWLIAPSRACTCVPPHPQTAFCNSDLVIRAKFVGTPEVNQTTLYQ |
| | RYEIKMTKMYKGFQALGDAADIRFVYTPAMESVCGYFHRSHNRSEEFLIAGKLQDGLLH |
| | ITTCSFVAPWNSLSLAQRRGFTKTYTVGCEECTVFPCLSIPCKLQSGTHCLWTDQLLQG |
| | SEKGFQSRHLACLPREPGLCTWQSLRSQIA |
| Tmsb4x | >AAA36745.1 thymosin beta-4 [Homo sapiens] |
| | MSDKPDMAEIEKFDKSKLKKTETQEKNPLPSKETIEQEKQAGES |
| Tpm4 | >AAH02827.1 Tropomyosin 4 [Homo sapiens] |
| | MAGLNSLEAVKRKIQALQQQADEAEDRAQGLQRELDGERERREKAEGDVAALNRRIQLF |
| | EEELDRAQERLATALQKLEEAEKAADESERGMKVIENRAMKDEEKMEIQEMQLKEAKHI |
| | AEEADRKYEEVARKLVILEGELERAEERAEVSELKCGDLEEELKNVTNNLKSLEAASEK |
| | YSEKEDKYEEEIKLLSDKLKEAETRAEFAERTVAKLEKTIDDLEEKLAQAKEENVGLHQ |
| | TLDQTLNELNCI |
| Trib2 | >NP_067675.1 tribbles homolog 2 [Homo sapiens] |
| | MNIHRSTPITIARYGRSRNKTQDFEELSSIRSAEPSQSFSPNLGSPSPPETPNLSHCVS |
| | CIGKYLLLEPLEGDHVFRAVHLHSGEELVCKVFDISCYQESLAPCFCLSAHSNINQITE |
| | IILGETKAYVFFERSYGDMHSFVRTCKKLREEEAARLFYQIASAVAHCHDGGLVLRDLK |
| | LRKFIFKDEERTRVKLESLEDAYILRGDDDSLSDKHGCPAYVSPEILNTSGSYSGKAAD |
| | VWSLGVMLYTMLVGRYPFHDIEPSSLFSKIRRGQFNIPETLSPKAKCLIRSILRREPSE |
| | RLTSQEILDHPWFSTDFSVSNSAYGAKEVSDQLVPDVNMEENLDPFFN |
| Vim | >AAA61279.1 vimentin [Homo sapiens] |
| | MSTRSVSSSSYRRMFGGPGTASRPSSSRSYVTTSTRTYSLGDALRPSTSRSLYASSPGG |
| | VYATRSSAVRLRSSVPGVRLLQDSVDFSLADAINTEFKNTRTNEKVELQELNDRFANYI |
| | DKVRFLEQQNKILLAELEQLKGQGKSRLGDLYEEEMRELRRQVDQLTNDKARVEVERDN |
| | LAEDIMRLREKLQEEMLQREEAENTLQSFRQDVDNASLARLDLERKVESLQEEIAFLKK |
| | LHEEEIQELQAQIQEQHVQIDVDVSKPDLTAALRDVRQQYESVAAKNLQEAEEWYKSKF |
| | ADLSEAANRNNDALRQAKQESTEYRRQVQSLTCEVDALKGTNESLERQMREMEENFAVE |
| | AANYQDTIGRLQDEIQNMKEEMARHLREYQDLLNVKMALDIEIATYRKLLEGEESRISL |
| | PLPNFSSLNLRETNLDSLPLVDTHSKRTFLIKTVETRDGQVINETSQHHDDLE |
| Ybx1 | >AAA61308.1 Y box binding protein-1 [Homo sapiens] |
| | MSSEAETQQPPAAPPAAPALSAADTKPGTTGSGAGSGGPGGLTSAAPAGGDKKVIATKV |
| | LGTVKWFNVRNGYGFINRNDTKEDVFVHQTAIKKNNPRKYLRSVGDGETVEFDVVEGEK |
| | GEEAANVTGPGGVPVQGSKYAADRNHYRRYPRRRGPPRNYQQNYQNSESGEKNEGSESA |
| | PEGQAQQRRPYRRRRFPPYYMRRPYGRRPQYSNPPVQGEVMEGADNQGAGEQGRPVRQN |
| | MYRGYRPRFRRGPPRQRQPREDGNEEDKENQGDETQGQQPPQRRYRRNFNYRRRRPENP |
| | KPQDGKETKAADPPAENSRSRG |
| Zfp804a | >NP_919226.1 zinc finger protein 804A [Homo sapiens] |
| | MECYYIVISSTHLSNGHFRNIKGVFRGPLSKNGNKTLDYAEKENTIAKALEDLKANFYC |
| | ELCDKQYYKHQEFDNHINSYDHAHKQRLKELKQREFARNVASKSRKDERKQEKALQRLH |
| | KLAELRKETVCAPGSGPMFKSTTVTVRENCNEISQRVVVDSVNNQQDFKYTLIHSEENT |
| | KDATTVAEDPESANNYTAKNNQVGDQAQGIHRHKIGFSFAFPKKASVKLESSAAAFSEY |
| | SDDASVGKGFSRKSRFVPSACHLQQSSPTDVLLSSEEKTNSFHPPEAMCRDKETVQTQE |
| | IKEVSSEKDALLLPSFCKFQLQLSSDADNCQNSVPLADQIPLESVVINEDIPVSGNSFE |
| | LLGNKSTVLDMSNDCISVQATTEENVKHNEASTTEVENKNGPETLAPSNTEEVNITIHK |
| | KTNFCKRQCEPFVPVLNKHRSTVLQWPSEMLVYTTTKPSISYSCNPLCFDFKSTKVNNN |
| | LDKNKPDLKDLCSQQKQEDICMGPLSDYKDVSTEGLTDYEIGSSKNKCSQVTPLLADDI |
| | LSSSCDSGKNENTGQRYKNISCKIRETEKYNFTKSQIKQDTLDEKYNKIRLKETHEYWF |
| | HKSRRKKKRKKLCQHHHMEKTKESETRCKMEAENSYTENAGKYLLEPISEKQYLAAEQL |
| | LDSHQLLDKRPKSESISLSDNEEMCKTWNTEYNTYDTISSKNHCKKNTILLNGQSNATM |
| | IHSGKHNLTYSRTYCCWKTKMSSCSQDHRSLVLQNDMKHMSQNQAVKRGYNSVMNESER |
| | FYRKRRQHSHSYSSDESLNRQNHLPEEFLRPPSTSVAPCKPKKKRRRKRGRFHPGFETL |
| | ELKENTDYPVKDNSSLNPLDRLISEDKKEKMKPQEVAKIERNSEQTNQLRNKLSFHPNN |
| | LLPSETNGETEHLEMETTSGELSDVSNDPTTSVCVASAPTKEAIDNTLLEHKERSENIN |
| | LNEKQIPFQVPNIERNFRQSQPKSYLCHYELAEALPQGKMNETPTEWLRYNSGILNTQP |
| | PLPFKEAHVSGHTFVTAEQILAPLALPEQALLIPLENHDKFKNVPCEVYQHILQPNMLA |
| | NKVKFTFPPAALPPPSTPLQPLPLQQSLCSTSVTTIHHTVLQQHAAAAAAAAAAAAAGT |
| | FKVLQPHQQFLSQIPALTRTSLPQLSVGPVGPRLCPGNQPTFVAPPQMPIIPASVLHPS |
| | HLAFPSLPHALFPSLLSPHPTVIPLQPLF |

TABLE B
nucleotide sequences for target genes of the disclosure.

| Gene | | Ensembl Gene |
|---|---|---|
| name | Exemplary polynucleotide sequence | Accession No. |
| Aebp1 | >D86479.1: 1-2538 Homo sapiens mRNA for AEBP1, complete | ENSG00000106624 |
| | cds | |
| | ATGGACTATTACTTTGGGCCTCCTCCGCCCCAGAAGCCCGATGC | |
| | TGAGCGCCAGACGGACGAAGAGAAGGAGGAGCTGAAGAAACCCA | |
| | AAAAGGAGGACAGCAGCCCCAAGGAGGAGACCGACAAGTGGGCA | |
| | GTGGAGAAGGGCAAGGACCACAAAGAGCCCCGAAAGGGCGAGGA | |
| | GTTGGAGGAGGAGTGGACGCCTACGGAGAAAGTCAAGTGTCCCC | |
| | CCATTGGGATGGAGTCACACCGTATTGAGGACAACCAGATCCGA | |
| | GCCTCCTCCATGCTGCGCCACGGCCTGGGGGCACAGCGCGGCCG | |
| | GCTCAACATGCAGACCGGTGCCACTGAGGACGACTACTATGATG | |
| | GTGCGTGGTGTGCCGAGGACGATGCCAGGACCCAGTGGATAGAG | |
| | GTGGACACCAGGAGGACTACCCGGTTCACAGGCGTCATCACCCA | |
| | GGGCAGAGACTCCAGCATCCATGACGATTTTGTGACCACCTTCT | |
| | TCGTGGGCTTCAGCAATGACAGCCAGACATGGGTGATGTACACC | |
| | AACGGCTATGAGGAAATGACCTTTCATGGGAACGTGGACAAGGA | |
| | CACACCCGTGCTGAGTGAGCTCCCAGAGCCGGTGGTGGCTCGTT | |
| | TCATCCGCATCTACCCACTCACCTGGAATGGCAGCCTGTGCATG | |
| | CGCCTGGAGGTGCTGGGGTGCTCTGTGGCCCCTGTCTACAGCTA | |
| | CTACGCACAGAATGAGGTGGTGGCCACCGATGACCTGGATTTCC | |
| | GGCACCACAGCTACAAGGACATGCGCCAGCTCATGAAGGTGGTG | |
| | AACGAGGAGTGCCCCACCATCACCCGCACTTACAGCCTGGGCAA | |
| | GAGCTCACGAGGCCTCAAGATCTATGCCATGGAGATCTCAGACA | |
| | ACCCTGGGGAGCATGAACTGGGGGAGCCCGAGTTCCGCTACACT | |
| | GCTGGGATCCATGGCAACGAGGTGCTGGGCCGAGAGCTGTTGCT | |
| | GCTGCTCATGCAGTACCTGTGCCGAGAGTACCGCGATGGGAACC | |
| | CACGTGTGCGCAGCCTGGTGCAGGACACACGCATCCACCTGGTG | |
| | CCCTCACTGAACCCTGATGGCTACGAGGTGGCAGCGCAGATGGG | |
| | CTCAGAGTTTGGGAACTGGGCGCTGGGACTGTGGACTGAGGAGG | |
| | GCTTTGACATCTTTGAAGATTTCCCGGATCTCAACTCTGTGCTC | |
| | TGGGGAGCTGAGGAGAGGAAATGGGTCCCCTACCGGGTCCCCAA | |
| | CAATAACTTGCCCATCCCTGAACGCTACCTTTCGCCAGATGCCA | |
| | CGGTATCCACGGAGGTCCGGGCCATCATTGCCTGGATGGAGAAG | |
| | AACCCCTTCGTGCTGGGAGCAAATCTGAACGGCGGCGAGCGGCT | |
| | AGTATCCTACCCCTACGATATGGCCCGCACGCCTACCCAGGAGC | |
| | AGCTGCTGGCCGCAGCCATGGCAGCAGCCCGGGGGGAGGATGAG | |
| | GACGAGGTCTCCGAGGCCCAGGAGACTCCAGACCACGCCATCTT | |
| | CCGGTGGCTTGCCATCTCCTTCGCCTCCGCACACCTCACCTTGA | |
| | CCGAGCCCTACCGCGGAGGCTGCCAAGCCCAGGACTACACCGGC | |
| | GGCATGGGCATCGTCAACGGGGCCAAGTGGAACCCCCGGACCGG | |
| | GACTATCAATGACTTCAGTTACCTGCATACCAACTGCCTGGAGC | |
| | TCTCCTTCTACCTGGGCTGTGACAAGTTCCCTCATGAGAGTGAG | |
| | CTGCCCCGCGAGTGGGAGAACAACAAGGAGGCGCTGCTCACCTT | |
| | CATGGAGCAGGTGCACCGCGGCATTAAGGGGGTGGTGACGGACG | |
| | AGCAAGGCATCCCCATTGCCAACGCCACCATCTCTGTGAGTGGC | |
| | ATTAATCACGGCGTGAAGACAGCCAGTGGTGGTGATTACTGGCG | |
| | AATCTTGAACCCGGGTGAGTACCGCGTGACAGCCCACGCGGAGG | |
| | GCTACACCCCGAGCGCCAAGACCTGCAATGTTGACTATGACATC | |
| | GGGGCCACTCAGTGCAACTTCATCCTGGCTCGCTCCAACTGGAA | |
| | GCGCATCCGGGAGATCATGGCCATGAACGGGAACCGGCCTATCC | |
| | CACACATAGACCCATCGCGCCCTATGACCCCCCAACAGCGACGC | |
| | CTGCAGCAGCGACGCCTACAACACCGCCTGCGGCTTCGGGCACA | |
| | GATGCGGCTGCGGCGCCTCAACGCCACCACCACCCTAGGCCCCC | |
| | ACACTGTGCCTCCCACGCTGCCCCCTGCCCCTGCCACCACCCTG | |
| | AGCACTACCATAGAGCCCTGGGGCCTCATACCGCCAACCACCGC | |
| | TGGCTGGGAGGAGTCGGAGACTGAGACCTACACAGAGGTGGTGA | |
| | CAGAGTTTGGGACCGAGGTGGAGCCCGAGTTTGGGACCAAGGTG | |
| | GAGCCCGAGTTTGAGACCCAGTTGGAGCCTGAGTTTGAGACCCA | |
| | GCTGGAACCCGAGTTTGAGGAAGAGGAGGAGGAGGAGAAAGAGG | |
| | AGGAGATAGCCACTGGCCAGGCATTCCCCTTCACAACAGTAGAG | |
| | ACCTACACAGTGAACTTTGGGGACTTCTGA | |
| Bace2 | >AF117892.1: 101-1657 Homo sapiens aspartic-like protease | ENSG00000182240 |
| | mRNA, complete cds | |
| | ATGGGCGCACTGGCCCGGGCGCTGCTGCTGCCTCTGCTGGCCCA | |
| | GTGGCTCCTGCGCGCCGCCCCGGAGCTGGCCCCCGCGCCCTTCA | |
| | CGCTGCCCCTCCGGGTGGCCGCGGCCACGAACCGCGTAGTTGCG | |
| | CCCACCCCGGGACCCGGGACCCCTGCCGAGCGCCACGCCGACGG | |
| | CTTGGCGCTCGCCCTGGAGCCTGCCCTGGCGTCCCCCGCGGGCG | |
| | CCGCCAACTTCTTGGCCATGGTAGACAACCTGCAGGGGGACTCT | |
| | GGCCGCGGCTACTACCTGGAGATGCTGATCGGGACCCCCCCGCA | |
| | GAAGCTACAGATTCTCGTTGACACTGGAAGCAGTAACTTTGCCG | |
| | TGGCAGGAACCCCGCACTCCTACATAGACACGTACTTTGACACA | |
| | GAGAGGTCTAGCACATACCGCTCCAAGGGCTTTGACGTCACAGT | |
| | GAAGTACACACAAGGAAGCTGGACGGGCTTCGTTGGGGAAGACC | |
| | TCGTCACCATCCCCAAAGGCTTCAATACTTCTTTTCTTGTCAAC | |
| | ATTGCCACTATTTTTGAATCAGAGAATTTCTTTTTGCCTGGGAT | |
| | TAAATGGAATGGAATACTTGGCCTAGCTTATGCCACACTTGCCA | |
| | AGCCATCAAGTTCTCTGGAGACCTTCTTCGACTCCCTGGTGACA | |
| | CAAGCAAACATCCCCAACGTTTTCTCCATGCAGATGTGTGGAGC | |
| | CGGCTTGCCCGTTGCTGGATCTGGGACCAACGGAGGTAGTCTTG | |
| | TCTTGGGTGGAATTGAACCAAGTTTGTATAAAGGAGACATCTGG | |
| | TATACCCCTATTAAGGAAGAGTGGTACTACCAGATAGAAATTCT | |
| | GAAATTGGAAATTGGAGGCCAAAGCCTTAATCTGGACTGCAGAG | |
| | AGTATAACGCAGACAAGGCCATCGTGGACAGTGGCACCACGCTG | |
| | CTGCGCCTGCCCCAGAAGGTGTTTGATGCGGTGGTGGAAGCTGT | |
| | GGCCCGCGCATCTCTGATTCCAGAATTCTCTGATGGTTTCTGGA | |
| | CTGGGTCCCAGCTGGCGTGCTGGACGAATTCGGAAACACCTTGG | |
| | TCTTACTTCCCTAAAATCTCCATCTACCTGAGAGATGAGAACTC | |
| | CAGCAGGTCATTCCGTATCACAATCCTGCCTCAGCTTTACATTC | |
| | AGCCCATGATGGGGGCCGGCCTGAATTATGAATGTTACCGATTC | |
| | GGCATTTCCCCATCCACAAATGCGCTGGTGATCGGTGCCACGGT | |
| | GATGGAGGGCTTCTACGTCATCTTCGACAGAGCCCAGAAGAGGG | |
| | TGGGCTTCGCAGCGAGCCCCTGTGCAGAAATTGCAGGTGCTGCA | |
| | GTGTCTGAAATTTCCGGGCCTTTCTCAACAGAGGATGTAGCCAG | |
| | CAACTGTGTCCCCGCTCAGTCTTTGAGCGAGCCCATTTTGTGGA | |
| | TTGTGTCCTATGCGCTCATGAGCGTCTGTGGAGCCATCCTCCTT | |
| | GTCTTAATCGTCCTGCTGCTGCTGCCGTTCCGGTGTCAGCGTCG | |
| | CCCCCGTGACCCTGAGGTCGTCAATGATGAGTCCTCTCTGGTCA | |
| | GACATCGCTGGAAATGA | |
| Bst2 | >D28137.1: 10-552 Homo sapiens mRNA for BST-2, complete cds | ENSG00000130303 |
| | ATGGCATCTACTTCGTATGACTATTGCAGAGTGCCCATGGAAGA | |
| | CGGGGATAAGCGCTGTAAGCTTCTGCTGGGGATAGGAATTCTGG | |
| | TGCTCCTGATCATCGTGATTCTGGGGGTGCCCTTGATTATCTTC | |
| | ACCATCAAGGCCAACAGCGAGGCCTGCCGGGACGGCCTTCGGGC | |
| | AGTGATGGAGTGTCGCAATGTCACCCATCTCCTGCAACAAGAGC | |
| | TGACCGAGGCCCAGAAGGGCTTTCAGGATGTGGAGGCCCAGGCC | |
| | GCCACCTGCAACCACACTGTGATGGCCCTAATGGCTTCCCTGGA | |
| | TGCAGAGAAGGCCCAAGGACAAAAGAAAGTGGAGGAGCTTGAGG | |
| | GAGAGATCACTACATTAAACCATAAGCTTCAGGACGCGTCTGCA | |
| | GAGGTGGAGCGACTGAGAAGAGAAAACCAGGTCTTAAGCGTGAG | |
| | AATCGCGGACAAGAAGTACTACCCCAGCTCCCAGGACTCCAGCT | |
| | CCGCTGCGGCGCCCCAGCTGCTGATTGTGCTGCTGGGCCTCAGC | |
| | GCTCTGCTGCAGTGA | |
| Cnn3 | >NM_001839.5: 217-1206 Homo sapiens calponin 3 (CNN3), | ENSG00000117519 |
| | transcript variant 1, mRNA | |
| | ATGACCCACTTCAACAAGGGCCCTTCCTATGGGCTCTCGGCCGA | |
| | AGTCAAGAACAAGATTGCTTCCAAGTATGATCATCAGGCAGAAG | |
| | AAGATCTTCGCAATTGGATAGAAGAGGTGACAGGCATGAGCATT | |
| | GGCCCCAACTTCCAGCTGGGCTTAAAGGATGGCATCATCCTCTG | |
| | CGAACTTATAAACAAGCTACAGCCAGGCTCAGTGAAGAAGGTCA | |
| | ACGAGTCCTCACTGAACTGGCCTCAGTTGGAGAATATTGGCAAC | |
| | TTTATTAAAGCTATTCAGGCTTATGGTATGAAGCCACATGACAT | |
| | ATTCGAAGCAAATGATCTTTTTGAGAATGGAAACATGACCCAGG | |
| | TTCAGACTACTCTGGTGGCTCTAGCAGGTCTGGCTAAAACAAAA | |
| | GGATTCCATACAACCATTGACATTGGAGTTAAGTATGCAGAAAA | |
| | ACAAACAAGACGTTTTGATGAAGGAAAATTAAAAGCTGGCCAAA | |
| | GTGTAATTGGTCTGCAGATGGGAACCAACAAATGTGCCAGCCAG | |
| | GCAGGTATGACAGCTTACGGGACTAGGAGGCATCTTTATGATCC | |
| | CAAAATGCAAACTGACAAACCTTTTGACCAGACCACAATTAGTC | |
| | TGCAGATGGGCACTAATAAAGGAGCCAGCCAGGCAGGGATGTTA | |
| | GCACCAGGTACCAGAAGAGACATCTATGATCAGAAGCTAACATT | |
| | ACAGCCGGTGGACAACTCGACAATTTCCCTACAGATGGGTACCA | |
| | ACAAAGTTGCTTCCCAGAAAGGAATGAGTGTGTATGGGCTTGGG | |
| | CGGCAAGTATATGATCCCAAATACTGTGCTGCTCCTACAGAACC | |
| | TGTCATTCACAACGGAAGCCAAGGAACAGGAACAAATGGTTCGG | |
| | AAATCAGTGATAGTGATTATCAGGCAGAATACCCTGATGAGTAT | |
| | CATGGCGAGTACCAGGATGACTACCCCAGAGATTACCAATATAG | |
| | CGACCAAGGCATTGATTATTAG | |
| Col4a1 | >AH002741.2:1869-1952, 2282-2341, 2450-2539, 2638-2682, | ENSG00000187498 |
| | 2837-2881, 2976-3038, 3375-3428, 3624-3650, 3857-3940, | |
| | 4027-4089, 4508-4543, 4810-4851, 4995-5081, 5474-5500, | |
| | 5652-5702, 5825-5869, 5956-6009, 6180-6221, 6372-6456, | |
| | 7011-7046, 7239-7403, 7934-8029, 8211-8294, 8749-8819, | |
| | 9013-9204, 9377-9545, 9969-10061, 10148-10252, 10434-10531, | |
| | 10836-10986, 11117-11230, 11423-11590, 11732-11821, | |
| | 12109-12261, 12423-12521, 12634-12723, 13178-13317, | |
| | 13778-13904, 14003-14083, 14364-14462, 14752-14802, | |
| | 15096-15281, 15644-15777, 16139-16211, 16350-16421, | |
| | 16645-16773, 16958-17056, 17257-17469, 17781-17958, | |
| | 18617-18731, 18874-19046, 19276-19357 Homo sapiens alpha-2 | |
| | type IV collagen gene, partial cds; and alpha-1 type | |
| | IV collagen (COL4A1) gene, complete cds | |
| | ATGGGGCCCCGGCTCAGCGTCTGGCTGCTGCTGCTGCCCGCCGC | |
| | CCTTCTGCTCCACGAGGAGCACAGCCGGGCCGCTGCGAAGGGTG | |
| | GCTGTGCTGGCTCTGGCTGTGGCAAATGTGACTGCCATGGAGTG | |
| | AAGGGACAAAAGGGTGAAAGAGGCCTCCCGGGGTTACAAGGTGT | |
| | CATTGGGTTTCCTGGAATGCAAGGACCTGAGGGGCCACAGGGAC | |
| | CACCAGGACAAAAGGGTGATACTGGAGAACCAGGACTACCTGGA | |
| | ACAAAAGGGACAAGAGGACCTCCGGGAGCATCTGGCTACCCTGG | |
| | AAACCCAGGACTTCCCGGAATTCCTGGCCAAGACGGCCCGCCAG | |
| | GCCCCCCAGGTATTCCAGGATGCAATGGCACAAAGGGGGAGAGA | |
| | GGGCCGCTCGGGCCTCCTGGCTTGCCTGGTTTCGCAGGAAATCC | |
| | CGGACCACCAGGCTTACCAGGGATGAAGGGTGATCCAGGTGAGA | |
| | TACTTGGCCATGTGCCCGGGATGCTGTTGAAAGGTGAAAGAGGA | |
| | TTTCCCGGAATCCCAGGGACTCCAGGCCCACCAGGACTGCCAGG | |
| | GCTTCAAGGTCCTGTTGGGCCTCCAGGATTTACCGGACCACCAG | |
| | GTCCCCCAGGCCCTCCCGGCCCTCCAGGTGAAAAGGGACAAATG | |
| | GGCTTAAGTTTTCAAGGACCAAAAGGTGACAAGGGTGACCAAGG | |
| | GGTCAGTGGGCCTCCAGGAGTACCAGGACAAGCTCAAGTTCAAG | |
| | AAAAAGGAGACTTCGCCACCAAGGGAGAAAAGGGCCAAAAAGGT | |
| | GAACCTGGATTTCAGGGGATGCCAGGGGTCGGAGAGAAAGGTGA | |
| | ACCCGGAAAACCAGGACCCAGAGGCAAACCCGGAAAAGATGGTG | |
| | ACAAAGGGGAAAAAGGGAGTCCCGGTTTTCCTGGTGAACCCGGG | |
| | TACCCAGGACTCATAGGCCGCCAGGGCCCGCAGGGAGAAAAGGG | |
| | TGAAGCAGGTCCTCCTGGCCCACCTGGAATTGTTATAGGCACAG | |
| | GACCTTTGGGAGAAAAAGGAGAGAGGGGCTACCCTGGAACTCCG | |
| | GGGCCAAGAGGAGAGCCAGGCCCAAAAGGTTTCCCAGGACTACC | |
| | AGGCCAACCCGGACCTCCAGGCCTCCCTGTACCTGGGCAGGCTG | |
| | GTGCCCCTGGCTTCCCTGGTGAAAGAGGAGAAAAAGGTGACCGA | |
| | GGATTTCCTGGTACATCTCTGCCAGGACCAAGTGGAAGAGATGG | |
| | GCTCCCGGGTCCTCCTGGTTCCCCCGGGCCCCCTGGGCAGCCTG | |
| | GCTACACAAATGGAATTGTGGAATGTCAGCCCGGACCTCCAGGT | |
| | GACCAGGGTCCTCCTGGAATTCCAGGGCAGCCAGGATTTATAGG | |
| | CGAAATTGGAGAGAAAGGTCAAAAAGGAGAGAGTTGCCTCATCT | |
| | GTGATATAGACGGATATCGGGGGCCTCCCGGGCCACAGGGACCC | |
| | CCGGGAGAAATAGGTTTCCCAGGGCAGCCAGGGGCCAAGGGCGA | |
| | CAGAGGTTTGCCTGGCAGAGATGGTGTTGCAGGAGTGCCAGGCC | |
| | CTCAAGGTACACCAGGGCTGATAGGCCAGCCAGGAGCCAAGGGG | |
| | GAGCCTGGTGAGTTTTATTTCGACTTGCGGCTCAAAGGTGACAA | |
| | AGGAGACCCAGGCTTTCCAGGACAGCCCGGCATGCCAGGGAGAG | |
| | CGGGTTCTCCTGGAAGAGATGGCCATCCGGGTCTTCCTGGCCCC | |
| | AAGGGCTCGCCGGGTTCTGTAGGATTGAAAGGAGAGCGTGGCCC | |
| | CCCTGGAGGAGTTGGATTCCCAGGCAGTCGTGGTGACACCGGCC | |
| | CCCCTGGGCCTCCAGGATATGGTCCTGCTGGTCCCATTGGTGAC | |
| | AAAGGACAAGCAGGCTTTCCTGGAGGCCCTGGATCCCCAGGCCT | |
| | GCCAGGTCCAAAGGGTGAACCAGGAAAAATTGTTCCTTTACCAG | |
| | GCCCCCCTGGAGCAGAAGGACTGCCGGGGTCCCCAGGCTTCCCA | |
| | GGTCCCCAAGGAGACCGAGGCTTTCCCGGAACCCCAGGAAGGCC | |
| | AGGCCTGCCAGGAGAGAAGGGCGCTGTGGGCCAGCCAGGCATTG | |
| | GATTTCCAGGGCCCCCCGGCCCCAAAGGTGTTGACGGCTTACCT | |
| | GGAGACATGGGGCCACCGGGGACTCCAGGTCGCCCGGGATTTAA | |
| | TGGCTTACCTGGGAACCCAGGTGTGCAGGGCCAGAAGGGAGAGC | |
| | CTGGAGTTGGTCTACCGGGACTCAAAGGTTTGCCAGGTCTTCCC | |
| | GGCATTCCTGGCACACCCGGGGAGAAGGGGAGCATTGGGGTACC | |
| | AGGCGTTCCTGGAGAACATGGAGCGATCGGACCCCCTGGGCTTC | |
| | AGGGGATCAGAGGTGAACCGGGACCTCCTGGATTGCCAGGCTCC | |
| | GTGGGGTCTCCAGGAGTTCCAGGAATAGGCCCCCCTGGAGCTAG | |
| | GGGTCCCCCTGGAGGACAGGGACCACCGGGGTTGTCAGGCCCTC | |
| | CTGGAATAAAAGGAGAGAAGGGTTTCCCCGGATTCCCTGGACTG | |
| | GACATGCCGGGCCCTAAAGGAGATAAAGGGGCTCAAGGACTCCC | |
| | TGGCATAACGGGACAGTCGGGGCTCCCTGGCCTTCCTGGACAGC | |
| | AGGGGGCTCCTGGGATTCCTGGGTTTCCAGGTTCCAAGGGAGAA | |
| | ATGGGCGTCATGGGGACCCCCGGGCAGCCGGGCTCACCAGGACC | |
| | AGTGGGTGCTCCTGGATTACCGGGTGAAAAAGGGGACCATGGCT | |
| | TTCCGGGCTCCTCAGGACCCAGGGGAGACCCTGGCTTGAAAGGT | |
| | GATAAGGGGGATGTCGGTCTCCCTGGCAAGCCTGGCTCCATGGA | |
| | TAAGGTGGACATGGGCAGCATGAAGGGCCAGAAAGGAGACCAAG | |
| | GAGAGAAAGGACAAATTGGACCAATTGGTGAGAAGGGATCCCGA | |
| | GGAGACCCTGGGACCCCAGGAGTGCCTGGAAAGGACGGGCAGGC | |
| | AGGACAGCCTGGGCAGCCAGGACCTAAAGGTGATCCAGGTATAA | |
| | GTGGAACCCCAGGTGCTCCAGGACTTCCGGGACCAAAAGGATCT | |
| | GTTGGTGGAATGGGCTTGCCAGGAACACCTGGAGAGAAAGGTGT | |
| | GCCTGGCATCCCTGGCCCACAAGGTTCACCTGGCTTACCTGGAG | |
| | ACAAAGGTGCAAAAGGAGAGAAAGGGCAGGCAGGCCCACCTGGC | |
| | ATAGGCATCCCAGGACTGCGTGGTGAAAAGGGAGATCAAGGGAT | |
| | AGCGGGTTTCCCAGGAAGCCCTGGAGAGAAGGGAGAAAAAGGAA | |
| | GCATTGGGATCCCAGGAATGCCAGGGTCCCCAGGCCTTAAAGGG | |
| | TCTCCCGGGAGTGTTGGCTATCCAGGAAGTCCTGGGCTACCTGG | |
| | AGAAAAAGGTGACAAAGGCCTCCCAGGATTGGATGGCATCCCTG | |
| | GTGTCAAAGGAGAAGCAGGTCTTCCTGGGACTCCTGGCCCCACA | |
| | GGCCCAGCTGGCCAGAAAGGGGAGCCAGGCAGTGATGGAATCCC | |
| | GGGGTCAGCAGGAGAGAAGGGTGAACCAGGTCTACCAGGAAGAG | |
| | GATTCCCAGGGTTTCCAGGGGCCAAAGGAGACAAAGGTTCAAAG | |
| | GGTGAGGTGGGTTTCCCAGGATTAGCCGGGAGCCCAGGAATTCC | |
| | TGGATCCAAAGGAGAGCAAGGATTCATGGGTCCTCCGGGGCCCC | |
| | AGGGACAGCCGGGGTTACCGGGATCCCCAGGCCATGCCACGGAG | |
| | GGGCCCAAAGGAGACCGCGGACCTCAGGGCCAGCCTGGCCTGCC | |
| | AGGACTTCCGGGACCCATGGGGCCTCCAGGGCTTCCTGGGATTG | |
| | ATGGAGTTAAAGGTGACAAAGGAAATCCAGGCTGGCCAGGAGCA | |
| | CCCGGTGTCCCAGGGCCCAAGGGAGACCCTGGATTCCAGGGCAT | |
| | GCCTGGTATTGGTGGCTCTCCAGGAATCACAGGCTCTAAGGGTG | |
| | ATATGGGGCCTCCAGGAGTTCCAGGATTTCAAGGTCCAAAAGGT | |
| | CTTCCTGGCCTCCAGGGAATTAAAGGTGATCAAGGCGATCAAGG | |
| | CGTCCCGGGAGCTAAAGGTCTCCCGGGTCCTCCTGGCCCCCCAG | |
| | GTCCTTACGACATCATCAAAGGGGAGCCCGGGCTCCCTGGTCCT | |
| | GAGGGCCCCCCAGGGCTGAAAGGGCTTCAGGGACTGCCAGGCCC | |
| | GAAAGGCCAGCAAGGTGTTACAGGATTGGTGGGTATACCTGGAC | |
| | CTCCAGGTATTCCTGGGTTTGACGGTGCCCCTGGCCAGAAAGGA | |
| | GAGATGGGACCTGCCGGGCCTACTGGTCCAAGAGGATTTCCAGG | |
| | TCCACCAGGCCCCGATGGGTTGCCAGGATCCATGGGGCCCCCAG | |
| | GCACCCCATCTGTTGATCACGGCTTCCTTGTGACCAGGCATAGT | |
| | CAAACAATAGATGACCCACAGTGTCCTTCTGGGACCAAAATTCT | |
| | TTACCACGGGTACTCTTTGCTCTACGTGCAAGGCAATGAACGGG | |
| | CCCATGGACAGGACTTGGGCACGGCCGGCAGCTGCCTGCGCAAG | |
| | TTCAGCACAATGCCCTTCCTGTTCTGCAATATTAACAACGTGTG | |
| | CAACTTTGCATCACGAAATGACTACTCGTACTGGCTGTCCACCC | |
| | CTGAGCCCATGCCCATGTCAATGGCACCCATCACGGGGGAAAAC | |
| | ATAAGACCATTTATTAGTAGGTGTGCTGTGTGTGAGGCGCCTGC | |
| | CATGGTGATGGCCGTGCACAGCCAGACCATTCAGATCCCACCGT | |
| | GCCCCAGCGGGTGGTCCTCGCTGTGGATCGGCTACTCTTTTGTG | |
| | ATGCACACCAGCGCTGGTGCAGAAGGCTCTGGCCAAGCCCTGGC | |
| | GTCCCCCGGCTCCTGCCTGGAGGAGTTTAGAAGTGCGCCATTCA | |
| | TCGAGTGTCACGGCCGTGGGACCTGCAATTACTACGCAAACGCT | |
| | TACAGCTTTTGGCTCGCCACCATAGAGAGGAGCGAGATGTTCAA | |
| | GAAGCCTACGCCGTCCACCTTGAAGGCAGGGGAGCTGCGCACGC | |
| | ACGTCAGCCGCTGCCAAGTCTGTATGAGAAGAACATAA | |
| Cst8 | >BC069496.1: 176-604 Homo sapiens cystatin 8 (cystatin-related | ENSG00000125815 |
| | epididymal specific), mRNA (cDNA clone MGC: 96991 | |
| | IMAGE: 7262200), complete cds | |
| | ATGCCCAGGTGCCGGTGGCTCTCCCTGATCCTCCTCACCA | |
| | TTCCCCTGGCCCTGGTGGCCAGGAAAGACCCAAAAAAGAATGAG | |
| | ACGGGGGTGCTGAGGAAATTAAAACCCGTCAATGCCTCAAATGC | |
| | CAACGTGAAGCAGTGTCTGTGGTTTGCCATGCAAGAATACAACA | |
| | AAGAGAGCGAGGACAAGTATGTCTTCCTGGTGGTCAAGACACTG | |
| | CAAGCCCAGCTTCAGGTCACAAATCTTCTGGAATACCTTATTGA | |
| | TGTAGAAATTGCCCGCAGCGATTGCAGAAAGCCTTTAAGCACTA | |
| | ATGAAATCTGCGCCATTCAAGAAAACTCCAAGCTGAAAAGGAAA | |
| | TTAAGCTGCAGCTTTTTGGTAGGAGCACTTCCCTGGAATGGTGA | |
| | ATTCACTGTGATGGAGAAAAAGTGTGAAGATGCTTAA | |
| Cts1 | >NM_001912.5: 291-1292 Homo sapiens cathepsin L (CTSL), | ENSG00000135047 |
| | transcript variant 1, mRNA | |
| | ATGAATCCTACACTCATCCTTGCTGCCTTTTGCCTGGGAATTGC | |
| | CTCAGCTACTCTAACATTTGATCACAGTTTAGAGGCACAGTGGA | |
| | CCAAGTGGAAGGCGATGCACAACAGATTATACGGCATGAATGAA | |
| | GAAGGATGGAGGAGAGCAGTGTGGGAGAAGAACATGAAGATGAT | |
| | TGAACTGCACAATCAGGAATACAGGGAAGGGAAACACAGCTTCA | |
| | CAATGGCCATGAACGCCTTTGGAGACATGACCAGTGAAGAATTC | |
| | AGGCAGGTGATGAATGGCTTTCAAAACCGTAAGCCCAGGAAGGG | |
| | GAAAGTGTTCCAGGAACCTCTGTTTTATGAGGCCCCCAGATCTG | |
| | TGGATTGGAGAGAGAAAGGCTACGTGACTCCTGTGAAGAATCAG | |
| | GGTCAGTGTGGTTCTTGTTGGGCTTTTAGTGCTACTGGTGCTCT | |
| | TGAAGGACAGATGTTCCGGAAAACTGGGAGGCTTATCTCACTGA | |
| | GTGAGCAGAATCTGGTAGACTGCTCTGGGCCTCAAGGCAATGAA | |
| | GGCTGCAATGGTGGCCTAATGGATTATGCTTTCCAGTATGTTCA | |
| | GGATAATGGAGGCCTGGACTCTGAGGAATCCTATCCATATGAGG | |
| | CAACAGAAGAATCCTGTAAGTACAATCCCAAGTATTCTGTTGCT | |
| | AATGACACCGGCTTTGTGGACATCCCTAAGCAGGAGAAGGCCCT | |
| | GATGAAGGCAGTTGCAACTGTGGGGCCCATTTCTGTTGCTATTG | |
| | ATGCAGGTCATGAGTCCTTCCTGTTCTATAAAGAAGGCATTTAT | |
| | TTTGAGCCAGACTGTAGCAGTGAAGACATGGATCATGGTGTGCT | |
| | GGTGGTTGGCTACGGATTTGAAAGCACAGAATCAGATAACAATA | |
| | AATATTGGCTGGTGAAGAACAGCTGGGGTGAAGAATGGGGCATG | |
| | GGTGGCTACGTAAAGATGGCCAAAGACCGGAGAAACCATTGTGG | |
| | AATTGCCTCAGCAGCCAGCTACCCCACTGTGTGA | |
| Emb | >NM_198449.3: 138-1121 Homo sapiens embigin (EMB), mRNA | ENSG00000170571 |
| | ATGCGCGCCCTCCCCGGCCTGCTGGAGGCCAGGGCGCGTACGCC | |
| | CCGGCTGCTCCTCCTCCAGTGCCTTCTCGCTGCCGCGCGCCCAA | |
| | GCTCGGCGGACGGCAGTGCCCCAGATTCGCCTTTTACAAGTCCA | |
| | CCTCTCAGAGAAGAAATAATGGCAAATAACTTTTCCTTGGAGAG | |
| | TCATAACATATCACTGACTGAACATTCTAGTATGCCAGTAGAAA | |
| | AAAATATCACTTTAGAAAGGCCTTCTAATGTAAATCTCACATGC | |
| | CAGTTCACAACATCTGGGGATTTGAATGCAGTAAATGTGACTTG | |
| | GAAAAAAGATGGTGAACAACTTGAGAATAATTATCTTGTCAGTG | |
| | CAACAGGAAGCACCTTGTATACCCAATACAGGTTCACCATCATT | |
| | AATAGCAAACAAATGGGAAGTTATTCTTGTTTCTTTCGAGAGGA | |
| | AAAGGAACAAAGGGGAACATTTAATTTCAAAGTCCCTGAACTTC | |
| | ATGGGAAAAACAAGCCATTGATCTCTTACGTAGGGGATTCTACT | |
| | GTCTTGACATGTAAATGTCAAAATTGTTTTCCTTTAAATTGGAC | |
| | CTGGTACAGTAGTAATGGGAGTGTAAAGGTTCCTGTTGGTGTTC | |
| | AAATGAATAAATATGTGATCAATGGAACATATGCTAACGAAACA | |
| | AAGCTGAAGATAACACAACTTTTGGAGGAAGATGGGGAATCTTA | |
| | CTGGTGCCGTGCACTATTCCAATTAGGCGAGAGTGAAGAACACA | |
| | TTGAGCTTGTGGTGCTGAGCTATTTGGTGCCCCTCAAACCATTT | |
| | CTTGTAATAGTGGCTGAGGTGATTCTTTTAGTGGCCACCATTCT | |
| | GCTTTGTGAAAAGTACACACAAAAGAAAAAGAAGCACTCAGATG | |
| | AGGGGAAAGAATTTGAGCAGATTGAACAGCTGAAATCAGATGAT | |
| | AGCAATGGTATAGAAAATAATGTCCCCAGGCATAGAAAAAATGA | |
| | GTCTCTGGGCCAGTGA | |
| F3 | >J02846.1: 922-1021, 2190-2301, 6392-6591, 9289-9467, 10075- | ENSG00000117525 |
| | 10234, 11955-12091 Human tissue factor gene, complete cds | |
| | ATGGAGACCCCTGCCTGGCCCCGGGTCCCGCGCCCCGAGACCGC | |
| | CGTCGCTCGGACGCTCCTGCTCGGCTGGGTCTTCGCCCAGGTGG | |
| | CCGGCGCTTCAGGCACTACAAATACTGTGGCAGCATATAATTTA | |
| | ACTTGGAAATCAACTAATTTCAAGACAATTTTGGAGTGGGAACC | |
| | CAAACCCGTCAATCAAGTCTACACTGTTCAAATAAGCACTAAGT | |
| | CAGGAGATTGGAAAAGCAAATGCTTTTACACAACAGACACAGAG | |
| | TGTGACCTCACCGACGAGATTGTGAAGGATGTGAAGCAGACGTA | |
| | CTTGGCACGGGTCTTCTCCTACCCGGCAGGGAATGTGGAGAGCA | |
| | CCGGTTCTGCTGGGGAGCCTCTGTATGAGAACTCCCCAGAGTTC | |
| | ACACCTTACCTGGAGACAAACCTCGGACAGCCAACAATTCAGAG | |
| | TTTTGAACAGGTGGGAACAAAAGTGAATGTGACCGTAGAAGATG | |
| | AACGGACTTTAGTCAGAAGGAACAACACTTTCCTAAGCCTCCGG | |
| | GATGTTTTTGGCAAGGACTTAATTTATACACTTTATTATTGGAA | |
| | ATCTTCAAGTTCAGGAAAGAAAACAGCCAAAACAAACACTAATG | |
| | AGTTTTTGATTGATGTGGATAAAGGAGAAAACTACTGTTTCAGT | |
| | GTTCAAGCAGTGATTCCCTCCCGAACAGTTAACCGGAAGAGTAC | |
| | AGACAGCCCGGTAGAGTGTATGGGCCAGGAGAAAGGGGAATTCA | |
| | GAGAAATATTCTACATCATTGGAGCTGTGGTATTTGTGGTCATC | |
| | ATCCTTGTCATCATCCTGGCTATATCTCTACACAAGTGTAGAAA | |
| | GGCAGGAGTGGGGCAGAGCTGGAAGGAGAACTCCCCACTGAATG | |
| | TTTCATAA | |
| Fdps | >J05262.1: 7-1068 Human farnesyl pyrophosphate synthetase | ENSG00000160752 |
| | mRNA, complete cds | |
| | ATGAACGGAGACCAGAATTCAGATGTTTATGCCCAAGAA | |
| | AAGCAGGATTTCGTTCAGCACTTCTCCCAGATCGTTAGGGTGCT | |
| | GACTGAGGATGAGATGGGGCACCCAGAGATAGGAGATGCTATTG | |
| | CCCGGCTCAAGGAGGTCCTGGAGTACAATGCCATTGGAGGCAAG | |
| | TATAACCGGGGTTTGACGGTGGTAGTAGCATTCCGGGAGCTGGT | |
| | GGAGCCAAGGAAACAGGATGCTGATAGTCTCCAGCGGGCCTGGA | |
| | CTGTGGGCTGGTGTGTGGAACTGCTGCAAGCTTTCTTCCTGGTG | |
| | GCAGATGACATCATGGATTCATCCCTTACCCGCCGGGGACAGAC | |
| | CTGCTGGTATCAGAAGCCGGGCGTGGGTTTGGATGCCATCAATG | |
| | ATGCTAACCTCCTGGAAGCATGTATCTACCGCCTGCTGAAGCTC | |
| | TATTGCCGGGAGCAGCCCTATTACCTGAACCTGATCGAGCTCTT | |
| | CCTGCAGAGTTCCTATCAGACTGAGATTGGGCAGACCCTGGACC | |
| | TCCTCACAGCCCCCCAGGGCAATGTGGATCTTGTCAGATTCACT | |
| | GAAAAGAGGTACAAATCTATTGTCAAGTACAAGACAGCTTTCTA | |
| | CTCCTTCTACCTTCCTATAGCTGCAGCCATGTACATGGCAGGAA | |
| | TTGATGGCGAGAAGGAGCACGCCAATGCCAAGAAGATCCTGCTG | |
| | GAGATGGGGGAGTTCTTTCAGATTCAGGATGATTACCTTGACCT | |
| | CTTTGGGGACCCCAGTGTGACCGGCAAAATTGGCACTGACATCC | |
| | AGGACAACAAATGCAGCTGGCTGGTGGTTCAGTGTCTGCAACGG | |
| | GCCACTCCAGAACAGTACCAGATCCTGAAGGAAAATTACGGGCA | |
| | GAAGGAGGCTGAGAAAGTGGCCCGGGTGAAGGCGCTATATGAGG | |
| | AGCTGGATCTGCCAGCAGTGTTCTTGCAATATGAGGAAGACAGT | |
| | TACAGCCACATTATGGCTCTCATTGAACAGTACGCAGCACCCCT | |
| | GCCCCCAGCCGTCTTTCTGGGGCTTGCGCGCAAAATCTACAAGC | |
| | GGAGAAAGTGA | |
| Fndc3b | >XM 024453716.2: 100-3714 PREDICTED: Homo sapiens | ENSG00000075420 |
| | fibronectin type III domain containing 3B (FNDC3B), transcript | |
| | variant X2, mRNA | |
| | ATGTACGTCACAATGATGATGACCGACCAAATCCCTCTGGAACT | |
| | GCCACCATTGCTGAACGGAGAGGTAGCCATGATGCCCCACTTGG | |
| | TGAATGGAGATGCAGCTCAGCAGGTTATTCTCGTTCAAGTTAAT | |
| | CCAGGTGAGACTTTCACAATAAGAGCAGAGGATGGAACACTTCA | |
| | GTGCATTCAAGGACCTGCTGAAGTTCCCATGATGTCACCCAATG | |
| | GATCCATTCCTCCCATTCATGTGCCTCCAGGTTATATCTCACAG | |
| | GTGATTGAAGATAGTACTGGAGTCCGCCGGGTGGTGGTCACACC | |
| | CCAGTCTCCTGAGTGTTATCCCCCAAGCTACCCCTCAGCCATGT | |
| | CTCCAACCCATCATCTCCCTCCCTATCTGACTCACCATCCACAT | |
| | TTTATTCATAACTCACACACGGCTTACTACCCACCTGTTACCGG | |
| | ACCTGGAGATATGCCGCCTCAGTTTTTTCCCCAGCATCATCTTC | |
| | CCCACACAATATATGGTGAGCAAGAAATTATACCATTTTATGGA | |
| | ATGTCAACCTACATCACCCGAGAAGACCAGTACAGCAAGCCTCC | |
| | GCACAAAAAACTGAAAGACCGCCAGATCGATCGCCAGAACCGCC | |
| | TCAACAGCCCTCCTTCTTCTATCTACAAAAGCAGCTGCACAACA | |
| | GTATACAATGGCTATGGGAAGGGCCATAGTGGTGGAAGTGGCGG | |
| | AGGCGGCAGCGGTAGTGGTCCCGGAATTAAGAAAACAGAGCGAC | |
| | GAGCAAGAAGCAGCCCAAAGTCGAATGATTCAGACTTGCAAGAA | |
| | TATGAGTTGGAAGTAAAGAGGGTGCAAGACATTCTTTCGGGAAT | |
| | AGAGAAACCACAGGTTTCTAATATTCAGGCAAGAGCAGTTGTGT | |
| | TGTCCTGGGCTCCCCCTGTTGGACTTTCCTGTGGACCCCACAGT | |
| | GGTCTTTCCTTCCCCTACAGTTACGAGGTGGCCTTATCAGACAA | |
| | AGGACGAGATGGAAAATACAAGATAATTTACAGTGGAGAAGAAT | |
| | TAGAATGTAACCTGAAAGATCTTAGACCAGCAACAGATTATCAT | |
| | GTGAGGGTGTATGCCATGTACAATTCCGTAAAGGGATCCTGCTC | |
| | CGAGCCTGTTAGCTTCACCACCCACAGCTGTGCACCCGAGTGTC | |
| | CTTTCCCCCCTAAGCTGGCACATAGGAGCAAAAGTTCACTAACC | |
| | CTGCAGTGGAAGGCACCAATTGACAACGGTTCAAAAATCACCAA | |
| | CTACCTTTTAGAGTGGGATGAGGGAAAAAGAAATAGTGGTTTCA | |
| | GACAGTGCTTCTTCGGGAGCCAGAAGCACTGCAAGTTGACAAAG | |
| | CTTTGTCCGGCAATGGGGTACACATTCAGGCTGGCCGCTCGAAA | |
| | CGACATTGGTACCAGTGGTTATAGCCAAGAGGTGGTGTGCTACA | |
| | CATTAGGAAATATCCCTCAGATGCCTTCTGCACCAAGGCTGGTT | |
| | CGAGCTGGCATCACATGGGTCACGTTGCAGTGGAGTAAGCCAGA | |
| | AGGCTGTTCACCCGAGGAAGTGATCACCTACACCTTGGAAATTC | |
| | AGGAGGATGAAAATGATAACCTTTTCCACCCAAAATACACTGGA | |
| | GAGGATTTAACCTGTACTGTGAAAAATCTCAAAAGAAGCACACA | |
| | GTATAAATTCAGGCTGACTGCTTCTAATACGGAAGGAAAAAGCT | |
| | GTCCAAGCGAAGTTCTTGTTTGTACGACGAGTCCTGACAGGCCT | |
| | GGACCTCCTACCAGACCGCTTGTCAAAGGCCCAGTTACATCTCA | |
| | TGGCTTTAGTGTCAAATGGGATCCCCCTAAGGACAATGGTGGTT | |
| | CAGAAATCCTCAAGTACTTGCTAGAGATTACTGATGGAAATTCT | |
| | GAAGCGAATCAGTGGGAAGTGGCCTACAGTGGGTCGGCTACCGA | |
| | ATACACCTTCACCCACTTGAAACCAGGCACTTTGTACAAACTCC | |
| | GAGCATGCTGCATCAGTACCGGCGGACACAGCCAGTGTTCTGAA | |
| | AGTCTCCCTGTTCGCACACTAAGCATTGCACCAGGTCAATGTCG | |
| | ACCACCGAGGGTTTTGGGTAGACCAAAGCACAAAGAAGTCCACT | |
| | TAGAGTGGGATGTTCCTGCATCGGAAAGTGGCTGTGAGGTCTCA | |
| | GAGTACAGCGTGGAGATGACGGAGCCCGAAGACGTAGCCTCGGA | |
| | AGTGTACCATGGCCCAGAGCTGGAGTGCACCGTCGGCAACCTGC | |
| | TTCCTGGAACCGTGTATCGCTTCCGGGTGAGGGCTCTGAATGAT | |
| | GGAGGGTATGGTCCCTATTCTGATGTCTCAGAAATTACCACTGC | |
| | TGCAGGGCCTCCTGGACAATGCAAAGCACCTTGTATTTCTTGTA | |
| | CACCTGATGGATGTGTCTTAGTGGGTTGGGAGAGTCCTGATAGT | |
| | TCTGGTGCTGACATCTCAGAGTACAGGTTGGAATGGGGAGAAGA | |
| | TGAAGAATCCTTAGAACTCATTTATCATGGGACAGACACCCGTT | |
| | TTGAAATAAGAGACCTGTTGCCTGCTGCACAGTATTGCTGTAGA | |
| | CTACAGGCCTTCAATCAAGCAGGGGCAGGGCCGTACAGTGAACT | |
| | TGTCCTTTGCCAGACGCCAGCGTCTGCCCCTGACCCCGTCTCCA | |
| | CTCTCTGTGTCCTGGAGGAGGAGCCCCTTGATGCCTACCCTGAT | |
| | TCACCTTCTGCGTGCCTTGTACTGAACTGGGAAGAGCCGTGCAA | |
| | TAACGGATCTGAAATCCTTGCTTACACCATTGATCTAGGAGACA | |
| | CTAGCATTACCGTGGGCAACACCACCATGCATGTTATGAAAGAT | |
| | CTCCTTCCAGAAACCACCTACCGGATCAGAATTCAGGCTATAAA | |
| | TGAAATTGGAGCTGGACCATTTAGTCAGTTCATTAAAGCAAAAA | |
| | CTCGGCCATTACCACCCTTGCCTCCTAGGCTAGAATGTGCTGCT | |
| | GCTGGTCCTCAGAGCCTGAAGCTAAAATGGGGAGACAGTAACTC | |
| | CAAGACACATGCTGCTGAGGACATTGTGTACACACTACAGCTGG | |
| | AGGACAGAAACAAGAGGTTTATTTCAATCTACAGAGGACCCAGC | |
| | CACACCTACAAGGTCCAGAGACTGACGGAATTCACATGCTACTC | |
| | CTTCAGAATCCAGGCAGCAAGCGAGGCTGGAGAAGGGCCCTTCT | |
| | CAGAAACCTATACCTTCAGCACAACCAAAAGTGTCCCCCCCACC | |
| | ATCAAAGCACCTCGAGTAACACAGTTAGAAGGAAATTCATGTGA | |
| | AATTTTATGGGAGACGGTACCATCAATGAAAGGTGACCCTGTTA | |
| | ACTACATTCTGCAGGTATTGGTTGGAAGAGAATCTGAGTACAAA | |
| | CAGGTGTACAAGGGAGAAGAAGCCACATTCCAAATCTCAGGCCT | |
| | CCAGACCAACACAGACTACAGGTTCCGCGTATGTGCGTGTCGTC | |
| | GCTGTTTAGACACCTCTCAGGAGCTAAGCGGAGCCTTCAGCCCC | |
| | TCTGCGGCTTTTGTATTACAACGAAGTGAGGTCATGCTTACAGG | |
| | GGACATGGGGAGCTTAGATGATCCCAAAATGAAGAGCATGATGC | |
| | CTACTGATGAACAGTTTGCAGCCATCATTGTGCTTGGCTTTGCA | |
| | ACTTTGTCCATTTTATTTGCCTTTATATTACAGTACTTCTTAAT | |
| | GAAGTAA | |
| Gas6 | >L13720.1: 135-2171 Homo sapiens growth-arrest-specific protein | ENSG00000183087 |
| | (gas) mRNA, complete cds | |
| | ATGGCCCCTTCGCTCTCGCCCGGGCCCGCCGCCCTGCGCCGCGC | |
| | GCCGCAGCTGCTGCTGCTGCTGCTGGCCGCGGAGTGCGCGCTTG | |
| | CCGCGCTGTTGCCGGCGCGCGAGGCCACGCAGTTCCTGCGGCCC | |
| | AGGCAGCGCCGCGCCTTTCAGGTCTTCGAGGAGGCCAAGCAGGG | |
| | CCACCTGGAGAGGGAGTGCGTGGAGGAGCTGTGCAGCCGCGAGG | |
| | AGGCGCGGGAGGTGTTCGAGAACGACCCCGAGACGGATTATTTT | |
| | TACCCAAGATACTTAGACTGCATCAACAAGTATGGGTCTCCGTA | |
| | CACCAAAAACTCAGGCTTCGCCACCTGCGTGCAAAACCTGCCTG | |
| | ACCAGTGCACGCCCAACCCCTGCGATAGGAAGGGGACCCAAGCC | |
| | TGCCAGGACCTCATGGGCAACTTCTTCTGCCTGTGTAAAGCTGG | |
| | CTGGGGGGGCCGGCTCTGCGACAAAGATGTCAACGAATGCAGCC | |
| | AGGAGAACGGGGGCTGCCTCCAGATCTGCCACAACAAGCCGGGT | |
| | AGCTTCCACTGTTCCTGCCACAGCGGCTTCGAGCTCTCCTCTGA | |
| | TGGCAGGACCTGCCAAGACATAGACGAGTGCGCAGACTCGGAGG | |
| | CCTGCGGGGAGGCGCGCTGCAAGAACCTGCCCGGCTCCTACTCC | |
| | TGCCTCTGTGACGAGGGCTTTGCGTACAGCTCCCAGGAGAAGGC | |
| | TTGCCGAGATGTGGACGAGTGTCTGCAGGGCCGCTGTGAGCAGG | |
| | TCTGCGTGAACTCCCCAGGGAGCTACACCTGCCACTGTGACGGG | |
| | CGTGGGGGCCTCAAGCTGTCCCAGGACATGGACACCTGTGAGGA | |
| | CATCTTGCCGTGCGTGCCCTTCAGCGTGGCCAAGAGTGTGAAGT | |
| | CCTTGTACCTGGGCCGGATGTTCAGTGGGACCCCCGTGATCCGA | |
| | CTGCGCTTCAAGAGGCTGCAGCCCACCAGGCTGGTAGCTGAGTT | |
| | TGACTTCCGGACCTTTGACCCCGAGGGCATCCTCCTCTTTGCCG | |
| | GAGGCCACCAGGACAGCACCTGGATCGTGCTGGCCCTGAGAGCC | |
| | GGCCGGCTGGAGCTGCAGCTGCGCTACAACGGTGTCGGCCGTGT | |
| | CACCAGCAGCGGCCCGGTCATCAACCATGGCATGTGGCAGACAA | |
| | TCTCTGTTGAGGAGCTGGCGCGGAATCTGGTCATCAAGGTCAAC | |
| | AGGGATGCTGTCATGAAAATCGCGGTGGCCGGGGACTTGTTCCA | |
| | ACCGGAGCGAGGACTGTATCATCTGAACCTGACCGTGGGAGGTA | |
| | TTCCCTTCCATGAGAAGGACCTCGTGCAGCCTATAAACCCTCGT | |
| | CTGGATGGCTGCATGAGGAGCTGGAACTGGCTGAACGGAGAAGA | |
| | CACCACCATCCAGGAAACGGTGAAAGTGAACACGAGGATGCAGT | |
| | GCTTCTCGGTGACGGAGAGAGGCTCTTTCTACCCCGGGAGCGGC | |
| | TTCGCCTTCTACAGCCTGGACTACATGCGGACCCCTCTGGACGT | |
| | CGGGACTGAATCAACCTGGGAAGTAGAAGTCGTGGCTCACATCC | |
| | GCCCAGCCGCAGACACAGGCGTGCTGTTTGCGCTCTGGGCCCCC | |
| | GACCTCCGTGCCGTGCCTCTCTCTGTGGCACTGGTAGACTATCA | |
| | CTCCACGAAGAAACTCAAGAAGCAGCTGGTGGTCCTGGCCGTGG | |
| | AGCATACGGCCTTGGCCCTAATGGAGATCAAGGTCTGCGACGGC | |
| | CAAGAGCACGTGGTCACCGTCTCGCTGAGGGACGGTGAGGCCAC | |
| | CCTGGAGGTGGACGGCACCAGGGGCCAGAGCGAGGTGAGCGCCG | |
| | CGCAGCTGCAGGAGAGGCTGGCCGTGCTCGAGAGGCACCTGCGG | |
| | AGCCCCGTGCTCACCTTTGCTGGCGGCCTGCCAGATGTGCCGGT | |
| | GACTTCAGCGCCAGTCACCGCGTTCTACCGCGGCTGCATGACAC | |
| | TGGAGGTCAACCGGAGGCTGCTGGACCTGGACGAGGCGGCGTAC | |
| | AAGCACAGCGACATCACGGCCCACTCCTGCCCCCCCGTGGAGCC | |
| | CGCCGCAGCCTAG | |
| Gsta4 | >Y13047.1 Homo sapiens mRNA for glutathione transferase A4-4 | ENSG00000170899 |
| | ATGGCAGCAAGGCCCAAGCTCCACTATCCCAACGGAAGAGGCCG | |
| | GATGGAGTCCGTGAGATGGGTTTTAGCTGCCGCCGGAGTCGAGT | |
| | TTGATGAAGAATTTCTGGAAACAAAAGAACAGTTGTACAAGTTG | |
| | CAGGATGGTAACCACCTGCTGTTCCAACAAGTGCCCATGGTTGA | |
| | AATTGACGGGATGAAGTTGGTACAGACCCGAAGCATTCTCCACT | |
| | ACATAGCAGACAAGCACAATCTCTTTGGCAAGAACCTCAAGGAG | |
| | AGAACCCTGATTGACATGTACGTGGAGGGGACACTGGATCTGCT | |
| | GGAACTGCTTATCATGCATCCTTTCTTAAAACCAGATGATCAGC | |
| | AAAAGGAAGTGGTTAACATGGCCCAGAAGGCTATAATTAGATAC | |
| | TTTCCTGTGTTTGAAAAGATTTTAAGGGGTCACGGACAAAGCTT | |
| | TCTTGTTGGTAATCAGCTGAGCCTTGCAGATGTGATTTTACTCC | |
| | AAACCATTTTAGCTCTAGAAGAGAAAATTCCTAATATCCTGTCT | |
| | GCATTTCCTTTCCTCCAGGAATACACAGTGAAACTAAGTAATAT | |
| | CCCTACAATTAAGAGATTCCTTGAACCTGGCAGCAAGAAGAAGC | |
| | CTCCCCCTGATGAAATTTATGTGAGAACCGTCTACAACATCTTT | |
| | AGGCCATAA | |
| Hmgcs1 | >X66435.1: 123-1685 H. sapiens mRNA for HMG-COA-synthase | ENSG00000112972 |
| | ATGCCTGGATCACTTCCTTTGAATGCAGAAGCTTGCTGGCCAAA | |
| | AGATGTGGGAATTGTTGCCCTTGAGATCTATTTTCCTTCTCAAT | |
| | ATGTTGATCAAGCAGAGTTGGAAAAATATGATGGTGTAGATGCT | |
| | GGAAAGTATACCATTGGCTTGGGCCAGGCCAAGATGGGCTTCTG | |
| | CACAGATAGAGAAGATATTAACTCTCTTTGCATGACTGTGGTTC | |
| | AGAATCTTATGGAGAGAAATAACCTTTCCTATGATTGCATTGGG | |
| | CGGCTGGAAGTTGGAACAGAGACAATCATCGACAAATCAAAGTC | |
| | TGTGAAGACTAATTTGATGCAGCTGTTTGAAGAGTCTGGGAATA | |
| | CAGATATAGAAGGAATCGACACAACTAATGCATGCTATGGAGGC | |
| | ACAGCTGCTGTCTTCAATGCTGTTAACTGGATTGAGTCCAGCTC | |
| | TTGGGATGGACGGTATGCCCTGGTAGTTGCAGGAGATATTGCTG | |
| | TATATGCCACAGGAAATGCTAGACCTACAGGTGGAGTTGGAGCA | |
| | GTAGCTCTGCTAATTGGGCCAAATGCTCCTTTAATTTTTGAACG | |
| | AGGGCTTCGTGGGACACATATGCAACATGCCTATGATTTTTACA | |
| | AGCCTGATATGCTATCTGAATATCCTATAGTAGATGGAAAACTC | |
| | TCCATACAGTGCTACCTCAGTGCATTAGACCGCTGCTATTCTGT | |
| | CTACTGCAAAAAGATCCATGCCCAGTGGCAGAAAGAGGCAAATG | |
| | ATAACGATTTTACCTTGAATGATTTTGGCTTCATGATCTTTCAC | |
| | TCACCATATTGTAAACTGGTTCAGAAATCTCTAGCTCGGATGTT | |
| | GCTGAATGACTTCCTTAATGACCAGAATAGAGATAAAAATAGTA | |
| | TCTATAGTGGCCTGAAGGCCTTTGGGGATGTTAAGTTAGAAGAC | |
| | ACCTACTTTGATAGAGATGTGGAGAAGGCATTTATGAAGGCTAG | |
| | CTCTGAACTCTTCAGTCAGAAAACAAAGGCATCTTTACTTGTAT | |
| | CAAATCAAAATGGAAATATGTACACATCTTCAGTATATGGTTCC | |
| | CTTGCATCTGTTCTAGCACAGTACTCACCTCAGCATTTAGCAGG | |
| | GAAGAGAATTGGAGTGTTTTCTTATGGTTCTGGTTTGGCTGCCA | |
| | CTCTGTACTCTCTTAAAGTCACACAAGATGCTACACCGGGGTCT | |
| | GCTCTTGATAAAATAACAGCAAGTTTATGTGATCTTAAATCAAG | |
| | GCTTGATTCAAGAACTGGTGTGGCACAAGATGTCTTCGCTGAAA | |
| | ACATGAAGCTCAGAGAGGACACCCATCATTTGGTCAACTATATT | |
| | CCCCAGGGTTCAATAGATTCACTCTTTGAAGGAACGTGGTACTT | |
| | AGTTAGGGTGGATGAAAAGCACAGAAGAACTTACGCTCGGCGTC | |
| | CCACTCCAAATGATGACACTTTGGATGAAGGAGTAGGACTTGTG | |
| | CATTCAAACATAGCAACTGAGCATATTCCAAGCCCTGCCAAGAA | |
| | AGTACCAAGACTCCCTGCTACAGCAGCAGAACCTGAAGCAGCAG | |
| | TTATTAGTAATGGGGTATGGTAA | |
| Kcnd2 | >AF121104.1 Homo sapiens potassium channel KV4.2 (KCND2) | ENSG00000184408 |
| | mRNA, complete cds | |
| | ATGGCGGCGGGGGTGGCAGCGTGGCTGCCTTTTGCAAGGGCAGC | |
| | GGCTATCGGGTGGATGCCTGTGGCCTCGGGGCCTATGCCGGCTC | |
| | CCCCGAGGCAGGAGAGGAAAAGGACCCAAGATGCTCTCATTGTG | |
| | CTGAATGTGAGTGGCACCCGCTTCCAGACGTGGCAGGACACCCT | |
| | GGAACGTTACCCAGACACTCTACTGGGCAGTTCTGAGAGGGACT | |
| | TTTTCTACCACCCAGAAACTCAGCAGTATTTCTTTGACCGTGAC | |
| | CCAGACATCTTCCGCCACATCCTGAATTTCTACCGCACTGGGAA | |
| | GCTCCACTATCCTCGCCACGAGTGCATCTCTGCTTACGATGAAG | |
| | AACTGGCCTTCTTTGGCCTCATCCCGGAAATCATCGGCGACTGC | |
| | TGTTATGAGGAGTACAAGGATCGCAGGCGAGAGAACGCCGAGCG | |
| | CCTGCAGGACGACGCGGATACCGACACCGCTGGGGAGAGCGCCT | |
| | TGCCCACCATGACTGCAAGGCAGAGGGTCTGGAGGGCCTTCGAG | |
| | AACCCCCACACCAGCACGATGGCCCTGGTGTTCTACTATGTCAC | |
| | GGGGTTTTTCATTGCCGTCTCTGTCATCGCGAATGTGGTGGAAA | |
| | CAGTGCCGTGCGGATCAAGCCCAGGTCACATTAAAGAACTGCCC | |
| | TGTGGAGAGCGGTATGCTGTGGCCTTCTTCTGCTTGGACACGGC | |
| | CTGCGTCATGATCTTCACAGTTGAGTATTTGCTTCGCCTGGCTG | |
| | CAGCGCCTAGTCGTTACCGTTTTGTGCGTAGTGTCATGAGTATC | |
| | ATCGACGTGGTGGCCATCCTGCCTTATTACATTGGGCTGGTGAT | |
| | GACAGACAATGAGGACGTCAGCGGAGCCTTTGTCACACTCCGAG | |
| | TCTTCCGGGTCTTCAGGATCTTTAAGTTTTCCCGCCACTCTCAA | |
| | GGCCTGCGCATCCTGGGGTACACACTGAAGAGTTGTGCCTCAGA | |
| | ATTGGGCTTCTTGCTTTTCTCGCTCACCATGGCTATCATCATTT | |
| | TCGCTACGGTTATGTTCTACGCAGAGAAGGGCTCTTCAGCAAGC | |
| | AAGTTCACCAGCATCCCTGCAGCCTTCTGGTACACCATCGTCAC | |
| | CATGACAACACTGGGGTATGGCGACATGGTACCAAAAACCATAG | |
| | CAGGGAAGATTTTCGGGTCTATCTGCTCACTGAGCGGAGTCTTG | |
| | GTCATCGCGCTACCCGTGCCTGTGATCGTGTCTAACTTCAGTCG | |
| | GATCTACCACCAAAACCAACGAGCGGACAAACGAAGGGCACAGA | |
| | AGAAAGCGAGGCTGGCCAGGATCCGGGCAGCCAAAAGTGGAAGT | |
| | GCAAATGCCTACATGCAGAGCAAGCGGAGTGGGTTACTGAGCAA | |
| | CCAACTGCAGTCCTCGGAGGATGAACCGGCCTTCGTTAGCAAAT | |
| | CTGGATCCAGCTTCGAGACGCAACACCACCACCTGCTTCACTGC | |
| | CTGGAGAAAACCACGAACCATGAGTTTGTGGATGAACAAGTCTT | |
| | TGAAGAAAGCTGCATGGAAGTGGCCACTGTTAATCGCCCTTCAA | |
| | GTCACAGCCCCTCCCTCTCTTCCCAACAAGGAGTCACCAGCACT | |
| | TGCTGCTCACGGAGACACAAAAAAACTTTCCGAATCCCAAATGC | |
| | CAATGTGTCAGGAAGTCATAGAGGCAGCGTGCAAGAACTCAGTA | |
| | CAATTCAGATCAGATGTGTGGAGAGAACTCCACTATCCAACAGC | |
| | CGATCCAGCTTAAATGCCAAAATGGAAGAGTGTGTCAAACTAAA | |
| | CTGTGAACAACCTTACGTGACCACAGCAATAATTAGCATTCCAA | |
| | CACCTCCAGTAACCACCCCAGAAGGCGACGACAGGCCCGAGTCT | |
| | CCTGAGTATTCGGGAGGAAACATCGTCAGGGTGTCTGCTTTGTA | |
| | A | |
| Kit | >X69301.1 H. sapiens KIT proto-oncogene for mast/stem cell | ENSG00000157404 |
| | growth factor receptor, exon 1 | |
| | GGGCTCAATTTCCTAACGCTCCCCTCCCCATCCCCATGCCACCT | |
| | CCACGAGCAGCGGCGTCCAGCCTCCTCCCGCCCGAACGTGCTCG | |
| | AGGGGGGGGCAGTCGACCTTTATTGTCTGGGGAGCACCTGGCAG | |
| | GTGGCGGGCCCGTGCCCTAACGTGTGCGTGGTGCCCAGCTTCAC | |
| | AAAGCGAGCGGGCAGCACCTCCTTGGTCCGGGAACGCCTCAGCC | |
| | TGGCCGTCCACATCCCAGGGGTGGAAAGGTGGAGAGAGAAAGGG | |
| | GCTCCGGAGTCAAGAGGGGGGAGAGAGGGCGCGCGCGCCCTCCT | |
| | CCTCCCGGCGGGCACAGCCCCCCGGCATTAACACGTCGAAAGAG | |
| | CAGGGGCCAGACGCCGCCGGGAAGAAGCGAGACCCGGGCGGGCG | |
| | CGAGGGAGGGGAGGCGAGGAGGGGCGTGGCCGGCGCGCAGAGGG | |
| | AGGGCGCTGGGAGGAGGGGCTGCTGCTCGCCGCTCGCGGCTCTG | |
| | GGGGCTCGGCTTTGCCGCGCTCGCTGCACTTGGGCGAGAGCTGG | |
| | AACGTGGACCAGAGCTCGGATCCCATCGCAGCTACCGCGATGAG | |
| | AGGCGCTCGCGGCGCCTGGGATTTTCTCTGCGTTCTGCTCCTAC | |
| | TGCTTCGCGTCCAGACAGGTGGGACACCGCGGCTGGCACCCCGA | |
| | CCGTGCGACTACTCGGCGAAGCCTGTGCCCGGGAGGTGGTACCC | |
| | GCCAGGGTGCATCCGGAGAGAGGACTGCGGGCCCTCAGT | |
| Lgals1 | >AF222695.1:95-922 Homo sapiens galectin-related inhibitor of | ENSG00000133317 |
| | proliferation isoform a (GRIP1) mRNA, complete cds | |
| | ATGGTCATGCTGCAAGGAGTGGTCCCTCTAGATGCACACAGGTT | |
| | TCAGGTGGACTTCCAGTGTGGCTGCAGCCTGTGTCCCCGGCCAG | |
| | ATATCGCCTTCCACTTCAACCCTCGCTTCCATACCACCAAGCCC | |
| | CATGTCATCTGCAACACCCTGCATGGTGGACGCTGGCAAAGGGA | |
| | GGCCCGGTGGCCCCACCTGGCCCTGCGAAGAGGCTCCAGCTTCC | |
| | TCATCCTCTTTCTCTTCGGGAATGAGGAAGTGAAGGTGAGTGTG | |
| | AATGGACAGCACTTTCTCCACTTCCGCTACCGGCTCCCACTGTC | |
| | TCATGTGGACACGCTGGGTATATTTGGTGACATCCTGGTAGAGG | |
| | CTGTTGGATTCCTGAACATCAATCCATTTGTGGAGGGCAGCAGA | |
| | GAGTACCCAGCTGGACATCCTTTCCTGCTGATGAGCCCCAGGCT | |
| | GGAGGTGCCCTGCTCACATGCTCTTCCCCAGGGTCTCTCGCCTG | |
| | GGCAGGTCATCATAGTACGGGGACTGGTCTTGCAAGAGCCGAAG | |
| | CATTTTACTGTGAGCCTGAGGGACCAGGCTGCCCATGCTCCTGT | |
| | GACACTCAGGGCCTCCTTCGCAGACAGAACTCTGGCCTGGATCT | |
| | CCCGCTGGGGGCAGAAGAAACTGATCTCAGCCCCCTTCCTCTTT | |
| | TACCCCCAGAGATTCTTTGAGGTGCTGCTCCTGTTCCAGGAGGG | |
| | AGGGCTGAAGCTGGCGCTCAATGGGCAGGGGCTGGGGGCCACCA | |
| | GCATGAACCAGCAGGCCCTGGAGCAGCTGCGGGAGCTCCGGATC | |
| | AGTGGAAGTGTCCAGCTCTACTGTGTCCACTCCTGA | |
| Mast4 | >BC033215.1:277-1029 Homo sapiens microtubule associated | ENSG00000069020 |
| | serine/threonine kinase family member 4, mRNA (cDNA clone | |
| | MGC: 45875 IMAGE: 5015614), complete cds | |
| | ATGGGGGAGAAAGTTTCGGAGGCGCCAGAGCCGGTGCCCCGCGG | |
| | CTGCAGTGGCCACGGCAGCCGGACTCCAGCCTCTGCGCTGGTCG | |
| | CCGCGTCCTCTCCGGGTGCTTCCTCGGCCGAGTCCTCCTCGGGC | |
| | TCAGAAACTCTGTCGGAGGAAGGGGAGCCCGGCGGCTTCTCCAG | |
| | AGAGCATCAGCCGCCGCCGCCGCCGCCGTTGGGAGGCACCCTGG | |
| | GCGCCCGGGCGCCCGCCGCGTGGGCTCCGGCAAGCGTGCTGCTG | |
| | GAGCGCGGAGTCCTTGCGCTGCCGCCGCCGCTTCCCGGAGGAGC | |
| | TGTGCCGCCCGCGCCCCGGGGCAGCAGCGCGTCCCAGGAGGAGC | |
| | AGGACGAGGAGCTTGACCACATATTATCCCCTCCACCCATGCCG | |
| | TTTCGGAAATGCAGCAACCCAGATGTGGCTTCTGGCCCTGGAAA | |
| | ATCACTGAAGTATAAAAGACAGCTGAGTGAGGATGGAAGACAGC | |
| | TAAGGCGAGGGAGCCTGGGAGGAGCCCTGACTGGGAGGTACCTT | |
| | CTTCCAAACCCGGTGGGGGGACAGGCCTGGCCGGCCTCTGCAGA | |
| | GACGTCCAACCTCGTGCGCATGCGCAGCCAGGCCCTGGGCCAGT | |
| | CGGCGCCCTCGCTCACCGCCAGCCTGAAGGAGCTGAGTCTCCCC | |
| | AGAAGAGGAAGTTTGATAGATTCCCAGAAGTGGAATTGCTTGGT | |
| | CAAACGCCCTGTGTGTCCAAATGCTGGGAGAACATCACCCCTTG | |
| | GATGA | |
| Mt2 | >U25341.1 Human Mellb-melatonin receptor (MTNR1B) mRNA, | ENSG00000134640 |
| | complete cds | |
| | GGAGAGTCTGCGATGTCAGAGAACGGCTCCTTCGCCAACTGCTG | |
| | CGAGGCGGGCGGGTGGGCAGTGCGCCCGGGCTGGTCGGGGGCTG | |
| | GCAGCGCGCGGCCCTCCAGGACCCCTCGACCTCCCTGGGTGGCT | |
| | CCAGCGCTGTCCGCGGTGCTCATCGTCACCACCGCCGTGGACGT | |
| | CGTGGGCAACCTCCTGGTGATCCTCTCCGTGCTCAGGAACCGCA | |
| | AGCTCCGGAACGCAGGTAATTTGTTCTTGGTGAGTCTGGCATTG | |
| | GCTGACCTGGTGGTGGCCTTCTACCCCTACCCGCTAATCCTCGT | |
| | GGCCATCTTCTATGACGGCTGGGCCCTGGGGGAGGAGCACTGCA | |
| | AGGCCAGCGCCTTTGTGATGGGCCTGAGCGTCATCGGCTCTGTC | |
| | TTCAATATCACTGCCATCGCCATTAACCGCTACTGCTACATCTG | |
| | CCACAGCATGGCCTACCACCGAATCTACCGGCGCTGGCACACCC | |
| | CTCTGCACATCTGCCTCATCTGGCTCCTCACCGTGGTGGCCTTG | |
| | CTGCCCAACTTCTTTGTGGGGTCCCTGGAGTACGACCCACGCAT | |
| | CTATTCCTGCACCTTCATCCAGACCGCCAGCACCCAGTACACGG | |
| | CGGCAGTGGTGGTCATCCACTTCCTCCTCCCTATCGCTGTCGTG | |
| | TCCTTCTGCTACCTGCGCATCTGGGTGCTGGTGCTTCAGGCCCG | |
| | CAGGAAAGCCAAGCCAGAGAGCAGGCTGTGCCTGAAGCCCAGCG | |
| | ACTTGCGGAGCTTTCTAACCATGTTTGTGGTGTTTGTGATCTTT | |
| | GCCATCTGCTGGGCTCCACTTAACTGCATCGGCCTCGCTGTGGC | |
| | CATCAACCCCCAAGAAATGGCTCCCCAGATCCCTGAGGGGCTAT | |
| | TTGTCACTAGCTACTTACTGGCTTATTTCAACAGCTGCCTGAAT | |
| | GCCATTGTCTATGGGCTCTTGAACCAAAACTTCCGCAGGGAATA | |
| | CAAGAGGATCCTCTTGGCCCTTTGGAACCCACGGCACTGCATTC | |
| | AAGATGCTTCCAAGGGCAGCCACGCGGAGGGGCTGCAGAGCCCA | |
| | GCTCCACCCATCATTGGTGTGCAGCACCAGGCAGATGCTCTCTA | |
| | GCCTG | |
| Pak3 | >AF068864.1 Homo sapiens p21-activated kinase 3 (PAK3) | ENSG00000077264 |
| | mRNA, complete cds | |
| | ATGTCTGACGGTCTGGATAATGAAGAGAAACCCCCGGCTCCTCC | |
| | ACTGAGGATGAATAGTAACAACCGGGATTCTTCAGCACTCAACC | |
| | ACAGCTCCAAACCACTTCCCATGGCCCCTGAAGAGAAGAATAAG | |
| | AAAGCCAGGCTTCGCTCTATCTTCCCAGGAGGAGGGGATAAAAC | |
| | CAATAAGAAGAAGGAGAAAGAGCGCCCAGAGATCTCTCTTCCTT | |
| | CAGACTTTGAGCATACGATTCATGTGGGGTTTGATGCAGTCACC | |
| | GGGGAATTCACTGGAATTCCAGAGCAATGGGCACGATTACTCCA | |
| | AACTTCCAACATAACAAAATTGGAACAGAAGAAGAACCCACAAG | |
| | CTGTTCTAGATGTTCTCAAATTCTATGATTCCAAAGAAACAGTC | |
| | AACAACCAGAAATACATGAGCTTTACATCAGGAGATAAAAGTGC | |
| | ACATGGATACATAGCAGCCCATCCTTCGAGTACAAAAACAGCAT | |
| | CTGAGCCTCCATTGGCCCCTCCTGTGTCTGAAGAAGAAGATGAA | |
| | GAGGAAGAAGAAGAAGAAGATGAAAATGAGCCACCACCAGTTAT | |
| | CGCACCAAGACCAGAGCATACAAAATCAATCTATACTCGTTCTG | |
| | TGGTTGAATCCATTGCTTCACCAGCAGTACCAAATAAAGAGGTC | |
| | ACACCACCCTCTGCTGAAAATGCCAATTCCAGTACTTTGTACAG | |
| | GAACACAGATCGGCAAAGAAAAAAATCCAAGATGACAGATGAGG | |
| | AGATCTTAGAGAAGCTAAGAAGCATTGTGAGTGTTGGGGACCCA | |
| | AAGAAAAAATACACAAGATTTGAAAAAATTGGTCAAGGGGCATC | |
| | AGGTACTGTTTATACAGCACTAGACATTGCAACAGGACAAGAGG | |
| | TGGCCATAAAGCAGATGAACCTTCAACAGCAACCCAAGAAGGAA | |
| | TTAATTATTAATGAAATTCTGGTCATGAGGGAAAATAAGAACCC | |
| | TAATATTGTTAATTATTTAGATAGCTACTTGGTGGGTGATGAAC | |
| | TATGGGTAGTCATGGAATACTTGGCTGGTGGCTCTCTGACTGAT | |
| | GTGGTCACAGAGACCTGTATGGATGAAGGACAGATAGCAGCTGT | |
| | CTGCAGAGAGTGCCTGCAAGCTTTGGATTTCCTGCACTCAAACC | |
| | AGGTGATCCATAGAGATATAAAGAGTGACAATATTCTTCTCGGG | |
| | ATGGATGGCTCTGTTAAATTGACTGACTTTGGGTTCTGTGCCCA | |
| | GATCACTCCTGAGCAAAGTAAACGAAGCACTATGGTGGGAACCC | |
| | CATATTGGATGGCACCTGAGGTGGTGACTCGAAAAGCTTATGGT | |
| | CCGAAAGTTGATATCTGGTCTCTTGGAATTATGGCAATTGAAAT | |
| | GGTGGAAGGTGAACCCCCTTACCTTAATGAAAATCCACTCAGGG | |
| | CATTGTATCTGATAGCCACTAATGGAACTCCAGAGCTCCAGAAT | |
| | CCTGAGAGACTGTCAGCTGTATTCCGTGACTTTTTAAATCGCTG | |
| | TCTTGAGATGGATGTGGATAGGCGAGGATCTGCCAAGGAGCTTT | |
| | TGCAGCATCCATTTTTAAAATTAGCCAAGCCTCTCTCCAGCCTG | |
| | ACTCCTCTGATTATCGCTGCAAAGGAAGCAATTAAGAACAGCAG | |
| | CCGCTAA | |
| Pik3c2g | >AJ000008.1: 142-4488 Homo sapiens mRNA for C2 domain | ENSG00000139144 |
| | containing PI3-kinase | |
| | ATGGCATATTCTTGGCAAACGGATCCAAATCCTAATGAATCACA | |
| | CGAAAAGCAGTATGAACACCAAGAATTTCTCTTTGTAAATCAAC | |
| | CCCATTCTTCTAGCCAAGTCAGTCTGGGTTTTGATCAGATAGTA | |
| | GATGAGATCAGTGGCAAAATTCCACACTACGAGAGTGAAATTGA | |
| | TGAAAACACCTTTTTTGTGCCCACTGCACCAAAATGGGACTCAA | |
| | CAGGGCATTCATTAAATGAAGCACACCAAATATCCTTGAATGAA | |
| | TTCACTTCTAAAAGCCGTGAACTCTCCTGGCATCAAGTTAGCAA | |
| | AGCACCAGCAATTGGTTTTAGTCCTTCTGTGTTACCAAAACCTC | |
| | AAAATACGAATAAAGAATGCTCCTGGGGAAGCCCCATAGGAAAA | |
| | CATCATGGTGCTGATGATTCCAGATTCAGTATTTTAGCTCCATC | |
| | ATTCACAAGTTTGGATAAAATTAATCTAGAGAAAGAATTAGAAA | |
| | ATGAAAATCATAACTACCATATAGGATTTGAAAGTAGCATTCCT | |
| | CCAACAAATTCATCCTTCTCAAGTGACTTCATGCCGAAAGAAGA | |
| | GAATAAAAGGAGTGGACATGTGAACATTGTGGAACCATCTTTGA | |
| | TGCTTTTGAAAGGCTCTCTTCAACCCGGAATGTGGGAAAGTACA | |
| | TGGCAGAAGAATATAGAGTCAATAGGTTGTTCCATTCAGCTAGT | |
| | GGAAGTACCTCAAAGCAGCAATACGAGTCTGGCCTCTTTTTGCA | |
| | ACAAAGTAAAAAAAATCAGAGAAAGATATCATGCAGCTGATGTT | |
| | AATTTCAATTCTGGGAAGATCTGGAGCACTACTACAGCATTTCC | |
| | GTATCAGCTCTTTTCTAAGACCAAGTTTAATATACATATTTTTA | |
| | TTGATAACTCAACACAACCTCTTCATTTTATGCCATGTGCTAAT | |
| | TATCTTGTCAAAGATCTAATTGCAGAAATTCTGCATTTTTGCAC | |
| | AAATGACCAGCTACTCCCCAAAGATCATATTCTAAGTGTATGTG | |
| | GCTCTGAAGAATTTTTACAAAACGACCACTGTTTGGGGAGCCAC | |
| | AAAATGTTTCAAAAAGATAAATCTGTTATTCAGCTCCACCTGCA | |
| | GAAAAGTAGGGAAGCTCCAGGAAAGCTATCTCGAAAGCATGAAG | |
| | AGGACCACAGTCAGTTTTATCTGAATCAACTTCTAGAATTTATG | |
| | CATATTTGGAAAGTATCCAGACAATGTCTCTTAACACTCATCAG | |
| | AAAATATGACTTCCACCTGAAATACCTATTGAAAACCCAGGAAA | |
| | ACGTGTATAATATTATTGAAGAAGTTAAAAAAATATGCAGTGTT | |
| | CTAGGGTGTGTGGAAACCAAACAAATTACAGATGCAGTAAATGA | |
| | ACTAAGTCTAATTCTTCAGAGAAAAGGAGAGAATTTTTATCAAA | |
| | GTTCAGAGACTTCAGCAAAAGGCTTGATAGAGAAGGTAACAACT | |
| | GAACTATCCACATCCATCTACCAGCTAATCAATGTCTACTGTAA | |
| | CAGCTTTTATGCAGATTTTCAGCCTGTAAATGTACCTAGATGCA | |
| | CTTCCTATCTAAATCCCGGGCTTCCTTCCCACCTCAGCTTCACA | |
| | GTGTATGCAGCACACAACATTCCAGAAACCTGGGTGCACAGGAT | |
| | CAATTTTCCCCTTGAAATAAAGTCACTTCCAAGGGAATCCATGC | |
| | TCACTGTAAAACTGTTTGGGATTGCCTGTGCAACCAACAATGCA | |
| | AATTTACTGGCGTGGACTTGTCTTCCACTGTTTCCAAAAGAAAA | |
| | ATCCATTCTCGGGTCTATGCTGTTCAGCATGACATTACAGAGTG | |
| | AGCCTCCCGTAGAAATGATAACTCCAGGAGTGTGGGATGTAAGT | |
| | CAGCCATCCCCGGTGACCCTGCAGATTGATTTTCCAGCTACTGG | |
| | GTGGGAGTATATGAAACCTGATTCTGAAGAGAATAGAAGTAATC | |
| | TTGAAGAGCCACTAAAGGAGTGTATAAAACATATTGCCAGACTT | |
| | TCACAGAAACAGACTCCCCTACTACTCTCTGAAGAAAAGAAAAG | |
| | ATATTTATGGTTTTATCGCTTCTACTGCAATAATGAAAACTGCT | |
| | CCCTTCCTTTAGTCCTGGGTAGTGCCCCTGGATGGGATGAAAGG | |
| | ACTGTTTCAGAAATGCATACCATTTTGAGAAGATGGACATTTTC | |
| | TCAACCTTTAGAGGCTCTTGGGCTTTTGACTTCCAGTTTTCCAG | |
| | ATCAAGAAATTCGTAAAGTGGCAGTTCAACAATTAGACAACCTC | |
| | TTGAATGATGAACTACTGGAATATCTCCCACAGCTAGTTCAGGC | |
| | TGTCAAGTTTGAATGGAACCTTGAGAGTCCTTTAGTGCAACTTC | |
| | TACTCCACCGCTCCTTGCAGAGCATCCAGGTTGCCCATCGTCTT | |
| | TACTGGCTGCTAAAAAATGCAGAAAATGAAGCTTATTTTAAAAG | |
| | CTGGTATCAGAAGCTACTAGCTGCTCTCCAATTCTGTGCAGGTA | |
| | AAGCCTTGAATGATGAGTTTTCCAAGGAGCAGAAACTTATCAAA | |
| | ATTCTGGGAGATATTGGGGAAAGAGTCAAGTCTGCCAGTGACCA | |
| | TCAAAGACAGGAGGTACTGAAGAAAGAAATTGGCAGACTAGAAG | |
| | AGTTCTTTCAAGATGTAAATACTTGTCATCTTCCTCTGAACCCT | |
| | GCCCTATGTATAAAAGGGATTGATCACGATGCATGTTCATATTT | |
| | TACATCTAATGCTTTGCCATTGAAGATTACTTTCATCAATGCTA | |
| | ATCTGATGGGCAAAAACATCAGCATTATTTTTAAGGCTGGAGAT | |
| | GATCTTCGTCAGGATATGCTTGTTCTGCAGCTTATTCAAGTGAT | |
| | GGACAATATTTGGCTGCAGGAAGGCTTGGATATGCAAATGATCA | |
| | TTTATAGATGTCTATCCACAGGAAAAGACCAACGATTGGTGCAG | |
| | ATGGTACCTGATGCTGTGACCCTAGCAAAGATTCATCGCCATTC | |
| | TGGACTGATAGGACCATTGAAAGAAAATACCATTAAAAAGTGGT | |
| | TCAGTCAGCACAACCACTTAAAGGCAGATTATGAAAAGGCCTTG | |
| | AGGAACTTTTTCTACTCCTGTGCTGGCTGGTGTGTGGTAACATT | |
| | CATCCTGGGAGTATGTGACCGTCACAATGATAATATCATGCTGA | |
| | CAAAGTCGGGCCACATGTTTCATATTGACTTTGGAAAATTCTTA | |
| | GGTCATGCACAAACATTTGGAGGGATAAAAAGGGACCGAGCTCC | |
| | TTTCATTTTTACTTCAGAGATGGAATACTTTATTACAGAGGGTG | |
| | GGAAAAACCCACAGCATTTTCAAGATTTTGTGGAACTTTGCTGT | |
| | CGTGCTTATAATATTATCAGAAAGCACAGCCAACTGCTCTTGAA | |
| | CCTGCTGGAAATGATGCTGTATGCAGGACTGCCTGAGCTAAGTG | |
| | GAATTCAAGACCTGAAATATGTGTATAATAATCTTCGTCCACAA | |
| | GACACAGACCTGGAAGCAACAAGTCATTTTACCAAGAAAATAAA | |
| | GGAAAGTCTGGAGTGTTTCCCTGTTAAATTGAATAACTTGATCC | |
| | ACACACTTGCACAAATGTCAGCCATAAGCCCTGCCAAATCTACT | |
| | TCACAGACTTTTCCTCAGGAATCCTGTTTGCTGAGTACAACTAG | |
| | GTCGATTGAAAGAGCAATTTTAGGGTTCAGCAAGAAATCCAGTA | |
| | ATCTGTATCTGATCCAGGTGACACACAGCAACAACGAAACAAGC | |
| | CTGACAGAAAAATCATTTGAGCAGTTTTCAAAACTTCACAGCCA | |
| | ACTTCAGAAGCAGTTTGCATCACTGACTCTCCCAGAGTTTCCTC | |
| | ATTGGTGGCACCTACCTTTTACAAATTCAGATCACAGAAGATTC | |
| | AGAGATCTAAATCATTACATGGAACAGATATTAAATGTATCACA | |
| | TGAAGTTACAAACAGTGATTGTGTACTTAGCTTTTTCCTCTCTG | |
| | AGGCTGTGCAACAAACAGTTGAATCATCACCTGTGTACCTAGGT | |
| | GAGAAGAAGTTTCCAGACAAGAAGCCTAAGGTGCAGTTAGTCAT | |
| | ATCCTACGAGGATGTGAAGCTGACCATACTAGTGAAACACATGA | |
| | AAAACATTCATCTCCCAGATGGCTCTGCGCCCAGTGCACATGTT | |
| | GAATTTTATCTTTTACCATATCCCAGTGAAGTTCTGAGGAGGAA | |
| | AACAAAATCTGTTCCAAAATGTACGGACCCCACTTACAATGAAA | |
| | TTGTAGTATATGATGAAGTCACAGAGCTCCAAGGACATGTCTTA | |
| | ATGCTTATTGTGAAGAGTAAAACTGTATTTGTGGGAGCAATTAA | |
| | CATCCGACTCTGTAGTGTCCCACTCGATAAAGAAAAATGGTATC | |
| | CATTAGGAAACAGTATAATTTCACCATTGCTATGA | |
| Rampl | >AJ001014.1: 33-479 Homo sapiens mRNA encoding RAMP1 | ENSG00000132329 |
| | ATGGCCCGGGCCCTGTGCCGCCTCCCGCGGCGCGGCCTCTGGCT | |
| | GCTCCTGGCCCATCACCTCTTCATGACCACTGCCTGCCAGGAGG | |
| | CTAACTACGGTGCCCTCCTCCGGGAGCTCTGCCTCACCCAGTTC | |
| | CAGGTAGACATGGAGGCCGTCGGGGAGACGCTGTGGTGTGACTG | |
| | GGGCAGGACCATCAGGAGCTACAGGGAGCTGGCCGACTGCACCT | |
| | GGCACATGGCGGAGAAGCTGGGCTGCTTCTGGCCCAATGCAGAG | |
| | GTGGACAGGTTCTTCCTGGCAGTGCATGGCCGCTACTTCAGGAG | |
| | CTGCCCCATCTCAGGCAGGGCCGTGCGGGACCCGCCCGGCAGCA | |
| | TCCTCTACCCCTTCATCGTGGTCCCCATCACGGTGACCCTGCTG | |
| | GTGACGGCACTGGTGGTCTGGCAGAGCAAGCGCACTGAGGGCAT | |
| | TGTGTAG | |
| Smoc2 | >BC047583.1: 193-1533 Homo sapiens SPARC related modular | ENSG00000112562 |
| | calcium binding 2, mRNA (cDNA clone MGC: 48409 | |
| | IMAGE: 4826950), complete cds | |
| | ATGCTGCTCCCCCAGCTCTGCTGGCTGCCGCTGCTCGCTGGGCT | |
| | GCTCCCGCCGGTGCCCGCGCAGAAGTTCTCGGCGCTCACGTTTT | |
| | TGAGAGTGGATCAAGATAAAGACAAGGATTGTAGCTTGGACTGT | |
| | GCGGGTTCGCCCCAGAAACCTCTCTGCGCATCTGACGGAAGGAC | |
| | CTTCCTTTCCCGTTGTGAATTTCAACGTGCCAAGTGCAAAGATC | |
| | CCCAGCTAGAGATTGCATATCGAGGAAACTGCAAAGACGTGTCC | |
| | AGGTGTGTGGCCGAAAGGAAGTATACCCAGGAGCAAGCCCGGAA | |
| | GGAGTTTCAGCAAGTGTTCATTCCTGAGTGCAATGACGACGGCA | |
| | CCTACAGTCAGGTCCAGTGTCACAGCTACACGGGATACTGCTGG | |
| | TGCGTCACGCCCAACGGGAGGCCCATCAGCGGCACTGCCGTGGC | |
| | CCACAAGACGCCCCGGTGCCCGGGTTCCGTAAATGAAAAGTTAC | |
| | CCCAACGCGAAGGCACAGGAAAAACAGATGATGCCGCAGCTCCA | |
| | GCGTTGGAGACTCAGCCTCAAGGAGATGAAGAAGATATTGCATC | |
| | ACGTTACCCTACCCTTTGGACTGAACAGGTTAAAAGTCGGCAGA | |
| | ACAAAACCAATAAGAATTCAGTGTCATCCTGTGACCAAGAGCAC | |
| | CAGTCTGCCCTGGAGGAAGCCAAGCAGCCCAAGAACGACAATGT | |
| | GGTGATCCCTGAGTGTGCGCACGGCGGCCTCTACAAGCCAGTGC | |
| | AGTGCCACCCCTCCACGGGGTACTGCTGGTGCGTCCTGGTGGAC | |
| | ACGGGGCGCCCCATTCCCGGCACATCCACAAGGTACGAGCAGCC | |
| | GAAATGTGACAACACGGCCAGGGCCCACCCAGCCAAAGCCCGGG | |
| | ACCTGTACAAGGGCCGCCAGCTACAAGGTTGTCCGGGTGCCAAA | |
| | AAGCATGAGTTTCTGACCAGCGTTCTGGACGCGCTGTCCACGGA | |
| | CATGGTCCACGCCGCCTCCGACCCCTCCTCCTCGTCAGGCAGGC | |
| | TCTCAGAACCCGACCCCAGCCATACCCTAGAGGAGCGGGTGGTG | |
| | CACTGGTACTTCAAACTACTGGATAAAAACTCCAGTGGAGACAT | |
| | CGGCAAAAAGGAAATCAAACCCTTCAAGAGGTTCCTTCGCAAAA | |
| | AATCAAAGCCCAAAAAATGTGTGAAGAAGTTTGTTGAATACTGT | |
| | GACGTGAATAATGACAAATCCATCTCCGTACAAGAACTGATGGG | |
| | CTGCCTGGGCGTGGCGAAAGAGGACGGCAAAGCGGACACCAAGA | |
| | AACGCCACACCCCCAGAGGTCATGTTGAAAGTACGTCTAATAGA | |
| | CAGCCAAGGAAACAAGGATAA | |
| Tac1 | >X54469.1: 82-471 Human mRNA for beta-preprotachykinin | ENSG00000006128 |
| | ATGAAAATCCTCGTGGCCTTGGCAGTCTTTTTTCTTGTCTCCAC | |
| | TCAGCTGTTTGCAGAAGAAATAGGAGCCAATGATGATCTGAATT | |
| | ACTGGTCCGACTGGTACGACAGCGACCAGATCAAGGAGGAACTG | |
| | CCGGAGCCCTTTGAGCATCTTCTGCAGAGAATCGCCCGGAGACC | |
| | CAAGCCTCAGCAGTTCTTTGGATTAATGGGCAAACGGGATGCTG | |
| | ATTCCTCAATTGAAAAACAAGTGGCCCTGTTAAAGGCTCTTTAT | |
| | GGACATGGCCAGATCTCTCACAAAAGACATAAAACAGATTCCTT | |
| | TGTTGGACTAATGGGCAAAAGAGCTTTAAATTCTGTGGCTTATG | |
| | AAAGGAGTGCAATGCAGAATTATGAAAGAAGACGTTAA | |
| Timp1 | >M12670.1: 49-672 Human fibroblast collagenase inhibitor | ENSG00000102265 |
| | mRNA, complete cds | |
| | ATGGCCCCCTTTGAGCCCCTGGCTTCTGGCATCCTGTTGTTGCT | |
| | GTGGCTGATAGCCCCCAGCAGGGCCTGCACCTGTGTCCCACCCC | |
| | ACCCACAGACGGCCTTCTGCAATTCCGACCTCGTCATCAGGGCC | |
| | AAGTTCGTGGGGACACCAGAAGTCAACCAGACCACCTTATACCA | |
| | GCGTTATGAGATCAAGATGACCAAGATGTATAAAGGGTTCCAAG | |
| | CCTTAGGGGATGCCGCTGACATCCGGTTCGTCTACACCCCCGCC | |
| | ATGGAGAGTGTCTGCGGATACTTCCACAGGTCCCACAACCGCAG | |
| | CGAGGAGTTTCTCATTGCTGGAAAACTGCAGGATGGACTCTTGC | |
| | ACATCACTACCTGCAGTTTCGTGGCTCCCTGGAACAGCCTGAGC | |
| | TTAGCTCAGCGCCGGGGCTTCACCAAGACCTACACTGTTGGCTG | |
| | TGAGGAATGCACAGTGTTTCCCTGTTTATCCATCCCCTGCAAAC | |
| | TGCAGAGTGGCACTCATTGCTTGTGGACGGACCAGCTCCTCCAA | |
| | GGCTCTGAAAAGGGCTTCCAGTCCCGTCACCTTGCCTGCCTGCC | |
| | TCGGGAGCCAGGGCTGTGCACCTGGCAGTCCCTGCGGTCCCAGA | |
| | TAGCCTGA | |
| Tmsb4x | >M17733.1: 78-212 Human thymosin beta-4 mRNA, complete cds | ENSG00000205542 |
| | ATGTCTGACAAACCCGATATGGCTGAGATCGAGAAATTCGATAA | |
| | GTCGAAACTGAAGAAGACAGAGACGCAAGAGAAAAATCCACTGC | |
| | CTTCCAAAGAAACGATTGAACAGGAGAAGCAAGCAGGCGAATCG | |
| | TAA | |
| Tpm4 | >BC002827.2: 49-795 Homo sapiens tropomyosin 4, mRNA | ENSG00000167460 |
| | (cDNA clone MGC: 3641 IMAGE: 3637300), complete cds | |
| | ATGGCCGGCCTCAACTCCCTGGAGGCGGTGAAACGCAAGATCCA | |
| | GGCCCTGCAGCAGCAGGCGGACGAGGCGGAAGACCGCGCGCAGG | |
| | GCCTGCAGCGGGAGCTGGACGGCGAGCGCGAGCGGCGCGAGAAA | |
| | GCTGAAGGTGATGTGGCCGCCCTCAACCGACGCATCCAGCTCTT | |
| | TGAGGAGGAGTTGGACAGGGCTCAGGAACGACTGGCCACGGCCC | |
| | TGCAGAAGCTGGAGGAGGCAGAAAAAGCTGCAGATGAGAGTGAG | |
| | AGAGGAATGAAGGTGATAGAAAACCGGGCCATGAAGGATGAGGA | |
| | GAAGATGGAGATTCAGGAGATGCAGCTCAAAGAGGCCAAGCACA | |
| | TTGCGGAAGAGGCTGACCGCAAATACGAGGAGGTAGCTCGTAAG | |
| | CTGGTCATCCTGGAGGGTGAGCTGGAGAGGGCAGAGGAGCGTGC | |
| | GGAGGTGTCTGAACTAAAATGTGGTGACCTGGAAGAAGAACTCA | |
| | AGAATGTTACTAACAATCTGAAATCTCTGGAGGCTGCATCTGAA | |
| | AAGTATTCTGAAAAGGAGGACAAATATGAAGAAGAAATTAAACT | |
| | TCTGTCTGACAAACTGAAAGAGGCTGAGACCCGTGCTGAATTTG | |
| | CAGAGAGAACGGTTGCAAAACTGGAAAAGACAATTGATGACCTG | |
| | GAAGAGAAACTTGCCCAGGCCAAAGAAGAGAACGTGGGCTTACA | |
| | TCAGACACTGGATCAGACACTAAACGAACTTAACTGTATATAA | |
| Trib2 | >NM_021643.4: 1373-2404 Homo sapiens tribbles pseudokinase 2 | ENSG00000071575 |
| | (TRIB2), transcript variant 1, mRNA | |
| | ATGAACATACACAGGTCTACCCCCATCACAATAGCGAGATATGG | |
| | GAGATCGCGGAACAAAACCCAGGATTTCGAAGAGTTGTCGTCTA | |
| | TAAGGTCCGCGGAGCCCAGCCAGAGTTTCAGCCCGAACCTCGGC | |
| | TCCCCGAGCCCGCCCGAGACTCCGAACTTGTCGCATTGCGTTTC | |
| | TTGTATCGGGAAATACTTATTGTTGGAACCTCTGGAGGGAGACC | |
| | ACGTTTTTCGTGCCGTGCATCTGCACAGCGGAGAGGAGCTGGTG | |
| | TGCAAGGTGTTTGATATCAGCTGCTACCAGGAATCCCTGGCACC | |
| | GTGCTTTTGCCTGTCTGCTCATAGTAACATCAACCAAATCACTG | |
| | AAATTATCCTGGGTGAGACCAAAGCCTATGTGTTCTTTGAGCGA | |
| | AGCTATGGGGACATGCATTCCTTCGTCCGCACCTGCAAGAAGCT | |
| | GAGAGAGGAGGAGGCAGCCAGACTGTTCTACCAGATTGCCTCGG | |
| | CAGTGGCCCACTGCCATGACGGGGGGCTGGTGCTGCGGGACCTC | |
| | AAGCTGCGGAAATTCATCTTTAAGGACGAAGAGAGGACTCGGGT | |
| | CAAGCTGGAAAGCCTGGAAGACGCCTACATTCTGCGGGGAGATG | |
| | ATGATTCCCTCTCCGACAAGCATGGCTGCCCGGCTTACGTAAGC | |
| | CCAGAGATCTTGAACACCAGTGGCAGCTACTCGGGCAAAGCAGC | |
| | CGACGTGTGGAGCCTGGGGGTGATGCTGTACACCATGTTGGTGG | |
| | GGCGGTACCCTTTCCATGACATTGAACCCAGCTCCCTCTTCAGC | |
| | AAGATCCGGCGTGGCCAGTTCAACATTCCAGAGACTCTGTCGCC | |
| | CAAGGCCAAGTGCCTCATCCGAAGCATTCTGCGTCGGGAGCCCT | |
| | CAGAGCGGCTGACCTCGCAGGAAATTCTGGACCATCCTTGGTTT | |
| | TCTACAGATTTTAGCGTCTCGAATTCAGCATATGGTGCTAAGGA | |
| | AGTGTCTGACCAGCTGGTGCCGGACGTCAACATGGAAGAGAACT | |
| | TGGACCCTTTCTTTAACTGA | |
| Vim | >M14144.1: 292-1692 Human vimentin gene, complete cds | ENSG00000026025 |
| | ATGTCCACCAGGTCCGTGTCCTCGTCCTCCTACCGCAGGATGTT | |
| | CGGCGGCCCGGGCACCGCGAGCCGGCCGAGCTCCAGCCGGAGCT | |
| | ACGTGACTACGTCCACCCGCACCTACAGCCTGGGCGACGCGCTG | |
| | CGCCCCAGCACCAGCCGCAGCCTCTACGCCTCGTCCCCGGGCGG | |
| | CGTGTATGCCACGCGCTCCTCTGCCGTGCGCCTGCGGAGCAGCG | |
| | TGCCCGGGGTGCGGCTCCTGCAGGACTCGGTGGACTTCTCGCTG | |
| | GCCGACGCCATCAACACCGAGTTCAAGAACACCCGCACCAACGA | |
| | GAAGGTGGAGCTGCAGGAGCTGAATGACCGCTTCGCCAACTACA | |
| | TCGACAAGGTGCGCTTCCTGGAGCAGCAGAATAAGATCCTGCTG | |
| | GCCGAGCTCGAGCAGCTCAAGGGCCAAGGCAAGTCGCGCCTAGG | |
| | GGACCTCTACGAGGAGGAGATGCGGGAGCTGCGCCGGCAGGTGG | |
| | ACCAGCTAACCAACGACAAAGCCCGCGTCGAGGTGGAGCGCGAC | |
| | AACCTGGCCGAGGACATCATGCGCCTCCGGGAAAAATTGCAGGA | |
| | GGAGATGCTTCAGAGAGAGGAAGCCGAAAACACCCTGCAATCTT | |
| | TCAGACAGGATGTTGACAATGCGTCTCTGGCACGTCTTGACCTT | |
| | GAACGCAAAGTGGAATCTTTGCAAGAAGAGATTGCCTTTTTGAA | |
| | GAAACTCCACGAAGAGGAAATCCAGGAGCTGCAGGCTCAGATTC | |
| | AGGAACAGCATGTCCAAATCGATGTGGATGTTTCCAAGCCTGAC | |
| | CTCACGGCTGCCCTGCGTGACGTACGTCAGCAATATGAAAGTGT | |
| | GGCTGCCAAGAACCTGCAGGAGGCAGAAGAATGGTACAAATCCA | |
| | AGTTTGCTGACCTCTCTGAGGCTGCCAACCGGAACAATGACGCC | |
| | CTGCGCCAGGCAAAGCAGGAGTCCACTGAGTACCGGAGACAGGT | |
| | GCAGTCCCTCACCTGTGAAGTGGATGCCCTTAAAGGAACCAATG | |
| | AGTCCCTGGAACGCCAGATGCGTGAAATGGAAGAGAACTTTGCC | |
| | GTTGAAGCTGCTAACTACCAAGACACTATTGGCCGCCTGCAGGA | |
| | TGAGATTCAGAATATGAAGGAGGAAATGGCTCGTCACCTTCGTG | |
| | AATACCAAGACCTGCTCAATGTTAAGATGGCCCTTGACATTGAG | |
| | ATTGCCACCTACAGGAAGCTGCTGGAAGGCGAGGAGAGCAGGAT | |
| | TTCTCTGCCTCTTCCAAACTTTTCCTCCCTGAACCTGAGGGAAA | |
| | CTAATCTGGATTCACTCCCTCTGGTTGATACCCACTCAAAAAGG | |
| | ACATTCCTGATTAAGACGGTTGAAACTAGAGATGGACAGGTTAT | |
| | CAACGAAACTTCTCAGCATCACGATGACCTTGAATAA | |
| Ybx1 | >J03827.1: 127-1080 Y box binding protein-1 (YB-1) mRNA | ENSG00000065978 |
| | ATGAGCAGCGAGGCCGAGACCCAGCAGCCGCCCGCCGCCCCCCC | |
| | CGCCGCCCCCGCCCTCAGCGCCGCCGACACCAAGCCCGGCACTA | |
| | CGGGCAGCGGCGCAGGGAGCGGTGGCCCGGGCGGCCTCACATCG | |
| | GCGGCGCCTGCCGGCGGGGACAAGAAGGTCATCGCAACGAAGGT | |
| | TTTGGGAACAGTAAAATGGTTCAATGTAAGGAACGGATATGGTT | |
| | TCATCAACAGGAATGACACCAAGGAAGATGTATTTGTACACCAG | |
| | ACTGCCATAAAGAAGAATAACCCCAGGAAGTACCTTCGCAGTGT | |
| | AGGAGATGGAGAGACTGTGGAGTTTGATGTTGTTGAAGGAGAAA | |
| | AGGGTGAGGAGGCAGCAAATGTTACAGGTCCTGGTGGTGTTCCA | |
| | GTTCAAGGCAGTAAATATGCAGCAGACCGTAACCATTATAGACG | |
| | CTATCCACGTCGTAGGGGTCCTCCACGCAATTACCAGCAAAATT | |
| | ACCAGAATAGTGAGAGTGGGGAAAAGAACGAGGGATCGGAGAGT | |
| | GCTCCCGAAGGCCAGGCCCAACAACGCCGGCCCTACCGCAGGCG | |
| | AAGGTTCCCACCTTACTACATGCGGAGACCCTATGGGCGTCGAC | |
| | CACAGTATTCCAACCCTCCTGTGCAGGGAGAAGTGATGGAGGGT | |
| | GCTGACAACCAGGGTGCAGGAGAACAAGGTAGACCAGTGAGGCA | |
| | GAATATGTATCGGGGATATAGACCACGATTCCGCAGGGGCCCTC | |
| | CTCGCCAAAGACAGCCTAGAGAGGACGGCAATGAAGAAGATAAA | |
| | GAAAATCAAGGAGATGAGACCCAAGGTCAGCAGCCACCTCAACG | |
| | TCGGTACCGCCGCAACTTCAATTACCGACGCAGACGCCCAGAAA | |
| | ACCCTAAACCACAAGATGGCAAAGAGACAAAAGCAGCCGATCCA | |
| | CCAGCTGAGAATTCCCGCTCCCGAGGCTGA | |
| Zfp804a | >NM_194250.2: 432-4061 Homo sapiens zinc finger protein 804A | ENSG00000170396 |
| | (ZNF804A), mRNA | |
| | ATGGAGTGTTACTACATTGTCATCAGCTCCACGCATCTCAGCAA | |
| | CGGACACTTTCGCAACATCAAGGGAGTTTTCCGGGGCCCTCTCA | |
| | GCAAGAACGGGAACAAAACTCTGGACTATGCTGAGAAGGAAAAT | |
| | ACCATAGCAAAAGCTCTGGAAGATCTGAAGGCAAATTTTTACTG | |
| | TGAACTCTGTGACAAGCAGTACTATAAGCACCAGGAGTTTGACA | |
| | ATCACATTAATTCATATGACCATGCTCACAAGCAGAGGCTCAAG | |
| | GAACTGAAACAAAGGGAATTTGCTCGAAATGTAGCATCTAAATC | |
| | CAGGAAAGATGAAAGAAAACAGGAAAAGGCACTCCAACGCCTGC | |
| | ACAAGCTGGCTGAGCTAAGAAAGGAAACTGTATGTGCTCCTGGA | |
| | AGTGGCCCCATGTTCAAATCAACAACTGTTACTGTGAGAGAAAA | |
| | CTGTAATGAAATTTCCCAACGAGTTGTTGTGGATTCAGTTAATA | |
| | ACCAGCAAGATTTCAAATATACTTTGATTCATAGTGAAGAGAAT | |
| | ACTAAAGATGCTACCACTGTTGCTGAAGATCCAGAAAGTGCAAA | |
| | TAATTATACAGCAAAAAATAACCAAGTTGGGGATCAAGCCCAGG | |
| | GGATTCACAGACACAAAATCGGCTTTTCTTTTGCATTTCCAAAG | |
| | AAAGCGTCCGTGAAGCTAGAGTCCTCAGCTGCAGCCTTCTCTGA | |
| | ATACAGTGATGATGCCTCAGTGGGAAAAGGATTTAGCAGAAAAA | |
| | GTAGATTTGTCCCCAGTGCTTGTCATCTTCAACAATCTTCACCA | |
| | ACAGATGTGCTTTTGAGTTCTGAGGAGAAAACTAACTCTTTTCA | |
| | TCCACCAGAGGCAATGTGCAGAGACAAAGAAACTGTTCAAACTC | |
| | AAGAGATAAAAGAAGTCTCTAGTGAAAAAGATGCATTATTATTA | |
| | CCTTCATTTTGCAAGTTTCAACTTCAGTTATCTTCTGATGCAGA | |
| | TAATTGTCAAAATTCAGTCCCATTAGCAGATCAAATACCACTAG | |
| | AGAGTGTTGTTATTAATGAAGACATACCTGTTAGTGGTAACAGT | |
| | TTTGAGTTGTTAGGAAATAAATCCACAGTTCTTGACATGTCTAA | |
| | TGATTGCATATCTGTGCAAGCTACCACAGAGGAAAATGTTAAGC | |
| | ATAACGAGGCATCCACAACTGAGGTTGAAAATAAAAATGGTCCC | |
| | GAGACATTGGCCCCTTCAAATACTGAAGAGGTTAACATAACTAT | |
| | ACATAAGAAAACAAATTTCTGCAAAAGACAATGTGAGCCATTTG | |
| | TACCTGTCCTTAACAAACACAGATCTACAGTTCTTCAGTGGCCA | |
| | TCAGAAATGCTGGTTTATACAACTACGAAACCATCAATTTCCTA | |
| | TAGCTGTAATCCTCTATGTTTTGACTTCAAGTCTACTAAAGTAA | |
| | ATAATAATCTAGATAAAAATAAGCCAGACTTAAAAGATCTTTGT | |
| | TCTCAGCAGAAGCAGGAAGACATTTGCATGGGACCACTTTCAGA | |
| | TTACAAGGATGTATCTACAGAAGGACTCACTGATTATGAAATTG | |
| | GAAGTAGCAAAAATAAATGCAGCCAAGTCACTCCTCTTTTGGCT | |
| | GATGATATTCTCTCCAGTAGTTGTGATTCTGGAAAAAATGAGAA | |
| | CACAGGTCAGAGGTATAAAAACATTTCCTGTAAGATCAGAGAAA | |
| | CAGAAAAGTATAATTTTACTAAAAGTCAAATAAAACAGGACACT | |
| | CTAGATGAAAAATACAACAAAATAAGGTTGAAAGAGACCCATGA | |
| | ATACTGGTTCCATAAAAGTAGAAGAAAGAAAAAAAGAAAAAAGT | |
| | TATGTCAGCATCATCATATGGAGAAAACCAAAGAATCAGAAACT | |
| | CGCTGCAAAATGGAAGCAGAGAATAGTTACACTGAAAATGCTGG | |
| | GAAATATCTATTGGAACCAATTTCAGAAAAGCAGTATTTAGCTG | |
| | CAGAGCAATTATTAGACTCACATCAGTTACTTGATAAAAGGCCC | |
| | AAATCAGAATCCATATCCTTAAGTGACAATGAAGAAATGTGTAA | |
| | AACATGGAATACTGAATACAACACTTATGATACTATCAGTTCTA | |
| | AAAACCACTGTAAAAAGAACACAATACTTTTAAATGGACAATCA | |
| | AATGCAACAATGATACATTCTGGGAAACATAATTTAACATATTC | |
| | TAGAACTTACTGTTGTTGGAAAACCAAAATGTCAAGCTGTAGTC | |
| | AGGATCACAGAAGCTTAGTTCTTCAAAATGATATGAAACACATG | |
| | AGTCAGAATCAGGCTGTTAAAAGAGGTTACAATTCTGTCATGAA | |
| | TGAATCAGAAAGATTCTATCGAAAACGTAGACAACATTCACATT | |
| | CTTATTCTTCAGATGAAAGTTTAAATCGACAGAATCATTTACCA | |
| | GAAGAATTTTTGAGGCCACCAAGTACTTCAGTTGCTCCCTGCAA | |
| | GCCTAAAAAGAAACGGAGGCGAAAAAGAGGCAGATTCCACCCCG | |
| | GATTTGAAACTTTAGAACTCAAAGAAAATACAGATTATCCCGTG | |
| | AAAGACAATTCTTCCTTAAATCCTCTGGATAGGTTAATAAGTGA | |
| | AGACAAAAAAGAGAAAATGAAACCACAAGAAGTTGCAAAAATCG | |
| | AAAGGAACTCAGAACAAACAAACCAATTAAGAAACAAACTGTCT | |
| | TTCCACCCTAACAATCTCCTTCCTTCTGAAACCAATGGTGAAAC | |
| | TGAGCATTTAGAAATGGAGACCACTTCTGGTGAATTGTCAGATG | |
| | TTTCCAATGATCCCACCACATCTGTCTGTGTAGCTAGTGCCCCA | |
| | ACAAAAGAAGCAATTGACAATACCCTGCTTGAACACAAAGAAAG | |
| | AAGTGAGAATATAAATCTTAATGAAAAGCAAATTCCTTTTCAGG | |
| | TGCCTAATATTGAAAGGAACTTTAGACAGTCACAGCCTAAATCC | |
| | TATCTTTGCCATTATGAACTGGCTGAGGCCCTTCCACAAGGAAA | |
| | GATGAATGAGACACCAACTGAGTGGCTGCGTTATAATTCAGGAA | |
| | TCCTTAACACACAACCACCATTACCATTCAAAGAAGCACATGTC | |
| | AGTGGTCATACTTTTGTAACAGCTGAGCAAATCCTGGCTCCATT | |
| | AGCTTTACCAGAGCAAGCATTATTGATCCCACTAGAAAACCATG | |
| | ACAAATTCAAAAATGTACCATGTGAGGTCTACCAGCACATTCTG | |
| | CAGCCAAACATGCTGGCCAACAAGGTTAAATTTACCTTTCCTCC | |
| | AGCTGCCCTCCCACCCCCTAGCACACCTCTGCAGCCTTTGCCTT | |
| | TGCAGCAGTCCTTATGTTCTACCTCTGTAACCACTATCCATCAC | |
| | ACTGTTTTGCAGCAGCACGCTGCAGCTGCTGCAGCTGCAGCTGC | |
| | AGCCGCAGCTGCAGGAACCTTTAAAGTGCTTCAGCCACACCAAC | |
| | AGTTTCTTTCCCAAATCCCAGCTCTCACCAGAACCTCATTACCT | |
| | CAGCTCTCAGTAGGACCAGTAGGACCGAGGCTTTGTCCTGGGAA | |
| | CCAGCCAACTTTTGTTGCTCCTCCTCAGATGCCAATCATTCCAG | |
| | CTTCCGTTCTTCATCCTAGCCATCTGGCTTTCCCATCTTTACCC | |
| | CATGCACTCTTTCCTTCACTGCTTTCCCCACACCCTACTGTCAT | |
| | CCCTTTGCAACCTCTCTTCTAG | |

By “agent” is meant a small molecule chemical compound, nucleic acid molecule, polypeptide, or fragments thereof.

By “alteration” is meant an increase or decrease in an analyte or clinical marker.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antisense nucleic acid”, it is meant a nucleic acid molecule that binds to target RNA by means of RNA-RNA or RNA-DNA interactions and alters the activity of the target RNA. See, for example, Stein and Cheng, Science 261:1004-1012, 1993; Woolf et al., U.S. Pat. No. 5,849,902. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of antisense strategies, see Schmajuk NA et al. J Biol Chem, 274(31):21783-21789, 1999; Delihas N et al., Nat Biotechnol. 15(8):751-753, 1997; Aboul-Fadl T, Curr Medicinal Chem 12:763-771, 2005).

By “biological sample” is meant any liquid, cell, or tissue obtained from a subject. In embodiments, the tissue is ovarian tissue.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

“Detect” refers to identifying the presence, absence or amount of the analyte to be detected.

By “effective amount” is meant the amount of an agent required to achieve a desired outcome. The effective amount of active compound(s) used to practice the methods of the present disclosure varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. In some embodiments, this portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “hormone” is meant a substance made by glands in the body and circulated in the bloodstream to control the actions of target cells and organs. Non-limiting examples of hormones include insulin, melatonin, estrogen, testosterone, and cortisol. Non-limiting examples of glands and their corresponding secreted hormones are listed in the following table:

TABLE 1
Glands and their corresponding secreted hormones.

| Gland | Secreted Hormone(s) |
|---|---|
| Adrenal glands | Aldosterone |
| Adrenal glands | Corticosteroid |
| Pituitary gland | Antidiuretic hormone |
| | (vasopressin) |
| Pituitary gland | Adrenocorticotropic |
| | hormone (ACTH) |
| Pituitary gland | Growth hormone (GH) |
| Pituitary gland | Luteinizing hormone |
| | (LH) and follicle- |
| | stimulating hormone |
| | (FSH) |
| Pituitary gland | Oxytocin |
| Pituitary gland | Prolactin |
| Pituitary gland | Thyroid-stimulating |
| | hormone (TSH) |
| Kidneys | Renin and angiotensin |
| Kidneys | Erythropoietin |
| Pancreas | Glucagon |
| Pancreas | Insulin |
| Ovaries | Estrogen |
| Ovaries | Progesterone |
| Parathyroid | Parathyroid hormone |
| glands | (PTH) |
| Thyroid gland | Thyroid hormone |
| Adrenal glands | Epinephrine |
| Adrenal glands | Norepinephrine |
| Testes (testicles) | Testosterone |
| Pineal gland | Melatonin |
| Hypothalamus | Growth hormone |
| | releasing hormone |
| | (GHRH) |
| Hypothalamus | Thyrotropin releasing |
| | hormone (TRH) |
| Hypothalamus | Gonadotropin releasing |
| | hormone (GnRH) |
| Hypothalamus | Corticotropin releasing |
| | hormone (CRH) |
| Thymus | Humoral factors |

By “non-hormonal” is meant not involving the use of any hormones.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increase” is meant to alter positively relative to a reference. An increase may be by 1%, 5%, 10%, 25%, 30%, 50%, 75%, 100%, or more, or by 1.5-fold, -fold 2-fold, 3-fold, 4-fold, 5-fold, 6-fold, 7-fold, 8-fold, 9-fold, 10-fold, 25-fold, 50-fold, 75-fold, 100-fold, or more.

By “inhibitory polynucleotide” or “inhibitory nucleic acid molecule” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the polynucleotides (e.g., genes) delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

For most applications, washing steps that follow hybridization will also vary in stringency. Wash stringency conditions can be defined by salt concentration and by temperature. As above, wash stringency can be increased by decreasing salt concentration or by increasing temperature. For example, in some embodiments, stringent salt concentration for the wash steps will be less than about 30 mM NaCl and 3 mM trisodium citrate, and less than about 15 mM NaCl and 1.5 mM trisodium citrate. In some cases, stringent temperature conditions for the wash steps will ordinarily include a temperature of at least about 25° C., at least about 42° C., or even at least about 68° C. In an embodiment, wash steps will occur at 25° C. in 30 mM NaCl, 3 mM trisodium citrate, and 0.1% SDS. In an embodiment, wash steps will occur at 42 C in 15 mM NaCl, 1.5 mM trisodium citrate, and 0.1% SDS. In another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “isolated polynucleotide” is meant a nucleic acid (e.g., a DNA) that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In some cases, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

By “polynucleotide” or “nucleic acid molecule” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides may be advantageous because of properties such as, for example, enhanced stability in the presence of nucleases.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the disclosure embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant to alter negatively relative to a reference. A reduction may be by 1%, 5%, 10%, 25%, 30%, 50%, 75%, 100%, or more, or by 1.5-fold, -fold 2-fold, 3-fold, 4-fold, 5-fold, 6-fold, 7-fold, 8-fold, 9-fold, 10-fold, 25-fold, 50-fold, 75-fold, 100-fold, or more.

By “reference” is meant a standard or control condition. In embodiments, a reference is a subject not treated according to a method provided herein. In some cases, a reference is a cell, organ, or subject not administered an agent of the present disclosure. Sometimes, a reference can be a subject, cell, or organ prior to a change in a treatment (e.g., dose).

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the disclosure, but which does not substantially recognize and bind other molecules in a sample, for example, a biological sample, which naturally includes a polypeptide of the disclosure.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 300 C, of at least about 370 C, or of at least about 420 C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In some embodiments, hybridization will occur at 300 C in 750 mM NaCl, 75 mM trisodium citrate, and 1% SDS. In another embodiment, hybridization will occur at 370 C in 500 mM NaCl, 50 mM trisodium citrate, 1% SDS, 35% formamide, and 100 μg/ml denatured salmon sperm DNA (ssDNA). In some cases, hybridization will occur at 420 C in 250 mM NaCl, 25 mM trisodium citrate, 1% SDS, 50% formamide, and 200 μg/ml ssDNA. Useful variations on these conditions will be readily apparent to those skilled in the art.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence may be at least 60%, at least 80%, at least 85%, at least 90%, at least 95%, or at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-³ and e-¹⁰° indicating a closely related sequence.

By “subject” is meant a mammal, including, but not limited to, a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A to 1E provide a schematic diagram, a uniform manifold approximation and projection (UMAP), a heatmap, images of multiplexed error-robust fluorescence in situ hybridization (MERFISH) ovary sections, and a stacked bar plot relating to a single-cell and spatial transcriptomic analysis of adult mouse ovaries throughout the time course of ovulation. FIG. 1A provides a schematic diagram depicting workflow for single cell and spatial transcriptomic analyses. Mice were hyperstimulated with pregnant mare serum gonadotropin (PMSG) and human chorionic gonadotropin (hCG) and ovaries were dissected 0h, 4h, or 12h post-hCG injection, processed, and submitted for scRNA-seq or MERFISH analysis. FIG. 1B provides a UMAP showing all 16 identified cell type clusters. FIG. 1C provides a heatmap showing five marker genes used to determine the identity of each cell cluster. FIGS. 1D-1 and 1D-2 (where FIG. 1D-2 is a continuation of FIG. 1D-1) provides a stacked bar plot showing the percent of cells in each cluster expressed at each time point. FIG. 1E provides images showing examples of 0h, 4h, and 12h MERFISH ovary sections with seven major cell types localized.

FIGS. 2A to 2E-2 provide a UMAP, a heatmap, a stacked bar plot, RNAScope images, and dot plots demonstrating that cumulus cells exhibited time-dependent changes in gene expression. FIG. 2A provides a UMAP showing the clustering of cumulus cells. FIG. 2B provides a heatmap depicting differential gene expression in Cumulus 1 (early) and Cumulus 2 (late) clusters. FIG. 2C provides a stacked bar plot showing the percent of cells in cumulus clusters expressed at each indicated time point. FIGS. 2D-1 and 2D-2 (where FIG. 2D-2 is a continuation of FIG. 2D-1) provide RNAScope images of cumulus cell genes of interests from the integration of single cell and spatial transcriptomics. FIGS. 2E-1 and 2E-2 provide dot plots showing top processes upregulated in early (FIG. 2E-1) and late (FIG. 2E-2) cumulus cells. The genes listed along the bottom of the heatmap of FIG. 2B from right-to-left are: Rnfl80, Star, Spsb1, Gsta4, Ddit41, F3, Ggct, Kcnd2, Tchhl1, Pgr, Suitle1, Btc, Areg, Vcan, Arhgefl2, Robo2, Rtl4, Inhba, Inha, Mast4, Bmp3, Zfp804a, Tac1, Ifi202b, Nudt4, Ramp1, Scp2, Lgals3, Pik3c2g, Nupr1, Enol, Sdc1, Spp1, Lox, Cck, Tspo, Chchd10, Gm10076, Taldo1, Lgals1, S100a6, Emp3, Igfbp4, Timp1, Anxa2, Fdps, Sdo4, Idh1, Fkbp5, and Akrld.

FIGS. 3A to 3E-4 provide a UMAP, a heatmap, a stacked bar plot, expression plots, and dot plots demonstrating that theca cells exhibited time-dependent changes in gene expression. FIG. 3A provides a UMAP showing the clustering of theca cells FIG. 3B provides a heatmap depicting differential gene expression in Theca 1 (early) and Theca 2 (late) clusters. FIG. 3C provides a stacked bar plot showing the percent of cells in theca clusters expressed at each indicated time point. FIGS. 3D-1 and 3D-2 (where FIG. 3D-2 is a continuation of FIG. 3D-1) provide expression plots of genes of interest from the integration of single cell and spatial transcriptomics. FIGS. 3E-1 to 3E-4 provides dot plots showing top processes upregulated in Theca 0hrs_1 (FIG. 3E-1), Theca 0hrs_2 (FIG. 3E-2), Theca 4 hrs (FIG. 3E-4), and Theca 12 hrs (FIG. 3E-4). The genes listed along the bottom of the heatmap of FIG. 3B from right-to-left are: Acsbg1, Akrlcl, Pak3, Gstm2, Lhcgr, Smoc2, Cxxc4, Rtl4, Gm42418, Aff2, Gab2, Trib2, AU020206, Gja1, Bst2, Gm48584, Kit, Folr1, Fabp3, Gas6, Tcaf1, Nckap5, Dnah2, Gm26691, Oca2, Cypl7a1, Mt1, Fdps, Star, A730049H05Rik, Rhox8, Timp1, Cd63, Tmsb4x, Vim, Col4a1, Uba52, Tpm4, Neat1, Mrap, Ybx1, Tnfrsfl2a, Cnn3, Ereg, Abil, Ephx2, Anxa2, Ldha, Junb, and Alas1.

FIGS. 4A to 4E-2 provide a UMAP, a heatmap, a stacked bar plot, expression plots, and dot plots demonstrating that stroma cells exhibited time-dependent changes in gene expression. FIG. 4A provides a UMAP showing the clustering of stroma cells. FIG. 4B provides a heatmap depicting differential gene expression in Stroma 1 (early) and Stroma 2 (late) clusters. FIG. 4C provides a stacked bar plot showing the percent of cells in theca clusters expressed at each indicated time point. FIGS. 4D-1 and 4D-2 (where FIG. 4D-2 is a continuation of FIG. 4D-1) provide expression plots of genes of interest from the integration of single cell and spatial transcriptomics FIGS. 4E-1 and 4E-2 Dot plots showing top processes upregulated in Stroma 1 (FIG. 4E-1) and Stroma 2 (FIG. 4E-2). The genes listed along the bottom of the heatmap of FIG. 4B from right-to-left are: Gm10076, Uba52, Rpl29, Sec61b, Timp1, Tagln2, Nme1, Manf, Hspa5, Pdia6, Mrap, Ybx1, Ldha, Cebpb, Neat1, Frmd5, Pde10a, Abi1, Pcsk5, Ifitm3, Plac8, Fdx1, Mgst1, Gapdh, Tenm4, Ptprd, Sncaip, Greb1, Ddit41, Au020206, Itm2b, Rora, Gab2, Gm42418, Camk1d, Gstm1, Gstm2, Mamdc2, Grm7, Gucyla1, Ogn, Rarres2, Fbxl7, Itih5, Dcn, Lama2, Tcf21, Col4a4, Egflam, and Ptch1.

FIGS. 5A to 5E-2 provide a UMAP, a heatmap, a stacked bar plot, expression plots, and dot plots demonstrating that luteal cells exhibited time-dependent changes in gene expression. FIG. 5A provides a UMAP showing the clustering of luteal cell clusters. FIG. 5B provides a heatmap depicting differential gene expression in luteal cells and active CL sub-clusters. FIG. 5C provides a stacked bar plot showing the percent of cells in luteal sub-clusters expressed at each indicated time point. FIGS. 5D-1 and 5D-2 (where FIG. 5D-2 is a continuation of FIG. 5D-1) provide expression plots of genes of interest from the integration of single cell and spatial transcriptomics. FIGS. 5E-1 and 5E-2 provide dot plots showing top processes upregulated in Active CL (FIG. 5E-1) and General Luteal (FIG. 5E-2). The genes listed along the bottom of the heatmap of FIG. 5B from right-to-left are: Bace2, Gm2a, Bhmt, Cst8, Clic3, Prlr, Cyp11a1, Tle5, Aebp1, Hmgcs1, Lhcgr, Ndufc2, Prdx6, Gamt, Mcrip2, Nrnl, Cstl2, Grebl, Bst2, Hsd3b1, Akrlcl, Hspdl, Mgarp, Kcnd2, Gm20629, Thrsp, Tnfrsfl2a, Tpd52ll, Timp1, Sdc4, Tmsb4x, Cited2, Ybx1, Tagln2, Gm12648, Pcsk5, Col4a1, Abi1, Runxl, Vcan, Slc7a8, Frmd5, Ifrdl, Ctsl, Mt2, Sox5, Fndc3b, Cnn3, Junb, and Jun.

FIGS. 6A-1 to 6E provide circle plots and dot plots showing cell-cell interactions between cell types change throughout ovulation. FIG. 6A-1, 6A-2, and 6A-3 provide circle plots showing the change of interactions between various cell types at 0 hr (FIG. 6A-1), 4 hr (FIG. 6A-2), and 12 hr (FIG. 6A-3) time points. FIGS. 6B-1, 6B-2, and 6B-3 provide scatterplots showing incoming interaction strength and outgoing interaction strength for all cell types present within the 0h (FIG. 6B-1), 4h (FIG. 6B-2), and 12h (FIG. 6B-3) timepoints. FIG. 6C provides dot plot showing scaled interaction strength between granulosa cells (sending cells) and cumulus cells (receiving cells) with upregulated interactions centered at 0h (top panel) and 4h (bottom panel). FIG. 6D provides dot plots showing scaled interaction strength between theca cells (sending cells) and luteal cells (receiving cells) with upregulated interactions centered at 0h (top panel), 4h (bottom-left panel), and 12h (bottom-right panel). FIG. 6E provides dot plots showing scaled interaction strength between granulosa cells (sending cells) and luteal cells (receiving cells) with upregulated interactions centered at 0h (top panel), 4h (bottom-left panel), and 12h (bottom-right panel).

FIGS. 7A to 7D provide a chart, a bar graph, and schematic diagrams showing a description of optimization protocols for ovary collection (FIGS. 7A and 7B) and multiplexed error-robust fluorescence in situ hybridization (MERFISH) (FIGS. 7C and 7D). FIG. 7A provides a chart depicting timing of hormone (pregnant mare serum gonadotropin (PMSG) and human chorionic gonadotropin (hCG)) injections and ovary collection in control and offset timing groups. FIG. 7B provides a bar plot showing the average number of cumulus-oocyte complexes (COCs) collected per mouse in control and offset timing groups. FIG. 7C provides a schematic where the top row shows that pregnant mare serum gonadotropin (PMSG) and human chorionic gonadotropin (hCG)-stimulated, flash-frozen mouse ovaries were collected as intact whole ovaries or contralaterally-halved ovaries. Samples were embedded into a pre-formed OCT tissue microarray scaffold (TMA) with the ovarian hilums (indicated by red asterisk *) pointing downward towards the tissue microarray base, enabling uniform tissue section collection onto fluorescent microsphere-coated functionalized coverslips. The bottom row of FIG. 7C shows corresponding images to bisect ovaries, embed into the trimethacrylate (TMA) scaffold, and 10×4′,6-diamidino-2-phenylindole (DAPI) imaging of the resulting tissue sections. FIG. 7D provides a shematic diagram showing a MERFISH protocol of mounting samples to undergo a series of staining and incubations (fixation, permeabilization, hybridization, polyacrylamide gel embedding, tissue clearing with detergents).

FIGS. 8A to 8F provide histograms, a uniform manifold approximation and projection (UMAP), violin plots, pie charts, and a clustered plot relating to quality checks. FIGS. 8A and 8B provide histograms showing quality metrics for a spatial transcriptomics dataset: number of transcripts per cell (FIG. 8B) and cell volume (FIG. 8A) post-filtering. FIG. 8C provides a UMAP of the single-cell dataset, including unknown clusters. FIG. 8D provides violin plots showing quality metrics for single-cell datasets: genes per cell (left panel of FIG. 8D) and percent mitochondrial (right panel of FIG. 8D) in the single-cell dataset post-filtering. FIG. 8E provides a pie chart of pathway classes identified in early vs. late ovulation timepoints. FIG. 8F provides a clustered dot plot comparing cell types found in both datasets using established markers (see Morris et al., eLife, 11, e77239 (2022), the disclosure of which is incorporated herein by reference in its entirety for all purposes).

FIGS. 9A to 9C provide images of hematoxylin and eosin stained (H&E-stained) sections of ovaries collected during an in vivo ovulation time course. Examples of ovaries collected 0h (FIG. 9A), 4h (FIG. 9B), or 12h (FIG. 9C) post human chorionic gonadotropin (hCG) injection (left) and insets labeling key cell types (right). O=oocyte, CC=cumulus cells, GC=granulosa cells, TC=theca cells, SC=stroma cells, LC=luteal cells, Epi=epithelial cells. Scale bars=200 μm.

FIGS. 10A to 10C-5 provide expression plots, images of ovaries, and heatmaps showing clustering and cell identification using a spatial transcriptomic dataset. FIG. 10A provides expression plots of known markers for cell types identified in all ovaries at three time points. FIG. 10B provides images of ovaries colored by cell types identified. (Region 1: 12 hrs post-hCG administration, Region 3, Region 5: 4 hrs post-hCG administration, Region 6, Region 8: 0hrs post-hCG administration). FIGS. 10C-1 to 10C-5 provide heatmaps showing three top marker genes used to determine the identity of each cell cluster for each ovary using spatial transcriptomics for Region 1 (FIG. 10C-1), Region 3 (FIG. 10C-2), Region 6 (FIG. 10C-3), Region 7 (FIG. 10C-4), and Region 8 (FIG. 10C-5).

FIGS. 11A to 11E provide bar graphs, plots, expression plots, and RNAScope images relating to an integration analysis of single-cell and spatial transcriptomics. FIG. 11A provides bar plots of training scores for training genes (left panel) and Scatter plot of test scores vs sparsity for test genes (right panel), for the three integrations at 0hrs (top), 4 hrs (middle), and 12 hrs (bottom). FIG. 11B provides expression plots of Colla2 for predicted and observed results, which each showed similar patterns. FIG. 11C provides RNAScope images (left panel) and predicted results from integration analysis (right panel) for the indicated genes, which showed similar expression patterns. Scale bars=200 pm. FIG. 11D provides expression plots showing expression of Adamts1 (top panel), a known marker for luteinizing mural granulosa cells, and expression of Sox5 (bottom panel), a potential novel marker showing similar patterns. FIG. 11E provides plots showing that expression of Pdzm3 (bottom panel), a potential novel marker, showed similar patterns to Dcn, a known marker for stromal cells.

FIGS. 12A to 12D provide a bar plot and heatmaps showing outgoing and incoming cell-cell interactions. FIG. 12A provides a bar plot of total number of interactions per ovulation time point. FIG. 12B provides a heatmap of incoming and outgoing signal patterns at the 0hr timepoint. FIG. 12C provides a heatmap of incoming and outgoing signal patterns at the 4 hr timepoint. FIG. 12D provides a heatmap of incoming and outgoing signal patterns at the 12 hr timepoint. The genes listed from top-to-bottom of each heatmap of FIG. 12B are: COLLAGEN, LAMININ, JAM, MK, THBS, ANGPTL, SEMA3, GAS, IGF, APP, HSPG, BMP, PTPRM, WNT, MIF, FN1, VEGF, AMH, TENASCIN, NECTIN, SEMA7, PTN, SEMA5, CDH, VISFATIN, MPZ, SEMA6, EPHA, ACTIIVIN, ncWNT, PROS, HH, TGFb, EGF, ESAM, VCAM, FGF, KIT, ANGPT, AGRN, CDH5, PDGF, PECAMI, EPHB, GALECTIN, CADM, NPR2, GRN, BST2, CD45, NOTCH, VISTA, TWEAK, NRG, CD39, CCL, ICAM, NCAM, COMPLEMENT, LIFR, SEMA4, NRXN, NGL, APELIN, RELN, CXCL, LAIR1, IL1, and CD46. The genes listed from top-to-bottom of each heatmap of FIG. 12C are: COLLAGEN, LAMININ, THBS, MK, FN1, ANGPTL, EGF, JAM, BMP, SEMA3, MIF, APP, HSPG, IGF, PTPRM, WNT, NECTIN, VEGF, GAS, TGFb, TENASCIN, MPZ, FGF, EPHA, VISFATIN, SPP1, SEMA6, PTN, PROS, AMH, CDH, EPHB, ACTIVIN, ESAM, SEMA7, NOTCH, AGRN, PDGF, ncWNT, TWEAK, SEMA5, ANGPT, CDH5, GALECTIN, PECAMI, HH, RESISTIN, VCAM, EPGN, CADM, NCAM, SELE, CD45, NRG, COMPLEMENT, VISTA, CCL, ICAM, BST2, CXCL, THY1, GRN, SEMA4, LIFR, CSF, NGL, NRXN, IL1, NPR2, SELPLG, APELIN, PTH, NPY, and LAIR1. The genes listed from top-to-bottom of each heatmap of FIG. 12D are: COLLAGEN, LAMININ, SPP1, THBS, ANGPTL, MK, JAM, FN1, EGF, TENASCIN, APP, IGF, BMP, HSPG, MIF, WNT, SEMA3, CDH, GAS, PTPRM, PTN, NECTIN, TGFb, VEGF, VISFATIN, MPZ, PROS, EDN, FGF, EPHA, TWEAK, AMH, NCAM, SEMA7, SEMA6, SEMA5, ESAM, PDGF, ACTIVIN, ncWNT, CDH5, VCAM, GALECTIN, EPHB, AGRN, NOTCH, HH, PECAMI, ANGPT, EPGN, CADM, THY1, NRG, SELE, CD45, CD39, GRN, COMPLEMENT, VISTA, CCL, CD200, CXCL, ICAM, LIFR, NGL, CALCR, NRXN, IL6, CSF, CD86, APELIN, PTH, and SELPLG.

FIGS. 13A to 13B-2 provide a UMAP and violin plots showing sub-clustering for luteal cells. FIG. 13A provides a UMAP for luteal cell clusters, including unknown clusters. FIGS. 13B-1 and 13B-2 provide violin plots showing quality metrics for the sub-clusters: percent mitochondrial (FIG. 13B-1) and number of features per cell (FIG. 13B-2) for each subcluster, resulting in the removal of the unknown cluster. In FIGS. 13B-1 and 13B-2, the plotted “violins” correspond from left-to-right to Active CL, Luteal Cells, Lutenizing Mural, Mitotic Antral, and Unknown, respectively.

### DETAILED DESCRIPTION

The disclosure features compositions and methods for altering ovulation in a subject. In particular embodiments, the methods involve administering to a female subject an agent that selectively reduces or eliminates the expression and/or activity of a target polypeptide and/or selectively kills and/or reduces the development, proliferation, or metabolism of a cell in the ovary of the female subject. In some embodiments, the methods involve administering to a female subject an agent that selectively increases the expression and/or activity of a target polypeptide and/or selectively increases the development, proliferation, or metabolism of a cell in an ovary of the female subject. In various embodiments, the method is a contraceptive method or a method for enhancing fertility.

Ovulation within the ovary is a spatiotemporally coordinated process that involves several tightly controlled tissue remodeling and maturation events, including oocyte meiotic maturation, cumulus expansion, follicle wall rupture, and remodeling of the ovarian stroma. To date, there are few studies that have detailed this process with true single cell resolution. The aspects and embodiments of the present disclosure is based, at least in part, upon discoveries made through the experiments and analyzes described in the Examples provided herein, where single-cell and single-cell imaging spatial transcriptomics of matched mouse ovaries across an ovulation time course was undertaken to map the spatiotemporal profile of ovarian cell types during this dynamic process. Many major ovarian cell types, such as cumulus, theca, stroma, granulosa, and luteal cells, exhibited time-dependent transcriptional states, were enriched for distinct functions across time, and had distinct localization profiles within the ovary. In addition, novel gene markers for ovulation-dependent cell states were discovered and validated using orthogonal methods. Finally, a detailed cell-cell interaction analysis was performed to identify ligand-receptor pairs that may drive ovulation, thereby revealing novel interactions that were essential for this process. Taken together, the data provided in the Examples of the disclosure provide a rich and comprehensive resource of ovulation in the mouse. Accordingly, the present disclosure provides in various embodiments compositions and methods for altering ovulation in a subject.

### Methods of Treatment

In one aspect, the present disclosure provides a method for altering ovulation and/or follicle activation and/or development in a subject. In embodiments, the method is a contraceptive method or a method for enhancing fertility.

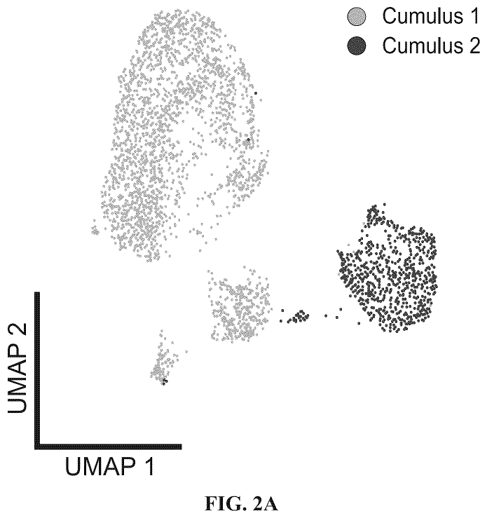

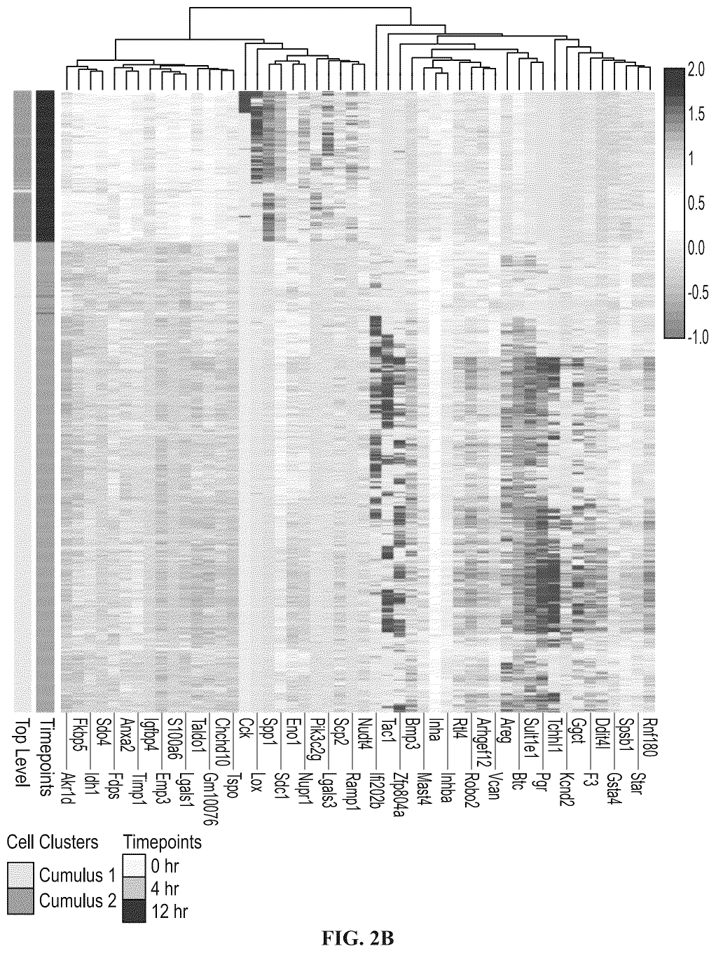

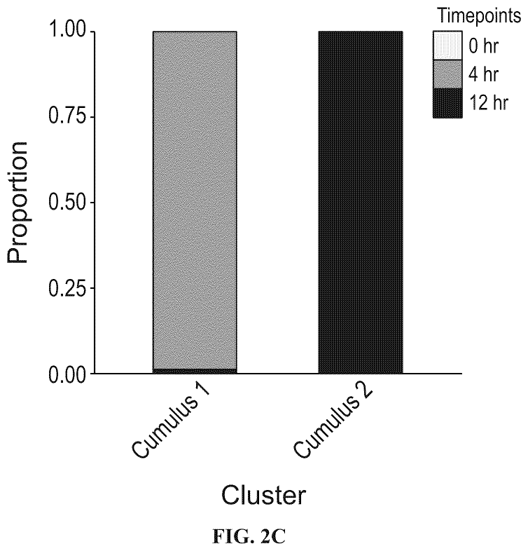

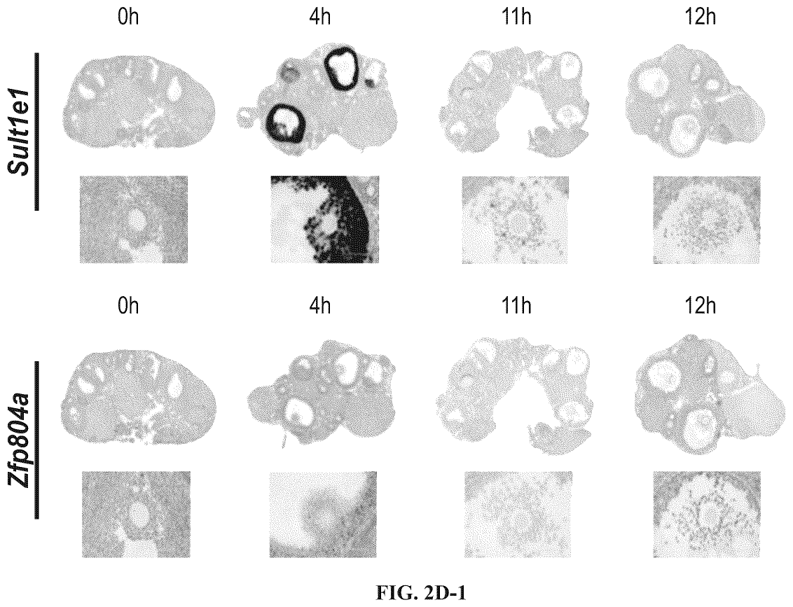

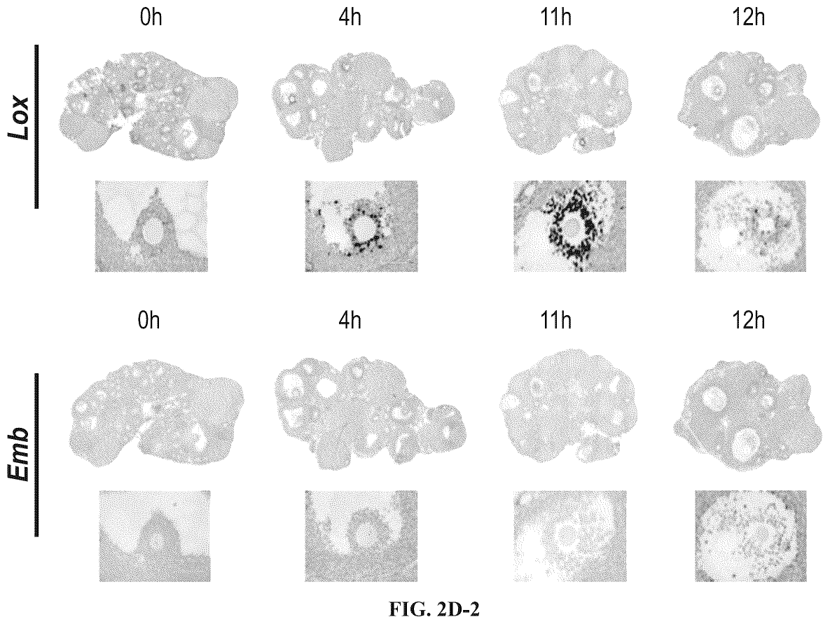

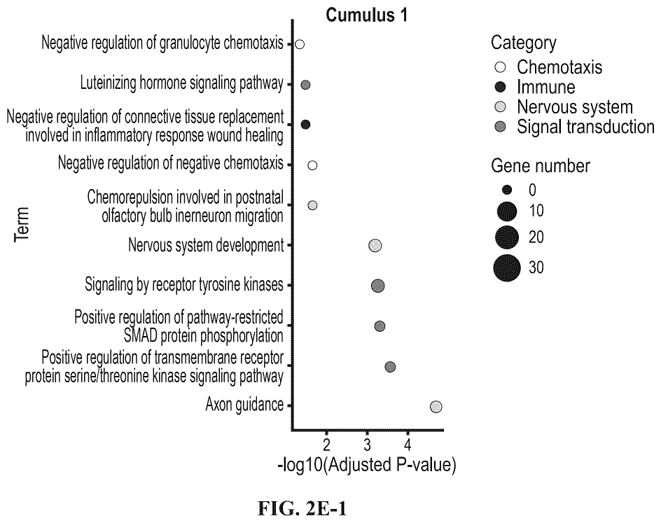

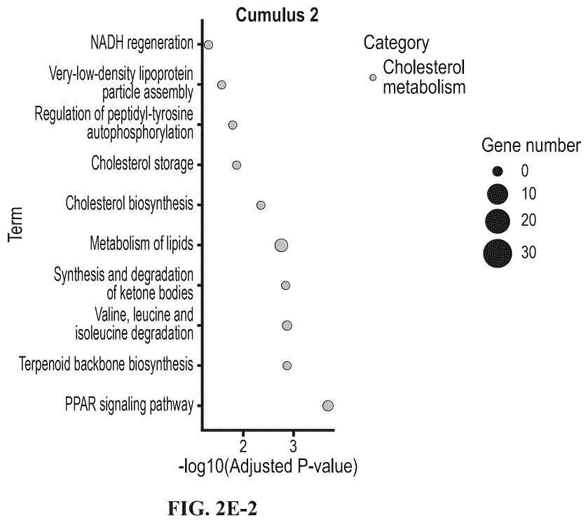

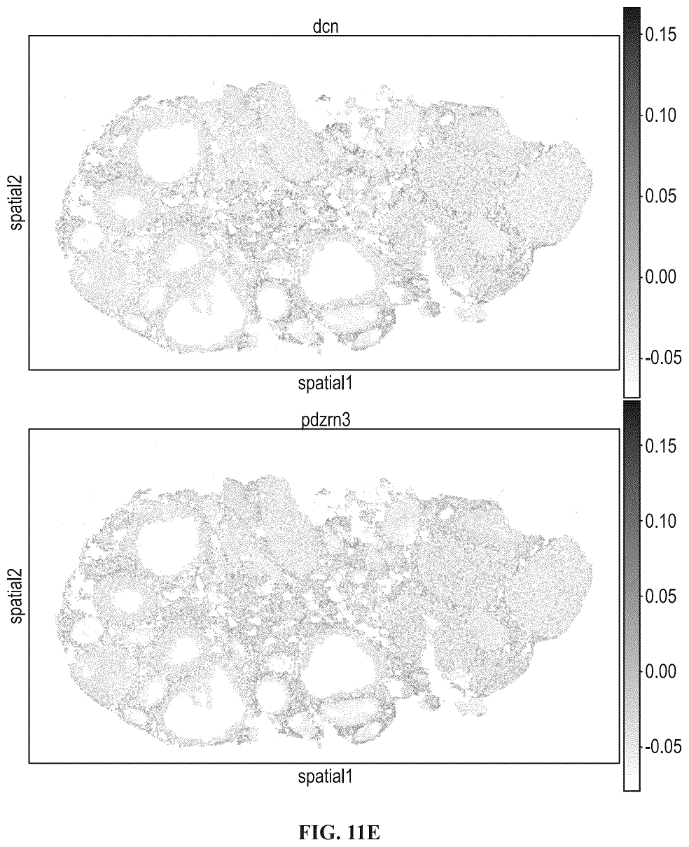

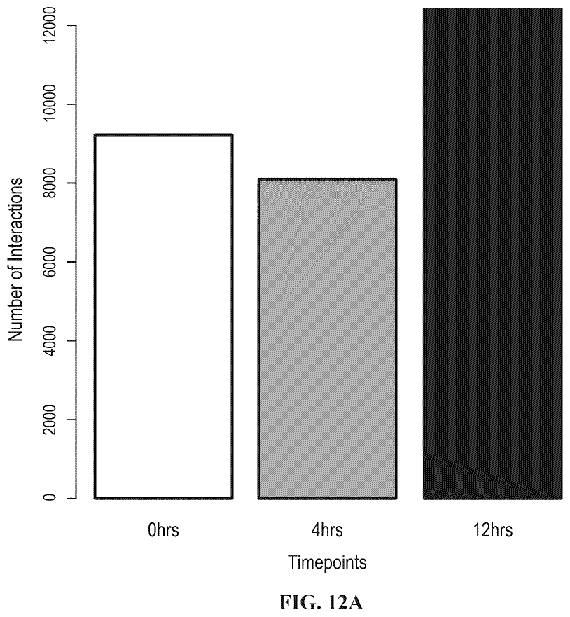

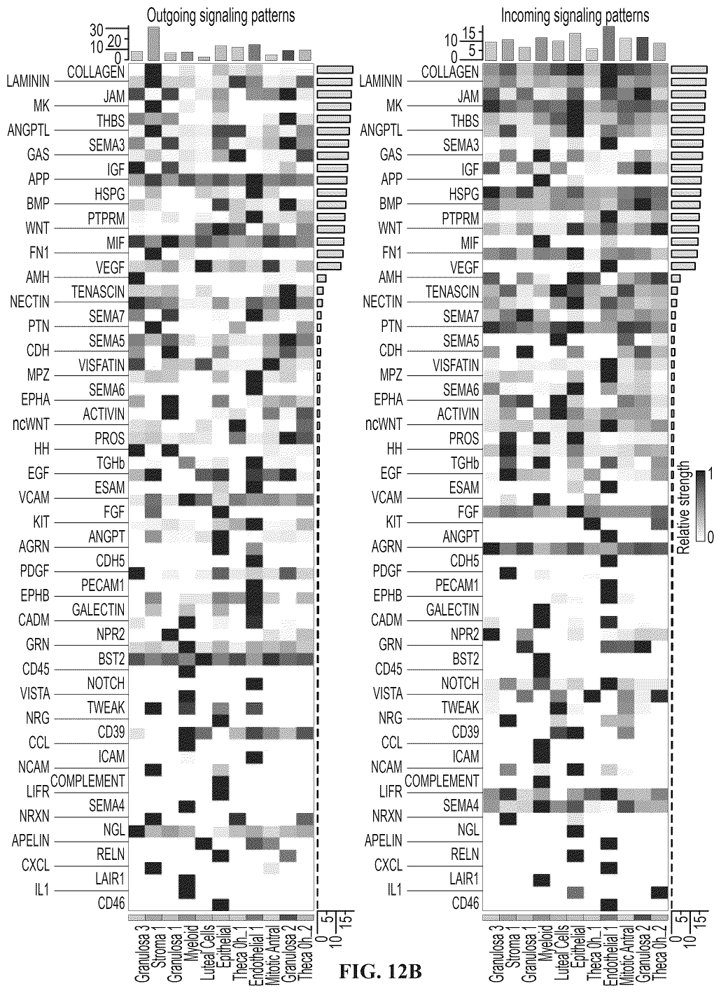

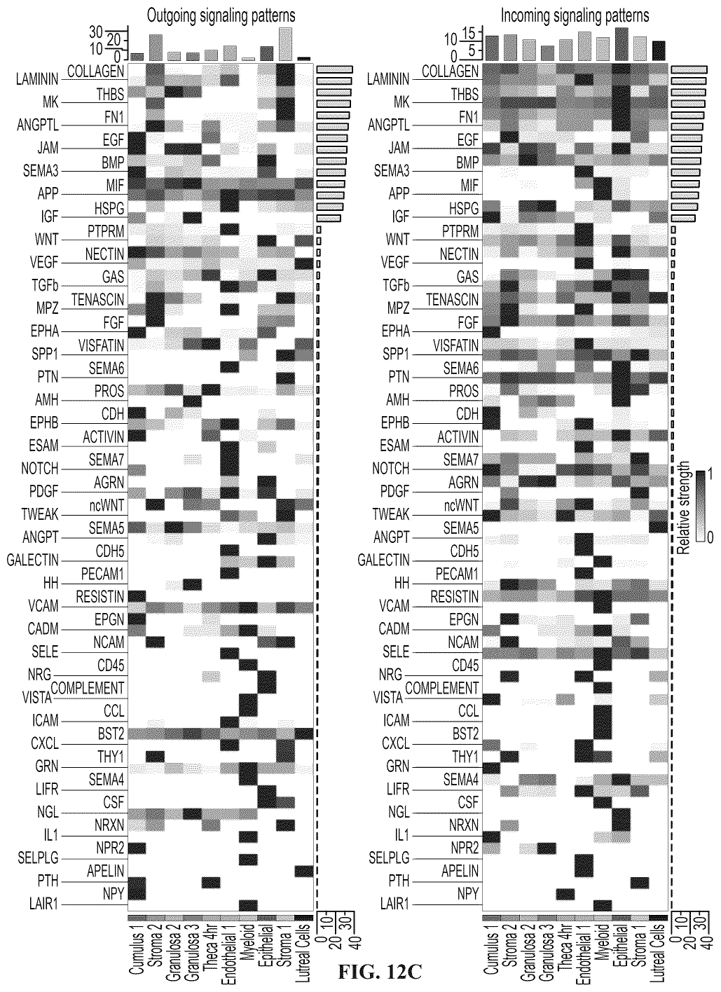

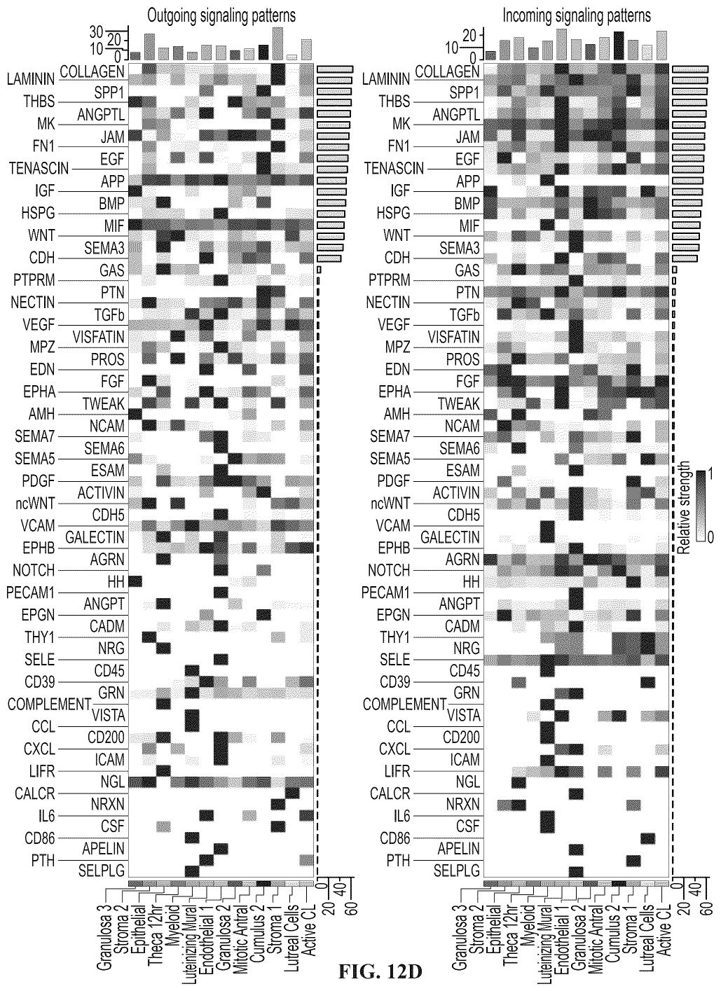

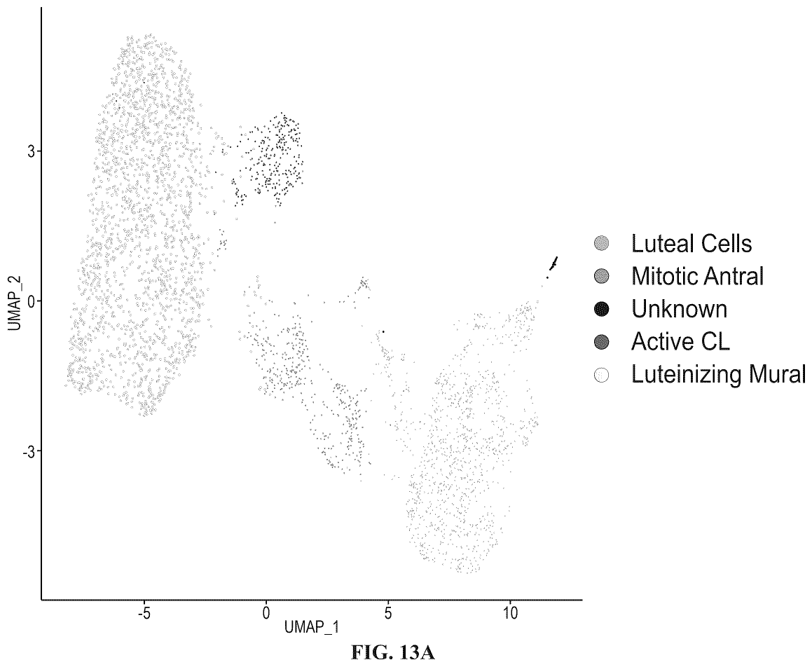

In various aspects, the methods of the disclosure involve altering the expression, expression level, amount, and/or activity of a gene or encoded polypeptide selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17b1, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9 in a cell in the ovary of a genotypic female subject. In some cases, the gene or encoded polypeptide is selected from Sdc1, Pgr, Spp1, Frmd5, Chchd10, Spsb1, Tspo, Gm10076, and Rnfl80. In some cases, the gene or encoded polypeptide is selected from Tac1, Gsta4, Mast4, F3, Kcnd2, Timp1, Pik3c2g, Fdps, Lgals1, Ramp1, and Emb. In some cases, the gene or encoded polypeptide is selected from Sdc1, Hsd3b1, Onecut2, Cnn3, Rplp1, Sox5, Frmd5, Cst8, Aebp1, and Rpl13a. In some instances, the gene or encoded polypeptide is selected from Tmsb4x, Timp1, Ybx1, Vim, Col4a1, Gas6, Pak3, Trib2, Smoc2, Kit, Tpm4, Fndc3b, Cnn3, and Zfp804a. In some embodiments, the gene or encoded polypeptide is selected from Sdc1, Akrc1, Star, Fdx1, Scarb1, Acsbg1, Ybx1, Gas6, Cobll1, and Acly. In some cases, the gene or encoded polypeptide is selected from Tmsb4x, Timp1, Ybx1, Mt2, Ctsl, Cst8, Bst2, Aebp1, Bace2, Hmgcs1. The alteration can be carried out according to any of the methods provided herein and/or using any of the compounds and/or compositions provided herein. The sequences for the genes referenced herein and their respective encoded polypeptide sequences are known in the art and are publicly available.

In various aspects, the methods of the disclosure involve altering activation, development, proliferation, and/or metabolism, or killing a cell in the ovary of a genotypic female, where the cell is selected from one or more of cumulus cells, endothelial cells, epithelial cells, granulosa cells, luteal cells, myeloid cells, oocyte cells, stroma cells, and theca cells. In some cases, the cell is selected from one or more of luteal cells, stroma cells, and thecal sells. In embodiments, the cells comprise cumulus cells. The alteration or killing can be carried out according to any of the methods provided herein and/or using any of the compounds and/or compositions provided herein.

In various embodiments, the methods of the disclosure result in a reduction in incidence of pregnancies in a subject by about or at least about 5%, 10%, 20%, 30%, 40%, 50%, 50%, 60%, 70%, 80%, 90%, 95%, 99%, or 100%. In various embodiments, the methods of the disclosure result in an increase in incidence of pregnancies in a subject by about or at least about 5%, 10%, 20%, 30%, 40%, 50%, 50%, 60%, 70%, 80%, 90%, 95%, 99%, or 100%.

The methods of the disclosure may be carried out at home, the doctor's office, a clinic, a hospital's outpatient department, a hospital, or any other suitable location. Treatment may begin under the supervision of a doctor so that the doctor can observe the treatment's effects closely and make any adjustments that are needed. Drug administration may be performed at different intervals (e.g., daily, weekly, or monthly) or performed only once.

In various embodiments, the methods of the disclosure involve altering the expression, expression level, amount, and/or activity of a gene or encoded polypeptide selected from one or more of those genes listed in any one of Tables 2-7 below using one or more of the agents listed in Tables 2-7, or otherwise provided herein, and suitable for modulating the expression, expression level, amount, and/or activity of the gene or encoded polypeptide. The genes listed in the below tables were identified among the top 600 genes identified as being upregulated or downregulated in the trajectory analyses described further in the Examples. The listed genes are druggable and/or have non-steroidogenicity.

TABLE 2
Representative genes that can be targeted for disrupting the
development or function of a cumulus cell or theca cell in
the ovary of a subject to modify fertility in the subject.
Representative Upregulated Genes - Cumulus Cell Cluster

| Gene | Proposed Function | Modulators (e.g., pharmacologic, biologic) |
|---|---|---|
| Tac1 | Encodes protachykinin-1 which | NK₁ (Tachykinin receptor) antagonist: |
| | creates four products of | Aprepitant/Fosaprepitant (pro-drug) |
| | tachykinins: substance P, | (MK0869, Emend) |
| | neurokinin A, neurokinin K, and | FDA-approved |
| | neurokinin γ. Tachykinins function | Used to manage and treat |
| | as neurotransmitters that bind to | chemotherapy-induced nausea & |
| | tachykinin receptors (NK₁, NK₂, | vomiting (CINV) and |
| | NK₃) at smooth muscle cells. | postoperative nausea & |
| | | vomiting (PONV) |
| | | Burapitant (SSR-240,600, Sanofi- |
| | | Aventis) |
| | | CP-96,345 (Tocris Bioscience) |
| | | Further modulators include those disclosed in |
| | | Barrett J, et al. Tachykinin receptors (version |
| | | 2019.4) in the IUPHAR/BPS Guide to |
| | | Pharmacology Database. IUPHAR/BPS Guide |
| | | to Pharmacology CITE. 2019; 2019(4), the |
| | | disclosure of which is incorporated herein by |
| | | reference in its entirety for all purposes. |
| Gsta4 | Encodes for glutathione S- | Antagonists: |
| | transferase alpha 4 and part of the | Ellagic acid |
| | glutathione S-transferase | Curcumin |
| | superfamily. Alpha class genes | Plant polyphenolic compounds |
| | encode enzymes with glutathione | (see, e.g., Hhayeshi, et al., “The |
| | peroxidase activity that function in | inhibition of human glutathione |
| | detoxification of lipid peroxidation | S-transferases activity by plant |
| | products. | polyphenolic compounds ellagic |
| | | acid and curcumin,” Food Chem |
| | | Toxicol., 45: 286-95 (2007), the |
| | | disclosure of which is |
| | | incorporated herein by reference |
| | | in its entirety for all purposes). |
| | | Pan-GST Inhibitors: |
| | | Ethacrynic acid (EA) |
| | | Ezatiostat/Terrapin 199 (SML1081) |
| | | Additional glutathione transferase inhibitors |
| | | and pro-drugs including those disclosed in |
| | | Hayeshi R, et al. The inhibition of human |
| | | glutathione S-transferases activity by plant |
| | | polyphenolic compounds ellagic acid and |
| | | curcumin. Food Chem Toxicol. 2007 |
| | | February; 45(2): 286-95. doi: |
| | | 10.1016/j.fct.2006.07.027. Epub 2006 Aug. 30. |
| | | PMID: 17046132, and Allocati, et al. |
| | | “Glutathione transferases: substrates, inhibitors |
| | | and pro-drugs in cancer and neurodegenerative |
| | | diseases,” Oncogenesis, 7, Article No. 8 |
| | | (2018), the disclosures of which are |
| | | incorporated herein by reference in their |
| | | entirety for all purposes. |
| Mast4 | Encodes for microtubule- | siRNA, esiRNA, shRNA |
| | associated serine/threonine kinase | See, e.g., EMNC013071, EHU065261, |
| | family member 4. Members of this | and EMU078801 available from Sigma- |
| | gene family contain a domain that | Aldrich and MAST4 siRNA and |
| | gives the kinase the ability to | shRNA plasmids (e.g., MAST4 siRNA |
| | determine its own scaffold to | (h); MAST4 (h)-PR; MAST4 shRNA |
| | control the effect of their kinase | Plasmid (h); and MAST4 shRNA (h) |
| | ability. | Lentiviral Particles) available from |
| | | Santa Cruz Biotechnology, Inc. |
| F3 | Encodes for coagulation factor III | Endogenous inhibitor: |
| | (also known as tissue factor [TF]), | Tissue factor pathway inhibitor |
| | which is a cell surface | Upstream inhibitor: |
| | glycoprotein that enables cells to | Mifepristone (RU 486) |
| | initiate blood coagulation | RU 486 blocks progesterone- |
| | cascades. Platelets and monocytes | stimulated F3 expression |
| | express F3 under procoagulatory | (LOCKWOOD et al., |
| | and proinflammatory stimuli. F3 is | “Biological Mechanisms |
| | upregulated by progesterone. | Underlying RU 486 Clinical |
| | | Effects: Inhibition of |
| | | Endometrial Stromal Cell Tissue |
| | | Factor Content,” The Journal of |
| | | Clinical Endocrinology & |
| | | Metabolism, September 1994, |
| | | Vol. 79, No. 3, pp. 786-790, the |
| | | disclosure of which is |
| | | incorporated herein by reference |
| | | in its entirety for all purposes) |
| Kcnd2 | Encodes for potassium voltage- | General Calcium Channel Blockers |
| | gated channel subfamily D | (Antagonists): |
| | member 2. Voltage-gated | Nicardipine |
| | potassium channels have diverse | Brand Name: Cardene |
| | functions, including | FDA-approved |
| | neurotransmitter release, heart | Used to manage chest pain |
| | rate, insulin secretion, smooth | (angina) or high blood pressure |
| | muscle contraction, and cell | (hypertension) |
| | volume. | Dihydropyridine Ca²⁺ channel |
| | | antagonists |

TABLE 3
Representative genes that can be targeted for disrupting
the development or function of a cumulus cell in the ovary
of a subject to modify fertility in the subject.
Representative Downregulated Genes - Cumulus Cell Cluster

| Gene | Proposed Function | Modulators (e.g., pharmacologic, biologic) |
|---|---|---|
| Timp1 | Encodes for TIMP | TIMP1 neutralizing antibody (AB770, |
| | metallopeptidase inhibitor 1 | Chemicon) |
| | and the TIMP gene family that | Enhanced angiogenesis (see, e.g., |
| | are natural inhibitors of matrix | REED et al., “Inhibition of TIMP1 |
| | metalloproteinases (MMPs). | Enhances Angiogenesis in Vivo |
| | TIMP1 is shown to promote | and Cell Migration in Vitro,” |
| | cell proliferation in a wide | Microvascular Research, 1 Jan. |
| | range of cell types. | 2003, Vol. 65, No. 1, pp. 9-17, the |
| | | disclosure of which is incorporated |
| | | herein by reference in its entirety |
| | | for all purposes) |
| | | Recombinant protein and esiRNA |
| | | available from Sigma (e.g., Product Nos. |
| | | RAB0598, RAB1833, RAB0471, |
| | | RAB0467, RAB0468, RAB0470, |
| | | RAB0469, RAB0466, and RAB0878, |
| | | MAB3300, AB770, HPA053417, |
| | | SAB5702863, SAB4502971, SAB2109118, |
| | | MAB13429, IM32, MAB3301, ZRB1944, |
| | | WH0007076M1, SRP3173, SRP6445, |
| | | CC1062, EMU005581, and EHU156431) |
| Pik3c2g | Encodes for | General PI3K Inhibitors (FDA-approved): |
| | phosphatidylinositol-4- | Copanlisib |
| | phosphate 3-kinase catalytic | Brand name: Aliqopa |
| | subunit type 2 gamma, which | Used to treat adults with |
| | is part of the phosphoinositide | relapsed follicular |
| | 3-kinase (PI3K) family. PI3- | lymphoma |
| | kinases play various roles in | Idelalisib (Gilead) |
| | signaling pathways involved in | Brand name: Zydelig |
| | cell proliferation, oncogenic | Used to treat relapsed |
| | transformation, cell survival | chronic lymphocytic |
| | and migration, and intracellular | leukemia and other blood |
| | protein trafficking. | cancers |
| | | Umbralisib |
| | | Brand name: Ukoniq |
| | | Used to treat marginal zone |
| | | and follicular lymphoma |
| | | Duvelisib |
| | | Brand name: Copiktra |
| | | Used to treat chronic |
| | | lymphocytic leukemia, |
| | | small lympocytic |
| | | lymphoma, and follicular |
| | | lymphoma |
| | | Alpelisib (Novartis) |
| | | Brand name: Piqray |
| | | Used to treat certain types |
| | | of breast cancer |
| | | Additional PI3K inhibitors include those |
| | | disclosed in MISHRA et al., “PI3K |
| | | Inhibitors in Cancer: Clinical Implications |
| | | and Adverse Effects,” International Journal |
| | | of Molecular Sciences, 27 Mar. 2021, |
| | | Vol. 22, No. 7, p. 3464, the disclosure of |
| | | which is incorporated herein by reference |
| | | in its entirety for all purposes |
| Fdps | Encodes for farnesyl | Inhibitors: |
| | diphosphate synthase which | Alendronate |
| | catalyzes the production of | FDA-approved |
| | geranyl pyrophosphate and | Brand name: Binosto, |
| | farnesyl pyrophosphate. | Fosamax |
| | Farnesyl pyrophosphate is a | Used to treat or prevent |
| | key intermediate in cholesterol | osteoporosis |
| | and sterol biosynthesis. | Zoledronate |
| | | FDA-approved |
| | | Brand name: Reclast |
| | | Used to treat bone |
| | | metastasis and high blood |
| | | calcium levels |
| | | Pamidronate disodium salt |
| | | CAS 57248-88-1 (Santa |
| | | Cruz Biotechnology) |
| | | Fdps inhibitors may reduce |
| | | glioblastoma formation |
| Lgals1 | Encodes for galectin-1, which | Inhibitors: OTX008 (HY-19756) |
| | is part of the family of beta- | Thiodigalactoside (HY-130208) |
| | galactoside-binding proteins | Non-metabolizable |
| | implicated for modulating cell- | disaccharide |
| | cell and cell-matrix | Further exemplary galectin-1 modulators |
| | interactions. | include those disclosed in BLANCHARD |
| | | et al., “Galectin-1 Inhibitors and Their |
| | | Potential Therapeutic Applications: A |
| | | Patent Review,” Expert Opinion on |
| | | Therapeutic Patents, May 2016, Vol. 26, |
| | | No. 5, pp. 537-554, the disclosure of |
| | | which is incorporated herein by reference |
| | | in its entirety for all purposes. |
| | | GB1107, OTX008, Thiodigalactoside, |
| | | Selvigaltin, G3-C12 TFA, Belapectin, |
| | | Galectin-8N-IN-1, Galectin-3 antagonist 1, |
| | | G3-C12, Galectin-3-IN-1, Galectin-8-IN-1, |
| | | Apoptosis inducer 8, Galectin-3-IN-2, β- |
| | | Lactose, Galectin-3 antagonist 2, Galectin- |
| | | 3-IN-1, and GB1490 available from |
| | | MedChemExpress |
| Ramp1 | Encodes for receptor activity | CGRP Antagonists: |
| | modifying protein 1, which is a | Olcegepant (HY-100095) |
| | member of the receptor | Studied for potential |
| | (calcitonin) activity modifying | treatment for migraines |
| | proteins (RAMPs). RAMPs are | BIBN-4096 (Bio-Techne) |
| | essential for transporting | MK-3207 (HY-10301) |
| | calcitonin-receptor-like | Further exemplary modulators |
| | receptor (CRLR) to the plasma | include those disclosed in |
| | membrane. In the presence of | DALLMAYER et al., “Targeting |
| | RAMP1, CRLR functions as a | the CALCB/RAMP1 Axis Inhibits |
| | calcitonin-gene-related peptide | Growth of Ewing Sarcoma,” Cell |
| | (CGRP receptor). | Death & Disease, 11 Feb. |
| | | 2019, Vol. 10, No. 2, pp. 1-13, 2, |
| | | the disclosure of which is |
| | | incorporated herein by reference in |
| | | its entirety for all purposes |
| Emb | Encodes for embigin, which is | siRNA: |
| | a transmembrane glycoprotein | See, e.g., those siRNAs disclosed in |
| | that is part of the | Ruma, et al. Cancers 10: 239 |
| | immunoglobulin superfamily. | (2018), the disclosure of which is |
| | Embigin has been shown to be | incorporated herein by reference in |
| | involved in cell growth and | its entirety for all purposes |
| | development via cell-ECM | See, e.g., those siRNAs disclosed in |
| | interactions. | Jung, et al. Molecular |
| | | Carcinogenesis, 55: 633-645 |
| | | (2016), the disclosure of which is |
| | | incorporated herein by reference in |
| | | its entirety for all purposes |

TABLE 4
Representative genes that can be targeted for disrupting
the development or function of a theca cell in the ovary
of a subject to modify fertility in the subject.
Representative Upregulated Genes - Theca Cell Cluster

| Gene | Proposed Function | Modulators (e.g., pharmacologic, biologic) |
|---|---|---|
| Tmsb4x | Encodes for thymosin beta 4 | Inhibitor: |
| | X-linked, which is an actin | Latrunculin A |
| | sequestering protein involved | Sponge toxin that prevents |
| | in regulation of actin | binding of TMSB4X to actin |
| | polymerization. TMSB4X is | (NUMMELA et al., |
| | involved in cell proliferation, | “Thymosin Beta4 Is a |
| | migration, and differentiation. | Determinant of the |
| | | Transformed Phenotype and |
| | | Invasiveness of S- |
| | | Adenosylmethionine |
| | | Decarboxylase-Transfected |
| | | Fibroblasts,” Cancer |
| | | Research, 15 Jan. 2006, |
| | | Vol. 66, No. 2, pp. 701-712, |
| | | the disclosure of which is |
| | | incorporated herein by |
| | | reference in its entirety for |
| | | all purposes) |
| | | ShRNA (see, e.g., CHI et al., “Global |
| | | Proteomics-Based Identification and |
| | | Validation of Thymosin Beta-4 X-Linked as |
| | | a Prognostic Marker for Head and Neck |
| | | Squamous Cell Carcinoma,” Scientific |
| | | Reports, 22 Aug. 2017, Vol. 7, No. 1, p. |
| | | 9031, 1, the disclosure of which is |
| | | incorporated herein by reference in its |
| | | entirety for all purposes) |
| | | Recombinant protein |
| | | Available at Cell Biologics, |
| | | Medchem, Biorbyt |
| Timp1 | Encodes for TIMP | TIMP1 neutralizing antibody (AB770, |
| | metallopeptidase inhibitor 1 | Chemicon) |
| | and the TIMP gene family | Enhanced angiogenesis (REED et |
| | that are natural inhibitors of | al., “Inhibition of TIMP1 Enhances |
| | matrix metalloproteinases | Angiogenesis in Vivo and Cell |
| | (MMPs). TIMP1 is shown to | Migration in Vitro,” Microvascular |
| | promote cell proliferation in a | Research, 1 Jan. 2003, Vol. 65, |
| | wide range of cell types. | No. 1, pp. 9-17, the disclosure of |
| | | which is incorporated herein by |
| | | reference in its entirety for all |
| | | purposes) |
| | | Recombinant protein and esiRNA available |
| | | from Sigma |
| Ybx1 | Encodes for Y-box binding | Inhibitors: |
| | protein 1 which is a highly | SU056 |
| | conserved cold shock domain | Azopodophyllotoxin small |
| | protein with broad nucleic | molecule |
| | acid binding properties (DNA | See, e.g., AILOR et al., “Y Box Binding |
| | and RNA). It is implicated in | Protein 1 Inhibition as a Targeted Therapy |
| | many cellular processes, | for Ovarian Cancer,” Cell Chemical |
| | including regulation of | Biology, 19 Aug. 2021, Vol. 28, No. 8, |
| | transcription, translation, pre- | pp. 1206-1220.e6, the disclosure of which is |
| | mRNA splicing, DNA | incorporated herein by reference in its |
| | reparation, and mRNA | entirety for all purposes |
| | packaging. | Additional YBX1 inhibitors include |
| | | Soyasaponin II, SU056, RSK-IN-1, YBX1 |
| | | Human Pre-designed siRNA Set A, and |
| | | LJI308 available from MedChemExpress |
| Vim | Encodes for vimentin which | Inhibitors: |
| | is a type III intermediate | Withaferin A |
| | filament protein. VIM is | Steroidal lactone derived |
| | responsible for maintaining | from Solanaceae family of |
| | cell shape and integrity of the | plants |
| | cytoplasm as well as | Causes aggregation of |
| | stabilizing cytoskeletal | vimentin to colocalize with |
| | interactions. | F-actin leading to apoptosis |
| | | Arylquin 1 (SML1263, Sigma) |
| | | Binds to vimentin and |
| | | displaces Par-4 from |
| | | vimention for secretion |
| | | FiVe1 |
| | | Irreversible inhibitor of |
| | | vimentin in mesenchymal |
| | | breast cancer cells (See, e.g., |
| | | BOLLONG et al., “A |
| | | Vimentin Binding Small |
| | | Molecule Leads to Mitotic |
| | | Disruption in Mesenchymal |
| | | Cancers,” Proceedings of the |
| | | National Academy of |
| | | Sciences of the United States |
| | | of America, 14 Nov. |
| | | 2017, Vol. 114, No. 46, pp. |
| | | E9903-E9912, the disclosure |
| | | of which is incorporated |
| | | herein by reference in its |
| | | entirety for all purposes) |
| | | Additional inhibitors for vimentin include |
| | | those disclosed in STROUHALOVA et al., |
| | | “Vimentin Intermediate Filaments as |
| | | Potential Target for Cancer Treatment,” |
| | | Cancers, January 2020, Vol. 12, No. 1, p. |
| | | 184, the disclosure of which is incorporated |
| | | herein by reference in its entirety for all |
| | | purposes |
| | | Modulators: |
| | | Silibin, resveratrol, and dioscin |
| | | Decreases Vim expression |
| Col4a1 | Encodes for collagen type IV | ShRNA: |
| | alpha 1 chain. Type IV | See, e.g., WANG et al., “COL4A1 |
| | collagen proteins are | Promotes the Growth and Metastasis |
| | important for basement | of Hepatocellular Carcinoma Cells |
| | membranes and interact with | by Activating FAK-Src Signaling,” |
| | other ECM proteins including | Journal of Experimental & Clinical |
| | perlecans, proteoglycans, and | Cancer Research, 3 Aug. 2020, |
| | laminins. | Vol. 39, No. 1, p. 148, the disclosure |
| | | of which is incorporated herein by |
| | | reference in its entirety for all |
| | | purposes |
| | | Modulator: |
| | | GFOGER peptide |
| | | Short six-amino-acid repeat |
| | | from the sequence of COL1 |
| | | gene (MANSOUR et al., |
| | | “GFOGER Peptide Modifies |
| | | the Protein Content of |
| | | Extracellular Vesicles and |
| | | Inhibits Vascular |
| | | Calcification,” Frontiers in |
| | | Cell and Developmental |
| | | Biology, 2020, Vol. 8, the |
| | | disclosure of which is |
| | | incorporated herein by |
| | | reference in its entirety for |
| | | all purposes) |
| | | Blocks cell adhesion |
| Zfp804a | Encodes for a zinc-finger | siRNA |
| | binding protein and predicted | See, e.g., those siRNAs disclosed in |
| | to enable metal ion binding | Deans, et al., Biol Psychiatry, 82: 49-61 |
| | activity. | (2017), the disclosure of which is |
| | | incorporated herein by reference in its |
| | | entirety for all purposes |
| | | shRNA |
| | | See, e.g., those shRNAs disclosed in |
| | | Huang, et al. Molecular Psychiatry, |
| | | 26: 2514-2532 (2021), the disclosure of |
| | | which is incorporated herein by |
| | | reference in its entirety for all purposes |

TABLE 5
Representative genes that can be targeted for disrupting
the development or function of a theca cell in the ovary
of a subject to modify fertility in the subject.
Representative Downregulated Genes - Theca Cell Cluster

| Gene | Proposed Function | Modulators (e.g., pharmacologic, biologic) |
|---|---|---|
| Gas6 | Encodes for growth arrest | Inhibitors: |
| | specific 6 protein, which is a | RU-301 (HY-119039) & RU-302 |
| | gamma-carboxyglutamic | (HY-124066) |
| | (Gla)-containing protein | pan-TAM inhibitors |
| | potentially involved in | previously used in cancer |
| | stimulating cell proliferation. | studies |
| | | Warfarin |
| | | Vitamin K antagonist |
| | | MYD1/MYD1-72 |
| | | Soluble receptor |
| | | AVB-500 |
| | | Soluble receptor (currently |
| | | Phase I/II) |
| | | Additional inhibitors for Gas6 |
| | | include those listed in TANAKA et |
| | | al., “Therapeutic Targeting of the |
| | | Gas6/Axl Signaling Pathway in |
| | | Cancer,” International Journal of |
| | | Molecular Sciences, 15 Sep. |
| | | 2021, Vol. 22, No. 18, p. 9953, the |
| | | disclosure of which is incorporated |
| | | herein by reference in its entirety |
| | | for all purposes |
| | | Agonist: |
| | | Recombinant protein (see, e.g., |
| | | TONG et al., “Recombinant Gas6 |
| | | Augments Axl and Facilitates |
| | | Immune Restoration in an |
| | | Intracerebral Hemorrhage Mouse |
| | | Model,” Journal of Cerebral Blood |
| | | Flow and Metabolism: Official |
| | | Journal of the International Society |
| | | of Cerebral Blood Flow and |
| | | Metabolism, June 2017, Vol. 37, |
| | | No. 6, pp. 1971-1981, the |
| | | disclosure of which is incorporated |
| | | herein by reference in its entirety |
| | | for all purposes |
| Pak3 | Encodes for P21 (RAC1) | PAK Inhibitors: |
| | activated kinase 3, which is a | FRAX597 (S7271), FRAX486 |
| | serine-threonine kinase that | (S7807), PF-3758309 (S7094) |
| | forms an activated complex | Available from Selleckchem |
| | with GTP-bound RAS-like | Additional PAK inhibitors available |
| | (P21), CDC2, and RAC1. | those available from Tocris |
| | | including AZ 13705339, FRAX |
| | | 486, FRAX 597, G 5555, GNE |
| | | 2861, IPA3, NVS PAK1 1, and PF |
| | | 3758309 dihydrochloride |
| Trib2 | Encodes for Tribbles | EGFR family inhibitors: |
| | pseudokinase 2, which | Lapatinib |
| | contains a Trb domain | Brand name: Tykreb |
| | (homologous to serine- | FDA-approved |
| | threonine kinase) but lacks | Used to treat breast cancer |
| | active site lysine and | Afatinib |
| | potentially lacks catalytic | Brand name: Gilotrif |
| | function. Tribbles family | FDA-approved |
| | interact and modulate activity | Used to treat non-small cell |
| | of signal transduction | lung carcinoma |
| | pathways. | Nertinib |
| | | Brand name: Nerlynx |
| | | FDA approved: |
| | | Used to treat breast cancer |
| | | Additional inhibitors include those |
| | | disclosed in FOULKES et al., “Covalent |
| | | Inhibitors of EGFR Family Protein Kinases |
| | | Induce Degradation of Human Tribbles 2 |
| | | (TRIB2) Pseudokinase in Cancer Cells,” |
| | | Science Signaling, 25 Sep. 2018, |
| | | Vol. 11, No. 549, p. eaat7951, the |
| | | disclosure of which is incorporated herein |
| | | by reference in its entirety for all purposes |
| Smoc2 | Encodes for SPARC-related | siRNA: |
| | modular calcium binding 2 | See, e.g., those siRNA molecules |
| | protein, which is highly | disclosed in GERARDUZZI et al., |
| | expressed during | “Silencing SMOC2 Ameliorates |
| | embryogenesis and wound | Kidney Fibrosis by Inhibiting |
| | healing. SMOC2 is a | Fibroblast to Myofibroblast |
| | matricellular protein that | Transformation,” JCI Insight, Vol. |
| | promotes matrix assembly and | 2, No. 8, p. e90299 or LIU, DI et |
| | can stimulate endothelial cell | al., “SMOC2 Promotes Aggressive |
| | proliferation and migration. | Behavior of Fibroblast-like |
| | | Synoviocytes in Rheumatoid |
| | | Arthritis through Transcriptional |
| | | and Post-Transcriptional Regulating |
| | | MYO1C,” Cell Death & Disease, |
| | | 13 Dec. 2022, Vol. 13, No. 12, |
| | | pp. 1-13, the disclosures of which |
| | | are incorporated herein by reference |
| | | in their entireties for all purposes |
| Kit | Encodes for a receptor tyrosine | Inhibitors: |
| | kinase which is referred to as | Semaxanib (SU5416, SUGEN) |
| | the proto-oncogene c-Kit. c-Kit | VEGF receptor kinase |
| | phosphorylates many | inhibitor |
| | intracellular proteins and plays | SU6668 |
| | a role in proliferation, | PDGF, VEGF, and FGF |
| | differentiation, migration, and | receptor inhibitor |
| | apoptosis across various cell | Imatinab (STI-571, Novartis) |
| | types. It plays an important | Brand name: Gleevec |
| | role in hematopoiesis, stem | FDA-approved |
| | cell maintenance, | Used to treat leukemia and |
| | gametogenesis, melanogenesis, | related cancers |
| | etc. | Additional clinically approved |
| | | inhibitors include those disclosed in |
| | | BAUER et al., “Early and Next- |
| | | Generation KIT/PDGFRA Kinase |
| | | Inhibitors and the Future of |
| | | Treatment for Advanced |
| | | Gastrointestinal Stromal Tumor,” |
| | | Frontiers in Oncology, 2021, Vol. |
| | | 11, the disclosure of which is |
| | | incorporated herein by reference in |
| | | its entirety for all purposes |
| | | Agonists: |
| | | Stem cell factor (SCF) variants/ |
| | | monomers (e.g., those disclosed in |
| | | TILAYOV et al., “Engineering |
| | | Stem Cell Factor Ligands with |
| | | Different C-Kit Agonistic |
| | | Potencies,” Molecules (Basel, |
| | | Switzerland), 21 Oct. 2020, Vol. |
| | | 25, No. 20, p. 4850, the disclosure |
| | | of which is incorporated herein by |
| | | reference in its entirety for all |
| | | purposes) |
| Tpm4 | Encodes for tropomyosin 4, | siRNA: |
| | which is typically involved in | See, e.g., those siRNAs disclosed in |
| | actin cytoskeleton organization | Jeong, et al., Oncotarget, 8: 33544- |
| | and contraction of non-muscle | 33559 (2017) |
| | issues. In non-mammalian | |
| | models, tropomyosin has been | |
| | shown to be involved in | |
| | follicle rupture. | |
| Fndc3b | Encodes for a transmembrane | miRNA: |
| | protein with repeated | miR-1225-5p |
| | fibronectin type III domains. | See, e.g., Wang, et al. Open |
| | Plays a role in cell migration, | Med (Wars), 15: 872-881 |
| | adhesion, and proliferation. | (2020), the disclosure of |
| | | which is incorporated herein |
| | | by reference in its entirety |
| | | for all purposes. |
| | | miR-192-5p |
| | | See, e.g., You, et al. Free |
| | | Radical Biology and |
| | | Medicine, 193: 808-819 |
| | | (2022), the disclosure of |
| | | which is incorporated herein |
| | | by reference in its entirety |
| | | for all purposes. |
| | | shRNA: |
| | | See, e.g., those shRNAs disclosed in Lin, et |
| | | al. Oncotarget, 7: 49498-49508. |
| Cnn3 | Encodes for a filament- | siRNA: |
| | associated protein that controls | See, e.g., those siRNAs disclosed in |
| | smooth muscle contraction. | Xia, et al. Sci Rep. 10: 2427 (2020), |
| | May be involved as a | the disclosure of which is |
| | component of vascular smooth | incorporated herein by reference in |
| | muscle within newly formed | its entirety for all purposes. |
| | vessels during ovulation. | shRNA: |
| | | See, e.g., those shRNAs disclosed |
| | | in Daimon, et al. Archives of |
| | | Dermatological Research, 305: 571- |
| | | 584 (2013), the disclosure of which |
| | | is incorporated herein by reference |
| | | in its entirety for all purposes. |

TABLE 6
Representative genes that can be targeted for disrupting
the development or function of a luteal or thecal cell in
the ovary of a subject to modify fertility in the subject.
Representative Upregulated Genes - Luteal Cell Cluster

| Gene | Proposed Function | Modulators (e.g., pharmacologic, biologic) |
|---|---|---|
| Tmsb4x | Encodes for thymosin beta 4 | Inhibitor: |
| | X-linked, which is an actin | Latrunculin A |
| | sequestering protein involved | Sponge toxin that prevents |
| | in regulation of actin | binding of TMSB4X to actin |
| | polymerization. TMSB4X is | (NUMMELA et al., |
| | involved in cell proliferation, | “Thymosin Beta4 Is a |
| | migration, and differentiation. | Determinant of the |
| | | Transformed Phenotype and |
| | | Invasiveness of S- |
| | | Adenosylmethionine |
| | | Decarboxylase-Transfected |
| | | Fibroblasts,” Cancer |
| | | Research, 15 Jan. 2006, |
| | | Vol. 66, No. 2, pp. 701-712, |
| | | the disclosure of which is |
| | | incorporated herein by |
| | | reference in its entirety for |
| | | all purposes |
| | | ShRNA: |
| | | See, e.g, CHI et al., “Global |
| | | Proteomics-Based Identification and |
| | | Validation of Thymosin Beta-4 X- |
| | | Linked as a Prognostic Marker for |
| | | Head and Neck Squamous Cell |
| | | Carcinoma,” Scientific Reports, 22 |
| | | Aug. 2017, Vol. 7, No. 1, p. 9031, |
| | | 1, the disclosure of which is |
| | | incorporated herein by reference in |
| | | its entirety for all purposes |
| | | Recombinant human thymosin-beta 4 |
| | | (TMSB4X) protein |
| | | Available at Cell Biologics, |
| | | Medchem, Biorbyt |
| Timp1 | Encodes for TIMP | TIMP1 neutralizing antibody (AB770, |
| | metallopeptidase inhibitor 1 | Chemicon) |
| | and the TIMP gene family | Enhanced angiogenesis (REED et |
| | that are natural inhibitors of | al., “Inhibition of TIMP1 Enhances |
| | matrix metalloproteinases | Angiogenesis in Vivo and Cell |
| | (MMPs). TIMP1 is shown to | Migration in Vitro,” Microvascular |
| | promote cell proliferation in a | Research, 1 Jan. 2003, Vol. 65, |
| | wide range of cell types. | No. 1, pp. 9-17, the disclosure of |
| | | which is incorporated herein by |
| | | reference in its entirety for all |
| | | purposes) |
| | | Recombinant protein and esiRNA available |
| | | from Sigma (e.g., Product Nos. RAB0598, |
| | | RAB0467, RAB0468, RAB0470, |
| | | RAB0469, HPA053417, MAB3300, |
| | | AB770, SAB5702863, SAB4502971, |
| | | SAB2019118, IM32, MAB3301, |
| | | MAB13429, ZRB1944, WH0007076M1, |
| | | SRP6445, SRP3173, CC1062, EMU005581, |
| | | EHU156431) |
| Ybx1 | Encodes for Y-box binding | Inhibitors: |
| | protein 1 which is a highly | SU056 |
| | conserved cold shock domain | Azopodophyllotoxin small |
| | protein with broad nucleic | molecule |
| | acid binding properties (DNA | See, e.g., TAILOR et al., “Y Box Binding |
| | and RNA). It is implicated in | Protein 1 Inhibition as a Targeted Therapy |
| | many cellular processes, | for Ovarian Cancer,” Cell Chemical |
| | including regulation of | Biology, 19 Aug. 2021, Vol. 28, No. 8, |
| | transcription, translation, pre- | pp. 1206-1220.e6, the disclosure of which is |
| | mRNA splicing, DNA | incorporated herein by reference in its |
| | reparation, and mRNA | entirety for all purposes |
| | packaging. | Additional YBX1 inhibitors include |
| | | Soyasaponin II, SU056, RSK-IN-1, YBX1 |
| | | Human Pre-designed siRNA Set A, and |
| | | LJI308 available from MedChemExpress |
| Mt2 | Encodes for melatonin | Antagonists: |
| | receptor 1B, which when | 4P-ADOT and 4-P-PDOT |
| | bound to melatonin produces | See, e.g., DUBOCOVICH et |
| | an integral G-coupled | al., “Selective MT2 |
| | membrane protein. It is | Melatonin Receptor |
| | involved in light-dependent | Antagonists Block |
| | functions in the retina. | Melatonin-Mediated Phase |
| | | Advances of Circadian |
| | | Rhythms,” FASEB Journal: |
| | | Official Publication of the |
| | | Federation of American |
| | | Societies for Experimental |
| | | Biology, September 1998, |
| | | Vol. 12, No. 12, pp. 1211- |
| | | 1220, the disclosure of which |
| | | is incorporated herein by |
| | | reference in its entirety for |
| | | all purposes |
| | | Luzindole |
| | | 25-fold affinity for MT2 over |
| | | MT1 receptor |
| | | K185 |
| | | See, e.g., LIU, D. et al., “The |
| | | MT2 Receptor Stimulates |
| | | Axonogenesis and Enhances |
| | | Synaptic Transmission by |
| | | Activating Akt Signaling,” |
| | | Cell Death & Differentiation, |
| | | April 2015, Vol. 22, No. 4, |
| | | pp. 583-596, the disclosure |
| | | of which is incorporated |
| | | herein by reference in its |
| | | entirety for all purposes |
| Ctsl | Encodes for cathepsin L, | Inhibitors |
| | which is a lysosomal cysteine | SID 26681509 |
| | proteinase involved in | Available from Bio-Techne |
| | intracellular protein | See, e.g., ZHAO et al., |
| | catabolismits substrates | “Cathepsin L Plays a Key |
| | include collagen and elastin. | Role in SARS-COV-2 |
| | | Infection in Humans and |
| | | Humanized Mice and Is a |
| | | Promising Target for New |
| | | Drug Development,” Signal |
| | | Transduction and Targeted |
| | | Therapy, 27 Mar. 2021, |
| | | Vol. 6, No. 1, pp. 1-12, the |
| | | disclosure of which is |
| | | incorporated herein by |
| | | reference in its entirety for |
| | | all purposes |
| | | Capthesin inhibitors: |
| | | Pierce ™ E-64 Protease Inhibitor |
| | | Available from Thermo |
| | | Fisher |

TABLE 7
Representative genes that can be targeted for disrupting
the development or function of a theca cell in the ovary
of a subject to modify fertility in the subject.
Representative Downregulated Genes - Luteal Cell Cluster

| Gene | Proposed Function | Modulators (e.g., pharmacologic, biologic) |
|---|---|---|
| Cst8 | Encodes cystatin 8, which is | Cystatin B/CST8 Protein, Human |
| | part of the cystatin | (HEK293, Fc): |
| | superfamily. Cystatin 8 is | Available from MedChemExpress |
| | similar to type 2 cystatins | |
| | which are typically cysteine | |
| | proteinase inhibitors found in | |
| | a variety of human fluids or | |
| | secretions. CST8 exhibits a | |
| | highly tissue-specific | |
| | expression in the reproductive | |
| | tract. | |
| Bst2 | Encodes for bone marrow | Inhibitors: |
| | stromal antigen 2. However, | B49/B49-Mod1 |
| | the function of this gene is | Representative inhibitors include |
| | still undetermined. | those disclosed in MAHAUAD- |
| | | FERNANDEZ et al., “B49, a BST- |
| | | 2-Based Peptide, Inhibits Adhesion |
| | | and Growth of Breast Cancer Cells,” |
| | | Scientific Reports, 9 Mar. 2018, |
| | | Vol. 8, No. 1, p. 4305 or |
| | | international patent application No. |
| | | WO2008127261, the disclosures of |
| | | which are incorporated herein by |
| | | reference in their entireties for all |
| | | purposes. |
| | | RNAi: |
| | | See, e.g., those RNAis disclosed in |
| | | Pham, et al. Anticancer Research, |
| | | 37: 2853-2860 (2017), the disclosure |
| | | of which is incorporated herein by |
| | | reference in its entirety for all |
| | | purposes. |
| Aebp1 | Encodes for AE binding | EsiRNA and Protein: |
| | protein 1, which is a member | Available from Sigma (e.g., Product |
| | of the carboxypeptidase A | Nos. HP A063595, HPA064970, |
| | family. AEBP1 is suggested to | HPA047724, AV31592, |
| | function as a transcriptional | EHU043741, and EMU050491) |
| | repressor and play a role in | |
| | adipogenesis and smooth | |
| | muscle cell differentiation. | |
| Bace2 | Encodes for beta-secretase 2, | Inhibitors: |
| | which is an integral membrane | BACE2-IN1 |
| | glycoprotein that functions as | Available from |
| | an aspartic protease. BACE2 | MedChemExpress |
| | cleaves amyloid precursor | Further representative inhibitors |
| | protein into amyloid beta | include those disclosed in GHOSH |
| | peptide which is a critical step | et al., “Highly Selective and Potent |
| | in the etiology of Alzheimer's | Human β-Secretase 2 (BACE2) |
| | disease and Down's | Inhibitors against Type 2 Diabetes: |
| | syndrome. | Design, Synthesis, X-Ray Structure |
| | | and Structure-Activity Relationship |
| | | Studies,” ChemMedChem, 5 Mar. |
| | | 2019, Vol. 14, No. 5, pp. 545-560, |
| | | the disclosure of which is |
| | | incorporated herein by reference in |
| | | its entirety for all purposes |
| Hmgcs1 | Encodes for 3-hydroxy-3- | Inhibitors: |
| | methylglutaryl-CoA synthase | Hymeglusin |
| | 1, which enables protein | See, e.g., ZHOU et al., |
| | homodimerization activity. | “HMGCS1 Drives Drug- |
| | HMGCS1 is predicted to be | Resistance in Acute Myeloid |
| | involved in the acetyl-CoA | Leukemia through |
| | metabolic process and | Endoplasmic Reticulum- |
| | farnesyl diphosphoate | UPR-Mitochondria Axis,” |
| | biosynthesis process. | Biomedicine & |
| | | Pharmacotherapy = |
| | | Biomedecine & |
| | | Pharmacotherapie, May |
| | | 2021, Vol. 137, No. p. |
| | | 111378, the disclosure of |
| | | which is incorporated herein |
| | | by reference in its entirety |
| | | for all purposes |
| | | Simvastatin |
| | | HMG-CoA reductase |
| | | inhibitor (statin) |
| | | Used to treat high |
| | | cholesterol and triglyceride |
| | | (fat) levels in the blood |
| | | Metformin |
| | | Commonly used biguanide |
| | | for controlling glucose levels |
| | | in circulation and for treating |
| | | insulin resistance |
| | | See, e.g., CHEN et al., |
| | | “Antidiabetic Drug |
| | | Metformin Suppresses |
| | | Tumorigenesis through |
| | | Inhibition of Mevalonate |
| | | Pathway Enzyme |
| | | HMGCS1,” The Journal of |
| | | Biological Chemistry, 8 |
| | | Nov. 2022, Vol. 298, |
| | | No. 12, p. 102678, the |
| | | disclosure of which is |
| | | incorporated herein by |
| | | reference in its entirety for |
| | | all purposes |
| | | Agonist: |
| | | Itraconazole |
| | | Type of azole antifungal |
| | | medication |
| | | Capsule usually to treat |
| | | fungal infections |

### Ovulation and Follicle Development

Ovulation is a dynamic process initiated by the luteinizing hormone, where the ovarian follicle undergoes a series of complex physiological changes that lead to the release of a mature oocyte. These changes, including cumulus oocyte complex (COC) expansion, are mediated by a variety of signaling pathways and involve the coordinated regulation of gene expression in different cell types within the ovary.

Ovulation is a physiologic process defined by the rupture and release of the dominant follicle from the ovary into the fallopian tube where it has the potential to become fertilized. The ovulation process is regulated by fluxing gonadotropic hormone (FSH/LH) levels. Ovulation is the third phase within the larger Uterine Cycle (i.e., Menstrual Cycle). The follicular release follows the Follicular phase (i.e., dominant follicle development) and precedes the Luteal phase (i.e., maintenance of corpus luteum) that progresses to either endometrial shedding or implantation. Follicular release occurs around 14 days prior to menstruation in a cyclic pattern if the hypothalamic-pituitary-ovarian axis function is well regulated.

Genotypic females (XX) develop two ovaries that sit adjacent to the uterine horns. Each ovary is anchored at the medial pole by the utero-ovarian ligament to the uterus. The lateral ovarian pole is anchored to the pelvic sidewall by the infundibulopelvic ligament (i.e., suspensory ligament of the ovary), which carries the ovarian artery and vein. Each ovary contains 1 to 2 million primordial follicles that each contain primary oocytes (i.e., eggs) that can supply that female with enough follicles until she reaches her fourth or fifth decades of life. These primordial follicles are arrested in Prophase I of meiosis until the onset of puberty. At the onset of pubescence, the gonadotropic hormones began to induce the maturation of the primordial follicle allowing for completion of Meiosis I forming a secondary follicle. The secondary follicle begins Meiosis II, but this phase will not be completed unless that follicle is fertilized. With each ovulatory cycle, the number of follicles decreases eventually leading to the onset of Menopause or the cessation of ovulatory function. Per each ovulation cycle, the average ovary loses 1,000 follicles to the process of selecting a dominant follicle that will be released. This process accelerates in an age-dependent manner as well. It is also a common thought that the right and left ovaries alternate follicular releases each month.

The ovary is an oval-shaped organ about the size of an almond. It is organized into germ cells (i.e., oocytes) and somatic cells (i.e., granulosa, theca, and stromal cells) that work together to develop dominant mature follicles that can be released through ovulation for possible fertilization. The actions of the ovary are regulated primarily by FSH and LH hormones produced by the anterior pituitary gland as previously mentioned. Those hormones act as ligands to two receptor types found on somatic cells. The actions of these cells propagate the development of the adjacent germ cells to mature by providing an estrogen-rich environment.

An oocyte is the germ cell within the ovary that progresses through a series of maturation steps. Primordial follicles are immature germ cells or primary follicles arrested in Prophase I of Meiosis. The onset of pubescence enables the completion of primordial follicles into primary oocytes through a process called folliculogenesis. Primary oocytes have a single layer of granulosa cells surrounding them. When the theca cell layer develops adjacent to the granulosa cells, the primary follicle develops into a secondary follicle. A mature (Graafian) follicle is characterized by the development of a liquid-filled cavity called the Antrum. Immediately prior to ovulation, the Graafian follicle begins Meiosis II and arrests at Metaphase II. This process is only completed if the oocyte is fertilized.

Granulosa cells are somatic cells that immediately surround the growing oocyte. They respond to follicle-stimulating hormone (FSH) released by the anterior pituitary by converting androgens to estrogen prior to the LH surge. The androgens used by the granulosa cells are provided by the Theca cells that lie outside of the granulosa cells. After the LH surge, the granulosa cells undergo a receptor transition called “luteinization”. Luteinization converts granulosa cells into cells that are receptive to the luteinizing hormone. This process enables granulosa cells to now produce Progesterone instead of estrogen as they previously did. After ovulation, granulosa cells in conjunction with the Theca-lutein cells create the Corpus Luteum which is primarily responsible for Progesterone.

Theca cells are somatic cells that appear as the follicle matures and are found immediately outside of the granulosa cells. Their main function is to synthesize androgens that diffuse into the near-by granulosa cells for conversion to estrogen. Theca cells are regulated by LH and these cells undergo a “luteinization” phase like the granulosa cells, where they become “theca-lutein” cells that directly produce progesterone as part of the Corpus Luteum.

Stromal cells are somatic cells that are the connective tissue cells that create the organizational scaffolding for the organ-specific cells. (i.e., fibroblasts, endothelial cells, epithelial cells, etc.) Stromal cells are a major source of malignant processes, especially in the ovary. In fact, epithelial cells are responsible for the most common type of ovarian cancer.

The prepubertal ovary contains primordial follicles, which consists of an oocyte surrounded by a single layer of granulosa cells. Following puberty, the anterior pituitary begins to secrete FSH and LH in response to GnRH release from the hypothalamus, and the dormant cells in the ovary begin to secrete steroid hormones in response.

Approximately 1,000 primordial follicles begin the process of maturation into primary follicles. At the onset of development, the granulosa cell layer that surrounds the oocyte increases in size and they begin estrogen production through FSH stimulation. FSH acts to initially propagate the beginning of estrogen synthesis; however, estrogen production becomes an autonomous process by granulosa cells. Thus, estrogen production and follicle development occur independently of FSH. The zona pellucida develops at this stage as well, and becomes the outermost portion of the oocyte, demarcating it from the granulosa cells. The zona pellucida in the protective casing through which sperm must penetrate in order to fertilize the egg following ovulation.

A subset of these primary follicles progress to the secondary follicle stage, during which the theca cell layer forms. Theca cells are stimulated by LH to synthesize androgens, which diffuse into the granulosa cells as estrogen precursors.

Next, the follicle develops a fluid-filled cavity surrounding the oocyte known as an antrum. At this stage, the follicle is referred to as an antral, or Graafian follicle. This stage can also be seen on ultrasound as a small, fluid-filled cyst on the ovary. The follicular phase of the menstrual cycle occurs when the antral follicle develops into a preovulatory follicle in preparation for ovulation. The follicular phase (i.e., follicle development) begins on day one which is characterized by the onset of menstruation and continues today 14 (i.e., ovulation) of a typical 28-day cycle. The antral follicle is dependent on FSH at this stage, and it begins to compete with the other developing follicles for FSH. The follicle that dominates this process is called the “dominant follicle” and all others will become atretic. The antral or “dominant” follicles secrete estrogen and inhibin, which exert negative feedback on FSH, thus “turning off” their neighboring antral follicles.

The majority of the follicles which began the process of maturation will undergo atresia (radical apoptosis of all cells within the follicle, including the oocyte) at some point during this process, leaving only one (rarely more) mature follicle to ovulate. If more than one follicle ovulates in a given cycle, this leads to non-identical multiple gestations, such as fraternal twins.

Ovulation occurs around day 14 of a typical 28-day cycle. Estrogen levels rise as a result of increased estrogen production by hormonally active granulosa cells within the follicle. Once estrogen levels reach a critical point and remain at the level for 2 days, estrogen transitions from a negative feedback modulator of GnRH to a positive feedback modulator on the hypothalamus. This transition point leads to an increased frequency of GnRH secretion onto the anterior pituitary, leading to an LH surge. The LH surge increases intrafollicular proteolytic enzymes, weakening the wall of the ovary and allowing for the mature follicle to pass through.

The surge also causes the luteinization of thecal and granulosa cells forming the Corpus Luteum, which is responsible for progesterone synthesis levels. Once the follicle is released, it is caught by the fimbriae of the fallopian tubes. The oocyte remains in metaphase II of meiosis II unless fertilization occurs.

The luteal phase lasts from day 14 to 28 of a typical cycle. It begins with the formation of the corpus luteum and ends in pregnancy or luteolysis (destruction of the corpus luteum). FSH and LH stimulate what remains of the mature follicle after ovulation to become the corpus luteum. The corpus luteum grows and secretes progesterone and some estrogen, which makes the endometrium more receptive to implantation. If fertilization does not occur, progesterone/estrogen levels fall, and the corpus luteum dies forming the corpus albicans. These falling hormone levels stimulate FSH to begin recruiting follicles for the next cycle. If fertilization does occur, human chorionic gonadotropin (hCG) produced by the early placenta preserves the corpus luteum, maintaining progesterone levels until the placenta is able to make sufficient progesterone to support the pregnancy.

Accordingly, the present disclosure provides non-hormonal methods for affecting follicle development in a genotypic (XX) female subject to prevent or facilitate contraception in the subject.

### Female Contraceptives

A female contraceptive is an agent that reduces the ability of a genotypic female to become pregnant. Female contraceptives may involve the use of hormones (i.e., hormonal contraceptives) or they may not involve the use of any hormones (i.e., non-hormonal contraceptives).

Drawbacks of hormonal birth control include various side effects, such as nausea, headaches, spotting, breast tenderness, weight gain, ovarian cysts, irregular periods, pain, depression or mood changes, skin reactions, and/or increased vaginal wetness. Drawbacks of hormonal birth control also include increased risk of cancer, stroke, heart attack, liver tumors, blood clots, uterine puncture, fevers, chills, and/or difficulty breathing. Many subjects discontinue hormonal birth control as a result of these drawbacks.

In embodiments, a non-hormonal contraceptive method has the advantage of having fewer undesired side effects than a hormonal contraceptive method. In some cases, a subjects administered a non-hormonal contraceptive are more likely to continue use thereof than use of a hormonal contraceptive.

Accordingly, the present disclosure provides non-hormonal compositions and methods for preventing pregnancy in genotypic females.

### Contraceptive Therapy

In embodiments, methods of the disclosure involve contraceptive therapy. In some cases, the methods involve administering to a subject an agent (e.g., polypeptide, polynucleotide, or fragment thereof) capable of reducing or increasing activity, expression, or levels in a cell in the ovary of a subject of a polypeptide encoded by a gene selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17b1, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, or any gene listed in FIGS. 2B, 3B, 4B, 5B, 12B, 12C, or 12D, or in Table 8 or 9. In some cases, the methods involve administering to a subject an agent capable of altering proliferation, development, activation, and/or metabolism in and/or capable of killing a cell selected from one or more of cumulus cells, endothelial cells, epithelial cells, granulosa cells, luteal cells, myeloid cells, oocyte cells, stroma cells, and theca cells. In some aspects, the disclosure provides methods involving administering to a subject a composition comprising an agent that inhibits or facilitates ovulation and/or follicle activation and/or development in a subject. Such an agent may be delivered to cells of a genotypically female subject.

### Polynucleotide Therapy

In embodiments, the methods of the disclosure involve polynucleotide therapy. In some cases, the polynucleotide therapy involves administering to a subject a polynucleotide that disrupts expression of a polypeptide, where the polypeptide may be encoded by a gene selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17b1, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9. In some cases, the polynucleotide therapy involves administering to a subject an inhibitory polynucleotide (e.g., antisense polynucleotide, siRNA) that alters expression, activity, and/or levels in a cell of a polypeptide encoded by a gene selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17b1, Hsd3b1, Ildr2, Kcnd2, Kdr, Kit, Krtl8, Krtl9, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9. In some cases, the methods involve administering to a subject a polynucleotide capable of altering proliferation, development, activation, and/or metabolism in and/or capable of killing a cell selected from one or more of cumulus cells, endothelial cells, epithelial cells, granulosa cells, luteal cells, myeloid cells, oocyte cells, stroma cells, and theca cells. In some aspects, the disclosure provides methods involving administering to a subject a composition comprising an inhibitory polynucleotide that inhibits ovulation and/or follicle activation and/or development in a subject.

Provided herein are inhibitory polynucleotides that reduce expression of a polypeptide encoded by a gene selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17b1, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9. Delivery or expression of such polynucleotides in an ovary cell of a subject, such as an ovary cell in a subject is expected to reduce or prevent ovulation and/or follicle activation and/or development in a subject. Such inhibitory polynucleotides can be delivered to cells of genotypically female subject that would like to avoid becoming pregnant while still having vaginal sex with a genotypic male subject. The inhibitory polynucleotides must be delivered to or expressed in the cells of a subject such that expression levels of the polypeptide in the cells are effectively reduced.

Also provided herein are polynucleotides that increase expression of a polypeptide encoded by a gene selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17b1, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9. Delivery or expression of such polynucleotides in an ovary cell of a subject, such as an ovary cell in a subject is expected to increase or faciliate ovulation and/or follicle activation and/or development in a subject. Such polynucleotides can be delivered to cells of genotypically female subject that would like to become pregnant. The polynucleotides must be delivered to or expressed in the cells of a subject such that expression levels of the polypeptide in the cells are effectively increased. In embodiments, the polynucleotides (e.g., an expression vector and/or mRNA) encode the polypeptide of interest. In some cases, the polynucleotides include a promoter (e.g., a constitutive promoter) driving expression of the encoded polypeptide.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a polypeptide or inhibitory polynucleotide that reduces expression of a polypeptide, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In some embodiments, a viral vector is used to administer an inhibitory polynucleotide that reduces expression of a polypeptide of interest in the ovary of a subject.

Non-viral approaches can also be employed for the introduction of the therapeutic to a cell of a patient in need of a contraceptive treatment or that wishes to become pregnant. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In some embodiments, the nucleic acids are administered in combination with a liposome and protamine. In some embodiments, the nucleic acids are administered in combination with lipid nanoparticles.

Liposomes can also be potentially beneficial for delivery of DNA into a cell. Administration of a polynucleotide (e.g., DNA) encoding a polypeptide or inhibitory polynucleotides (e.g., siRNA) into the affected tissues of a patient can also be accomplished by administering a polynucleotide encoding the polypeptide or inhibitory polynucleotide to the ovary of a subject.

Polypeptide or inhibitory polynucleotide expression from a polynucleotide can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Delivery of polynucleotides of the disclosure may also include or be performed in combination with gene or genome editing methods, such as CRISPR-Cas systems, to introduce polynucleotides encoding a polypeptide or inhibitory polynucleotide into a cell. Gene or genome editing methods such as CRISPR-Cas systems are further described in for example, Sander et al. (2014), Nature Biotechnology 32, 347-355; Hsu et al. (2014), Cell 157(6): 1262-1278.

Naked oligonucleotides or polynucleotides are capable of entering cells and expressing or inhibiting the expression of a polypeptide of interest. Nonetheless, it may be desirable to utilize a formulation that aids in the delivery of an inhibitory nucleic acid molecule or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Inhibitory Polynucleotides

RNA interference (RNAi) is a method for decreasing the cellular expression of specific proteins of interest using an inhibitory polynucleotide (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). In RNAi, gene silencing is typically triggered post-transcriptionally by the presence of double-stranded RNA (dsRNA) in a cell. This dsRNA is processed intracellularly into shorter pieces called small interfering RNAs (siRNAs). The introduction of siRNAs into cells either by transfection of dsRNAs or through expression of shRNAs using a plasmid-based expression system is currently being used to create loss-of-function phenotypes in mammalian cells.

Inhibitory nucleic acid molecules are nucleobase oligomers that may be employed as single-stranded or double-stranded nucleic acid molecule to decrease expression of a target polypeptide. In one approach, the inhibitory nucleic acid molecule is a double-stranded RNA used for RNA interference (RNAi)-mediated knock-down of expression of a target polypeptide. In one embodiment, a double-stranded RNA (dsRNA) molecule is made that includes between eight and twenty-five (e.g., 8, 10, 12, 15, 16, 17, 18, 19, 20, 21, 22, 23, 24, 25) nucleobases. The dsRNA can be two complementary strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference. An inhibitory nucleic acid molecule that “corresponds” to a target gene comprises at least a fragment of the double-stranded gene, such that each strand of the double-stranded inhibitory nucleic acid molecule is capable of binding to the complementary strand of the target gene. The inhibitory nucleic acid molecule need not have perfect correspondence to the reference gene sequence. In one embodiment, an siRNA has at least about 85%, 90%, 95%, 96%, 97%, 98%, or even 99% sequence identity with the target nucleic acid. For example, a 19 base pair duplex having 1-2 base pair mismatch is considered useful in the methods of the disclosure. In other embodiments, the nucleobase sequence of the inhibitory nucleic acid molecule exhibits 1, 2, 3, 4, 5 or more mismatches.

The inhibitory nucleic acid molecules provided by the disclosure are not limited to siRNAs but include any nucleic acid molecule sufficient to decrease the expression of a target polypeptide. The disclosure further provides catalytic RNA molecules or ribozymes. Such catalytic RNA molecules can be used to inhibit expression of a target polypeptide in a cell. The inclusion of ribozyme sequences within an antisense RNA confers RNA-cleaving activity upon the molecule, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference. In various embodiments of this disclosure, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the disclosure and those skilled in the art will recognize that all that is important in an enzymatic nucleic acid molecule of this disclosure is that it has a specific substrate binding site which is complementary to one or more of the target gene RNA regions, and that it has nucleotide sequences within or surrounding that substrate binding site which impart an RNA cleaving activity to the molecule.

In one embodiment, the inhibitory nucleic acid molecules of the disclosure are administered systemically. In some embodiments the nucleic acid molecules are administered locally.

### Modified Inhibitory Nucleic Acid Molecules

A desirable inhibitory nucleic acid molecule is one based on 2′-modified oligonucleotides containing oligodeoxynucleotide gaps with some or all intemucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

Inhibitory nucleic acid molecules include nucleobase oligomers containing modified backbones or non-natural intemucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest- ers, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic intemucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety. In other nucleobase oligomers, both the sugar and the intemucleoside linkage, i.e., the backbone, are replaced with novel groups. The nucleobase units are maintained for hybridization with a VPS4A or VPS4B nucleic acid molecule. Methods for making and using these nucleobase oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

### Antibodies

In other aspects, the disclosure provides a method of increasing or decreasing ovulation and/or follicular development and/or activation in the ovaries of a subject by selectively interfering with the function of a polypeptide. In some embodiments, the interference with the polypeptide function is achieved using an antibody, or an antigen-binding fragment thereof, binding to the polypeptide.

Antibodies can be made by any of the methods known in the art utilizing a polypeptide of the disclosure, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a polypeptide of the disclosure or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding the polypeptide, or immunogenic fragments thereof, can be expressed in cells in vitro, followed by isolation of the polypeptide and administration of the polypeptide to a suitable host in which antibodies are raised.

Alternatively, antibodies against the polypeptide may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

### Genome Editing

Therapeutic gene editing is a major focus of biomedical research, embracing the interface between basic and clinical science. The methods of the disclosure may involve knocking out (e.g., by deletion) or inhibiting expression of a target gene(s) in a cell or tissue of a subject (e.g., Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17b1, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9). The development of novel “gene editing” tools provide the ability to manipulate the DNA sequence of a cell (e.g., to knock out a target gene) at a specific chromosomal locus, without introducing mutations at other sites of the genome. This technology effectively enables the researcher to manipulate the genome of a subject's cells.

In one embodiment, gene editing involves targeting an endonuclease (an enzyme that causes DNA breaks internally within a DNA molecule) to a specific site of the genome and thereby triggering formation of a chromosomal double strand break (DSB) at the chosen site. If, concomitant with the introduction of the chromosome breaks, a donor DNA molecule may be introduced (for example, by plasmid or oligonucleotide introduction), interactions between the broken chromosome and the introduced DNA can occur, especially if the two sequences share homology. In this instance, a process termed “gene targeting” can occur, in which the DNA ends of the chromosome invade homologous sequences of the donor DNA by homologous recombination (HR). By using the donor plasmid sequence as a template for HR, a seamless repair of the chromosomal DSB can be accomplished. In some embodiments, no donor DNA molecule is introduced and the double-stranded break is repaired by the error-prone non-homologous end joining NHEJ pathway leading to knock-out or deletion of the target gene (e.g., through the introduction of indels or nonsense mutations). In some embodiments, an endonuclease(s) can be targeted to at least two distinct chosen sites located within a gene sequence so that chromosomal double strand breaks at the distinct sites leads to excision and deletion of a nucleotide sequence flanked by the two distinct sites.

In some embodiments, the chosen site is associated with or disposed within a nucleotide sequence encoding a gene selected from one or more of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17b1, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, Zfp804a, and any gene listed in FIGS. 2B, 3B, 4B, 5B, 12B, 12C, or 12D, or in Table 8 or 9. In some embodiments, more than one chosen site is selected. In some embodiments the chosen sites are associated with at least 1, 2, 3, 4, 5, 6, or all of the foregoing genes.

Current genome editing tools use the induction of double strand breaks (DSBs) to enhance gene manipulation of cells, including the deletion or knockout of genes. Such methods include zinc finger nucleases (ZFNs; described for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, and U.S. Pat. Publ. Nos. 20030232410 and US2009020314, which are incorporated herein by reference), Transcription Activator-Like Effector Nucleases (TALENs; described for example in U.S. Pat. Nos. 8,440,431, 8,440,432, 8,450,471, 8,586,363, and 8,697,853, and U.S. Pat. Publ. Nos. 20110145940, 20120178131, 20120178169, 20120214228, 20130122581, 20140335592, and 20140335618, which are incorporated herein by reference), and the CRISPR (Clustered Regularly Interspaced Short Palindromic Repeats)/Cas9 system (described for example in U.S. Pat. Nos. 8,697,359, 8,771,945, 8,795,965, 8,871,445, 8,889,356, 8,906,616, 8,932,814, 8,945,839, 8,993,233, and 8,999,641, and U.S. Pat. Publ. Nos. 20140170753, 20140227787, 20140179006, 20140189896,20140273231,20140242664,20140273232,20150184139,20150203872, 20150031134, 20150079681, 20150232882, and 20150247150, which are incorporated herein by reference). In some embodiments a CRISPR/Cas12 system can be used for gene editing. In some embodiments, the Cas12 polypeptide is Cas12b. In some embodiments any Cas polypeptide can be used for gene editing (e.g., CasX). In various embodiments, the Cas polypeptide is selected so that a nucleotide encoding the Cas polypeptide can fit within an adeno-associated virus (AAV) capsid. For example, ZFN DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. ZFNs and TALENs entail use of modular sequence-specific DNA binding proteins to generate specificity for −18 bp sequences in the genome. CRISPR/Cas9, TALENs, and ZFNs have all been used in clinical trials (see, e.g., Li., H, et al., “Applications of genome editing technology in the targeted therapy of human diseases: mechanisms, advances and prospects”, Signal Transduct Target Ther., 5:1 (2020), DOI: 10.1038/s41392-019-0089-y).

RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus (Mali et al., Science. 2013 Feb. 15; 339(6121):823-6). Genetic manipulation using engineered nucleases has been demonstrated in tissue culture cells and rodent models of diseases.

CRISPR has been used in a wide range of organisms including baker's yeast (S. cerevisiae), zebra fish, nematodes (C. elegans), plants, mice, and several other organisms. Additionally, CRISPR has been modified to make programmable transcription factors that allow scientists to target and activate or silence specific genes. Libraries of tens of thousands of guide RNAs are now available.

Since 2012, the CRISPR/Cas system has been used for gene editing (silencing, enhancing or changing specific genes) that even works in eukaryotes like mice and primates. By inserting a plasmid containing Cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of Cas genes and repeat structures have been used to define 8 CRISPR subtypes (E. coli, Y. pest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (about 30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. co/i) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. co/i requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes. See also U.S. Patent Publication 2014/0068797, which is incorporated by reference in its entirety.

### Cas9

Cas9 is a nuclease, an enzyme specialized for cutting DNA, with two active cutting sites, one for each strand of the double helix. The team demonstrated that they could disable one or both sites while preserving Cas9's ability to locate its target DNA. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug. 17; 337(6096):816-21).

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) generated mice with mutations. Delivery of Cas9 DNA sequences also is contemplated.

Cas9 variants have been developed or discovered that can fit into an adeno-associated virus (AAV) capsid with sgRNA. Non-limiting examples of such variants (e.g., Cas9 orthologs) suitable for use in embodiments of the disclosure of the disclosure include saCas9 (Staphylococcus aureus Cas9), cjCas9 (Camphylobacter jejuni Cas9), NmeCas9 (Neisseria meningitidis Cas9), and spCas9 (Streptococcus pyrogenes Cas 9). An example of a saCas9 suitable for delivery by an AAV vector is provided in Ann Ran, F. et al. “In vivo genome editing using Staphylococcus aureus Cas9”, Nature, 9:186-91, DOI: 10.1038/nature14299.
gRNA

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type 21 promoter U6). Synthetic gRNAs are slightly over 100 bp at the minimum length and contain a portion that targets the 20 protospacer nucleotides immediately preceding the PAM sequence NGG; gRNAs do not contain a PAM sequence.

### CRISPR Interference

In some embodiments, a target gene can be inhibited using CRISPR interference (CRISPRi). CRISPRi is a technique where expression of a target gene is inhibited by the binding of a nuclease-inactive CRISPR system (a CRISPRi system), optionally comprising transcriptional repressors. In some embodiments, the method of CRISPRi involves designing an sgRNA complementary to a promoter or exonic sequence of a target gene. In some embodiments, CRISPRi involves guiding a transcriptional repressor to a transcription start site of a target gene. CRISPRi has been successfully used for the repression of gene expression in mice and an exemplary method for using CRISPRi to repress a gene is provided in MacLeod, et al., “Effective CRISPR interference of an endogenous gene via a single transgene in mice”, Scientific Reports, 9:17312 (2019).

### Pharmaceutical Compositions

The disclosure provides therapeutic compositions that alter ovulation and/or follicle development and/or activation in the ovaries of a female subject. In embodiments, the therapeutic compositions contain an agent, such as a small molecule, polypeptide, and/or polynucleotide provided herein. Agents of the disclosure may be administered as part of a pharmaceutical composition. The compositions should be sterile and contain a therapeutically effective amount of the polypeptides or nucleic acid molecules in a unit of weight or volume suitable for administration to a subject.

An agent of the present disclosure may be administered within a pharmaceutically-acceptable diluents, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracistemal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agents include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition. The dosage of an agent of the disclosure may depend on such variables as the type and extent of the disorder or therapeutic objective, the overall health status of the particular patient, the formulation of the compound excipients, and/or route of administration.

Typically, an effective amount is sufficient to alter ovulation and/or follicle development and/or activation in the ovary of a subject. Generally, doses of an agent would be from about 0.01 mg/kg per day to about 1000 mg/kg per day. It is expected that doses ranging from about 50 to about 2000 mg/kg will be suitable. Lower doses will result from certain forms of administration, such as intravenous or local administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic or local levels (e.g., levels in an ovary) of an agent of the present disclosure.

A variety of administration routes are available. The methods of the disclosure, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Kits

The disclosure provides kits suitable for use in any of the methods provided herein, such as methods for altering ovulation and/or follicle activation and/or development in a subject. In one embodiment, the kit contains an agent provided herein. In some embodiments, the kit comprises a sterile container which contains an agent of the disclosure or composition of the disclosure; such containers can be boxes, ampoules, bottles, vials, tubes, bags, pouches, blister-packs, or other suitable container forms known in the art. The containers can be made of plastic, glass, laminated paper, metal foil, or other materials suitable for holding medicaments.

A kit as described herein may be provided together with instructions for administering a composition of the kit to a subject wishing to avoid pregnancy or who desires to become pregnant. The instructions will generally include information about the use of the composition for the treatment or prevention of the disease or disorder. In other embodiments, the instructions include at least one of the following: description of the therapeutic agent; dosage schedule and administration methods; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the aspects and embodiments of the disclosure. Particularly useful techniques for specific embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### Examples

### Example 1: a Single-Cell Temporal Reference of Cell Types in the Adult Mouse Ovary Across Ovulation Reveals “Early” and “Late” Cell States

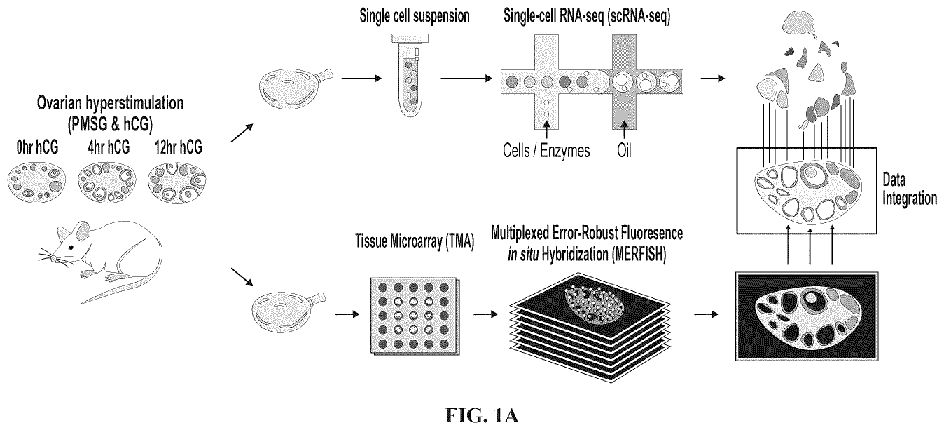

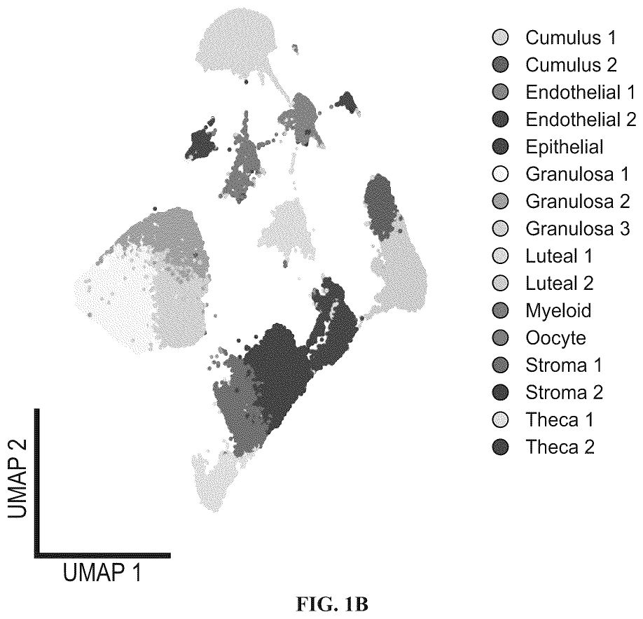

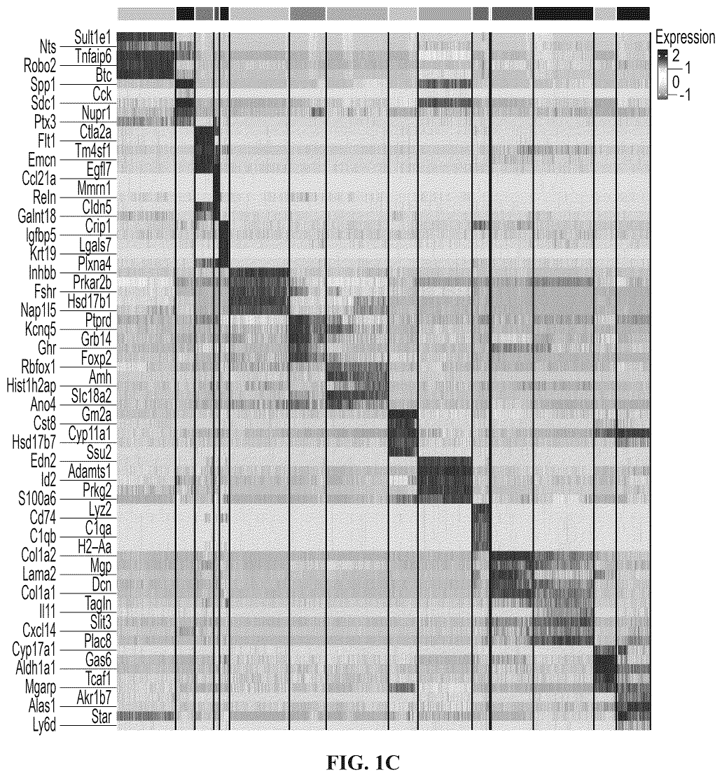

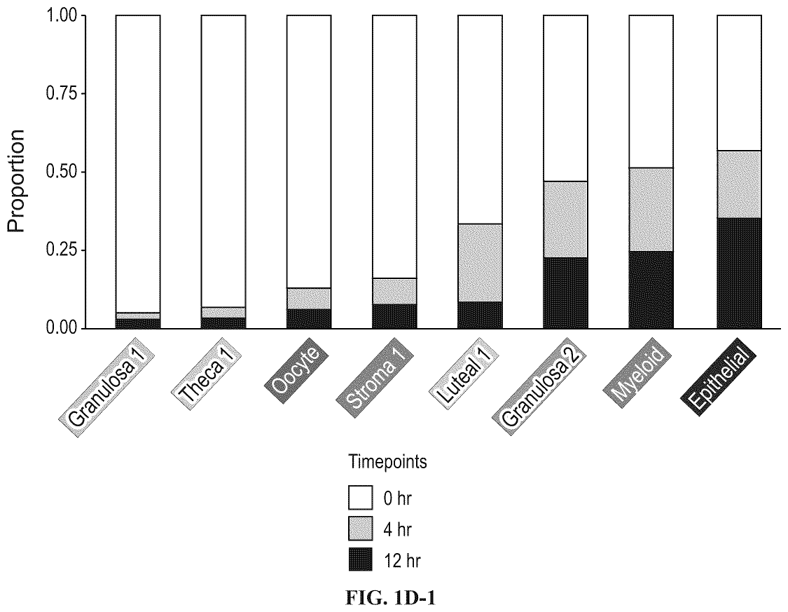

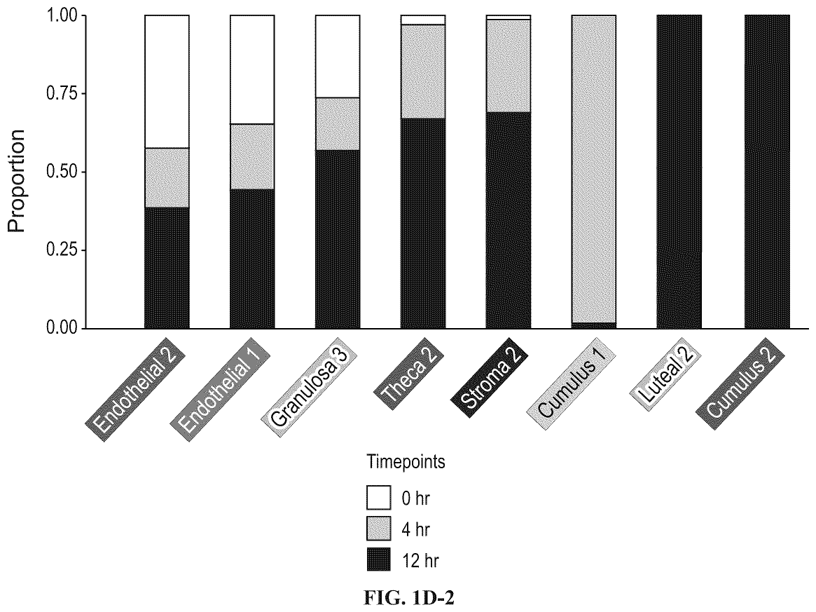

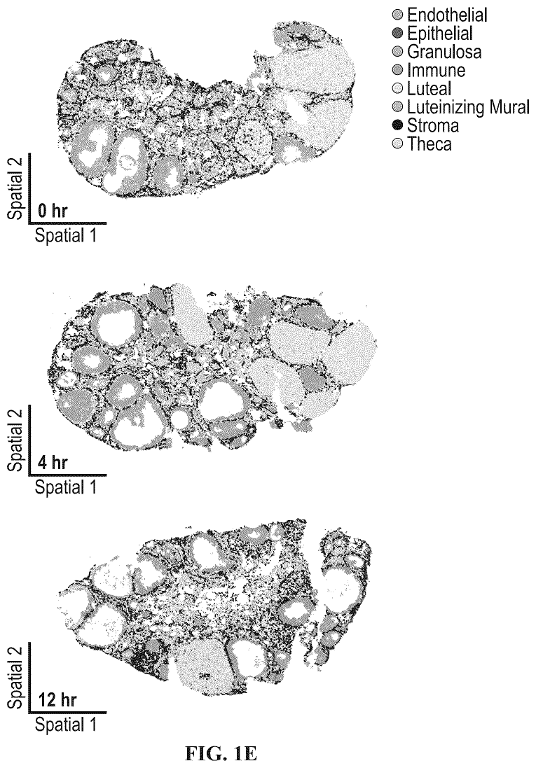

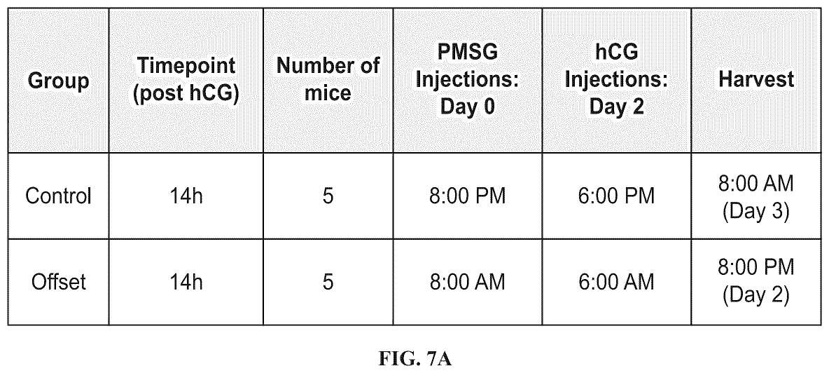

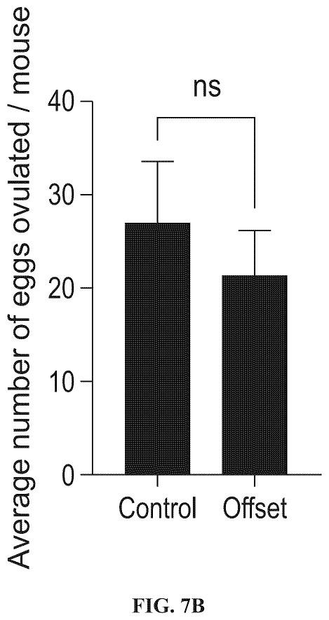

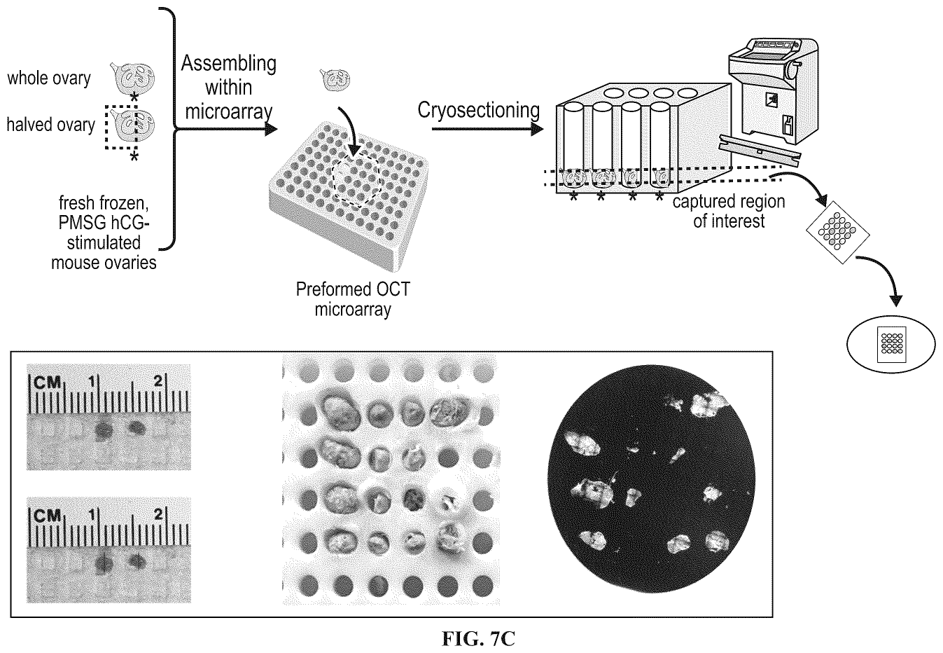

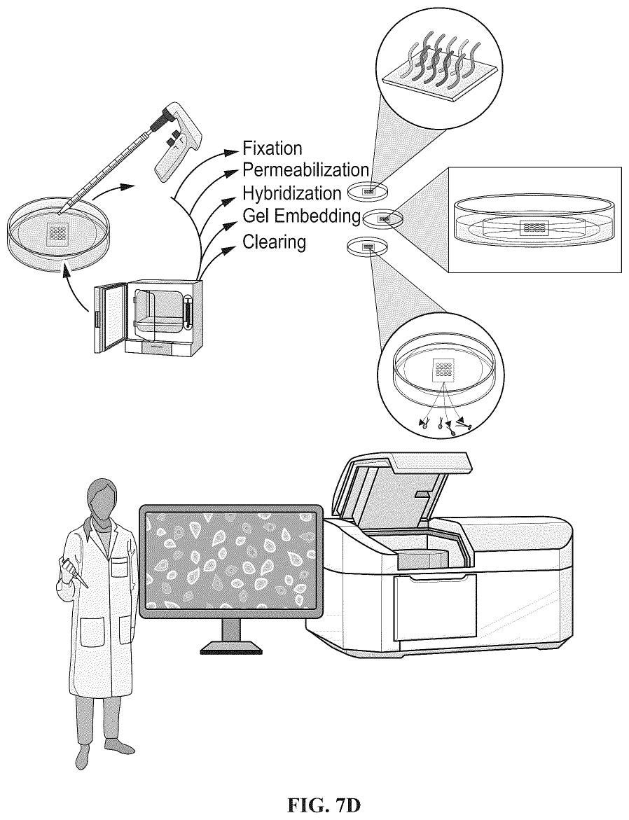

To deeply profile the mouse ovary across ovulation using scRNA-seq and spatial transcriptomics, ovulation was induced in a synchronized fashion following hyperstimulation, and ovaries were collected at 0h, 4h, or 12h after induction. The 4h timepoint is early in the ovulation process and represents the peak of the luteinizing hormone surge when expression of key ovulation regulators is highest. The 12h timepoint represents a later stage when follicular rupture is underway. At every timepoint, one ovary per mouse was used for scRNA-seq, and the contralateral ovary was used for spatial transcriptomics array (FIGS. 1A, and 7A-7D).

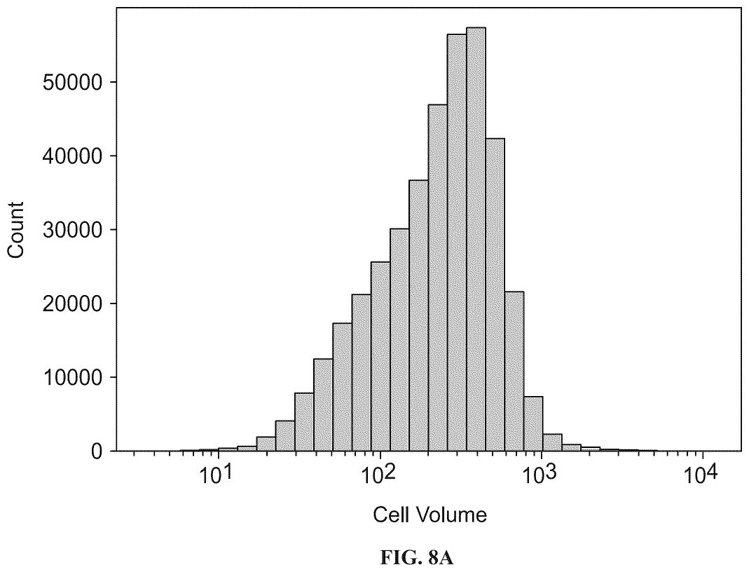

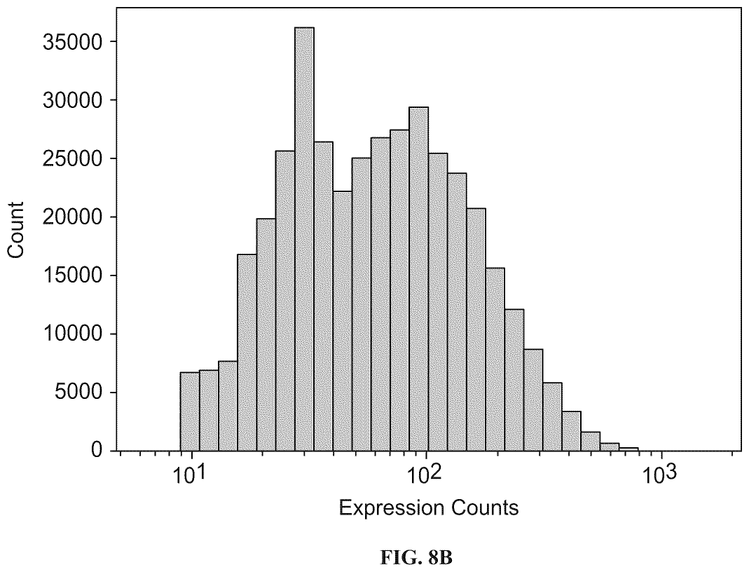

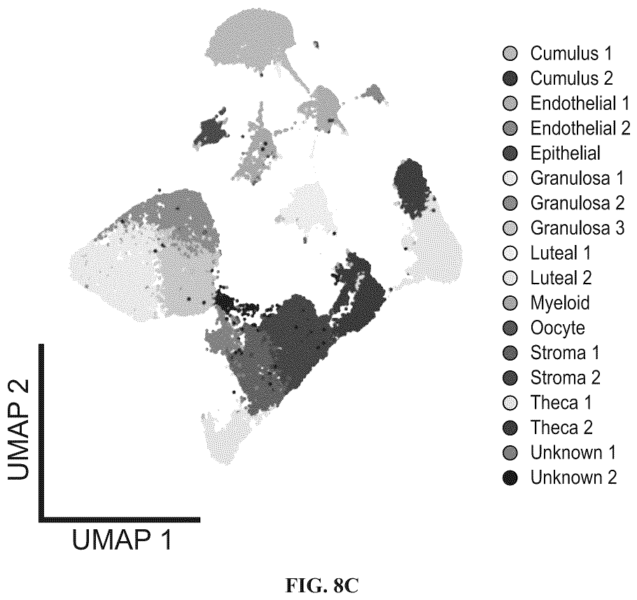

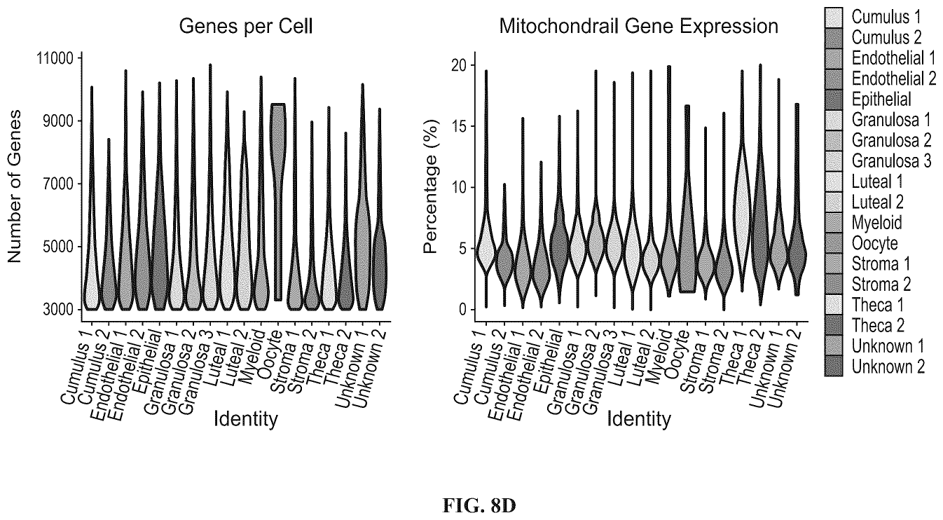

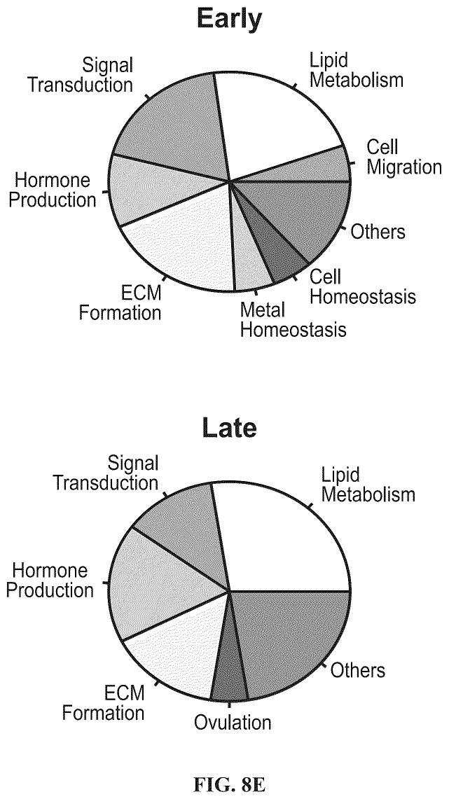

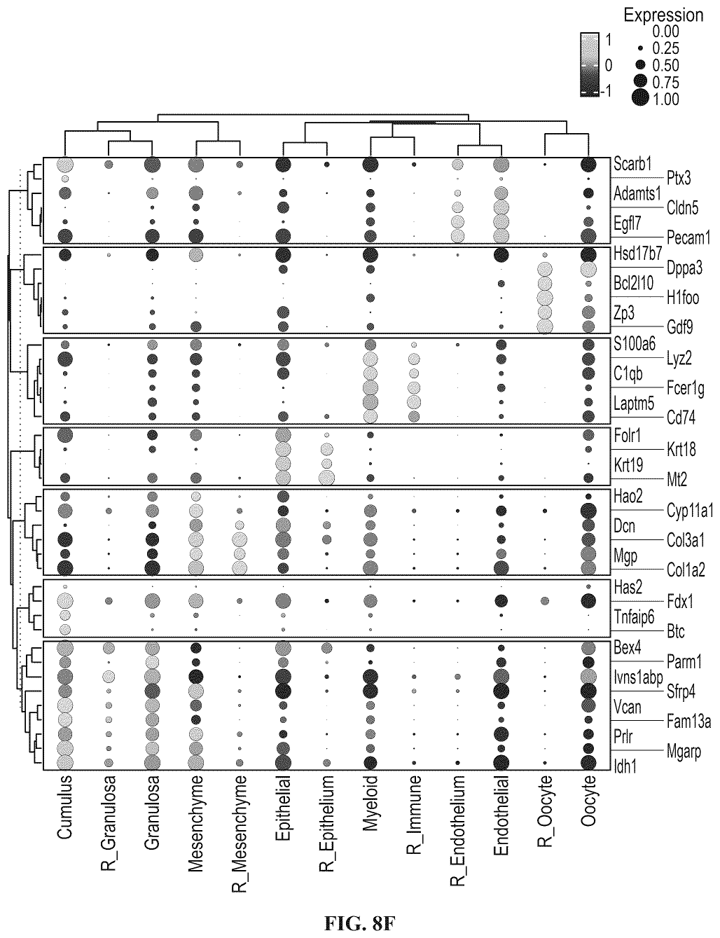

To determine major top-level ovarian cell identities across ovulation and their associated marker genes, standard computational workflows were used, including dimensionality reduction, clustering, and marker gene identification across the scRNA-seq and iST dataset after removing cells that did not meet quality benchmarks (FIGS. 8A-8D). In total, for the scRNA-seq dataset, 26,411 cells were analyzed and eight major cell types were identified in the ovary: cumulus cells, endothelial cells, epithelial cells, granulosa cells, luteal cells, myeloid cells, stromal cells, and theca cells (FIG. 1B). Additionally, a very small cluster of oocytes (n=16 cells) was identified that were not further analyzed given the limited number of cells present due to their large size. The major cell types were characterized by unique marker genes (FIG. 1C and Table 8) and compared to an existing scRNA-seq reference of the ovary throughout the estrous cycle (Morris et al., eLife, 11, e77239 (2022)) (FIG. 8E). Within each major cell type, the classification was refined and several unique cell subclusters were identified for cumulus (Cumulus 1 and 2), endothelial (Endothelial 1 and 2), granulosa (Granulosa 1, 2, and 3), luteal (Luteal 1 and 2), stroma (Stroma 1 and 2), and theca (Theca 1 and 2) cells (FIG. 1B). Several known marker genes were present: el (cumulus), Inhbb and Amh (granulosa), Lhcgr (luteal), Dcn (stroma), and Cyp17al (theca). Several new marker genes were also identified including Zpf804a (cumulus), Oca2 (theca), Mrap](stroma), and Cnn3 (luteal) (FIG. 1C, Table 8). Notably, two cumulus cell clusters were identified that were unique to ovulation given their temporal emergence, which were identified based on cumulus cell expansion markers and gene ontology (GO) pathway analysis results. These cumulus cell clusters were not present in a reference scRNA-seq dataset of the adult mouse ovary captured throughout the estrous cycle demonstrating their unique relevance to ovulation (Morris et al., eLife, 11, e77239 (2022)) (FIGS. 8E and 8F).

TABLE 8
Gene list in each identified top level cell
cluster based on soft clustering analysis.

| | Cluster 1 | Cluster 2 | Cluster 3 | Cluster 4 | Cluster 5 |
|---|---|---|---|---|---|
| | Cumulus 1 | Cumulus 2 | Endothelial 1 | Endothelial 2 | Epithelial |
| 1 | Sult1e1 | Spp1 | Ctla2a | Ccl21a | Crip1 |
| 2 | Nts | Sdc1 | Flt1 | Mmrn1 | Igfbp5 |
| 3 | Tnfaip6 | Nupr1 | Tm4sf1 | Reln | Lgals7 |
| 4 | Robo2 | Ptx3 | Emcn | Gng1I | Krt19 |
| 5 | Btc | Lox | Egfl7 | Cldn5 | Plxna4 |
| 6 | Pgr | Cited4 | Igfbp7 | Galnt18 | Lgals2 |
| 7 | Areg | Rgcc | Ramp2 | Prox1 | Lgals1 |
| 8 | Tac1 | Lgals3 | Eng | Aqp1 | Krt18 |
| 9 | Alcam | Pik3c2g | Ehd4 | Lyve1 | Upk3b |
| 10 | Ptgs2 | Ier3 | Ptprb | Cavin2 | Epcam |

| | Cluster 6 | Cluster 7 | Cluster 8 | Cluster 9 | Cluster 10 |
|---|---|---|---|---|---|
| | Granulosa 1 | Granulosa 2 | Granulosa 3 | Luteal 1 | Luteal 2 |
| 1 | Inhbb | Ptprd | Rbfox1 | Gm2a | Edn2 |
| 2 | Prkar2b | Kcnq5 | Amh | Cst8 | Adamts1 |
| 3 | Fshr | Grb14 | Hist1h2ap | Cyp11a1 | Id2 |
| 4 | Hsd17b1 | Ghr | Slc18a2 | Hsd17b7 | Prkg2 |
| 5 | Nap1l5 | Foxp2 | Ano4 | Ssu2 | S100a6 |
| 6 | Inhba | Sema5a | Tanc2 | Akr1c18 | Psap |
| 7 | Grem2 | Foxo1 | Hist1h2ae | Lhcgr | Rgcc |
| 8 | Tnni3 | Zfp385b | Fam13a | Ptgfr | Ephx2 |
| 9 | Inha | Pde7b | Mctp1 | Idh1 | Mt2 |
| 10 | Fam13a | Tenm4 | Hist1h1b | Sfrp4 | Slco2a1 |

| | Cluster 11 | Cluster 12 | Cluster 13 | Cluster 14 | Cluster 15 |
|---|---|---|---|---|---|
| | Myeloid | Stroma 1 | Stroma 2 | Theca 1 | Theca 2 |
| | Lyz2 | Col1a2 | Tagln | Cyp17a1 | Akr1b7 |
| 2 | Cd74 | Mgp | Il11 | Gas6 | Alas1 |
| 3 | Apoe | Lama2 | Slit3 | Aldh1a1 | Cyp11a1 |
| 4 | C1qa | Dcn | Cxcl14 | Tcaf1 | Star |
| 5 | C1qb | Col1a1 | Plac8 | Mgarp | Ly6d |
| 6 | H2-Aa | Ogn | Pdzrn3 | Fabp3 | Hao2 |
| 7 | H2-Ab1 | Igfbp7 | Cald1 | Dnajc15 | Rhox8 |
| 8 | Tyrobp | Col3a1 | Lhfp | Pank1 | Abca1 |
| 9 | Fcer1g | Acta2 | Abi1 | Nckap5 | Cyp51 |
| 10 | C1qc | Cped1 | Tenm3 | Akr1cl | Me1 |

In addition to differences in gene expression, some cell clusters differentiated temporally into “early” and “late” groups relative to the ovulation time course, while other clusters were present throughout all of ovulation (FIGS. 1D-1 and 1D-2). Specifically, “early” clusters included Theca 1, Granulosa 1, Stroma 1, and Luteal 1, which were expressed almost exclusively at 0h and/or 4h. “Late” clusters were expressed predominantly at 12 h and included Cumulus 2, Luteal 2, Stroma 2, and Theca 2. Clusters expressed throughout ovulation included Granulosa 2, Granulosa 3, Endothelial 1, Endothelial 2, Epithelial, and Myeloid. To determine if there were underlying functional differences across clusters, pathway analysis was performed on each list of marker genes for each identified cluster of cells. The results suggested that each cluster was enriched for unique biological pathways (Table 9).

TABLE 9
Gene list in each identified cell subcluster
based on soft clustering analysis.

| | Theca | | | |
|---|---|---|---|---|
| | | Subcluster 2 | | Subcluster 4 |
| | Subcluster 1 | Steroidogenic | Subcluster 3 | Early |
| | Theca (12 h) | Theca (0 hr) | Theca (4 hr) | Theca (0 hr) |
| 1 | Spp1 | Gas6 | Mast4 | Mctp1 |
| 2 | Timp1 | Akr1cl | Gtdc1 | Nr5a2 |
| 3 | Tmsb4x | Pak3 | Areg | Fshr |
| 4 | S100a6 | Tcaf1 | Rnf180 | Inhba |
| 5 | Plac8 | Lhcgr | Odc1 | Tanc2 |
| 6 | Sbsn | Cyp17a1 | Jarid2 | Bmpr1b |
| 7 | Ephx2 | AU020206 | Pcsk5 | Serpine2 |
| 8 | Ybx1 | Gab2 | Slit2 | Fst |
| 9 | Mt1 | Acsbg1 | Tnfaip6 | Kcnq5 |
| 10 | Fdps | Fabp3 | Gm12648 | Foxo1 |

| | Luteal | | | |
|---|---|---|---|---|
| | Subcluster 1 | Subcluster 2 | | |
| | Luteinizing | Mature/ | Subcluster 3 | Subcluster 4 |
| | Mural | Regressing CL | Mitotic Antral | Early CL |
| 1 | Spp1 | Gm2a | Inha | Igfbp7 |
| 2 | Adamts1 | Hsd3b1 | Nap115 | Col3a1 |
| 3 | Edn2 | Cst8 | Fam13a | Ifitm3 |
| 4 | Vcan | Ssu2 | Mctp1 | Mgp |
| 5 | Prkg2 | Hsd17b7 | Hsd17b1 | Plac8 |
| 6 | Sox5 | Aebp1 | Foxo1 | Cald1 |
| 7 | Rhox8 | Lhcgr | Tenm4 | Tagln |
| 8 | Mt2 | Bhmt | Rbfox1 | Slit3 |
| 9 | Slco2a1 | Ptgfr | Prkar2b | Rbms3 |
| 10 | Frmd5 | Bst2 | Tanc2 | Acta2 |

### Example 2: Single-Cell Spatial Transcriptomic Analysis of Adult Mouse Ovaries Throughout Ovulation Reveals Temporally-Driven Processes

To map the spatiotemporal nature of ovulation, imaging spatial transcriptomic data on contralateral ovaries was also generated (FIG. 1A). Following standard quality control filters and initial filtering based on transcript count and cell area (FIGS. 8A-8B), expression of 205 genes was analyzed using MERFISH probes in 391,584 cells across 8 samples.

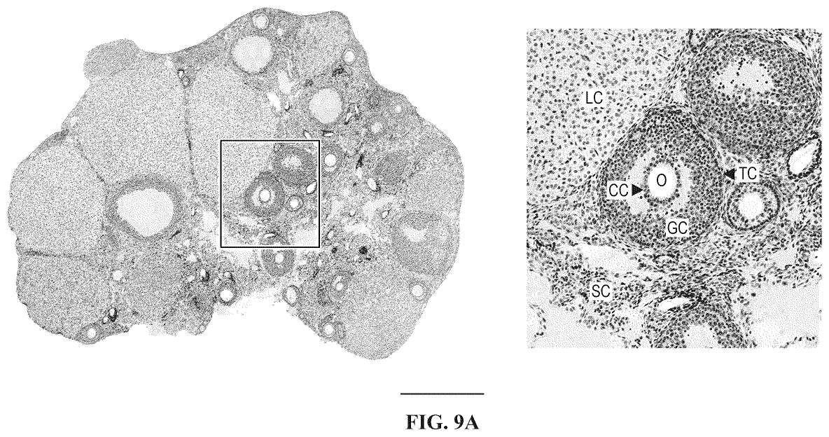

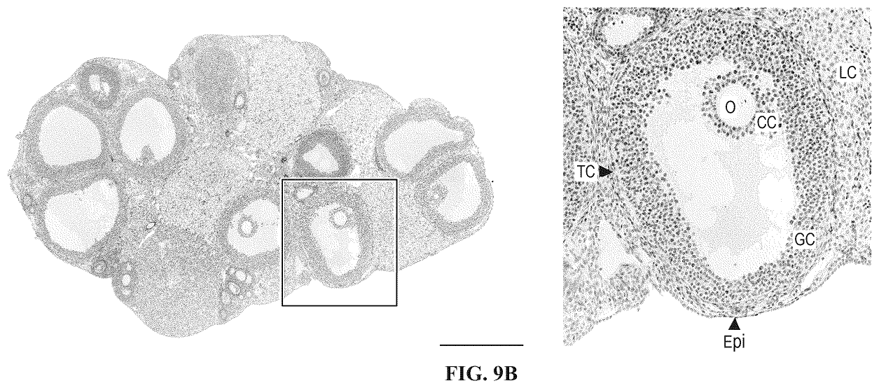

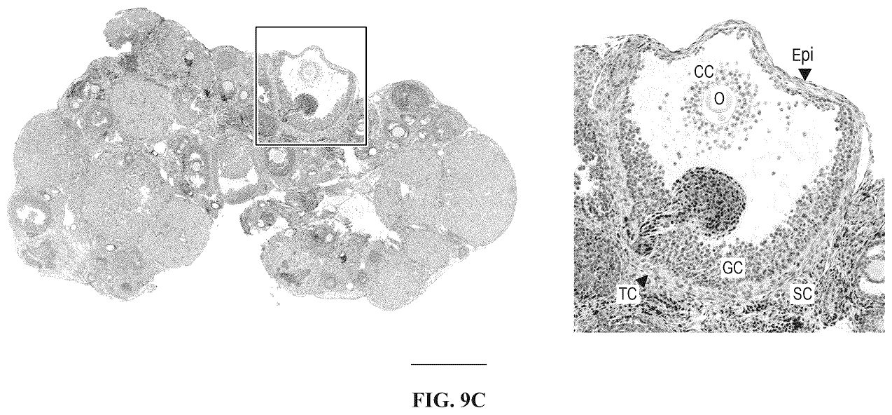

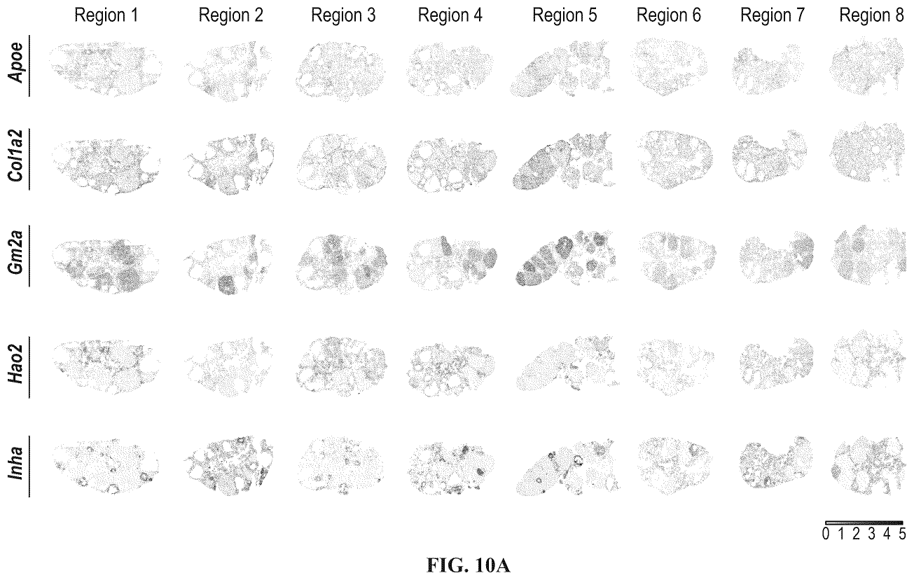

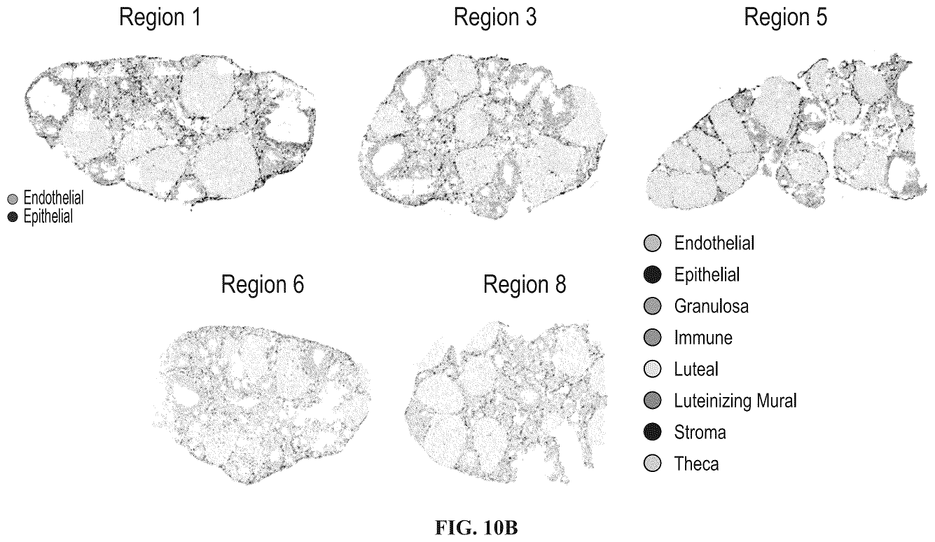

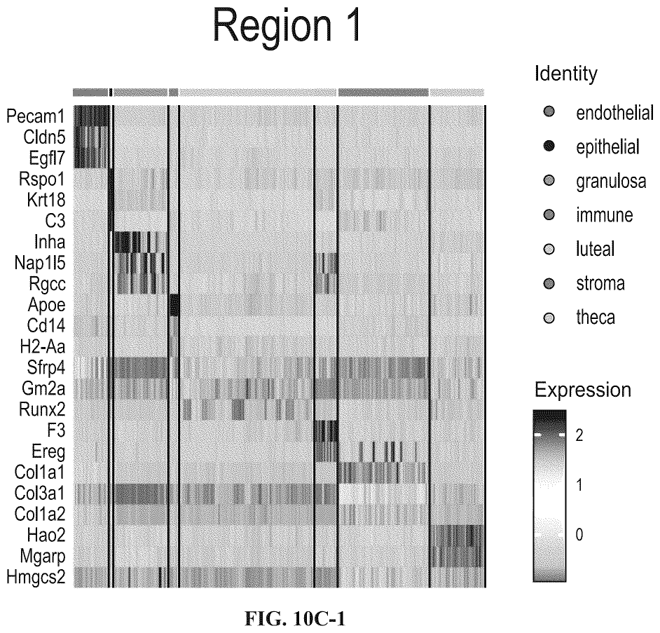

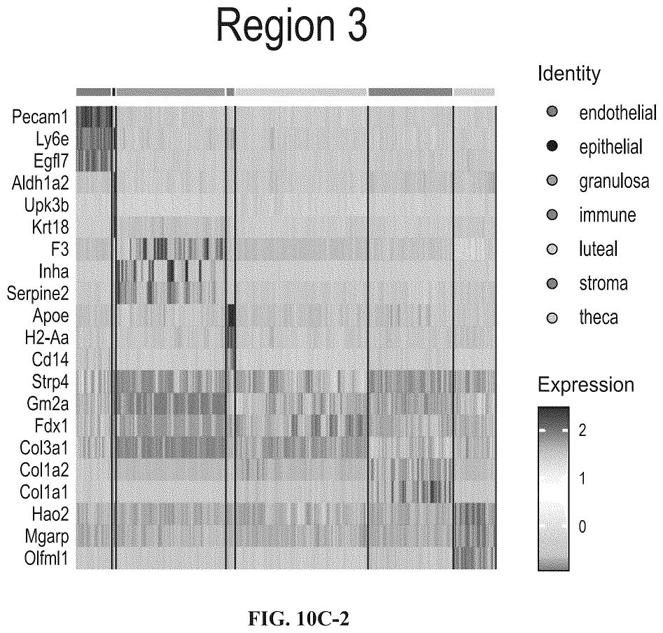

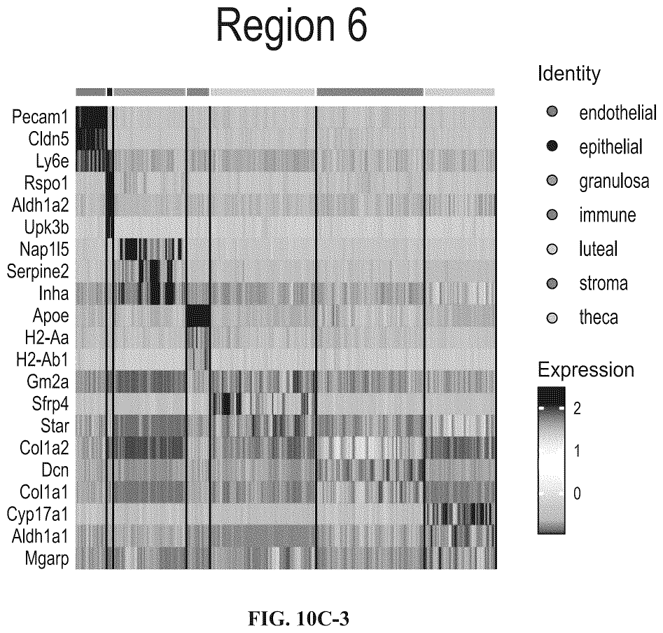

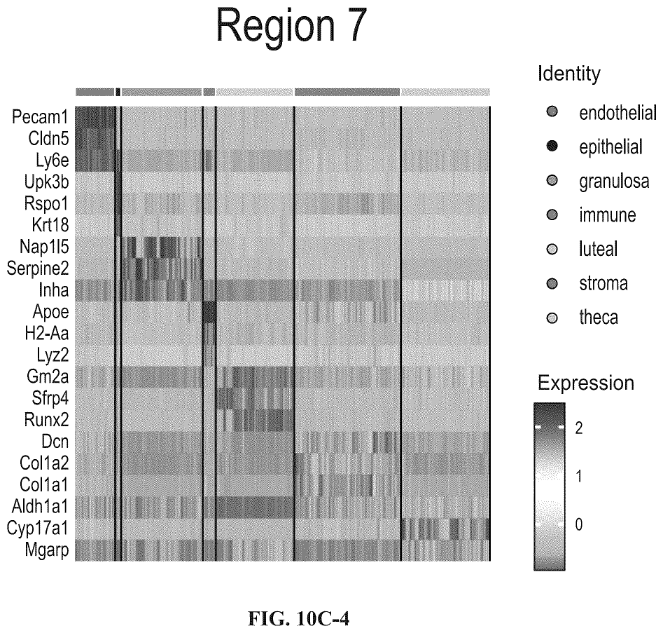

Ovaries that were fixed and stained with hematoxylin and eosin were also collected to validate the superovulation protocol and ensure the MERFISH sections resembled the histological sections. The anatomical features of the MERFISH sections were very similar to that observed via histology (FIGS. 9A-9C). Antral follicles with multiple layers of granulosa cells were observed surrounding the antral cavity with cumulus cells surrounding the oocyte. Theca cells first appeared in secondary follicles and were separated from granulosa cells by a basement membrane. Luteal cells overlapped with corpora lutea that were either actively luteinizing or recently completed the luteinization process. In the extrafollicular region, stromal cells constituted the ovarian parenchyma between follicles. Finally, it was found that endothelial and immune cells tended to be evenly dispersed across the ovarian section and epithelial cells distinctly outlined the edge of the ovary as part of the ovarian surface epithelium. Importantly, major gross morphological phenotypes and alterations were observed in ovaries across the course of ovulation. Between 0h and 4h post-hCG injection, there was an increase in the number of large ovulatory antral follicles. By 12h post-human chorionic gonadotropin (hCG) injection, antral follicles were seen that had either recently ruptured or were near rupture based on the thinning of the mural cell layers and the position of the cumulus-oocyte complex (COC) near the ovarian surface. Besides observation on morphology, clustering was also performed and marker genes were used to annotate the major top-level cell types with expression plots and heatmaps, including theca cells, stromal cells, granulosa cells, luteal cells, immune cells, endothelial cells, and epithelial cells (FIGS. 1E and 10A to 10C-5).

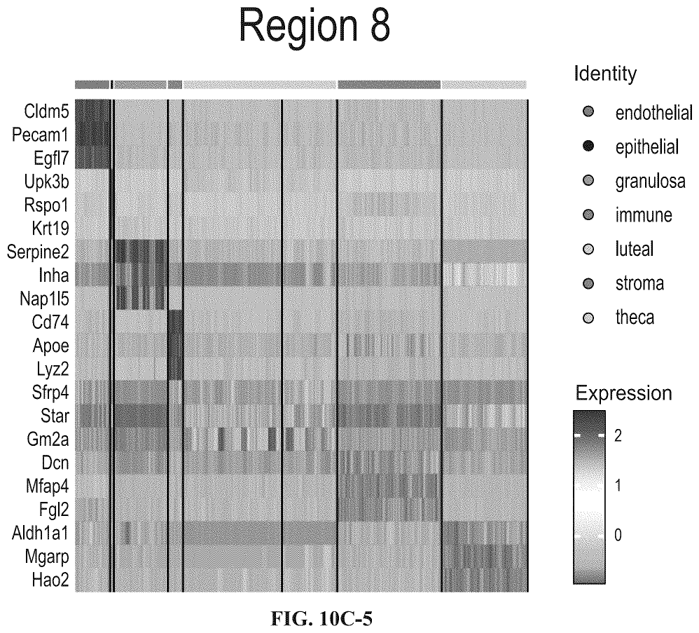

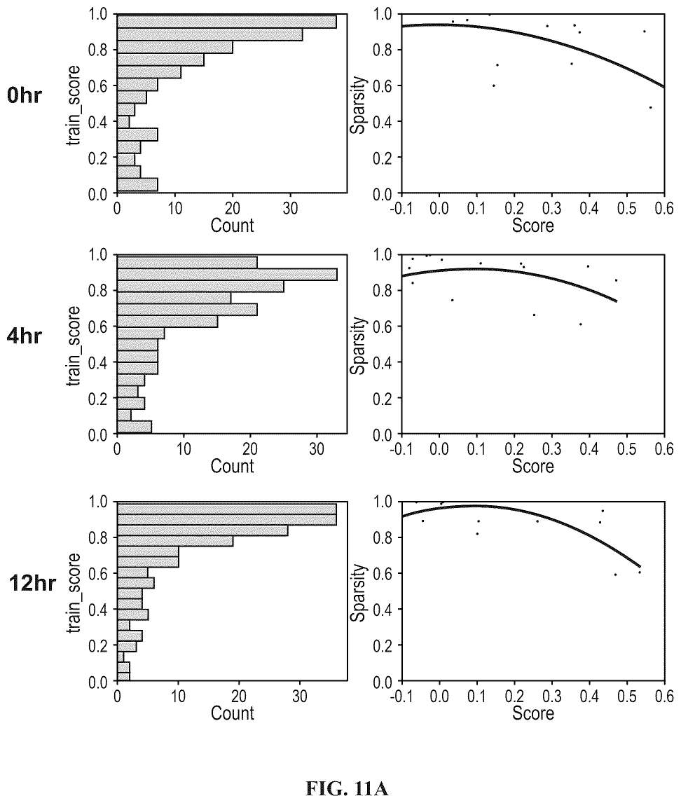

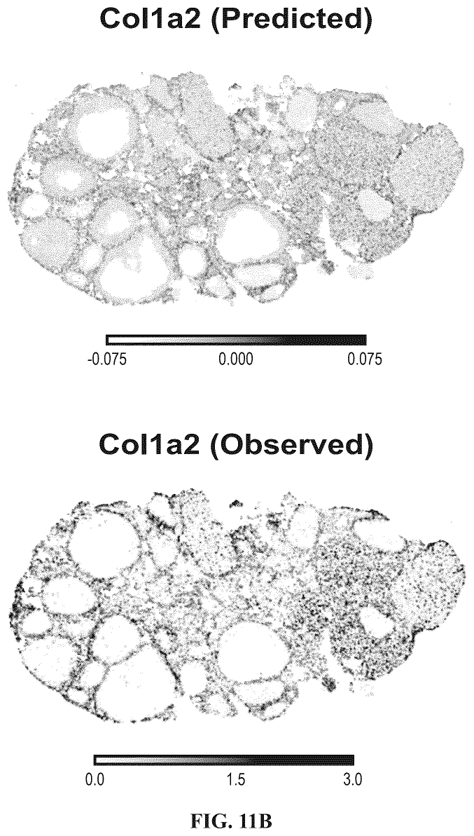

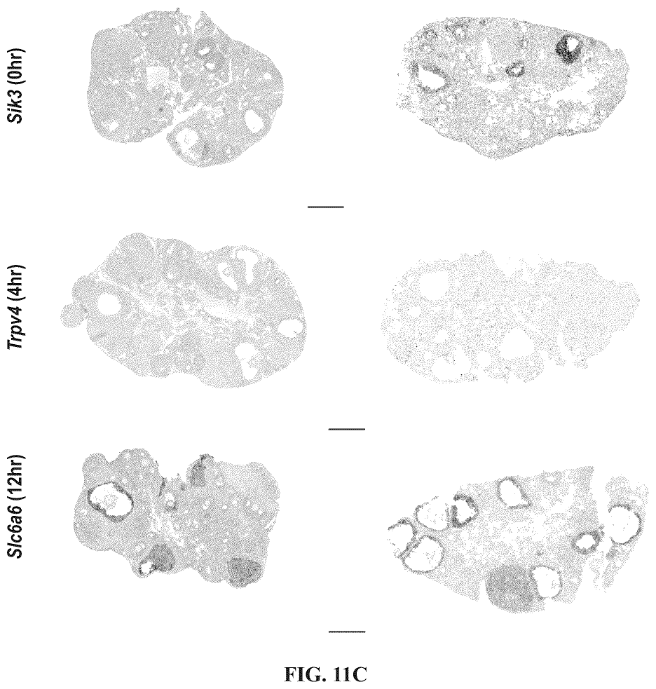

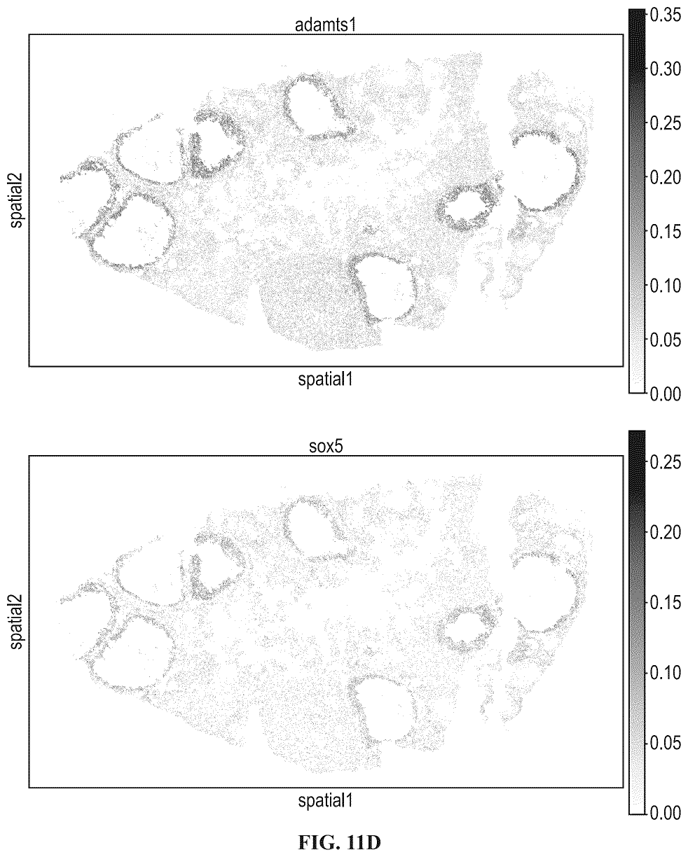

To further investigate the gene expression changes and their spatial distribution during ovulation, the single-cell and spatial transcriptomes were integrated together with an established machine learning approach. This approach generated high training scores and testing scores. When comparing the predicted expression profile versus actual expression profile from the spatial transcriptome, high similarity was found for genes such as Colla2, which was highly enriched in mesenchymal cells (FIGS. 11A and 11B). Lastly, predicted gene expression profiles were compared with actual RNA expression detected using RNAScope on histological ovarian sections as validation, where the predicted expression profile of Slc6a6 being primarily present in luteal cells at 0h and in cumulus and mural granulosa cells at 12h was recapitulated in RNAScope images (FIG. 11C). Finally, Adamts1 has been recognized as a marker for luteinizing mural cells, whereas Sox5, exhibiting similar spatial expression patterns, emerged as a promising alternative (FIG. 11D). Moreover, Pdzrn3 was a potential novel stromal cell marker candidate, having expressions akin to the documented Dcn (FIG. 11E).

Taken together, these findings are consistent with established timelines of murine ovulation and expected changes in follicular morphology, and combined with the scRNA-seq and iST data, all major ovarian cell types and their spatial location have been captured across ovulation. To date, this is the most comprehensive dataset describing the events of ovulation in the mouse. The integrated data resource allows one to precisely identify high-resolution spatiotemporal changes in genes beyond those initially targeted by MERFISH probes. As a result, the expression patterns of over 25,000 genes across 150,026 cells can be inferred at 0hrs, 4 hrs, and 12 hrs post-hCG. This integration stands as a valuable resource, allowing for inference of novel functions and applications, serving both as a validation tool and a means to uncover genes and patterns defining ovarian processes.

### Example 3: Analysis of Time-Dependent Cell Types Reveals Unique Expression Patterns and Putative Functions of Early and Late Cell Clusters

To further investigate the time-dependent changes in gene expression for each cell type, pathway analyses on cluster marker genes and differential gene expression analysis (DEA) between early and late cell subclusters were performed for time-varying cell types.

Stroma cells: Time-dependent changes in gene expression were observed throughout ovulation and two stromal cell subclusters were identified: early stromal (Stroma 1) and late stromal (Stroma 2) cells (FIGS. 2A-C). Data envelopment analysis (DEA) performed between the early and late stromal subclusters indicated that the top upregulated genes in early stromal cells were Lama2, Grm7, Ptprd, Tcf21, and Tenm4. Additionally, the top upregulated genes in late stromal cells were Timp1, Ereg, Il11, Pde10a, and Mrap (FIG. 2B).

Within each subcluster, the expression profiles of known ovarian stromal cell markers were further characterized. Dcn, or decorin, is a proteoglycan associated with extracellular matrices in a variety of tissues and was highly enriched in early stromal cells (FIG. 2C). In addition, Timp1, a matrix metalloproteinase inhibitor, exhibited increased expression in late stromal cells. In addition, genes were identified that were highly enriched but not well characterized in the ovarian stroma, such as Egtoflam (early stromal) and Abi1 (late stromal). Egflam, also known as pikachurin, is an extracellular matrix-like protein that interacts with dystroglycan and has been implicated in the photoreceptor synapse function. Abi1 encodes an adapter protein involved implicated to facilitate signal transduction and promote actin polymerization (FIGS. 2D-1 and 2D-2).

Gene ontology (GO) Analysis of top differentially expressed genes between the early stromal (Stroma 1) and late stromal (Stroma 2) subclusters revealed that early stromal cells were enriched in pathways related to ECM formation, smooth muscle relaxation, and the estrous cycle (FIGS. 2E-1 and 2E-2). Late stroma cells were unique in pathways related to angiogenesis, cell differentiation, hormone production, and protein modification. Not intending to be bound by theory, these differences in function suggested that early stroma cells may drive the morphologic and transcriptomic changes necessary for the transition from proestrus to estrus in response to LH signaling. On the other hand, late stroma cells are likely involved in steroid hormone production; although not a classic function of stroma cells, subpopulations of stroma cells capable of producing hormones have been documented in several species. In addition, late stroma cells potentially increase blood vessel formation to accommodate the transport of these hormones to the rest of the body.

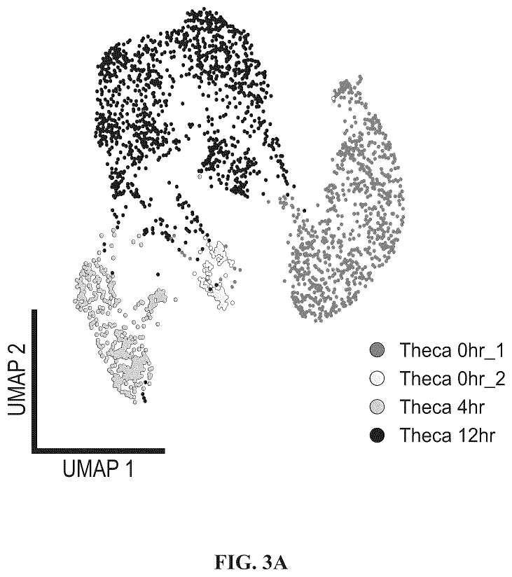

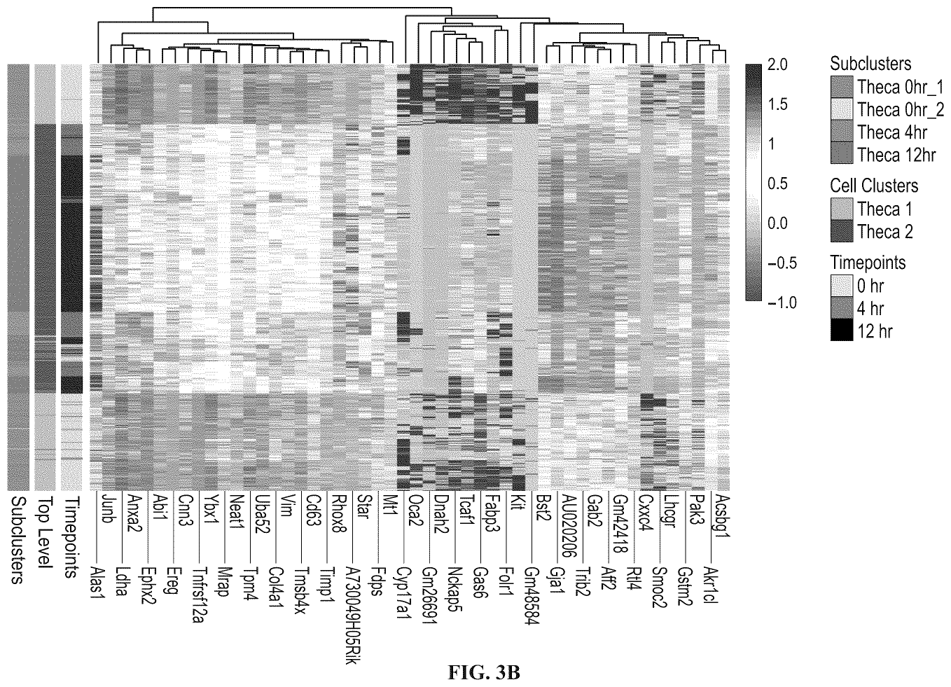

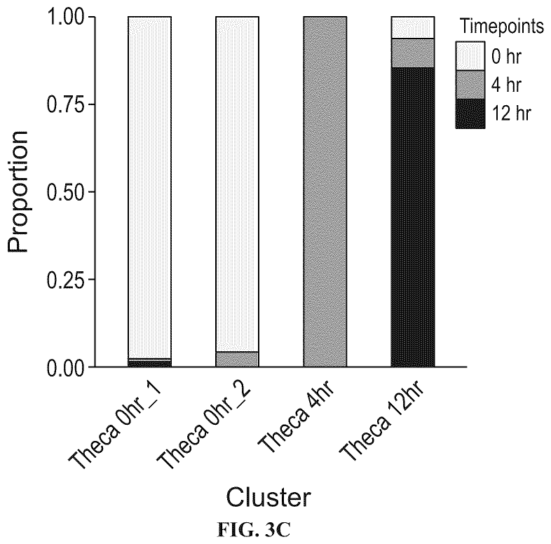

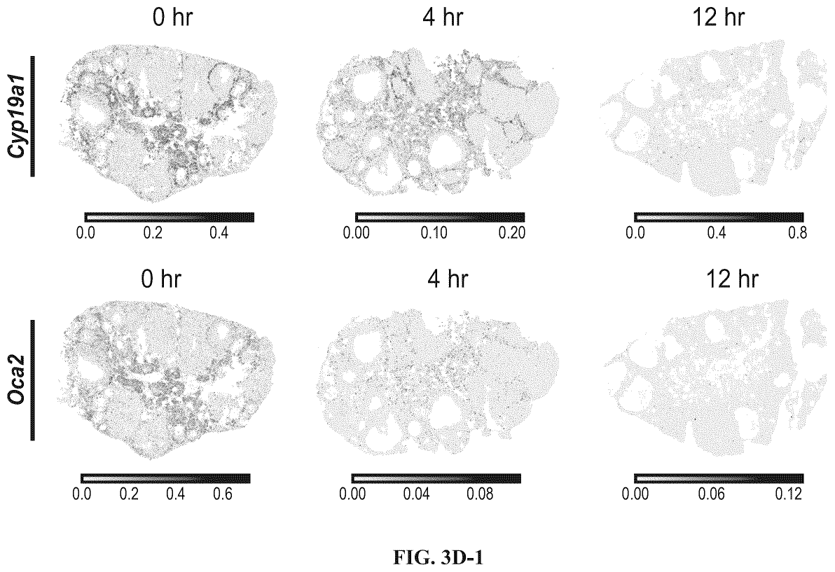

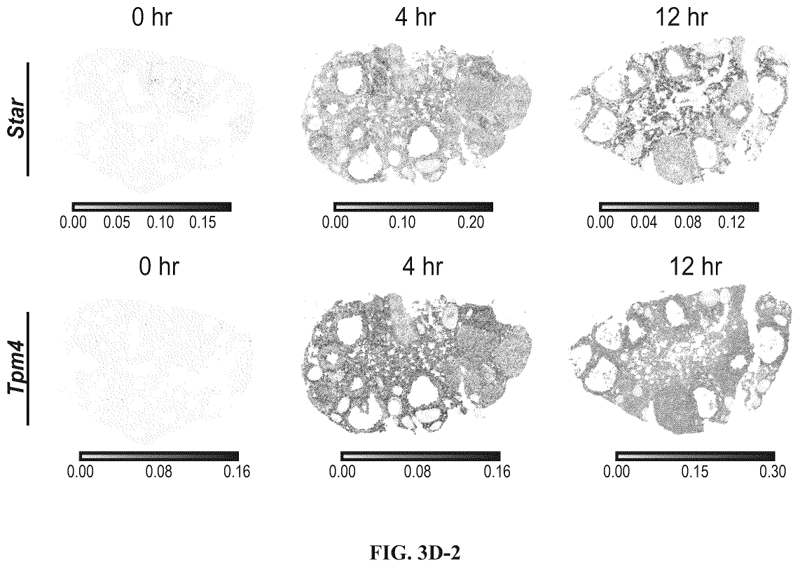

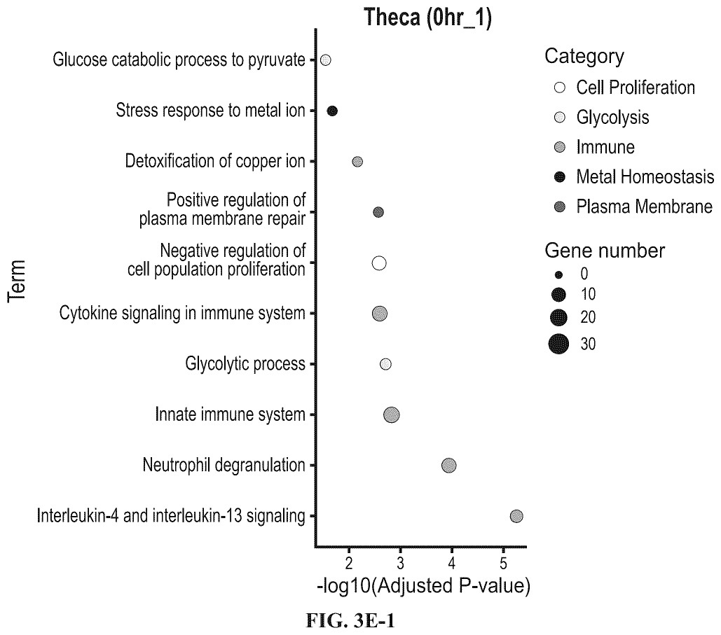

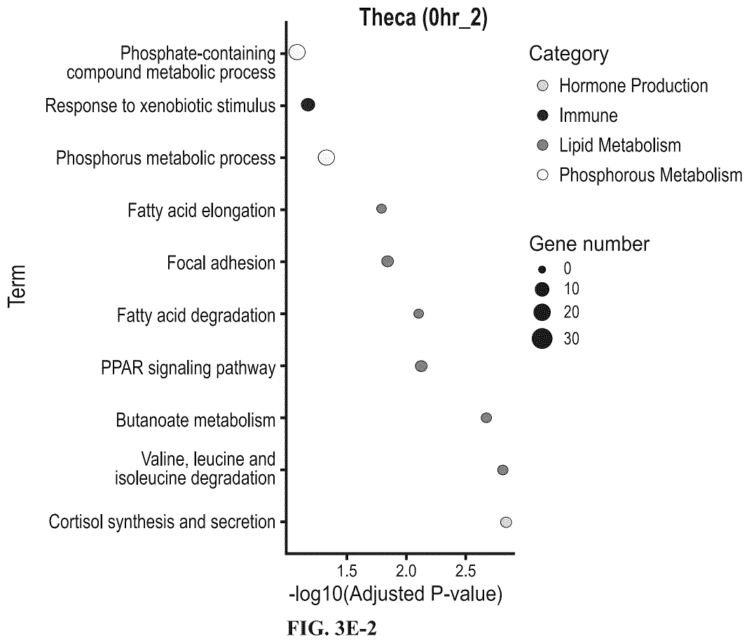

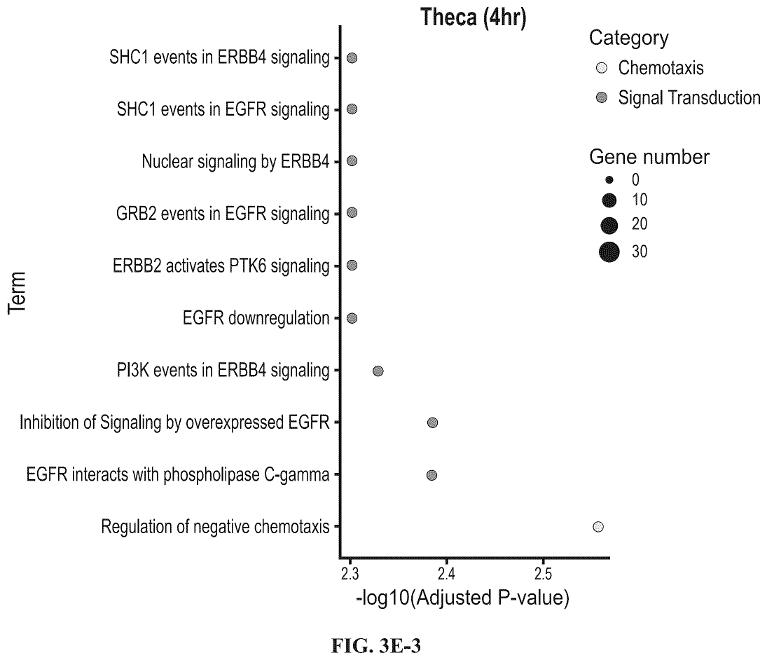

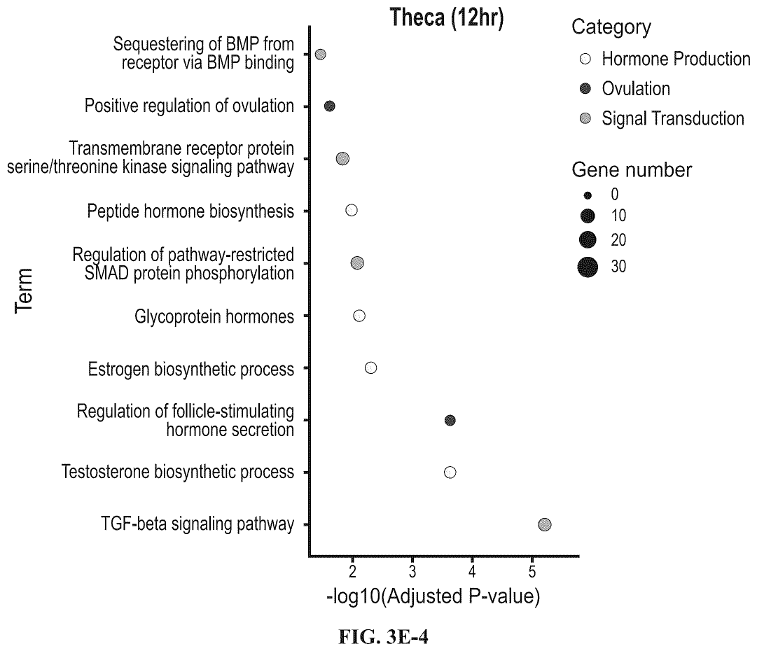

Theca cells: Theca cells were further differentiated into four subclusters with expression at one specific time point (0h, 4h, or 12h); two subclusters were expressed predominantly at 0h while the remaining two subclusters were expressed at 4h or 12h (FIGS. 3A-C, Table 10). The top differentially expressed genes in the theca subclusters are shown in FIG. 3B. The genes driving the differentiation between subclusters were further characterized into known and undescribed genes in the theca cells. Genes known to be active in theca cells included Star and Cyp17a1, expressed in early and late theca cells, respectively (FIGS. 3D-1 and 3D-2). More specifically, Star expression distinguished the 4 h and 12h theca subclusters from the 0h clusters, which was marked by Cyp17a1 expression. Star, encoding steroidogenic acute regulatory protein, promotes steroid production by facilitating cholesterol transport between the outer and inner mitochondrial membranes, thus driving the synthesis of pregnenolone from cholesterol. Similarly, Cyp17a1 is a cytochrome P450 enzyme that catalyzes critical steps in androgen synthesis. In addition, several genes upregulated in theca subclusters were identified that have yet to be described in this subtype, including Oca2 (at 0h) and Tpm4 (at 12h) (FIGS. 3D-1 and 3D-2). Oca2 encodes P protein, which is integrated into the cell membrane and functions by maintaining pH and transporting small molecules. Tpm4, or tropomyosin 4, is typically involved in actin cytoskeleton organization and contraction of non-muscle tissues. In non-mammalian models, tropomyosin has been shown to be essential for follicle rupture. Not intending to be bound by theory, since vasoconstriction of the theca interna is critical for follicle rupture, Tpm4 may play a similar role in facilitating ovulation.

TABLE 10
Curated list of top pathway analysis results for all clusters.

| | | |
|---|---|---|
| Database | Term | Category |

Cumulus 1

| | | |
|---|---|---|
| KEGG 2021 | Axon guidance | Nervous system |
| Reactome 2022 | Nervous System Development | Nervous system |
| Reactome 2022 | Signaling By ERBB2 | Signal transduction |
| Reactome 2022 | EPH-ephrin Mediated Repulsion Of | Cell-cell interaction |
| | Cells | |
| Reactome 2022 | EGFR Interacts With | Signal transduction |
| | Phospholipase C-gamma | |
| Reactome 2022 | Glycosaminoglycan Metabolism | Glycosaminoglycan |
| | | metabolism |
| Panther | negative regulation of negative | Cell migration |
| | chemotaxis | |
| Panther | ovarian cumulus expansion | Ovulation |
| Panther | regulation of connective tissue | Immune |
| | replacement involved in | |
| | inflammatory response wound | |
| | healing | |
| Panther | fused antrum stage | Ovulation |

Cumulus 2

| | | |
|---|---|---|
| GO BP 2023 | Regulation Of Cell Migration | Cell migration |
| Reactome 2022 | Metabolism Of Lipids | Lipid metabolism |
| KEGG 2021 | PPAR signaling pathway | Lipid metabolism |
| Reactome 2022 | TGF-beta Receptor Signaling | Signal transduction |
| | Activates SMADs | |
| Reactome 2022 | Neutrophil Degranulation | Immune |
| Reactome 2022 | Innate Immune System | Immune |
| Panther | positive regulation of intracellular | Lipid metabolism |
| | cholesterol transport | |
| Panther | ovarian cumulus expansion | Ovulation |
| Panther | connective tissue replacement | Immune |
| | involved in inflammatory response | |
| | wound healing | |
| Panther | positive regulation of prostaglandin | Lipid metabolism |
| | biosynthetic process | |

Endothelial 1

| | | |
|---|---|---|
| GO BP 2023 | Regulation Of Angiogenesis | Angiogenesis |
| Reactome 2022 | Integrin Cell Surface Interactions | ECM formation |
| GO BP 2023 | Positive Regulation Of Vasculature | Angiogenesis |
| | Development | |
| GO BP 2023 | Regulation Of Blood Vessel | Cell migration |
| | Endothelial Cell Migration | |
| Panther | endothelial cell-matrix adhesion | Cell-cell adhesion |
| Panther | antigen processing and presentation | Immune |
| | of endogenous peptide antigen via | |
| | MHC class I via ER pathway, TAP- | |
| | dependent | |
| Panther | adrenomedullin receptor signaling | Signal transduction |
| | pathway | |
| Panther | positive regulation of | Angiogenesis |
| | vasculogenesis | |
| Panther | xenobiotic detoxification by | Detoxification |
| | transmembrane export across the | |
| | plasma membrane | |
| Panther | endothelial cell morphogenesis | Angiogenesis |

Endothelial 2

| | | |
|---|---|---|
| GO BP 2023 | Blood Vessel Morphogenesis | Angiogenesis |
| GO BP 2023 | Regulation Of Angiogenesis | Angiogenesis |
| GO BP 2023 | Regulation Of Endothelial Cell | Cell migration |
| | Migration | |
| GO BP 2023 | Regulation Of Endothelial Cell | Cell proliferation |
| | Proliferation | |
| Reactome 2022 | Platelet Activation, Signaling And | Immune |
| | Aggregation | |
| GO BP 2023 | Positive Regulation Of Vasculature | Angiogenesis |
| | Development | |
| Panther | adrenomedullin receptor signaling | Signal transduction |
| | pathway | |
| Panther | antigen processing and presentation | Immune |
| | of endogenous peptide antigen via | |
| | MHC class I via ER pathway, TAP- | |
| | dependent | |
| Panther | calcitonin family receptor signaling | Signal transduction |
| | pathway | |
| Panther | lymphatic endothelial cell | Development |
| | differentiation | |

Epithelial

| | | |
|---|---|---|
| KEGG 2021 | Tight junction | Cell-cell adhesion |
| GO BP 2023 | Cellular Response To CAMP | Signal transduction |
| GO BP 2023 | Protein Localization To Membrane | Protein localization |
| GO BP 2023 | Microvillus Assembly | Cell organization |
| GO BP 2023 | Regulation Of Protein | Protein modification |
| | Phosphorylation | |
| Panther | response to zinc ion | Metal homeostasis |
| Panther | regulation of homotypic cell-cell | Cell-cell adhesion |
| | adhesion | |
| Panther | negative regulation of wound | Immune |
| | healing | |
| Panther | regulation of cell shape | Cell homeostasis |
| Panther | epithelial cell development | Development |
| Database | Term | Category |
| KEGG 2021 | TGF-beta signaling pathway | Signal transduction |
| GO BP 2023 | Gonad Development | Development |
| GO BP 2023 | Estrogen Biosynthetic Process | Hormone production |
| KEGG 2021 | Ovarian steroidogenesis | Hormone production |
| GO BP 2023 | Positive Regulation Of Pathway- | Signal transduction |
| | Restricted SMAD Protein | |
| | Phosphorylation | |
| GO BP 2023 | Proteoglycan Metabolic Process | Proteoglycan |
| | | metabolism |
| Panther | positive regulation of ovulation | Ovulation |
| Panther | sequestering of BMP from receptor | Signal transduction |
| | via BMP binding | |
| Panther | regulation of follicle-stimulating | Ovulation |
| | hormone secretion | |
| Panther | testosterone biosynthetic process | Hormone production |

Granulosa 2

| | | |
|---|---|---|
| KEGG 2021 | Axon guidance | Nervous system |
| GO BP 2023 | Regulation Of Cell Migration | Cell migration |
| GO BP 2023 | Cellular Component Assembly | Cell organization |
| GO BP 2023 | Extracellular Structure | ECM formation |
| | Organization | |
| GO BP 2023 | External Encapsulating Structure | ECM formation |
| | Organization | |
| Panther | uterus development | Development |
| Panther | basement membrane organization | ECM formation |
| GO BP 2023 | Endochondral Bone Morphogenesis | Development |
| Panther | regulation of cardiac muscle | Signal transduction |
| | contraction by regulation of the | |
| | release of sequestered calcium ion | |
| Panther | heart valve development | Development |

Granulosa 3

| | | |
|---|---|---|
| Reactome 2022 | Cell Cycle, Mitotic | Cell cycle |
| Reactome 2022 | Cell Cycle Checkpoints | Cell cycle |
| GO BP 2023 | DNA Conformation Change | Cell cycle |
| GO BP 2023 | Positive Regulation Of Cell Cycle | Cell cycle |
| | Process | |
| Reactome 2022 | Mitotic Metaphase And Anaphase | Cell cycle |
| Panther | pyrimidine deoxyribonucleoside | Nucleic acid |
| | triphosphate metabolic process | metabolism |
| Panther | mitotic spindle midzone assembly | Cell cycle |
| Panther | mitotic spindle elongation | Cell cycle |
| Panther | mitotic chromosome condensation | Cell cycle |
| Panther | positive regulation of mitotic sister | Cell cycle |
| | chromatid separation | |

Luteal 1

| | | |
|---|---|---|
| Reactome 2022 | Metabolism Of Lipids | Lipid metabolism |
| Reactome 2022 | Metabolism Of Steroids | Hormone production |
| Reactome 2022 | Regulation Of Cholesterol | Lipid metabolism |
| | Biosynthesis By SREBP (SREBF) | |
| Reactome 2022 | Fatty Acid Metabolism | Lipid metabolism |
| GO BP 2023 | Cholesterol Metabolic Process | Lipid metabolism |
| Panther | cellular response to prostaglandin D | Signal transduction |
| | stimulus | |
| Panther | regulation of testosterone | Hormone production |
| | biosynthetic process | |
| Panther | NADPH oxidation | Electron transport |
| | | chain |
| Panther | tertiary alcohol metabolic process | Alcohol metabolism |
| Panther | very long-chain fatty acid | Lipid metabolism |
| | biosynthetic process | |

Luteal 2

| | | |
|---|---|---|
| Reactome 2022 | Glycosaminoglycan Metabolism | Glycosaminoglycan |
| | | metabolism |
| Reactome 2022 | Chondroitin Sulfate/Dermatan | ECM formation |
| | Sulfate Metabolism | |
| Reactome 2022 | Extracellular Matrix Organization | ECM formation |
| Reactome 2022 | Activation Of TFAP2 (AP-2) | Signal transduction |
| | Family Of Transcription Factors | |
| Reactome 2022 | Metabolism | Metabolism |
| Panther | cell volume homeostasis | Cell homeostasis |
| Panther | response to estrogen | Signal transduction |
| Panther | positive regulation of stress fiber | ECM formation |
| | assembly | |
| Panther | transforming growth factor beta | Signal transduction |
| | receptor signaling pathway | |
| Panther | positive regulation of actin filament | ECM formation |
| | bundle assembly | |

Myeloid

| | | |
|---|---|---|
| Reactome 2022 | Innate Immune System | Immune |
| Reactome 2022 | Neutrophil Degranulation | Immune |
| KEGG 2021 | Lysosome | Cell homeostasis |
| Reactome 2022 | Adaptive Immune System | Immune |
| GO BP 2023 | Positive Regulation Of | Immune |
| | Phagocytosis | |
| KEGG 2021 | Chemokine signaling pathway | Immune |
| GO BP 2023 | Positive Regulation Of Tumor | Immune |
| | Necrosis Factor Superfamily | |
| | Cytokine Production | |
| Panther | positive regulation of antigen | Immune |
| | processing and presentation of | |
| | peptide or polysaccharide antigen | |
| | via MHC class II | |
| Panther | antigen processing and presentation | Immune |
| | of exogenous peptide antigen via | |
| | MHC class I | |
| Panther | myeloid dendritic cell activation | Immune |
| | involved in immune response | |

Stroma 1

| | | |
|---|---|---|
| Reactome 2022 | Extracellular Matrix Organization | ECM formation |
| Reactome 2022 | Collagen Chain Trimerization | ECM formation |
| Reactome 2022 | Assembly Of Collagen Fibrils And | ECM formation |
| | Other Multimeric Structures | |
| Reactome 2022 | Collagen Biosynthesis And | ECM formation |
| | Modifying Enzymes | |
| KEGG 2021 | ECM-receptor interaction | ECM formation |
| KEGG 2021 | Focal adhesion | ECM formation |
| KEGG 2021 | Protein digestion and absorption | Protein metabolism |
| Reactome 2022 | Smooth Muscle Contraction | Muscle contraction |
| Panther | positive regulation of cell | Cell proliferation |
| | proliferation by VEGF-activated | |
| | platelet derived growth factor | |
| | receptor signaling pathway | |
| Panther | positive regulation of nitric oxide | Signal transduction |
| | mediated signal transduction | |

Stroma 2

| | | |
|---|---|---|
| Reactome 2022 | Extracellular Matrix Organization | ECM formation |
| GO BP 2023 | Negative Regulation Of Cell | Cell migration |
| | Migration | |
| Reactome 2022 | Binding And Uptake Of Ligands | Cell homeostasis |
| | By Scavenger Receptors | |
| Reactome 2022 | Smooth Muscle Contraction | Muscle contraction |
| Reactome 2022 | Collagen Chain Trimerization | ECM formation |
| Reactome 2022 | Collagen Biosynthesis And | ECM formation |
| | Modifying Enzymes | |
| Panther | cellular response to heparin | Signal transduction |
| Panther | positive regulation of calcium ion | Signal transduction |
| | transmembrane transport via high | |
| | voltage-gated calcium channel | |
| Panther | negative regulation of SMAD | Signal transduction |
| | protein complex assembly | |
| Reactome 2022 | Regulation Of IGF Transport And | Protein metabolism |
| | Uptake By IGFBPs | |

Theca 1

| | | |
|---|---|---|
| KEGG 2021 | Valine, leucine and isoleucine | Amino acid |
| | degradation | metabolism |
| KEGG 2021 | PPAR signaling pathway | Lipid metabolism |
| Reactome 2022 | Metabolism Of Lipids | Lipid metabolism |
| Reactome 2022 | Mitochondrial Fatty Acid Beta- | Lipid metabolism |
| | Oxidation Of Saturated Fatty Acids | |
| KEGG 2021 | Cortisol synthesis and secretion | Hormone production |
| Panther | acetaldehyde metabolic process | Aldehyde |
| | | metabolism |
| Panther | fatty acid beta-oxidation using acyl- | Lipid metabolism |
| | CoA dehydrogenase | |
| Panther | phospholipid homeostasis | Lipid metabolism |
| Panther | C21-steroid hormone metabolic | Hormone production |
| | process | |
| Panther | fatty acid catabolic process | Lipid metabolism |
| Database | Term | Category |
| Reactome 2022 | Cholesterol Biosynthesis | Lipid metabolism |
| GO BP 2023 | Sterol Metabolic Process | Hormone production |
| Reactome 2022 | Metabolism Of Steroid Hormones | Hormone production |
| GO BP 2023 | Steroid Biosynthetic Process | Hormone production |
| Panther | isoprenoid biosynthetic process via | Lipid metabolism |
| | mevalonate | |
| Panther | farnesyl diphosphate biosynthetic | Lipid metabolism |
| | process, mevalonate pathway | |
| Panther | detoxification of copper ion | Metal homeostasis |
| Panther | stress response to copper ion | Metal homeostasis |
| Panther | glucocorticoid biosynthetic process | Hormone production |
| Panther | terpenoid biosynthetic process | Lipid metabolism |

GO analysis of top differentially expressed genes between each theca subcluster was conducted. This analysis revealed that pathways upregulated in the first 0h subcluster were related to cell proliferation, immune processes, metal homeostasis, and plasma membrane function (FIGS. 3E-1 to 3E-4). Metal homeostasis specifically is a novel function of theca cells first documented in this Example. One of the primary functions of theca cells is steroid hormone production, a process localized to the mitochondria. Mitochondria also participate in metal homeostasis by taking in metals and metalating proteins. One such metalloprotein is superoxide dismutase, which reduces reactive oxygen species (ROS). Importantly, steroidogenesis itself generates reactive oxygen proteins, and thus metal homeostasis in theca cells may be critical to the processing of metalloproteins with roles in ROS reduction. In the second 0h subcluster, pathways upregulated in theca cells included hormone production, immune function, and metabolism of lipids/phosphorus. Theca cells at 4h showed upregulated pathways including chemotaxis and signal transduction. At 12 h, theca cells appeared to be involved in processes related to hormone production, ovulation, and signal transduction. Taken together, the results of the GO analysis validated the theca cell clusters by recapitulating known functions of theca cells (e.g. hormone production). In addition, these results revealed functions of theca cells that are new, such as metal homeostasis, thus presenting testable hypotheses about time-dependent functions of theca cells throughout ovulation.

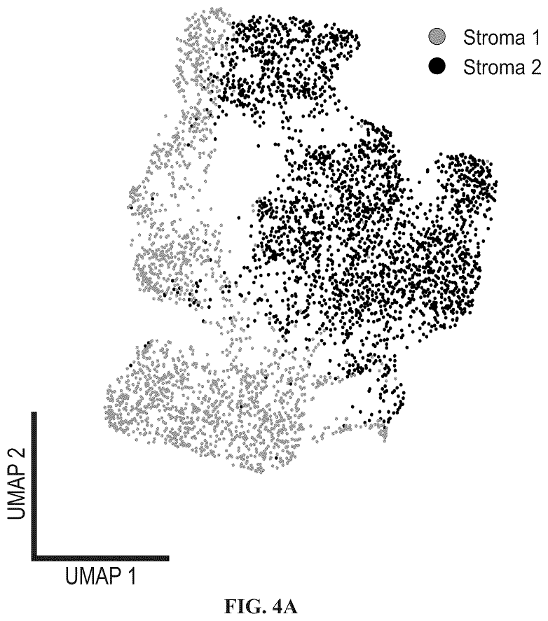

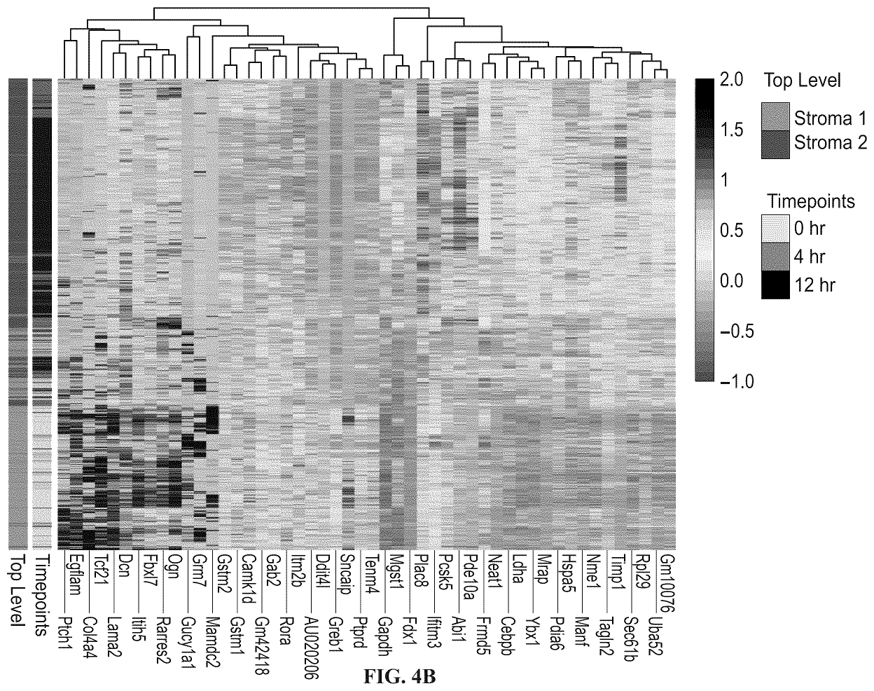

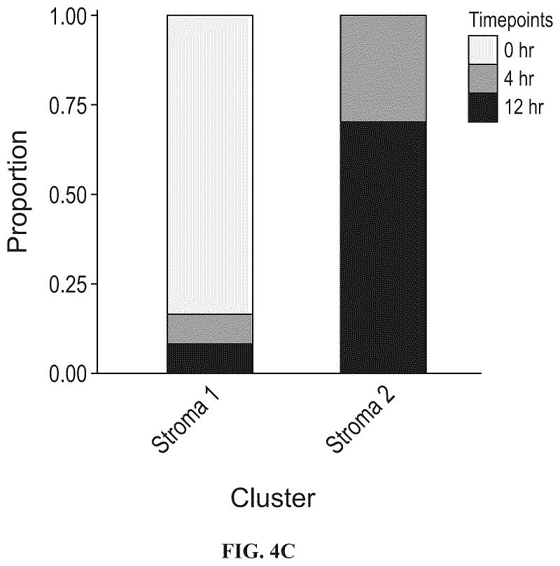

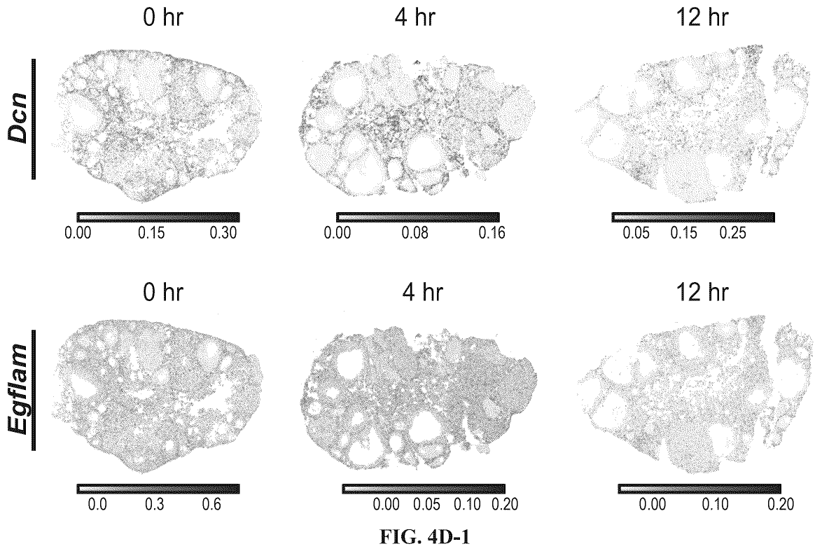

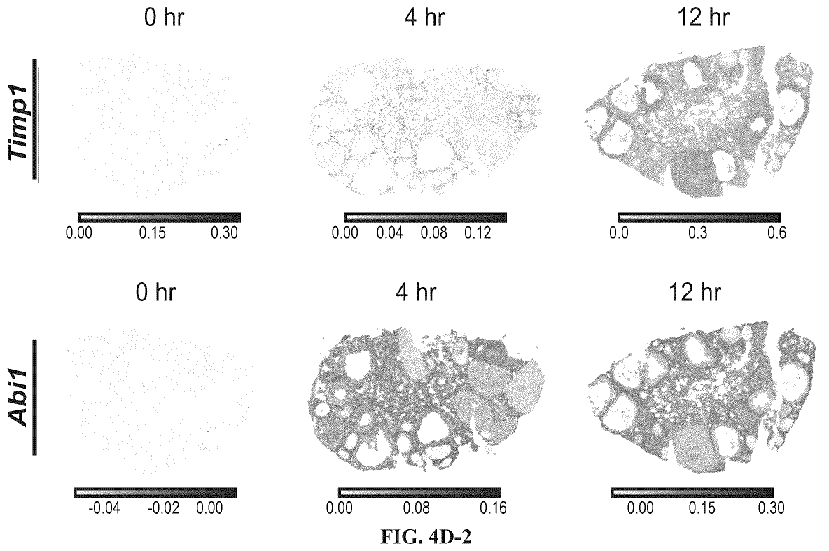

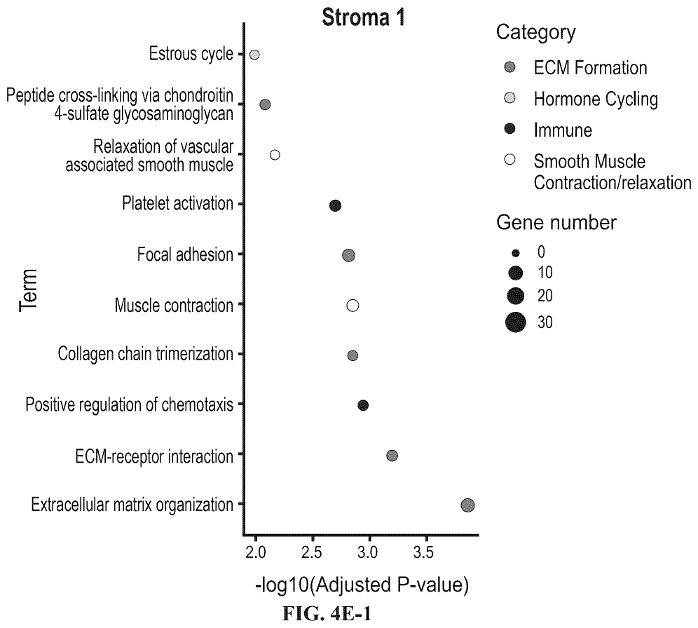

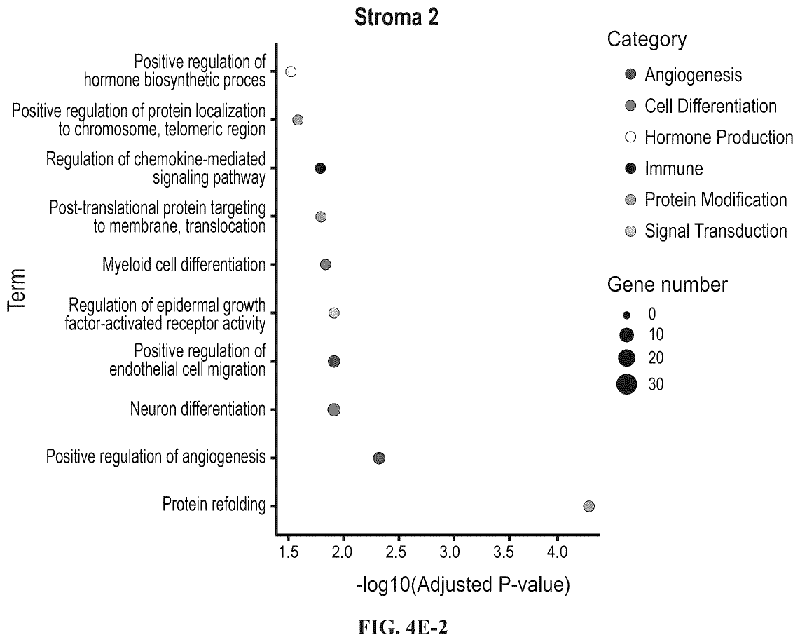

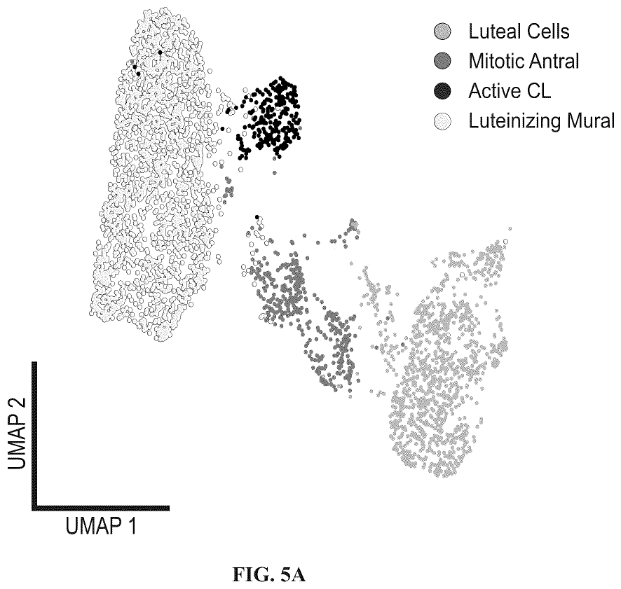

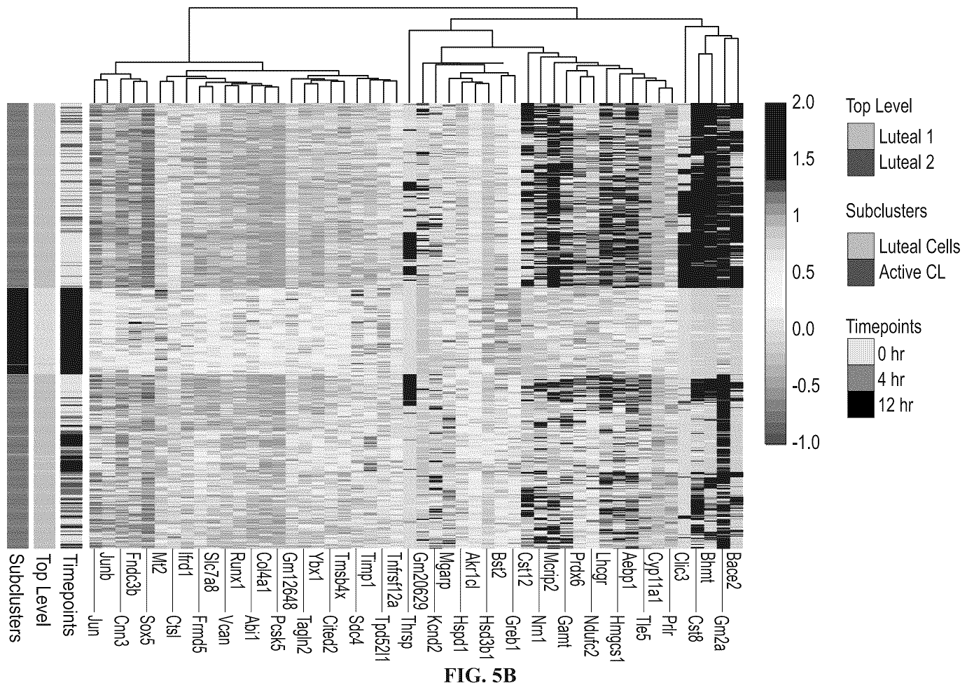

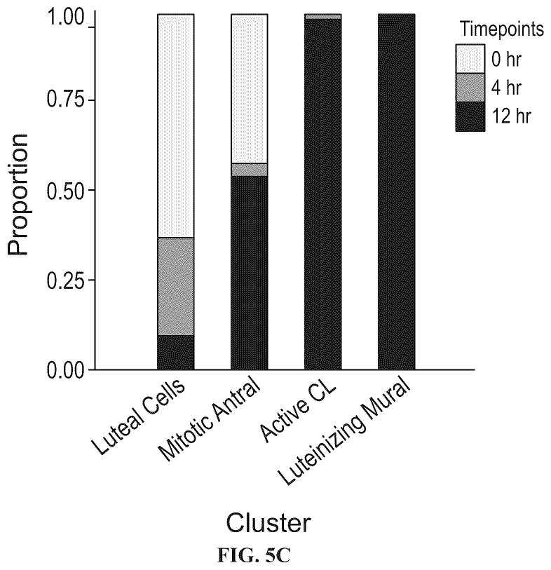

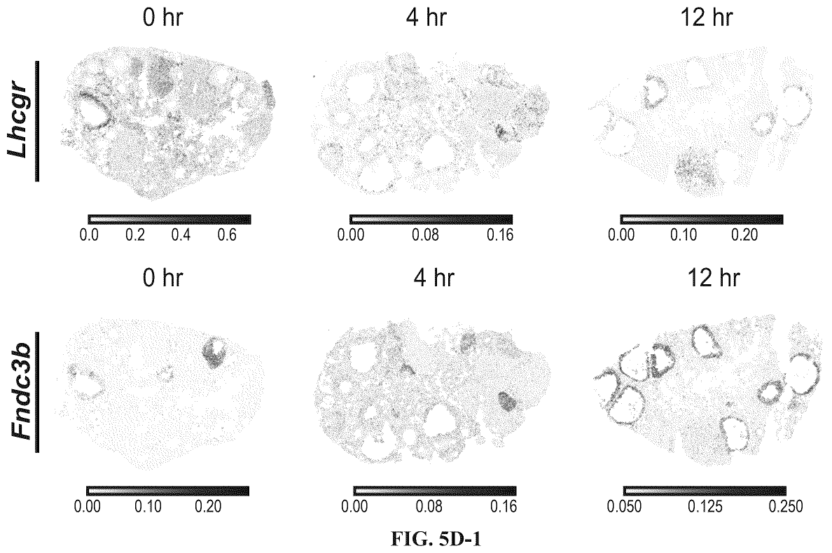

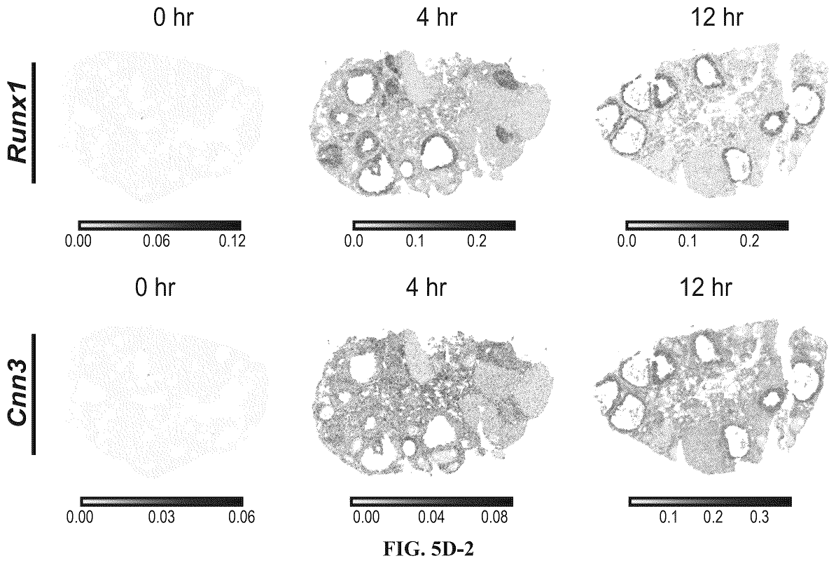

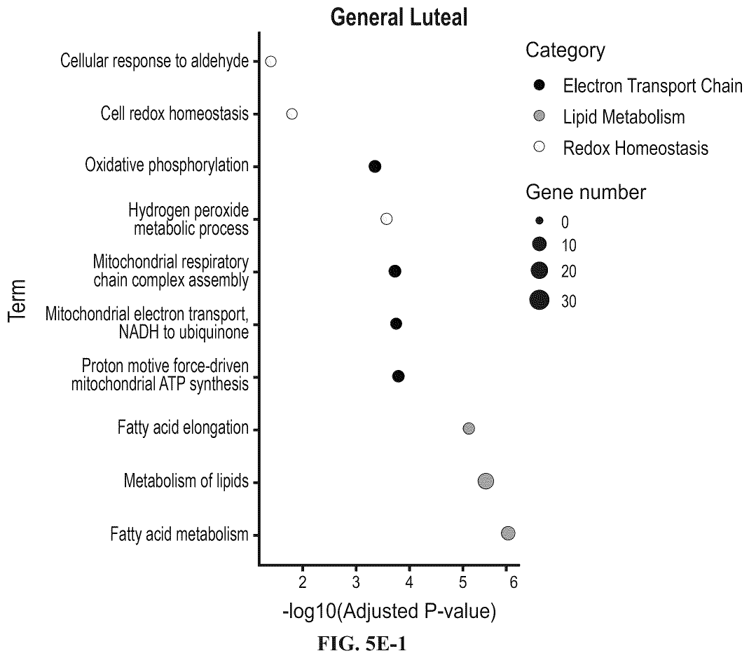

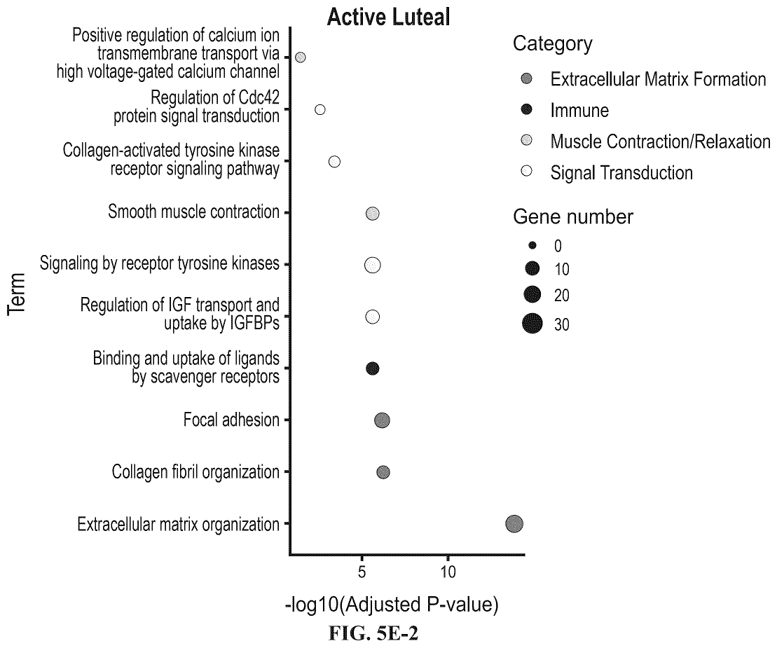

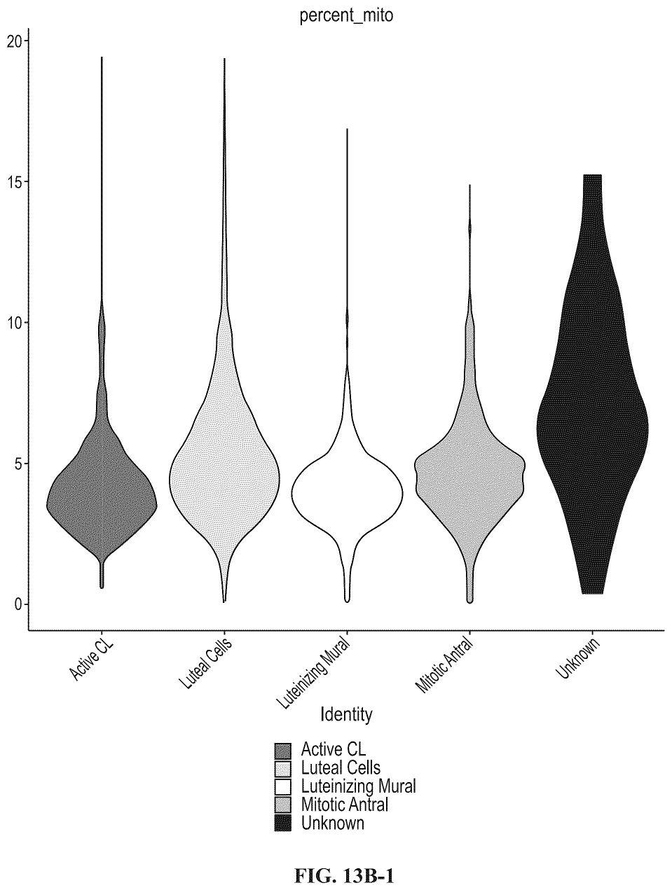

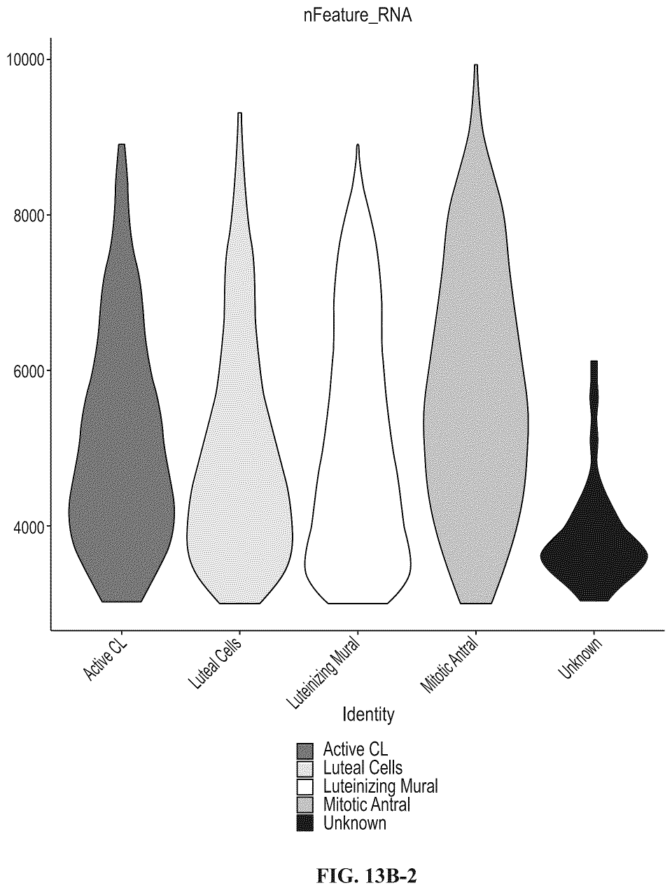

Luteal cells: Using published luteal cell subtype markers (Morris et al., eLife, 11, e77239 (2022)), four luteal subclusters were identified: active luteal cells, general luteal cells, luteinizing mural cells, and mitotic antral cells (FIGS. 4A and 4B, 13A, 13B-1, 13B-2 Table 10). The top differentially expressed genes in the subclusters with specific luteal identity are shown in FIG. 5B. The luteal subclusters exhibited temporal differences with general luteal cells predominately presented in 0h and 4h timepoints whereas active luteal and luteinizing mural cells were primarily found at 12h (FIG. 4C).

The differential gene expression profiles between general and active luteal cells was further investigated. Upregulation of Lhcgr in general luteal cells was observed (FIGS. 4D-1 and 4D-2) where it binds LH to promote androgen synthesis. In addition, Runx1, a DNA-binding protein known to be an essential transcriptional regulator of ovulation and luteinization, was upregulated at 12h in active luteal cell subcluster. Novel genes with temporal expression patterns in luteal cells were also identified (FIGS. 4D-1 And 4D-2). Fndc3b, also enriched in active luteal cells, belongs to the fibronectin type III domain containing family of myokines and adipokines with general roles in migration, adhesion, and proliferation of cells. FDNC3B has roles in bone and tumor development, but no documented functions in the ovary. However, recent studies on another member of this myokine/adipokine family, FNDC5 or irisin, has been shown to regulate the steroid hormone production and secretion in various granulosa cell models. As such, not intending to be bound by theory, Fndc3b may function in the production of progesterone within the corpus luteum. In addition, Cnn3 was expressed in active luteal cells and encodes a filament-associated protein that controls smooth muscle contraction. In the corpus luteum, upregulation of angiogenesis is critical for the transport of progesterone to the bloodstream. Therefore, Cnn3 may be a component of vascular smooth muscle within newly formed vessels. Interestingly, Cnn3 was also a top upregulated gene in the late theca subcluster, particularly at the 4 hr timepoint (FIG. 3B). Not intending to be bound by theory, since vasoconstriction of the theca interna is critical for follicle rupture and new capillary growth following LH surge, Cnn3 may contribute to ovulation and the luteinization of theca cells.

Gene ontology (GO) analysis was performed on the top level Luteal 1 and 2 clusters using the marker genes for each cluster. Early luteal (Luteal 1) cells were highly enriched in pathways related to hormone production, consistent with the role of luteal cells in synthesizing progesterone. Late luteal (Luteal 2) cells exhibited a range of upregulated pathways, including those involved in extracellular matrix organization and signal transduction. “Response to estrogen” and “cell volume homeostasis” were also found as two additional pathways of interest in the Luteal 2 cluster. High levels of estrogen trigger ovulation, which is followed by luteinization of granulosa and theca cells. In addition, luteinization is marked by hypertrophy of luteal cells, a modulation of cell volume. In interpreting these results, it is important to note that the Luteal 1 cluster (which contains cells from all three time points but mostly 0h and 4h) likely contains existing corpora lutea from previous cycles. In mice, corpora lutea persist for multiple cycles and receive repeated luteolytic signals and may continue to produce progesterone. On the contrary, the Luteal 2 cluster was expressed exclusively at 12 h and may represent actively forming or newly formed corpora lutea, supporting the presence of pathways related to the organization of luteinizing granulosa and theca cells into a functional corpus luteum.

Lastly, GO analysis was conducted on the general luteal and active luteal cells using the differentially expressed genes between each subcluster. General luteal cells showed several pathways related to the electron transport chain, lipid metabolism, and redox homeostasis whereas active luteal cells were enriched in pathways related to extracellular matrix formation, immune function, muscle contraction, and signal transduction (FIGS. 5E-1 and 5E-2). Lipid metabolism is a key step in steroid hormone synthesis, which occurs in the mitochondria, the cellular organelle in which the electron transport chain and redox homeostatic pathways are active. Thus, while not intending to be bound by theory, this data suggests that general luteal cells exhibit a primary function of steroid hormone production, consistent with the synthesis of progesterone within mature corpus lutea from previous cycles. In contrast, active luteal cells may represent newly forming corpus lutea that are undergoing extensive extracellular matrix (ECM) remodeling and vascularization.

Cumulus cells: For cumulus cells, two time-dependent subclusters were identified: early cumulus (Cumulus 1) and late cumulus (Cumulus 2) (FIGS. 5A-B). Both subclusters were distinct temporally with early and cumulus cells primarily at 4 h and 12h, respectively (FIG. 5C). The top differentially expressed genes between the early and late cumulus cell subclusters were Sultle1, Robo2, Pgr, Rnf180, and Tac1 (upregulated in early cumulus cells) as well as Spp1, S100a6, Cck, Timp], and Chchd10 (upregulated in late cumulus cells; FIG. 5B). Given that the cumulus subclusters were not previously identified in the reference scRNA-seq dataset (Morris et al., eLife, 11, e77239 (2022)) (FIG. 8E), the predicted expression pattern from the integrated single cell and spatial transcriptomic datasets was validated with RNA quantification using RNAScope on histological ovarian sections (FIGS. 5D-1 and 5D-2). Sultle1, a known cumulus cell marker gene involved in estrogen metabolism, was high at 4h, and Lox, a known gene involved in ECM formation during follicle development, was high at 12h. In addition, the transcriptomic analysis identified two genes, Zfp804a and Emb, which also showed similar expression patterns to Sultle1 and Lox, respectively. Indeed, similar expression patterns were observed with RNAScope validation (FIGS. 5D-1 and 5D-2). Not intending to be bound by theory, with Zfp804a and Emb being previously known as neuronal genes, the findings suggest that the integrated single cell and spatial dataset can be used to identify novel genes involved in ovarian function during ovulation.

Gene ontology (GO) analysis of top marker genes in the cumulus 1 and 2 clusters revealed “ovarian cumulus expansion” as a top biological process in both clusters, further validating the cell type identification. Early cumulus cells also showed upregulation of “glycosaminoglycan metabolism,” “fused antrum stage,” and “EGFR interacts with phospholipase C-gamma” processes. These pathways are consistent with known biological processes occurring in the cumulus-oocyte complex (COC) during ovulation, when epidermal growth factor (EGF) stimulates production of glycosaminoglycans (e.g., hyaluronan) in cumulus cells to promote COC expansion and fluid accumulation within the antral follicle. In addition, hyaluronan is secreted by granulosa cells into the follicular fluid, where it contributes to an osmotic gradient that draws fluid from theca cells into the antral space. Early cumulus cells also exhibited several pathways related to the nervous system, including “axon guidance” and “nervous system development”. Genes that drove the “axon guidance” pathway included Epha5, Robo2, Epha4, Epha7, Alcam, Sema3c, and Efna5. Genes including Epha5, Robo2, Epha4, Epha7, Trpc5, Yes], Adgrvl, Arhgefl2, Ncbpl, Itga2, Prkca, Efna, Robol, Alcam, and Rps6ka2 drove the “nervous system development” pathway (Table 9). Upregulation of genes important to nervous system function has previously been documented in COCs, but their role in cumulus cells remains unclear. Since cumulus cells undergo substantial cellular changes during the LH surge, such as cell migration and retraction of transzonal projections from the oocyte, they may possess neural-like plasticity during expansion. Late cumulus cells (Cumulus 2) exhibited enrichment of genes for “positive regulation of prostaglandin biosynthetic process,” which is consistent with two processes occurring just before and after ovulation of the COC near 12h post-hCG. Prostaglandins both stimulate COC expansion and promote dissolution of the COC ECM to facilitate fertilization of the oocyte by sperm. The Cumulus 2 cluster also exhibits several pathways related to cholesterol production and immune function (Table 9).

To identify time-dependent changes in biological processes within cumulus cells across ovulation, a second GO analysis was conducted using the top differentially expressed genes between early and late cumulus cells. The transcriptome of early cumulus cells (Cumulus 1) exhibited enrichment of genes related to the nervous system, chemotaxis, immune function, and signal transduction, with the latter representing key pathways driving COC expansion including lutenizing hormone (LH) and receptor tyrosine kinase signaling. Late cumulus cells (Cumulus 2) exhibited enrichment in genes related to fatty acid metabolism and cholesterol/hormone synthesis (FIGS. 5E-1 and 5E-2). Steroid hormone synthesis by cumulus cells has been documented in several species, including mice and humans, and is thought to be important for oocyte maturation and quality. A recent Interactome analysis performed between cumulus and mural granulosa cells as senders and receivers, respectively, revealed a number of interactions related to steroidogenesis.

Progesterone produced by cumulus cells also serves as a sperm chemoattractant. Therefore, while not intending to be bound by theory, the upregulation of steroidogenic pathways in late cumulus cells may be supporting several processes that occur near the completion of ovulation, including hormone production in luteinizing mural cells and fertilization by sperm.

### Example 4: Cell-Cell Interaction (CCI) Analysis Reveals Significant Interactions that Change Over the Time Course of Ovulation

To characterize the signaling pathways that drive molecular functions across ovulation, cell-cell interaction analysis was conducted for each cell type at each timepoint (FIGS. 6A-1, 6A-2, and 6A-3). Interestingly, a pattern of distinct and consistent cell subclusters that sent and received signals across the ovulation time course was observed. At 0h, it was found that the primary senders were Stroma 1, Epithelial, Granulosa 1, and Granulosa 2. Notably, the cell subcluster that mainly received signals at 0h was General Luteal. For the 4h timepoint, the primary senders were Stroma 1, Stroma 2, Cumulus 1, Epithelial, and Theca 1 whereas General Luteal, Endothelial 1, and Epithelial were primarily receivers. At the 12h timepoint, the major senders were Cumulus 2, Stroma 1, Stroma 2, and Active Luteal subclusters. In contrast, most cell subclusters exhibited similar proportions of received signals. Not intending to be bound by theory, these patterns suggest that stromal and cumulus cells remain active communicators throughout ovulation after 4 hr, in contrast to luteal cells which primarily receive signals from other cell types. Overall, increased incoming interaction was observed in the 12h compared to earlier timepoints. Notably, at 12 h, the three cell subclusters with the highest incoming signal strength were Cumulus 2, Active Luteal, and Luteinizing Mural (FIGS. 6B-1, 6B-2, 6B-3, and 12A to 12D). It was also observed that other luteal subclusters present from previous regressing corpus lutea, such as general luteal cells, were less active due to low incoming and outgoing signal strength.

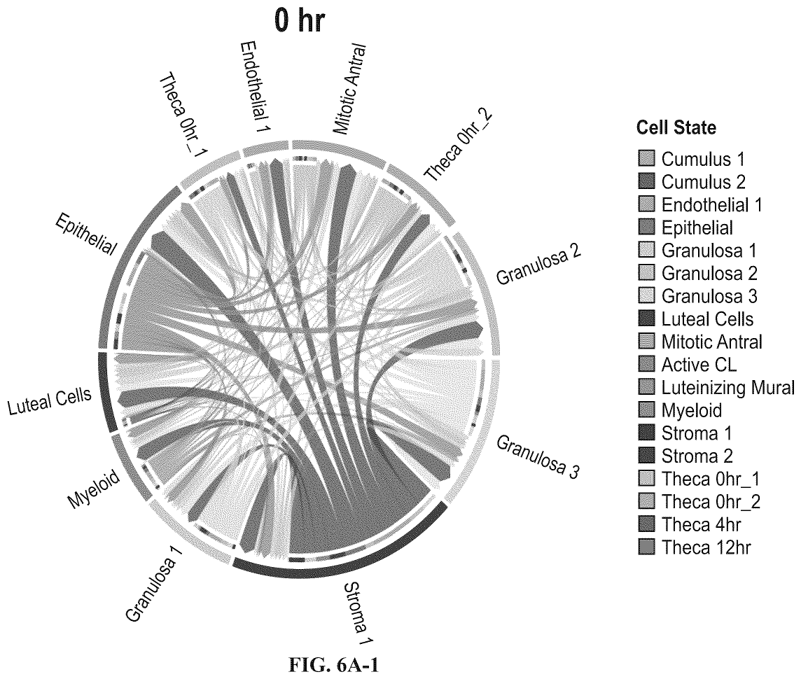

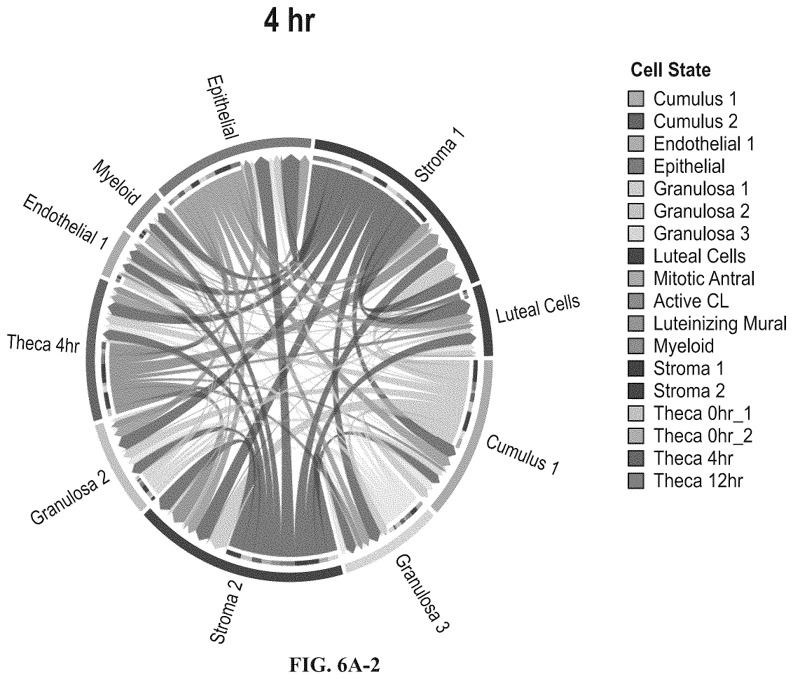

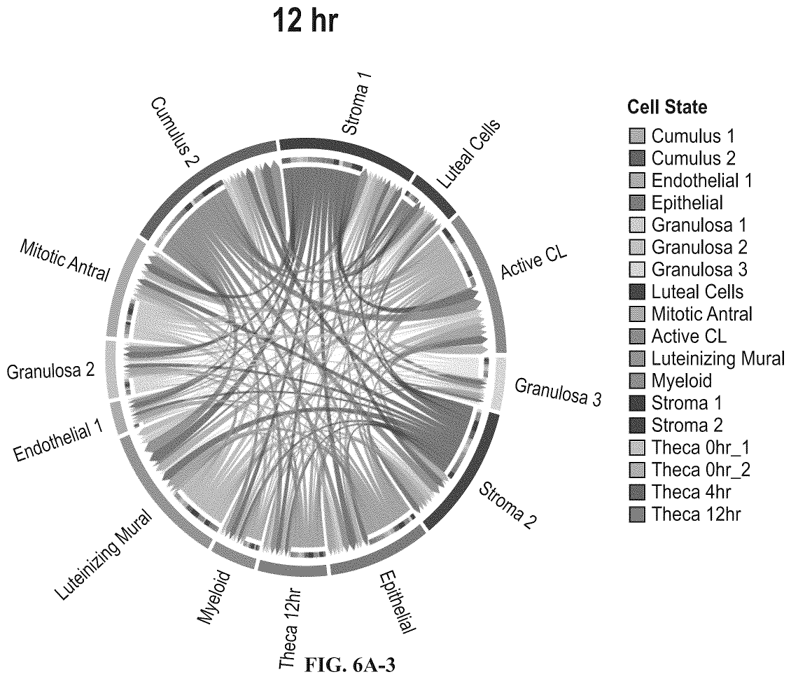

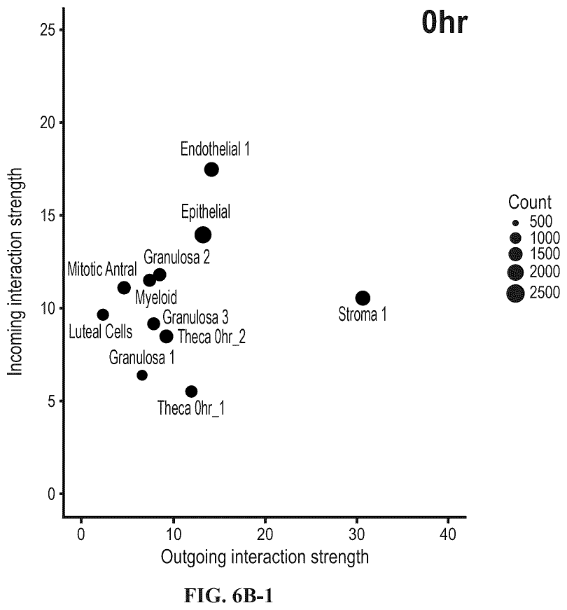

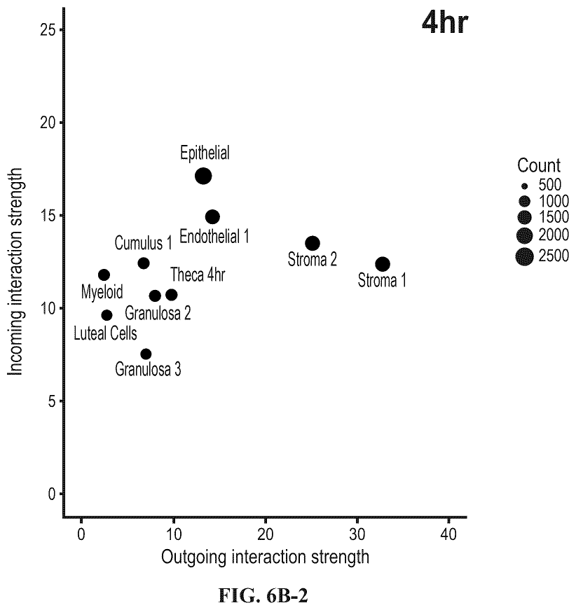

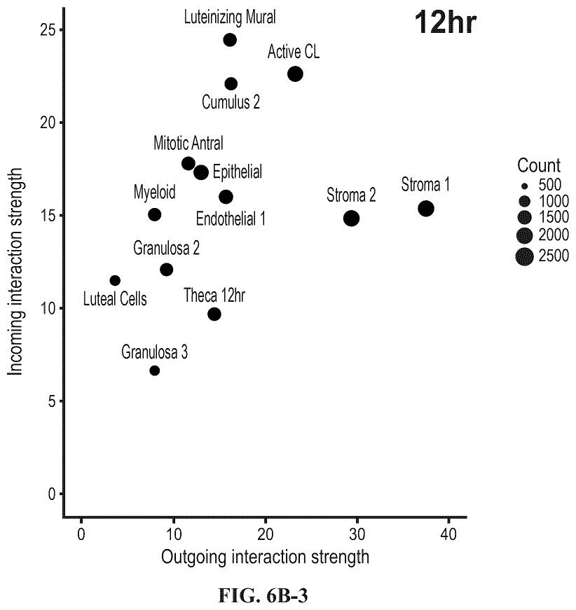

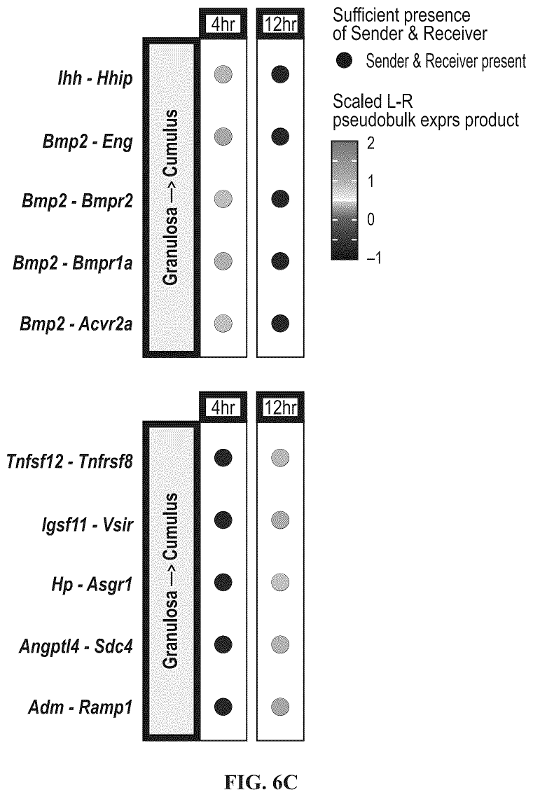

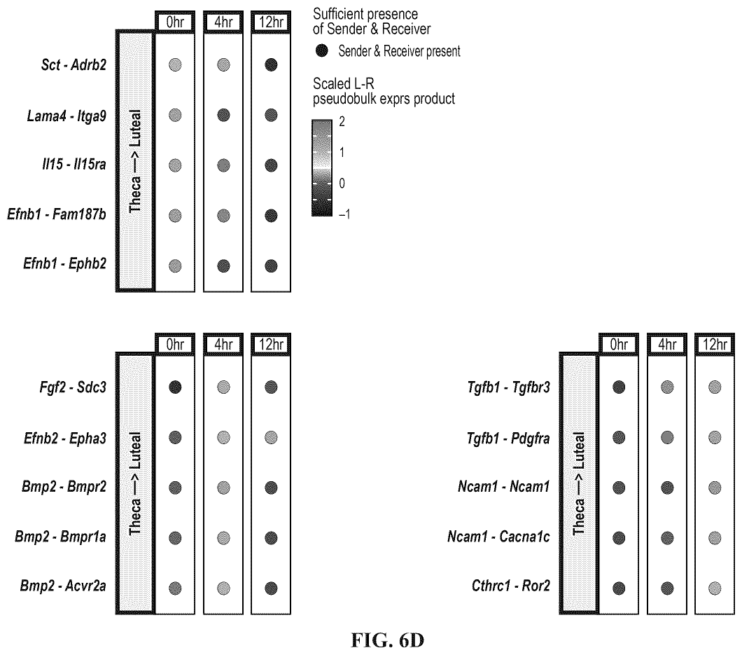

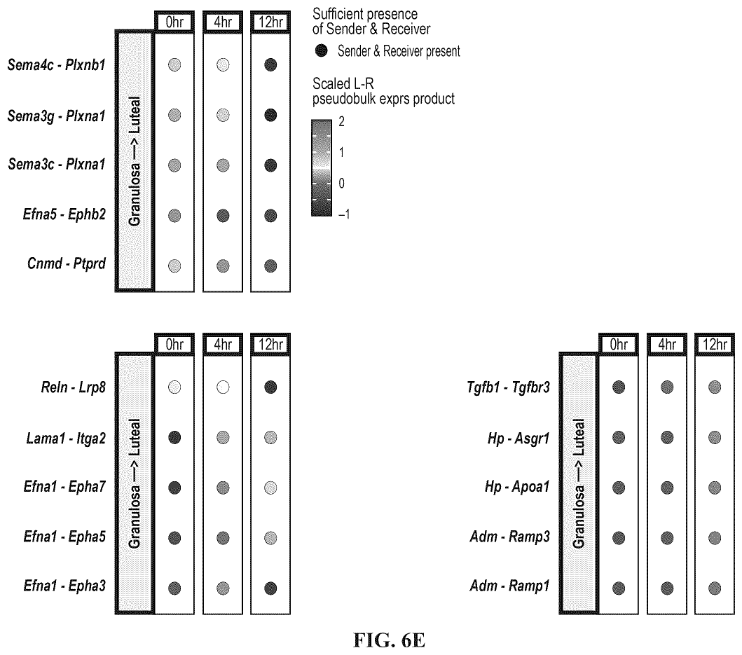

The relationships between pairs of ovarian cell types were further evaluated to determine their most enriched ligand-receptor interactions (FIG. 6C). Specifically, interactions between cumulus, granulosa, theca, stromal, and luteal cells were investigated. One known and one novel interaction was highlighted per each pair of cell types, which validated the analysis or revealed potential interactions driving known processes, respectively. Communication between granulosa and cumulus cells is critical for processes including granulosa cell differentiation and proliferation, cumulus cell layer expansion, oocyte growth and meiotic progression, and follicle rupture. As such, the communication between granulosa cells as the senders, and cumulus cells as the receivers, was analyzed throughout ovulation (FIG. 6C). At 4h post-hCG, interactions between bone metabolic proteins (BMPs) and their receptors were highly upregulated. In addition, hedgehog signaling between granulosa and cumulus cells (Ihh-Hhip) was also upregulated at this time point. Several studies have documented the role of hedgehog signaling in regulating COC expansion so, while not intending to be bound by theory, it is likely that this interaction modulates expansion of cumulus cells 4h post-hCG when expression of expansion-related genes peak. On the contrary, a previously undocumented interaction between granulosa (sender) and cumulus (receiver) cells identified was ANGPTL (ligand) and SDC4 (receptor) at 12h (FIG. 6C). Although ANGPTL signaling has not been documented between these two cell types, SDC4 itself is associated with COC expansion, and expression in the COC is positively associated with pregnancy rates. In addition, SDC4 is considered a “late response gene” as it is induced post-ovulation, consistent with the upregulation in ANGPTL-SDC4 signaling at 12h in the dataset.

Interactions were also assessed between theca cells (as senders) and luteal cells (as receivers) throughout the ovulation time course (FIG. 6D). At 0h post-hCG, there was an enriched interaction between laminin a4 (ligand) and integrin u9 (receptor). Laminins localize in theca cells to form the basement membrane across all follicle stages. Not intending to be bound by theory, although integrin u9 and its interaction with laminin being largely uncharacterized in the ovary, they may play a similar role in mature corpus lutea present at the 0hr time point from previous cycles. At the 4h time point, communication was observed between theca and luteal cells via BMP2 (ligand) and its receptors, including BMPR1B, BMPR2, and ACVR2A (FIG. 6D). Lastly, the interaction between NCAM1 (sender) and CACNAlC (receiver) was enriched at 12h post-hCG (FIG. 6D).

Finally, the communication between granulosa cells (as senders) and luteal cells (as receivers) was evaluated throughout the ovulation time course (FIG. 6E). Notably, the interaction between semaphorins (ligand) and plexins (receptors) was enriched at 0h. Sempahorins are extracellular cell guidance proteins that modulate cell migration through cytoskeletal reorganization, adhesion, and cell proliferation. The observed enrichment of semaphorin and plexin interactions at 0h suggested the communication between granulosa and luteal cells as part of tissue remodeling and maintenance of the mature corpus lutea from the previous cycle or luteolysis prior to ovulation. At the 4h timepoint, enriched interaction between Ephrin Al (ligand) and its respective ephrin (Eph) receptors (receivers) was observed (FIG. 6E). Direct cell-cell interaction between ephrin and Eph receptor pairs facilitates cell adhesion and migration leading to morphogenesis and angiogenesis. Other ephrins, such as Ephrin A5, modulate mouse granulosa cell morphology and adhesion as a critical factor in the LH-mediated ovulatory response. In addition, Ephrin B1 is present in regressing corpus lutea and upregulated in luteinizing granulosa cells in mouse and human ovaries during ovulation. Not intending to be bound by theory, given that the 4h primarily constitutes the general luteal cell subcluster (FIG. 5C), it suggests that Ephrin Al and its Eph receptor pairs may also be involved in both luteolysis and luteinization. At 12h, it was found that granulosa cells were highly enriched for two sender ligands, haptoglobin (HP) and adrenomedullin (ADM) (FIG. 6E). HP is a known inflammation biomarker that primarily functions to capture free plasma hemoglobin and prevent oxidative damage during hemolysis. Another enriched receiver for Hp is the asialoglycoprotein receptor 1 (ASGR1), which plays a critical role in glycoprotein homeostasis by mediating clearance from circulation. However, the relevance of this interaction and potential clearance of Hp during ovulation remains unclear. ADM, a vasodilator peptide hormone, is expressed in both rodent and human granulosa cells and corpus lutea. ADM interacts with receiver activity-modifying proteins (RAMPs) and calcitonin receptor-like receptors (CLR) to regulate cell proliferation, migration, angiogenesis, and sex hormone secretion. Not intending to be bound by theory, at the 12h time point, ADM-RAMPs may modulate vascular remodeling and steroidogenesis as part of the newly formed corpus luteum.

Ovulation is a highly coordinated spatiotemporal event that is fundamental to fertilization and endocrine function. To better understand the dynamic changes occurring during ovulation, the analyzes of the above Examples were conducted to prepare an integrated single cell RNA sequencing (scRNA-seq) and imaging spatial transcriptomics (iST) resource that fully maps this landscape. An integrated framework was developed that combines gene expression profiles from single-cell sequencing with spatial information to build a comprehensive map of ovarian cell types and their presence across various ovarian structures. With this resource, the transcriptional dynamics of ovulation was interrogated, novel gene markers for ovulation-dependent cell types were identified, new biological pathways that may contribute to normal ovarian function were revealed, and novel CCIs that may be important for orchestrating these processes were uncovered.

The integration of scRNA-seq and iST data is pivotal for advancing the understanding of complex biological processes, such as ovulation. While single-cell analysis enables the dissection of cellular heterogeneity and identification of molecular signatures within cell populations, it lacks the crucial spatial context necessary for comprehending the organization and interactions of cells within tissues. On the other hand, imaging spatial transcriptomics (iST) provides valuable information about the precise locations of gene expressions, offering insights into the spatial dynamics of tissues. However, iST relies on transcript signal mapping as well as cell segmentation pipelines to gain true single-cell resolution, which generates false positives and negatives. Moreover, the current iST technologies can only accommodate profiling up to a hundreds of genes. Therefore, by combining these two cutting-edge techniques, one may generate a more nuanced and detailed view of biological events. In the context of ovulation, this integration allows for the simultaneous examination of gene expression variations at the single-cell level and the spatial distribution of thousands of transcripts within the ovary. The experiments of the Examples of the present disclosure offer an ideal environment for data integration of single-cell and spatial transcriptomics, as contralateral ovaries were used to minimize differences in biological replicates between the two technologies.

The data revealed time-dependent subclusters of major ovarian cell types during the course of ovulation, including cumulus, theca, stroma, luteal, and granulosa cells. Investigations in the mouse ovary also identified time-dependent granulosa subclusters across the estrous cycle, such as the prevalence of luteinizing mural cells in the estrus phase (Morris et al., eLife, 11, e77239 (2022)). Notably, two time-varying cumulus cell subclusters were observed whereas previous studies found cumulus cells to be clustered together with granulosa cells from preantral follicles (Morris et al., eLife, 11, e77239 (2022)) or only one cumulus cell cluster across the ovulation time course (Morris et al., eLife, 11, e77239 (2022)). Not intending to be bound by theory, these differences may be driven by an increased number of cumulus cells present in the dataset, whether through the hyperstimulated mouse model versus ovulatory follicles naturally found during the estrous cycle or a higher number of ovaries per timepoint. Notably, the gene signature of the previously identified cumulus cell cluster aligned with the late cumulus cluster (Cumulus 2). As such, the identification of the early cumulus subcluster (Cumulus 1), which are uniquely enriched for neuronal pathways, may provide additional insight into its function and role in the preovulatory follicle. Additionally, time-varying subclusters in myeloid and epithelial cells were not identified, which aligns with previous studies evaluating the mouse estrous cycle.

Significant ligand-receptor interactions between several ovarian cell types were also identified across the ovulation time course. Between cumulus and granulosa cells, interactions between BMP2 and ACVRA as well as ANGPTL and SDC4 were observed at 4 h and 12h, respectively. Signaling via BMP2 and ACVR2A has been documented in the ovary, but the significance of the interaction in granulosa and cumulus cells is unclear. BMP2 is expressed in human granulosa cells, and expression of BMP2 in human cumulus cells is associated with improved quality of oocytes and resulting embryos, and successful fertilization. Expression of Acvr2a has also been documented in murine cumulus cells. In primary human granulosa-lutein cells, the interaction between BMP2 and ACVR2A negatively regulates the expression of PTX3, a key gene involved in cumulus cell layer expansion. On the contrary, incubation of primary human granulosa-lutein cells with BMP2 increases the expression of Has2, thus promoting the synthesis of hyaluronan, the main component of the COC ECM.

During ovulation, the communication between theca cells and luteal cells is critical for the former's transition during luteinization. Between these two cell types, significant interactions were observed between ECM components (laminin and integrin) at 0h, between BMP2 and its receptors (BMP1R1B, BMP2R, AVCRA) at 4h, and between NCAM1 and CACNA1C at 12h. Although laminins are known to be present in theca cells as part of the basement membrane, they are also present in the corpus luteum with the preference of certain subunits differing across species. Laminin a4 is present in the subendothelial basal lamina during the mid-, late, and regressing stages in humans whereas laminin α1, β1, and γ1 chains are prevalent in mice. Laminin-integrin interactions have been shown to facilitate granulosa cell proliferation, survival, and steroidogenesis. Notably, laminin enhances progesterone secretion through its interaction with integrin α6β1. In addition to its role in granulosa-cumulus cell interactions, BMP2 is also localized in theca cells in porcine and bovine antral follicles as well as in theca lutein cells of human corpus lutea. Similar to other BMPs, such as BMP4 and BMP6, BMP2 is suggested to be produced primarily by granulosa cells and attenuate androgen production in theca cells. Although the role of theca cell-induced BMP2 signaling on luteal cells is less clear, it may be involved in luteolysis. BMP2 expression is most prevalent in luteolytic corpus lutea in mice and humans. Administration of hCG also suppresses the expression of BMP2, BMPR1B, and BMPR2, suggesting its role as an inhibitor of luteinization and formation of the early corpus luteum. NCAM-CACNA1 binding has been suggested to localize to lipid rafts and facilitate Ca²⁺ intake for activation of calmodulin-dependent protein kinase IIα (CaMK11α) signaling. In rodents, NCAMs are present in theca cells of large antral follicles and luteal cells of forming and active corpus lutea; however, they are undetected in regressing corpus lutea. CACNAlC function is not well-documented in the ovary but shown to be primarily involved in smooth muscle contraction. Notably, smooth muscle cells are present in the theca external layer which constricts to increase intrafollicular pressure during the process of ovulation. This hypothesis is further supported by the enrichment of both ovulation pathways in theca cells (FIGS. 3E-1, 3E-2, 3E-3, and 3E-4) as well as calcium channel and smooth muscle contraction pathways in early corpus luteal cells (FIGS. 4E-1 and 4E-2) at 12 hours post-hCG.

Interactions between granulosa cells and luteal cells are essential for facilitating the development of new corpus lutea as well as degradation of preexisting ones as part of luteolysis. Interactions across the ovulation time course were observed (semaphorins-plexins at 0hr, Ephrin Al-Eph receptor at 4h, and HP-ASGR1 at 12h) that may play a role in substantial cell remodeling and steroidogenesis. Several semaphorins have been shown to be enriched in primordial follicles and preantral follicles to promote follicle activation and growth, respectively. In addition, SEMA4C and SEMA7A are implicated in actin cytoskeleton reorganization and steroidogenesis in granulosa cells as part of their role in ovarian tissue remodeling. Notably, while not intending to be bound by theory, the downregulation of Sema7a expression in response to ovulatory stimulation may contribute to the remodeling of the follicle structure for ovulation and the formation of the corpus luteum. Given follicle rupture and ovulation involve inflammatory processes, Hp may play a role in managing oxidative stress within the newly created corpus luteum. This premise is further supported by the observation that Hp interacts strongly with the high-density lipoprotein component apoprotein Al (Apo-A1). Apo-Al is a major component of high-density lipoprotein (HDL) that is present in the corpus luteum and facilitates cholesterol uptake for steroidogenesis. HP binding to Apo-Al has been shown to provide two functions: to protect the latter from hydroxyl radicals during oxidative stress and to retain high cholesterol levels in cells by inhibiting reverse cholesterol uptake back into HDLs.

In summary, the analyzes of the above Examples outline the dynamic spatiotemporal profile of mouse ovaries across the ovulation time course by combining single-cell resolution with spatial localization. Time-varying cell subclusters for major ovarian cell types were identified with enrichment of established and novel markers. Furthermore, cell-cell interaction analyses were conducted between ovarian cell types throughout ovulation, which revealed previously undescribed ligand-receptor interactions. This comprehensive dataset provides the framework to further investigate ovarian cell states during ovulation and may provide implications to better understand anovulatory conditions and drive discovery for new contraceptive targets for women.

The following materials and methods were employed in the above Examples.

### Animals

Female CD1 mice were purchased from Inotiv (West Lafayette, IN, USA) and used when reproductively adult (6-12 weeks). Mice were housed within a controlled barrier facility (Chicago, IL, USA) and kept at a constant temperature and humidity in a light cycle of 14h light and 10h dark. Mice were provided with food and water ad libitum and fed a specific chow that excludes soybean meal (Teklad Global 2916 chow, Envigo, Madison, WI).

### Optimization of Ovulation Timing for Workflow Compatibility

Physiological ovulation in mice typically occurs in the middle of the night. Therefore, it was determined whether the timing of ovulation in mice could be offset by 12h without affecting egg yield. Mice were hyperstimulated with pregnant mare serum gonadotropin (PMSG; ProSpec, #HOR-272) 12h apart to stimulate follicle growth. Mice were then injected with human chorionic gonadotropin (hCG; Sigma Aldrich, #C1063) 46h after relative PMSG injections to induce ovulation, and 14h post-hCG injection, ovulated COCs were collected from the oviduct. COCs were denuded of cumulus cells, and the number of MII eggs was compared between the control and offset groups (FIGS. 7A-7D). Similar egg numbers were collected across groups demonstrating that ovulation was not impacted by this shift in superovulation timing.

Generation of Single-Cell Suspensions from Mouse Ovaries

Ovulation induction was offset by 12h as described above. As detailed in FIG. 1A, mice received an intraperitoneal injection of 5 I.U. pregnant mare serum gonadotropin (PMSG) to stimulate follicle growth. A second intraperitoneal injection of 5 I.U. human chorionic gonadotropin (hCG) was given 46h following PMSG injection to induce ovulation. Ovary dissection occurred 0, 4, or 12h post hCG injection, with two independent operators dissecting ovaries from 3 mice each. One ovary from each mouse was pooled for single-cell isolation, with a total of three ovaries per suspension (labeled ABC or DEF). The contralateral ovary of each mouse was used for MERFISH analysis.

The pooled ovaries were cut into quarters using insulin syringe needles and enzymatically digested in 2 mL aMEM-Glutamax supplemented with 1 mg/mL bovine serum albumin (BSA; Sigma-Aldrich, #A3311) and 1x insulin-transferrin-selenium (Sigma-Aldrich, #1884) containing 40 μg/L liberase DH (Sigma-Aldrich, #05401089001), 0.4 mg/mL collagenase IV (Sigma-Aldrich, #C5138), and 0.2 mg/mL DNAse I (Sigma-Aldrich, #9003-98-9) for 15 minutes with gentle agitation at 37° C. and 5% C02 for 15 minutes. Ovaries were then mechanically digested via trituration using a 1000P wide bore tip, and the suspension was strained through a pre-wet 30 pm strainer directly into DMEM-GlutaMAX™ containing 10% FBS to quench the enzymes. Any remaining pieces of ovary were returned to the incubator in 2 mL fresh digestion media for another 15 minutes, followed by repeat mechanical digestion and straining. Once the enzymatic and mechanical digestions were complete, the cell suspension was centrifuged at 300g for 10 minutes at 4° C. The supernatant was removed, and the remaining cell pellet was resuspended in 100 μL Red Blood Cell Lysis solution (Miltenyi Biotec, #130-107-677) and incubated for 10 min at 4° C. After red blood cell removal, the suspension was centrifuged again at 300g for 10 minutes at 4° C. Following supernatant removal, the resulting pellet was resuspended in 100 μL of 0.025% BSA in phosphate-buffered saline without calcium or magnesium, and transferred to lo-bind Eppendorf tubes. The suspension was placed on ice and transferred immediately to the Northwestern University Sequencing Core (Chicago, IL, USA).

### Single Cell Library Preparation and Sequencing

Cell number and viability were analyzed using a fluorescent automated cell counter (Nexcelom Cellometer Auto2000) with a acridine orange/propidium iodide (AO/PI) fluorescent staining method. Sixteen thousand cells were loaded into a Chromium iX Controller (10X Genomics, PN-1000328) on a Chromium Next GEM Chip G (10X Genomics, PN-1000120), and processed to generate single-cell gel beads in the emulsion (GEM) according to the manufacturer's protocol. The cDNA and library were generated using the Chromium Next GEM Single Cell 3′ Reagent Kits v3.1 (10X Genomics, PN-1000286) and Dual Index Kit TT Set A (10X Genomics, PN-1000215) according to the manufacturer's manual. Quality control for the constructed library was performed by Agilent Bioanalyzer High Sensitivity DNA kit (Agilent Technologies, 5067-4626) and Qubit™ DNA HS assay kit for qualitative and quantitative analysis, respectively. The multiplexed libraries were pooled and sequenced on an Illumina Novaseq6000 sequencer with 100 cycle kits using the following read length: 28 bp Read1 for cell barcode and unique molecular identifier (UMI), and 90 bp Read2 for transcript.

Single-Cell RNA-Sequencing (scRNAseq) Analysis

Raw sequencing data, in base call format (.bcl) was demultiplexed using Cell Ranger from 10x Genomics, converting the raw data into FASTQ format. Cell Ranger was also used for the alignment of the FASTQ files to the reference genome and counting the number of reads from each cell that aligned to each gene. R version 4.2.2 and Seurat version 4 were used for all downstream analyses unless specified otherwise. For initial quality filtering, cells with greater than 20% mitochondrial gene expression (percent.mt) and less than 3000 expression counts (n_FeatureRNA) were removed. Standard Seurat pipelines were used to scale, find variables, and normalize the dataset. The identified list of variable genes was used to perform the principal component analysis. Cell clusters were identified with Find-Neighbors function with dims=1:20 and FindClusters function with resolution=0.5. Subclusters were found with iterative clustering with different resolutions of 0.5, 0.1, and 0.3. DoubleFinder_v3 was used to remove doublets with an approximate 5% expected ratio. MAST was used to perform all differential expression analyses. Cell-cell interaction analysis was performed with CellChat and Multinichenet packages.

### Gene Ontology Analysis

The online knowledgebase published by the Gene Ontology Consortium was utilized to perform GO analyses on each cell cluster. Gene IDs for the top 150 genes upregulated in each cluster were inputted into the GO Enrichment Analysis tool, selecting “biological process” and “Mus musculus” as search filters.

### Preparation of Ovaries and Microarray Assembly for Multiplexed Error-Robust Fluorescence In Situ Hybridization (MERFISH) Analysis

Ovaries intended for MERFISH analysis were collected in RNAse-sterile conditions as intact whole ovaries and stored in cryo-safe tubes and flash frozen in liquid nitrogen before tissue-microarray (TMA) construction and MERFISH processing. In RNAse-sterile conditions, samples were embedded and frozen in a pre-formed scaffold of Optimal Cutting Temperature media, oriented so that the ovarian hilum was at the base of the microarray and stored at −80° C. until sectioning. The ovaries were assembled in this tissue-microarray (TMA) as three whole ovaries per each timepoint (0h, 4h, and 12h) for a total of 9 ovaries. 10 pm-thick sections of the TMA were obtained using a cryostat at −20° C., mounted on fluorescent microsphere-coated, functionalized coverslips, fixed in 4% PFA in 1×PBS, and permeabilized in 70% ethanol overnight.

Following permeabilization in 70% ethanol, the TMA section was stained with Vizgen's Cell Boundary Stain Kit (PN 10400009). The section was washed briefly with 1×PBS before being incubated for one hour and room temperature in 100 μL of Cell Boundary Blocking Buffer Premix (PN 20300012) and 5 μL of murine RNase inhibitor, with a 2×2 cm square of parafilm over the sample to spread the mixture and prevent drying. The section was then incubated for another hour at room temperature in a mixture of 100 μL Cell Boundary Blocking Buffer, 5 μL RNase inhibitor, and 1 μL of Cell Boundary Primary Stain Mix (PN 20300010) with parafilm as described above. The section was washed three times with 5 ml 1X PBS on a rocker before a final 1-hour incubation at room temperature in 100 μL Cell Boundary Blocking Buffer Premix, 5 μL RNase inhibitor, and 3 μL Cell Boundary Secondary Stain Mix (PN 20300011) with parafilm as described above. The section was washed three times with 5 ml 1X PBS on a rocker at room temperature, then fixed again in 4 ml of 4% PFA in 1×PBS, followed by two 5 ml 1X PBS, all at room temperature. To hybridize the MERFISH probes to the section, the sample was first briefly washed in 2X saline-sodium citrate (SSC) at room temperature and incubated in 30% formamide in 2×SSC at 37° C. for 30 minutes. 50 μL of the probe mixture and 1 μL of RNase inhibitor were added on top of the sample and covered with paraflm as described above, and the section was incubated for 48 h at 37° C. After two 30 minute incubations at 37° C. in 30% formamide in 2×SSC, the sample embedded in a polyacrylamide gel solution (3.9 ml nuclease-free water, 0.5 ml 40% acrylamide/bis solution 19:1, 0.3 ml 5M NaCl, 0.3 ml Tris pH8, 25 μL 10% w/v ammonium persulfate in nuclease-free water, 2.5 μl of N,N,N′,N′-tetramethylethylenediamine). To embed the section, excess formamide was first removed with a 2-minute incubation of 2×SSC while the gel mixture was prepared. Once the gel was prepared, the sample was incubated in 5 ml of the gel mixture for one minute. Then the sample was transferred to a clean petri dish, the excess gel mixture was wicked away with a delicate task wiper (Kimwipe®), and 50 μL of reserved gel mixture was added on top of the section. A 20 mm glass coverslip that was cleaned and treated with 50 μL of GelSlick® solution was inverted on top of the sample, spreading out the gel evenly. The excess gel mixture was wicked away with a delicate task wiper (Kimwipe®), and the sample was left at room temperature for 2h while the gel was set. To clear excess proteins from the sample, the 20 mm coverslip was removed after the gel had completely set, and the sample was incubated in 5 ml of clearing mixture (3.4 ml nuclease-free water, 1 ml 10% sodium dodecyl sulfate (SDS), 0.5 ml 20×SSC, and 0.1 ml 25% Triton-X) for 3 days at 37° C.

### Selection of MERFISH Genes

A MERFISH panel of 205 genes consisting of marker genes, genes known to be involved in ovulation, and additional genes-of-interest were constructed based on published literature or preliminary data. Marker genes were chosen to facilitate the identification of cell types including granulosa, luteal, germ, mesenchymal, endothelial, epithelial, and immune cells.

Construction of MERFISH probes

A fluorescently tagged oligo probe library for 198 combinatorial genes and 3 sequential genes was designed, with each probe encoding the barcodes assigned to specific target RNA transcripts in the library. RNA targets were selected based on increasing the success of probe binding and ensuring gene expression fell within optically appropriate parameters for MERFISH imaging.

### MERFISH Imaging

The cleared sample was briefly washed three times with 5 ml of 2X saline-sodium citrate (SSC) wash buffer, stained with 3 ml of Vizgen DAPI and PolyT Staining Reagent (PN 20300021) for 10 minutes on a rocker, incubated in 30% formamide in 2×SSC for fifteen minutes, and then transferred to 2×SSC while the MERSCOPE® high-resolution, in situ spatial imaging platform combining single-cell and spatial multiomics analysis in an integrated system instrument was prepared. The MERSCOPE® flow chamber was cleaned with RNaseZap™ RNase Decontamination Solution and 70% ethanol. A Vizgen MERSCOPE® 300 Gene Imaging Cartridge (PN 20300017) was thawed and activated by adding 250 μL of Vizgen Imaging Buffer Activator (PN 20300022) and 100 μL of RNase inhibitor. 15 ml of mineral oil was added on top of the imaging solution in the cartridge to prevent oxidation. The MERSCOPE® instrument was initialized and primed, the section was loaded into the flow chamber, and the flow chamber was attached to the MERSCOPE® fluidics system and wetted, checking for bubbles before proceeding. A 10X overview was first acquired of the entire imageable area, and regions of interest were selected before moving to the 60X, which was cleaned and oiled before acquiring the MERFISH® images. Once imaging was complete, a cell boundary stain was selected for cell segmentation and image analysis was performed on the MERSCOPE using Vizgen's MERlin scalable and extensible MERFISH analysis software pipeline to acquire transcript count and cell segmentation data.

### MERFISH Analysis

Cell segmentation was performed on the MERSCOPE® high-resolution, in situ spatial imaging platform combining single-cell and spatial multiomics analysis in an integrated system, using Vizgen's provided pipeline, which utilize CellPose, a generalist algorithm for cellular segmentation, and MERlin scalable and extensible MERFISH analysis software to acquire cell and transcript data. Python™ version 3.7.12 was used to perform all analysis unless specified otherwise. Cells with less than 10 transcript counts were removed. Scanpy was used to find the variables, normalize, scale, perform principal component analysis (PCA), find neighborhoods, and cluster cells with the Leiden algorithm. To identify the cell type for each cluster, the number of transcripts were counted for each gene in every cluster and it was determined whether known markers occurred in the top 10. The differential expression analysis was used using Seurat v3 in R to find the markers for each Leiden cluster. Squidpy, a tool for the analysis and visualization of spatial molecular data, along with anndata, a Python™ package for handling annotated data matrices, and scanpy, a scalable toolkit for analyzing single-cell gene expression data, was used to visualize the regions spatially. Cell-cell interaction analysis was performed using CellPhoneDB, a repository of ligands, receptors, and their interactions.

MERFISH-10X Integration Analysis

Integration of MERFISH and 10X single-cell datasets was performed using the package Tangram. Out of the 198 genes in the MERFISH gene probes, 18 test genes were randomly selected to be left out of the training process, including Hmgcs2, Colla2, Ly6e, Krt18, Nr2f2, Oca2, Sultle1, Ccr7, Ptprc, Cd14, Wnt5a, Kitl, Pbk, Rasd1, Folr1, Rnd3, Mro, and Cldn5, to later assess the performance of the integration. The learning model used the leave-one-out validation strategy, where the remaining 180 genes were partitioned into 179 training genes and a single validation gene. The algorithm repeated the training 180 times, each time leaving out a different validation gene, to obtain a prediction for each gene. The overall performance of the analysis was evaluated in three ways: 1) Training and testing scores were obtained to quantify the deep learning model performance. 2) Picking out genes randomly from the test set, the expression from the MERFISH dataset was compared with the predicted expression of test genes that were originally in the MERFISH probes and were deliberately left out of the training process. 3) Picking out genes randomly from the result, RNAScope images were obtained on genes that were not originally in the MERFISH probes, which showed similar patterns with the integration results.

### Histological Processing and Staining

Immediately after collection, mouse ovaries were placed in tubes containing 1 mL Modified Davidson's solution (Electron Microscopy Services, Hatfield, PA) and rocked for 2-4h at room temperature. Ovaries were then stored overnight at 4° C. with gentle rocking. The next morning, ovaries were washed in 70% ethanol three times with 10 minutes per wash. Using standard processing protocols, an automated tissue processor (Leica Bioscystems. Buffalo Grove, IL) was used to process, dehydrate, and embed the ovaries in paraffin wax. Ovaries were then serially sectioned at 5-μm-thick intervals until approximately half of the ovary was sectioned, and sections were placed on glass slides. The slide containing the approximate midsection of the ovary was stained with hematoxylin and eosin using a standard hematoxylin and eosin (H&E) staining protocol. Stained sections were cleared using 3 5-minute-incubations with Citrisolv™ (Decon Laboratories Inc., King of Prussia, PA) and then mounted with Cytoseal™ XYL (ThermoFisher Scientific).

### RNA in Sito Hybridization

The expression of mouse Sultlel (ACD, #900181), Lox (ACD, #425311), Emb (ACD, #462011), Zfp804a (ACD, #1161171-C1), Trpv4 (ACD, #406071), Sik3 (ACD, #526431), and Slc6a6 (ACD, #544751) in whole ovary sections was detected using the RNAscope™ 2.5 HD RED Assay (Advanced Cell Diagnostics (ACD)). Samples were incubated with positive (Ppib; ACD, #313911) and negative (Dapb; ACD, #310043) control probes or target probes for 2 h at 40° C. and counterstained with hematoxylin and ammonia water. The EVOS® FL Auto imaging system was used to scan whole ovary sections at 20x resolution and image COCs at 40× magnification. The full, step-wise protocol for this assay can be found on the ACD website (acdbio.com/sites/default/files/322360-USM %20RNAscope %202.5%20HD %20RED %20Pt2_11052015.pdf)

### Resources

The following table provides a list of resources for materials, software, and algorithms used in the above Examples.

TABLE 11
Resources table

| REAGENT or RESOURCE | SOURCE | IDENTIFIER |
|---|---|---|
| Chemicals, peptides, and | | |
| recombinant proteins | | |
| Collagenase IV | Sigma-Aldrich | Cat# C5138 |
| Harris hematoxylin | EK Industries | Cat#4797 |
| Eosin Y | Thomas Scientific | Cat# |
| | | C310Q20 |
| Modified Davidson's | Electron Microscopy Services | Cat#64133-50 |
| Pregnant mare serum gonadotropin | ProSpec Bio | Cat#HOR-272 |
| Human chorionic gonadotropin | Sigma-Aldrich | Cat#C1063 |
| Dulbecco's phosphate-buffered | Fisher Scientific | Cat#14190-144 |
| saline, no calcium, no magnesium | | |
| Dulbecco's phosphate-buffered | Fisher Scientific | Cat#14040-133 |
| saline, calcium, magnesium | | |
| Minimum Essential Media α | Thermo Fisher Scientific | Cat#32561-037 |
| Glutamax ™, no nucleosides | | |
| Bovine serum albumin | Sigma-Aldrich | Cat#A3311 |
| 1x insulin-transferrin-selenium | Sigma-Aldrich | Cat#1884 |
| Liberase DH | Sigma-Aldrich | Cat#05401089001 |
| DNAse I | Sigma-Aldrich | Cat#9003-98-9 |
| Red blood cell lysis solution | Miltenyi Biotec | Cat##130-107-677 |
| Fetal bovine serum | Thermo Fisher Scientific | Cat#10082139 |
| Critical commercial assays | | |
| RNAscope 2.5 HD Reagent Kit- | ACD | Cat#322350 |
| RED | | |
| Chromium Next GEM Single Cell | 10X Genomics | Cat# PN-1000286 |
| 3′ Reagent Kits v3.1 | | |
| Dual Index Kit TT Set A | 10X Genomics | Cat# PN-1000215 |
| MERSCOPE Cell Boundary | Vizgen | Cat#PN-10400009 |
| Staining Kit | | |
| MERSCOPE 140 Gene Imaging | Vizgen | Cat#PN-10400004 |
| Kit, 1 sample | | |
| Experimental models: | | |
| Organisms and strains | | |
| Mouse: CD1 | Inotiv | Cat#030 |
| Oligonucleotides | | |
| RNAscope probe Sult1e1 | ACD | Cat#900181 |
| RNAscope probe Zfp804a | ACD | Cat#1161171-C1 |
| RNAscope probe Emb | ACD | Cat#462011 |
| RNAscope probe Lox | ACD | Cat#425311 |
| RNAscope probe Trpv4 | ACD | Cat#406071 |
| RNAscope probe Sik3 | ACD | Cat#526431 |
| RNAscope probe Slc6a6 | ACD | Cat#544751 |
| RNAscope Positive Control probe | ACD | Cat#313911 |
| PPib | | |
| RNAscope Negative Control probe | ACD | Cat#310043 |
| Dapb | | |
| Software and algorithms | | |
| Cellranger | 10x Genomics | support.10xgenomics.com/ |
| | | single-cell-gene- |
| | | expression/software/ |
| | | overview/welcome |
| Seurat | Hao et al., Cell, 184(13), 3573- | cran.r-project.org/ |
| | 3587.e29 | web/packages/Seurat/ |
| | | index.html |
| NicheNet | Browaeys et al., Nat Methods | github.com/saeyslab/ |
| | (2019) doi: 10.1038/s41592-019- | nichenetr |
| | 0667-5 | |
| Squidpy | Palla et al., Nature methods, 19(2), | github.com/scverse/ |
| | 171-178 | squidpy |
| Tangram | Biancalani et al., Nature methods, | github.com/ |
| | 18(11), 1352-1362 | broadinstitute/Tangram |
| MAST | Finak et al., Genome biology, 16, | github.com/RGLab/ |
| | 278 | MAST |
| DEseq2 | Love et al., Genome biology, | github.com/thelovelab/ |
| | 15(12), 550 | DESeq2 |
| Scanpy | Virshup et al., Nature | github.com/scverse/ |
| | biotechnology, 41(5), 604-606 | scanpy |
| Enrichr | Kuleshov et al., Nucleic acids | github.com/wjawaid/ |
| | research, 44(W1), W90-W97 | enrichR |
| CellChatDB | Jin et al., Nature communications, | www.cellchat.org/ |
| | 12(1), 1088 | cellchatdb |
| MultiNicheNet | Browaeys et al., Nat Methods | github.com/saeyslab/ |
| | (2019) doi: 10.1038/s41592-019- | multinichenetr |
| | 0667-5 | |

### OTHER EMBODIMENTS

From the foregoing description, it will be apparent that variations and modifications may be made to the aspects and embodiments described herein to adopt them to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for altering ovulation in a female subject, the method comprising administering to the subject an agent that selectively modulates the expression and/or activity in an ovary of the subject of a polypeptide encoded by a gene selected from the group consisting of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cypl7a1, Cypl9a1, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17b1, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, and Zfp804a, thereby altering ovulation in the female subject.

2. The method of claim 1, wherein the gene is selected from the group consisting of:
Tac1, Gsta4, Mast4, F3, Kcnd2, Timp1, Pik3c2g, Fdps, Lgals1, Ramp1, and Emb;
Tmsb4x, Timp1, Ybx1, Vim, Col4a1, Gas6, Pak3, Trib2, Smoc2, Kit, Tpm4, Fndc3b, Cnn3, and Zfp804a; and
Tmsb4x, Timp1, Ybx1, Mt2, Ctsl, Cst8, Bst2, Aebp1, Bace2, and Hmgcs1.

3. The method of claim 1, wherein the agent is a polypeptide, polynucleotide, small molecule, or is selected from those agents listed in Tables 2-7.

4. The method of claim 1, wherein the method reduces follicle activation or maturation in the ovary.

5. The method of claim 1, wherein the method selectively disrupts the development or function of a cell in an ovary of the subject, wherein the cell is selected from the group consisting of cumulus cells, endothelial cells, epithelial cells, granulosa cells, luteal cells, myeloid cells, oocyte cells, stroma cells, and theca cells, and the agent selectively modulates the expression and/or activity in an ovary of the subject of a polypeptide encoded by the gene.

6. The method of claim 1, wherein the agent selectively prevents proliferation of the cells.

7. The method of claim 1, wherein the agent selectively kills the cells.

8. The method of claim 1, wherein the agent selectively prevents development of the cells.

9. A method for altering fertility in a female subject, the method comprising administering to the subject an agent that selectively increases the expression and/or activity in an ovary of the subject of a polypeptide encoded by a gene selected from the group consisting of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cyp17al, Cyp19al, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17bl, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Nap115, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, and Zfp804a, thereby increasing fertility in the female subject.

10. The method of claim 9, wherein the gene is selected from the group consisting of:
Tac1, Gsta4, Mast4, F3, Kcnd2, Timp1, Pik3c2g, Fdps, Lgals1, Ramp1, and Emb;
Tmsb4x, Timp1, Ybx1, Vim, Col4a1, Gas6, Pak3, Trib2, Smoc2, Kit, Tpm4, Fndc3b, Cnn3, and Zfp804a; and
Tmsb4x, Timp1, Ybx1, Mt2, Ctsl, Cst8, Bst2, Aebp1, Bace2, and Hmgcs1.

11. The method of claim 9, wherein the agent is selected from those agents listed in Tables 2-7.

12. A method for reducing or eliminating ovulation in a female subject, the method comprising administering to the subject a non-hormonal agent that selectively modulates the expression and/or activity in an ovary of the subject of a polypeptide encoded by a gene selected from the group consisting of Acly, Acsbg1, Adamts1, Aebp1, Akrc1, Alcam, Aldhla1, Aldhla2, Areg, Bace2, Bgn, Bhmt, Birc5, Bst2, Btc, Cd52, Cd74, Cd93, Cdh5, Cdknla, Chchd10, Cldn5, Cnn3, Cobll1, Colla1, Colla2, Col3a1, Col4a1, Cst8, Ctla2a, Ctsl, Cyp17al, Cyp19al, Dcn, Edn2, Egfl7, Emb, Ereg, Esam, F3, Fam13a, Fcerlg, Fdps, Fdx1, Flt1, Fndc3b, Frmd5, Gas6, Gm10076, Gm2a, Gpm6a, Grem1, Gsta4, H2-Aa, H2-Ab1, Hao2, Hmgcs1, Hsd17bl, Hsd3b1, Ildr2, Kcnd2, Kdr, Kit, Krt18, Krt19, Krt7, Laptm5, Lgals1, Lgals7, Lhcgr, Lox, Lum, Lyz2, Mast4, Mgp, Mt2, Napl15, Nppc, Nts, Nupr1, Ogn, Onecut2, Pak3, Parm1, Pdgfra, Pecam1, Pgr, Pik3c2g, Plxna4, Ptgs2, Ptprc, Ptx3, Ramp1, Rnfl80, Rpl13a, Rplp1, Scarb1, Sdc1, Sfrp2, Slc18a2, Slc26a7, Smoc2, Sox5, Spp1, Spsb1, Star, Sultle1, Tac1, Tcf21, Timp1, Tpm4, Tmsb4x, Tnc, Tnfaip6, Tomll1, Top2a, Trib2, Tspo, Ube2c, Upk3b, Vim, Ybx1, and Zfp804a, and that selectively kills and/or reduces the development, proliferation, or metabolism of a cell in an ovary of the subject, wherein the cell is selected from the group consisting of cumulus cells, endothelial cells, epithelial cells, granulosa cells, luteal cells, myeloid cells, oocyte cells, stroma cells, and theca cells.

13. The method of claim 12, wherein the cells comprise cumulus cells.

14. The method of claim 12, wherein the cells comprise luteal cells, stroma cells, and/or thecal cells.

15. The method of claim 12, wherein the agent comprises a small molecule compound.

16. The method of claim 15, wherein the small molecule compound selectively increases proliferation and/or mediates development of the cells.

17. The method of claim 12, wherein the agent is selected from those agents listed in Tables 2-7.

18. The method of claim 12, wherein the agent comprises a polynucleotide.

19. The method of claim 18, wherein the method comprises administering to the subject a vector comprising the polynucleotide.

20. The method of claim 12, wherein the method increases follicle activation or maturation in the ovary.
